(19) United States
(12) Patent Application Publication
(10) Pub. No.: US 2023/0368862 A1
(43) Pub. Date: Nov. 16, 2023

(54) BINDING SITE IN TYPE 2 RYANODINE RECEPTOR

(71) Applicant: The Trustees of Columbia University in the City of New York, New york, NY (US)

(72) Inventor: Andrew R. MARKS, New York, NY (US)

(21) Appl. No.: 18/063,048

(22) Filed: Dec. 7, 2022

Related U.S. Application Data
(60) Provisional application No. 63/286,861, filed on Dec. 7, 2021.

(51) Int. Cl. G16B 15/30; C07K 14/705; G01N 33/68

(52) U.S. Cl. CPC G16B 15/30; C07K 14/705; G01N 33/6872; C07K 2299/00; G01N 2500/04

(57) ABSTRACT

The present disclosure relates to methods and compositions useful for the identification of a ryanodine receptor modulator binding site in ryanodine receptor type 2 (RyR2). The present disclosure also provides compositions useful for the analysis of the ryanodine receptor modulator binding site in RyR2 via cryo-EM. The present disclosure further provides computational methods for identifying compounds that bind to RyR2.

### CROSS REFERENCE

This application claims the benefit of U.S. Provisional Patent Application No. 63/286,861, filed on Dec. 7, 2021, the content of which is incorporated by reference herein in its entirety.

### STATEMENT AS TO FEDERALLY SPONSORED RESEARCH

This present disclosure was made with government support under R01HL1 R01HTL145473, R01DK118240, R01HTL142903, R01HTL140934, R01AR070194, R25HL156002R25, R25NS076445 and T32 HL120826, awarded by the National Institutes of Health (NIH). The government has certain rights in the disclosure.

### SEQUENCE LISTING

The instant application contains a Sequence Listing which has been submitted electronically in XML format and is hereby incorporated by reference in its entirety. Said XML copy, created on Dec. 7, 2022, is named 44010111US-PAT.xml and is 14,741 bytes in size.

### BACKGROUND

The ryanodine receptor (RyR) is required for excitation-contraction coupling. Although RyR is tightly regulated, inherited mutations and stress-induced post-translational modifications can result in a Ca²⁺ leak in skeletal myopathies, heart failure, and exercise-induced sudden death. Compounds known as Rycals® repair the leaky RyR and are effective in preventing and treating disease symptoms and restoring normal RyR function.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In some embodiments, the present disclosure provides a composition comprising a complex suspended in a solid medium, the solid medium comprising vitreous ice, wherein the complex comprises a protein and a synthetic compound, wherein the protein is a ryanodine receptor 2 protein (RyR2) or a mutant thereof.

In some embodiments, the synthetic compound is

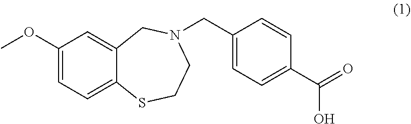 [SMILES: COc1ccc2c(c1)CN(Cc1ccc(C(=O)O)cc1)CCS2]

or an ionized form thereof.

In some embodiments, the protein is a mutant RyR2, for example a RyR2 protein containing at least one mutation that is associated with Catecholaminergic Polymorphic Ventricular Tachycardia (CPVT). In some embodiments, the RyR2 protein is post-translationally-modified RyR2 protein, for example a RyR2 protein containing at least one post-translational modification selected from phosphorylation, oxidation and nitrosylation. In some embodiments, the post-translationally modified RyR2 protein is associated with heart failure. In some embodiments, the RyR2 protein is a mutated and post-translationally modified RyR2 protein.

In some embodiments, the present disclosure provides a method for predicting a docked position of a target ligand in a binding site of a biomolecule, the method comprising:
receiving a template ligand-biomolecule structure, the template ligand-biomolecule structure comprising a template ligand docked in the binding site of the biomolecule;
comparing a pharmacophore model of the template ligand to a pharmacophore model of the target ligand;
overlapping the pharmacophore model of the target ligand with the pharmacophore model of the template ligand while the template ligand is in the binding site of the biomolecule; and
predicting the docked position of the target ligand in the binding site of the biomolecule based on a position of the pharmacophore model of the target ligand when overlapped with the pharmacophore model of the template ligand,
wherein the biomolecule is a RY1&2 domain of RyR2, wherein the template ligand-biomolecule structure is obtained by a process comprising subjecting a complex of the biomolecule and the template ligand to single-particle cryogenic electron microscopy analysis.

In some embodiments, the present disclosure provides a method of identifying a plurality of potential lead compounds, the method comprising the steps of:
(a) analyzing, using a computer system, an initial lead compound known to bind to a biomolecular target, the analyzing comprising partitioning, by providing a database of known reactions, the initial lead compound into atoms defining partitioned lead compound comprising a lead compound core and atoms defining a lead compound non-core, wherein the initial lead compound is partitioned using a computational retrosynthetic analysis of the initial lead compound;
(b) identifying, using the computer system, a plurality of alternative cores to replace the lead compound core in the initial lead compound, thereby generating a plurality of potential lead compounds each having a respective one of the plurality of alternative cores;
(c) calculating, using the computer system, a difference in binding free energy between the partitioned lead compound and each potential lead compound;
(d) predicting, using the computer system, whether each potential lead compound will bind to the biomolecular target and identifying a predicted active set of potential lead compounds based on the prediction;
(e) obtaining a synthesized set of at least some of the potential leads of the predicted active set to establish a first of potential lead compounds; and
(f) determining, empirically, an activity of each of the first set of synthesized potential lead compounds,
wherein the biomolecular target is a RY1&2 domain of RyR2, and the structure of the biomolecular target used in the predicting of (d) is obtained by a process comprising subjecting a complex of the biomolecular target and the initial lead compound to single-particle cryogenic electron microscopy analysis.

In some embodiments, the present disclosure provides a computer-implemented method of quantifying binding affinity between a ligand and a receptor molecule, the method comprising:
receiving by one or more computers, data representing a ligand molecule,
receiving by one or more computers, data representing a receptor molecule domain, using the data representing the ligand molecule and the data representing the receptor molecule domain in computer analysis to identify ring structure within the ligand, the ring structure being an entire ring or a fused ring;
using the data representative of the identified ligand ring structure to designate a first ring face and a second ring face opposite to the first ring face, and classifying the ring structure by:
a) determining proximity of receptor atoms to atoms on the first face of the ligand ring; and
b) determining proximity of receptor atoms to atoms on the second face of the ligand ring;
c) determining solvation of the first face of the ligand ring and solvation of the second face of the ligand ring;
classifying the identified ligand ring structure as buried, solvent exposed or having a single face exposed to solvent based on receptor atom proximity to and solvation of the first ring face and receptor atom proximity to and solvation of the second ring face;
quantifying the binding affinity between the ligand and the receptor molecule domain based at least in part on the classification of the ring structure; and
displaying, via computer, information related to the classification of the ring structure,
wherein the receptor molecule domain is a RY1&2 domain of RyR2, wherein the data representing a ligand molecule and the data representing a receptor molecule domain are obtained by a process comprising subjecting a complex comprising the ligand molecule and the receptor molecule domain to single-particle cryogenic electron microscopy analysis.

In some embodiments, the present disclosure provides a method comprising:
(a) determining an open probability (Pₒ) of a first RyR2 protein, wherein the first RyR2 protein is treated with a test compound, and
(b) determining an open probability (Pₒ) of a second RyR2 protein, wherein the second RyR2 protein is not treated with the test compound.

In some embodiments, the present disclosure provides a method comprising:
(a) contacting a first RyR2 protein with a test compound;
(b) providing a second RyR2 protein;
(c) subsequent to the contacting the first RyR2 protein with the test compound, measuring an open probability (Pₒ) of the first RyR2 protein; and
(d) measuring an open probability (Pₒ) of the second RyR2 protein.

In some embodiments, the present disclosure provides a method of identifying a compound having RyR2 modulatory activity, the method comprising:
(a) determining an open probability (Pₒ) of a RyR2 protein;
(b) contacting the RyR2 protein with a test compound;
(c) determining an open probability (Pₒ) of the RyR2 protein in the presence of the test compound; and
(d) determining a difference between the Pₒ of the RyR2 protein in the presence and absence of the test compound;
wherein a reduction in the Pₒ of the RyR2 protein in the presence of the test compound relative to the Pₒ of the RyR2 protein in the absence of the test compound is indicative of the compound having RyR2 modulatory activity.

In some embodiments, the present disclosure provides a method for identifying a compound having RyR2 modulatory activity, comprising:
(a) contacting a RyR2 protein with a ligand having known RyR2 modulatory activity to create a mixture, wherein the RyR2 protein is a leaky RyR2, the leaky RyR2 comprising mutant RyR2 protein, post-translationally modified RyR2, or a combination thereof;
(b) contacting the mixture of step (a) with a test compound; and
(c) determining the ability of the test compound to displace the ligand from the RyR2 protein.

In some embodiments, the present disclosure provides a method for identifying a compound that preferentially binds to a mutated, post-translationally modified RyR2 or a combination thereof, comprising:
(a) determining binding affinity of a test compound to a first RyR2 protein, wherein the first RyR2 protein is a wild-type RyR2 protein;
(b) determining binding affinity of a test compound to a second RyR2 protein, wherein second first RyR2 protein is a mutant RyR2 protein, a post-translationally modified RyR2, or a combination thereof; and
(c) selecting a compound having a higher binding affinity to the second RyR2 protein relative to the first RyR2 protein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts an immunoblot (left, top) and SDS-PAGE (left, middle) of human recombinant RyR2 expressed in HEK293 cells. Ratio of normalized intensities of the pS2808 and total RyR2 bands (right). FIG. 1B depicts an immunoblot (left, top) and SDS-PAGE (left, bottom) of dephosphorylated human recombinant RyR2 expressed in HEK293 cells.

FIGS. 2A-2J provide GSFSCs (top) and viewing angle distributions (middle) of the global nonuniform refinements performed in cryoSPARC before any local refinement for each structure, and FSC model-map performed in PHENIX (bottom) for DeP-RyR2-C (FIG. 2A), DeP-RyR2-O (FIG. 2B), P-RyR2-C (FIG. 2C), P-RyR2-O (FIG. 2D), P-RyR2+CaM-C (FIG. 2E), P-RyR2-R2474S-Pr, (FIG. 2F), P-RyR2-R2474S-O (FIG. 2G), P-RyR2-R2474S+Cpd1-C (FIG. 211), P-RyR2-R2474S+CaM-C (FIG. 2I), and P-RyR2-R2474S+CaM-O (FIG. 2J).

FIG. 3 depicts representative high-resolution details of cryo-EM maps showing the holes in proline and aromatic residues, precise side-chain conformations, and stabilized water molecules (blue arrows) for “primed” PKA RyR2-R2474S.

FIG. 4A and FIG. 4B depict local refinement cryo-EM maps colored by local resolution shown for “primed” PKA RyR2-R2474S.

FIGS. 5A-5K are flowcharts that summarize cryoSPARC processing of cryo-EM datasets to obtain final composite maps of DeP-RyR2 (FIG. 5A and FIG. 5B), P-RyR2 (FIG. 5C), P-RyR2+CaM (FIG. 5D and FIG. 5E), P-RyR2-R2474S (FIG. 5F and FIG. 5G), P-RyR2-R2474S+Cpd1 (FIG. 5H and FIG. 5I), and P-RyR2-R2474S+CaM (FIG. 5J and FIG. 5K).

FIGS. 6A-6C depict aligned atomic models of structures resolved by cryo-EM, focused on the pore and TM domains.

FIGS. 7A-7F show pore radii estimation calculated with HOLE for each structure resolved by cryo-EM.

FIG. 8A and FIG. 8B depict models of RyR2 with their respective cryo-EM maps centered on the ligand binding site of the closed (FIG. 8A) and open (FIG. 8B) state of representative RyR2 structures.

FIG. 9A shows overlapped models of open PKA-phosphorylated RyR2 (P-RyR2-0, PDB: 7U9R, yellow) and closed PKA-phosphorylated RyR2 (P-RyR2-C, PDB: 7U9Q, gray). FIG. 9B shows overlapped models of closed PKA-phosphorylated RyR2 (P-RyR2-C, PDB: 7U9Q, gray) and primed PKA-phosphorylated RyR2-R2474S (P-RyR2-R2474S-Pr, PDB: 7U9X, magenta). FIG. 9C shows overlapped models of primed PKA-phosphorylated RyR2-R2474S (P-RyR2-R2474S-Pr, PDB: 7U9X, magenta) and closed PKA-phosphorylated RyR2-R2474S+Compound 1 (P-RyR2-R2474S+Cpd1-C, PDB: 7UA1, cyan). FIG. 9D shows overlapped models of primed PKA-phosphorylated RyR2-R2474S (P-RyR2-R2474S-Pr, PDB: 7U9X, magenta) and closed PKA-phosphorylated RyR2-R2474S+CaM (P-RyR2-R2474S+CaM-C, PDB: 7UA3, cyan).

FIGS. 10A-10K depict pairwise comparisons of the cytosolic domains of all structures resolved by cryo-EM. Domains are labelled. Conformational changes are shown with arrows. The size of the arrows represents the amount of changes observed.

FIG. 11A depicts cryo-EM maps of closed PKA-phosphorylated RyR2 (gray) and primed PKA-phosphorylated RyR2-R2474S (magenta) from the side (left) and top (right) views. Conformation changes are shown with arrows. FIG. 11B depicts aligned models of closed PKA-phosphorylated RyR2 (PDB: 7U9Q, gray), open PKA-phosphorylated RyR2 (PDB: 7U9R, yellow), and primed PKA-phosphorylated RyR2-R2474S (PDB: 7U9X, magenta). FIG. 11C provides a close-up view of the region around residue 2474 of closed PKA-phosphorylated RyR2 (left) and primed PKA-phosphorylated RyR2-R2474S (right). Conformational changes are shown with arrows. Distances between closed PKA-phosphorylated RyR2 and primed PKA-phosphorylated RyR2-R2474S, and between closed and open PKA-phosphorylated RyR2 (in parentheses) are labeled.

FIG. 12A depicts cryo-EM maps of closed PKA-phosphorylated RyR2 (gray), closed PKA-phosphorylated RyR2-R2474S+Compound 1 (P-RyR2-R2474S+Cpd1-C, PDB: 7UA1, cyan), primed PKA-phosphorylated RyR2-R2474S (magenta) from the side (left) and top (right) views. Conformation changes are shown with arrows. FIG. 12B depicts aligned models of closed PKA-phosphorylated RyR2 (PDB: 7U9Q, gray), closed PKA-phosphorylated RyR2-R2474S+Compound 1 (P-RyR2-R2474S+Cpd1-C, PDB: 7UA1, cyan), open PKA-phosphorylated RyR2 (PDB: 7U9R, yellow), and primed PKA-phosphorylated RyR2-R2474S (PDB: 7U9X, magenta). FIG. 12C shows aligned models of closed PKA-phosphorylated RyR2 (P-RyR2-C, PDB: 7U9Q, gray), primed PKA-phosphorylated RyR2-R2474S (P-RyR2-R2474S-Pr, PDB: 7U9X, magenta), and closed PKA-phosphorylated RyR2-R2474S+Compound 1 (P-RyR2-R2474S+Cpd1-C, PDB: 7UA1, cyan). Conformational changes of the RYR1&2 and BSol domains are shown with arrows.

FIG. 13A depicts normalized differences in RMSD of primed PKA-phosphorylated RyR2-R2474S. FIG. 13B depicts normalized differences in RMSD of closed PKA-phosphorylated RyR2-R2474S+Cpd1.

FIG. 14A and FIG. 14B depict cryo-EM maps of local refinement cryoSPRAC jobs before 3D variability of “primed” PKA RyR2-R2474S (magenta), and closed PKA RyR2-R2474S+Compound 1 (cyan) from different views and map levels. FIG. 14C shows the closed PKA RyR2 model (PDB:7U9Q) with the aligned cryo-EM map centered on the RY1&2 domain from the top (top) and side (middle) views. FIG. 14D shows a close up of closed PKA R2474S RyR2+Compound 1 (P-RyR2-R2474S-C, PDB:7UA1) centered on the ryanodine receptor channel modulator binding site. FIG. 14E shows closed PKA RyR2-R2474S+Compound 1 (P-RyR2-R2474S-C, PDB:7UA1) centered on the BSol1-RY1&2 interface. Candidate residues involved in the BSol1-RY1&2 interaction are labeled. FIG. 14F depicts the same comparison provided in FIG. 14E but with distances between sidechains of candidate residues labeled in yellow.

FIG. 15, Panel A shows aligned cryo-EM maps of closed PKA-phosphorylated RyR2 (P-RyR2-C, gray) and closed PKA-phosphorylated RyR2+CaM (cyan). Panel B depicts JSol and CSol models of closed PKA-phosphorylated RyR2 (PDB: 7U9Q, gray) and open PKA-phosphorylated RyR2 (PDB: 7U9R, yellow). Panel C depicts JSol and CSol models of closed PKA-phosphorylated RyR2 (PDB: 7U9Q, gray) and closed PKA-phosphorylated RyR2+CaM (PDB: 7U9T, cyan). Panel D shows aligned cryo-EM maps of primed PKA-phosphorylated RyR2-R2474S (magenta) and closed PKA-phosphorylated RyR2-R2474S+CaM (cyan). Panel E depicts a model with cryo-EM map of closed PKA-phosphorylated RyR2-R2474S+CaM (PDB: 7UA3) centered on the BSol3 domain that is stabilized by CaM.

FIG. 16, Panel A illustrates a RMSD analysis of the closed PKA phosphorylated RyR2 (P-RyR2-C, PDB:7U9Q) vs. the closed (DeP-RyR2-C, PDB:7UA5) and open (DeP-RyR2-0, PDB:7UA9) states of dephosphorylated RyR2. Panel B depicts a 3D variability analysis of the RyR2 structures showing that the dynamic behavior of the RY3&4 domain is independent of the phosphorylation state, pore state, and mutation state. Panel C shows views of the “primed” PKA RyR2-R2474S model (P-RyR2-R2474S-Pr, PDB:7U9X) with the aligned cryo-EM map centered on the RY3&4 domain. Panel D shows the “primed” PKA RyR2-R2474S model (P-RyR2-R2474S-Pr, PDB:7U9X) centered on the interface between the RY3&4 and BSol1/SPRY3 domains. Panel E shows the atomic model of the RY3&4 domain of P-RyR2-R2474S-Pr centered on the poorly resolved phosphorylation loop. Panel F depicts cryo-EM maps of the particles in the closed state with destabilized RY3&4 domain (gray) and stabilized RY3&4 domain (magenta). Panel G depicts cryo-EM maps of the particles in the open state with destabilized RY3&4 domain (gray) and stabilized RY3&4 domain (magenta).

FIG. 17, Panel A and Panel B are cryo-EM maps of the closed particles with destabilized (gray) and stabilized (magenta) RY3&4 domain. A downward shift in surrounding domains was observed. Individual domains are labeled. Panel C and Panel D provide different points of view of the cryo-EM maps depicted in Panel A and Panel B, respectively.

FIG. 18 shows aligned models of the closed state of RyR2 with destabilized (gray) and stabilized (magenta) RY3&4 domain, and open state (yellow).

FIG. 19 is a chart illustrating an RMSD analysis of the closed state of RyR2 with stabilized RY3&4 domain.

FIG. 20A and FIG. 20B show a model with overlapped cryo-EM map of PKA-phosphorylated RyR2 (PDB: 7U9Q) highlighting intramembrane helices laterally positioned and encircling the TM domain from the side view (FIG. 20A) and bottom view (FIG. 20B).

FIG. 21A, shows sequence alignment of residues 4231-4320 between the secondary structure predicted by Jpred and the secondary structure from the cryo-EM-resolved model. FIG. 21B depicts the RyR2 model highlighting Sx helices. FIG. 21C depicts RyR2 model highlighting the interaction between Sx helices and the neighbor helical elements. FIG. 21D depicts RyR2 model highlighting a lysine rich linker. FIG. 21E depicts cryo-EM maps of closed PKA RyR2+CaM (gray), open PKA RyR2 (yellow), open PKA RyR2-R2474S (magenta), and open PKA RyR2-R2474S+CaM (cyan) centered on the Sx helices.

FIG. 22 shows single-channel current recordings traces from recombinant RyR2 at Ca²+150 nM, before (Panel A) and after (Panel B) the addition of xanthine 10 μM.

FIG. 23 depicts representative telemetric electrocardiogram (ECG) recordings of Ryr^{2R2474S/WT} mice (n=4) during arrhythmia provocation stress testing by epinephrine injection (1 mg/kg epinephrine).

FIG. 24 shows SR Ca²⁺ leak measured in microsomes from Ryr^{2R2474S/WT} mouse heart lysates. The Ca²⁺ leak was compared for hearts isolated from control Ryr^{2R2474S/WT} mice (gray), Ryr^{2R2474S/WT} mice treated with epinephrine (magenta), and Ryr^{2R2474S/WT} mice treated with epinephrine and Compound 1 (cyan).

FIG. 25 illustrates sequence coverage of RyR2 provided in the mass spectroscopic analysis of hyperphosphorylated channels described in EXAMPLE 10.

FIG. 26, Panels A-C illustrate the proposed mechanism of CPVT-related RyR2 variants, other gain-of-function mutants, and heart failure. Panel A is a schematic representation of the normal function of RyR2. Panel B is a schematic representation of the CPVT-related Ca²⁺ leak during diastole under intense exercise or stress conditions. Panel C is a schematic representation of the heart failure-related primed state and Ca²⁺ leak.

### DETAILED DESCRIPTION

Located on the sarco/endoplasmic reticulum (SR/ER) membrane, the ryanodine receptor (RyR) is the largest known ion channel, at over two megadaltons, and is the primary mediator of the Ca²⁺ release required for excitation-contraction coupling in cardiac and skeletal muscle. RyR is required for excitation-contraction coupling. RyR1 is the primary isoform in skeletal muscle while RyR2 is the predominant cardiac isoform. RyR1 and RyR2 are also found in neurons. RyR3 is present where RyR1 and RyR2 are each present, but with significantly lower expression levels. Beyond their expression pattern, RyR1 and RyR2 are unique in how each is activated. In skeletal muscle, RyR1 is activated by the direct, mechanical interaction with the dihydropyridine receptor (DHPR). RyR2 is instead activated by Ca²⁺ in the process termed calcium-induced calcium release (CICR) in which Ca²⁺ binding to RyR2 creates a cascade effect as the release of Ca²⁺ through the RyR creates a high local concentration of Ca²⁺, which can cause neighboring RyR channels to open. RyR, a tetramer, forms tetrads in muscle tissue and under normal conditions, undergoes cooperative activation through the process termed coupled gating.

The correct activation of RyR, and thus activation of the appropriate downstream Ca²⁺ signaling pathways, is regulated by multiple ligands and protein interactions. Aside from Ca²⁺, ATP, and caffeine, RyR also binds calmodulin (CaM). CaM is an inhibitor of ryanodine receptor type 2 (RyR2). CaM can act as either an activator of ryanodine receptor type 1 (RyR1) under low Ca²⁺ conditions (˜150 nM), such as those at rest, or an inhibitor of RyR1 under high Ca²⁺ conditions (>1 μM). High Ca²⁺ conditions occur locally following intracellular Ca²⁺ release. Calstabin, a second accessory protein, also binds the RyR. This interaction stabilizes the closed state of the channel. In disease states, RyR can be nitrosylated, oxidized and/or phosphorylated to cause calstabin to dissociate from the channel. This dissociation results in Ca²⁺ leaking into the cytosol and inappropriate triggering of downstream Ca²⁺ signaling pathways.

RyR comprises three major segments, each composed of several domains. The first, the cytosolic shell, consists of the N-terminal domain (NTD) with two segments (A & B) and an N-terminal solenoid, three SPRY domains, two RYR domains (RY1&2 and RY3&4), and the junctional and bridging solenoids (J-Sol and Br-Sol). The cytosolic shell also houses the calstabin binding site, which binds in a pocket formed by the Br-Sol and the SPRY domains, specifically SPRY1, and calmodulin, which binds on the other side of the Br-Sol from calstabin, with the N-terminal domain of CaM binding along the face of the Br-Sol while the C-terminal domain binds a peptide within a pocket of the Br-Sol.

Although RyR is tightly regulated, inherited mutations and stress-induced post-translational modifications (e.g., phosphorylation, nitrosylation and oxidation) can result in a Ca²⁺ leak. As a key player in Ca²⁺ signaling, leaky RyR channels are associated with a wide variety of disease states including skeletal muscle myopathies such as RyR-related myopathy (RYR-RM), dystrophies such as muscular dystrophy (e.g., Duchenne Muscular Dystrophy), cardiac diseases such as heart failure and catecholaminergic polymorphic ventricular tachycardia (CPVT), diabetes, and neurological disorders such as post-traumatic stress disorders (PTSD) and Alzheimer's disease.

Compounds known as ryanodine receptor modulators (also known as Rycals®) can repair leaky RyR and are effective in preventing and treating disease symptoms and restoring normal RyR function. Ryanodine receptor modulators can have efficacy in a host of diseases, both in vitro and in vivo using animal models. Ryanodine receptor modulators can repair the Ca²⁺ leak by preferentially binding to leaky RyR compared to normal RyR, and causing reassociation of calstabin, thus restabilizing the closed state of the channel. Mutations in RyR have been linked to rare genetic forms of cardiac and skeletal muscle disorders and ryanodine receptor modulators be effective in animal models in these disorders.

Given the structure of several ryanodine receptor modulator compounds, which contain aromatics and charged groups, ryanodine receptor modulators were initially hypothesized to bind near the caffeine binding site based on early cryo-electron microscopy (cryo-EM) structures with limited resolution. Advances in cryo-EM, and particularly direct detection cameras and novel processing methods including local refinement, have dramatically improved the resolution of cryo-EM maps, allowing unambiguous identification of ligand binding sites, including identification of a novel ATP binding site as described herein, and binding sites for Ca²⁺, and caffeine.

In some embodiments, the present disclosure utilizes cryo-EM techniques to generate a high resolution model of RyR2. In some embodiments, a high resolution model of RyR2 includes a ryanodine receptor modulator (e.g., Compound 1) bound to a ryanodine receptor modulator binding site in the RY1&2 domain of RyR2. In some embodiments, a ryanodine receptor modulator compound binds cooperatively with ATP and stabilizes the closed state of RyR2.

Ca²⁺, ATP, and xanthine are known to bind within the C-terminal domain (CTD) of RyR2. Disclosed herein is the identification of an additional ATP-binding site, in the periphery of the cytosolic shell of the RyR, in the RY1&2 domain that is comprised within the SPRY domain. In some embodiments, this region is also the ryanodine receptor modulator (Rycal) binding site. As demonstrated herein, ryanodine receptor modulator binding to RyR2 can increase in the presence of ATP. In some embodiments, Compound 1 binds in the RY1&2 domain cooperatively with ATP and stabilizes the closed state of the RyR2 channel despite the presence of activating ligands (Ca²⁺, ATP, and xanthine).

The present disclosure relates to methods and compositions useful for the identification of a binding site for ryanodine receptor modulators (Rycals) in ryanodine receptor type 2 (RyR2). The present disclosure also provides compositions useful for the analysis of the ryanodine receptor modulator binding site in RyR2 via cryo-EM. The present disclosure further provides methods (e.g., computational methods) for identifying compounds that bind to RyR2. The present disclosure further provides methods for screening for compounds that bind to RyR2 by utilizing a cryo-EM model of RyR2.

### Methods of Structural Determination.

Cryogenic electron microscopy (cryo-EM) is a cryomicroscopy technique applied on samples cooled to cryogenic temperatures and embedded in an environment of vitreous water. An aqueous sample solution is applied to a grid-mesh and plunge-frozen in a cryogenic fluid such as liquid ethane or a mixture of liquid ethane and propane.

The structures of the disclosure can be determined using cryo-EM with a sample frozen at a temperature of from about −40° C. to about −280° C. In some embodiments, the cryo-EM used for structural determination uses a sample frozen at a temperature of from about −40° C. to about −100° C., from about −100° C., to about −150° C., from about −150° C. to about −175° C., from about −175° C. to about −200° C., from about −200° C. to about −225° C., from about −225° C. to about −250° C., or from about −250° C. to about −280° C. In some embodiments, the cryo-EM used for structural determination uses a sample frozen at a temperature of from about −40° C. to about −100° C. In some embodiments, the cryo-EM used for structural determination uses a sample frozen at a temperature of from about −150° C. to about −175° C. In some embodiments, the cryo-EM used for structural determination uses a sample frozen at a temperature of from about −175° C. to about −200° C. In some embodiments, the cryo-EM used for structural determination uses a sample frozen at a temperature of from about −250° C. to about −280° C. In some embodiments, the cryo-EM used for structural determination uses a sample frozen at a temperature of about −150° C., about −175° C., about −200° C., about −250° C., or about −280° C. In some embodiments, the cryo-EM used for structural determination uses a sample frozen at a temperature of about −175° C. In some embodiments, the cryo-EM used for structural determination uses a sample frozen at a temperature of about −200° C. In some embodiments, the cryo-EM used for structural determination uses a sample frozen in liquid nitrogen. In some embodiments, the cryo-EM used for structural determination uses a sample frozen in liquid helium. In some embodiments, the cryo-EM used for structural determination uses a sample frozen in liquid ethane. In some embodiments, the cryo-EM used for structural determination uses a sample frozen in liquid propane. In some embodiments, the cryo-EM used for structural determination uses a sample frozen in mixture of liquid nitrogen and liquid propane.

The structures of the disclosure can be determined using a protein concentration of from about 50 nM to about 5 μM. In some embodiments, a structure of the disclosure can be determined using a protein concentration of from about 50 nM to about 250 nM, from about 250 nM to about 500 nM, from about 500 nM to about 750 nM, from about 750 nM to about 1 μM, or from about 1 μM to about 5 μM. In some embodiments, a structure of the disclosure can be determined using a protein concentration of from about 50 nM to about 250 nM. In some embodiments, a structure of the disclosure can be determined using a protein concentration of from about 250 nM to about 500 nM. In some embodiments, a structure of the disclosure can be determined using a protein concentration of from about 500 nM to about 750 nM. In some embodiments, a structure of the disclosure can be determined using a protein concentration of from about 750 nM to about 1 μM. In some embodiments, a structure of the disclosure can be determined using a protein concentration of from about 1 μM to about 5 μM.

The structures of the disclosure can be determined using a sample solution with a pH of about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In some embodiments, the sample solution has a pH of about 7.0. In some embodiments, the sample solution has a pH of about 7.1. In some embodiments, the sample solution has a pH of about 7.2. In some embodiments, the sample solution has a pH of about 7.3. In some embodiments, the sample solution has a pH of about 7.4. In some embodiments, the sample solution has a pH of about 7.5.

The structures of the disclosure (e.g., compositions comprising RyR2 and a ryanodine receptor modulator such as compound 1 bound to a ryanodine receptor modulator binding site on RyR2, and optionally an ATP molecule bound to an ATP binding site on the RyR2) can be determined at a resolution of from about 15 Å to about 2 Å. In some embodiments, the structures of the disclosure can be determined at a resolution of from about 15 Å to about 12 Å, from about 12 Å to about 9 Å, from about 9 Å to about 6 Å, from about 6 Å to about 5 Å, from about 5 Å to about 4 Å, from about 4 Å to about 3 Å, or from about 3 Å to about 2 Å. In some embodiments, the structures of the disclosure can be determined at a resolution of about 2.45 Å. In some embodiments, the structures of the disclosure is determined at a resolution of about 3.1 Å. In some embodiments, the structures of the disclosure is determined at a resolution from about 2 Å to about 3.5 Å, from about 2 Å to about 3.4 Å, from about 2 Å to about 3.3 Å, from about 2 Å to about 3.2 Å, from about 2 Å to about 3.1 Å, from about 2 Å to about 3 Å, from about 2 Å to about 2.9 Å, from about 2 Å to about 2.8 Å, from about 2 Å to about 2.7 Å, from about 2 Å to about 2.6 Å, from about 2 Å to about 2.5 Å, from about 2.1 Å to about 2.5 Å, from about 2.2 Å to about 2.5 Å, from about 2.3 Å to about 2.5 Å, or from about 2.4 Å to about 2.5 Å.

### Compositions Containing Complexes of RyR2.

In some embodiments, the present disclosure provides compositions useful for the determination of the ryanodine receptor modulator binding site in RyR2 via methods such as cryo-EM. In some embodiments, the present disclosure provides a composition comprising a complex suspended in a solid medium, wherein the complex comprises a biomolecule (e.g., a protein) and a synthetic compound, wherein the protein is a ryanodine receptor 2 protein (RyR2) or a mutant thereof.

In some embodiments, the present disclosure provides a composition comprising a complex suspended in a solid medium, the solid medium is or comprises a cryo-electron microscopy grid, wherein the complex comprises a biomolecule (e.g., a protein) and a synthetic compound, wherein the protein is a ryanodine receptor 2 protein (RyR2) or a mutant thereof.

In some embodiments, the present disclosure provides a composition comprising a complex suspended in a non-biological solid medium, wherein the complex comprises a biomolecule (e.g., a protein) and a synthetic compound, wherein the protein is a ryanodine receptor 2 protein (RyR2) or a mutant thereof, optionally with one or more proteins associated with RyR2.

In some embodiments, the composition is prepared by a process, the process comprising vitrifying an aqueous solution that is applied to an electron microscopy grid, wherein the aqueous solution comprises the protein and the synthetic compound.

An electron microscopy grid is a support structure used to insert specimens, for example, for use in an electron microscope. The grid structures can be flat with various suitable materials (e.g., copper, gold, rhodium, nickel, molybdenum, ceramic, etc.) for the grids themselves. In some cases, the grid structure can have plating (e.g., rhodium), coating (e.g., carbon, gold, plastic, silicon nitride, etc.), a suitable thickness (e.g., from 20 to 50 micron), and a suitable diameter (e.g., 3 mm). The grid structures generally have crossing bars and spacings/holes between the bars (e.g., nanometer to micrometer scale holes). The bars can come in various suitable sizes or pitch, patterns (e.g., regular or irregular), and shapes (e.g., numbers or letters built into the grid bars).

In some embodiments, prior to the vitrifying, the aqueous solution is applied to the electron microscopy grid, and excess aqueous solution is removed from the electron microscopy grid by blotting the excess aqueous solution.

In some embodiments, the aqueous solution is dispensed onto the electron microscopy grid from a dispensing apparatus located on the side of the electron microscopy grid opposed to the side abutting blotting material. Once the liquid sample is dispensed onto the cryo-EM grid, the blotting material can pull excess solution through the electron microscopy grid to produce a thin liquid film of the aqueous solution on the electron microscopy grid.

In some embodiments, the vitrifying comprises plunge freezing the aqueous solution applied to the electron microscopy grid into liquid ethane chilled with liquid nitrogen.

In some embodiments, the aqueous solution further comprises a buffering agent. Suitable buffering agents can include, for example, zwitterionic amines, such as TAPS ([tris(hydroxymethyl)methylamino]propanesulfonic acid), Bicine (2-(bis(2-hydroxyethyl)amino)acetic acid), Tris (2-amino-2-(hydroxymethyl)propane-1,3-diol), and Tricine (N-[tris(hydroxymethyl)methyl]glycine), as well as zwitterionic sulfonic acids, such as TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), MOPS (3-(N-morpholino)propanesulfonic acid), PIPES (piperazine-N,N′-bis(2-ethanesulfonic acid)), and MES (2-(N-morpholino)ethanesulfonic acid). In some embodiments, the buffering agent is HEPES. In some embodiments, the buffering agent is EGTA.

In some embodiments, the aqueous solution further comprises a phospholipid. In some embodiments, the phospholipid is a phosphatidylcholine, such as, for example, 1,2-dioleoyl-sn-glycero-3-phosphocholine (DOPC), 1,2-dilinoleoyl-sn-glycero-3-phosphocholine (DLPC), 1,2-dihexanoyl-sn-glycero-3-phosphocholine (DHPC), 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), or 1,2-distearoyl-sn-glycero-3-phosphocholine (DSPC). In some embodiments, the phospholipid is a phosphatidylserine, such as, for example, 1-palmitoyl-2-oleoyl-sn-glycero-3-phospho-L-serine (sodium salt) (POPS), or 1,2-dioleoyl-sn-glycero-3-phospho-L-serine (sodium salt) (DOPS). In some embodiments, the phospholipid is DOPS.

In some embodiments, the aqueous solution further comprises a surfactant. Surfactants can be used in a composition disclosed herein to increase the solubility of a protein (e.g. RyR2). In some embodiments, the surfactant is a zwitterionic surfactant, such as, for example, 3-[(3-cholamidopropyl)dimethylammonio]-1-propanesulfonate (CHAPS) or 3-([3-Cholamidopropyl]dimethylammonio)-2-hydroxy-1-propanesulfonate (CHAPSO). In some embodiments, the zwitterionic surfactant is CHAPS.

In some embodiments, the aqueous solution further comprises a disulfide-reducing agent, which can be, for example, tris (2-carboxyethyl) phosphine hydrochloride (TCEP), beta-mercaptoethanol (BME), tributylphosphine (TBP). or dithiothreitol (DTT). In some embodiments, the disulfide-reducing agent is TCEP.

In some embodiments, the aqueous solution further comprises a protease inhibitor. Suitable protease inhibitors can include, for example, 4-(2-aminoethyl)benzenesulfonyl fluoride (AEBSF), phenylmethylsulfonyl fluoride (PMSF), leupeptin, N-ethylmaleimide, antipain, pepstatin, alpha 2-macro-globulin, EDTA, bestatin, amastatin, and benzamidine. In some embodiments, the protease inhibitor is AEBSF. In some embodiments, the protease inhibitor is benzamidine hydrochloride.

In some embodiments, the aqueous solution further comprises xanthine. The concentration of xanthine in the aqueous solution can be, for example, about 1 mM to about 1000 μM, about 1 μM to about 900 μM, about 1 μM to about 800 μM, about 1 μM to about 700 μM, about 1 μM to about 600 μM, about 100 μM to about 1000 μM, about 100 μM to about 900 μM, about 100 μM to about 800 μM, about 100 μM to about 700 μM, about 100 μM to about 600 μM, about 200 μM to about 1000 μM, about 200 μM to about 900 μM, about 200 μM to about 800 μM, about 200 μM to about 700 μM, or about 200 μM to about 600 μM. In some embodiments, caffeine is present at a concentration of from about 200 μM to about 600 μM.

In some embodiments, xanthine is present at a concentration of about 100 μM, about 200 μM, about 300 μM, about 400 μM, about 500 μM, about 600 μM, about 700 μM, about 800 μM, about 900 μM, or about 1000 μM. In some embodiments, caffeine is present at a concentration of about 500 μM.

In some embodiments, the aqueous solution further comprises dissolved Ca²⁺. The concentration of dissolved Ca²⁺ in the aqueous solution can be, for example, about 1 μM to about 400 μM, about 1 μM to about 350 μM, about 1 μM to about 300 μM, about 1 μM to about 250 μM, about 1 μM to about 200 μM, about 50 μM to about 400 μM, about 50 μM to about 350 μM, about 50 μM to about 300 μM, about 50 μM to about 250 μM, or about 50 μM to about 200 μM. In some embodiments, dissolved Ca²⁺ is present at a concentration from about 50 μM to about 250 μM. In some embodiments, dissolved Ca²⁺ is present at a concentration from about 100 μM to about 200 μM.

In some embodiments, dissolved Ca²⁺ is present at a concentration of about 100 μM, about 110 μM, about 120 μM, about 130 μM, about 140 μM, about 150 μM, about 160 μM, about 170 μM, about 180 μM, about 190 μM, or about 200 μM. In some embodiments, dissolved Ca²⁺ is present at a concentration of about 150 μM.

The concentration of the protein in the aqueous solution can be, for example about 1 mg/mL to about 20 mg/mL, 2 mg/mL to about 20 mg/mL, 3 mg/mL to about 20 mg/mL, 4 mg/mL to about 20 mg/mL, 4 mg/mL to about 20 mg/mL, 1 mg/mL to about 15 mg/mL, 2 mg/mL to about 15 mg/mL, 3 mg/mL to about 15 mg/mL, 4 mg/mL to about 15 mg/mL, 1 mg/mL to about 10 mg/mL, 2 mg/mL to about 10 mg/mL, or 3 mg/mL to about 10 mg/mL. In some embodiments, the protein is present in the aqueous solution at a concentration from about 1 mg/mL to about 15 mg/mL. In some embodiments, the protein is present in the aqueous solution at a concentration from about 1 mg/mL to about 10 mg/mL. In some embodiments, the protein is present in the aqueous solution at a concentration from about 4 mg/mL to about 8 mg/mL.

In some embodiments, the protein is present in the aqueous solution at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some embodiments, the aqueous solution further comprises sodium adenosine triphosphate (NaATP). The concentration of NaATP in the aqueous solution can be, for example, about 1 mM to about 15 mM, about 1 mM to about 50 mM, about 1 mM to about 30 mM, about 1 mM to about 30 mM, about 2 mM to about 30 mM, about 3 mM to about 30 mM, about 4 mM to about 30 mM, about 5 mM to about 30 mM, about 6 mM to about 30 mM, about 7 mM to about 30 mM, about 8 mM to about 30 mM, about 9 mM to about 30 mM, about 10 mM to about 30 mM, 1 mM to about 15 mM, about 2 mM to about 15 mM, about 3 mM to about 15 mM, about 4 mM to about 15 mM, or about 5 mM to about 15 mM. In some embodiments, NaATP is present in the aqueous solution at a concentration from about 3 mM to about 15 nM.

In some embodiments, NaATP is present in the aqueous solution at a concentration of about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, or about 50 mM. In some embodiments, the concentration of NaATP is about 10 mM.

In some embodiments, the aqueous solution further comprises cAMP (cyclic adenosine monophosphate). The concentration of cAMP in the aqueous solution can be, for example, about 1 μM to about 400 μM, about 1 μM to about 350 μM, about 1 μM to about 300 μM, about 1 μM to about 250 μM, about 1 μM to about 200 μM, about 50 μM to about 400 μM, about 50 μM to about 350 μM, about 50 μM to about 300 μM, or about 50 μM to about 250 μM. In some embodiments, cAMP is present the aqueous solution at a concentration from about 100 μM to about 300 μM. In some embodiments, cAMP is present at the aqueous solution a concentration from about 150 μM to about 250 μM.

In some embodiments, the aqueous solution is substantially free of cellular membrane. Prior to adding protein to the solution, the protein can be separated from cellular membranes by homogenization of cells containing the protein, and subjecting the resulting homogenate to chromatography.

In some embodiments, the aqueous solution further comprises calmodulin. In some embodiments, the calmodulin is human calmodulin. In some embodiments, the calmodulin has a sequence according to SEQ ID NO: 1.

The aqueous solution can be prepared or stored in a vessel. In some embodiments, the vessel is a vial, ampule, test tube, or microwell plate.

In some embodiments, the complex further comprises a nucleoside-containing molecule. In some embodiments, the nucleoside-containing molecule is a purine nucleoside-containing molecule. In some embodiments, the nucleoside-containing molecule is a nucleotide or nucleoside polyphosphate. In some embodiments, the nucleoside-containing molecule is an adenosine triphosphate (ATP) molecule.

In some embodiments, the nucleoside-containing molecule and the synthetic compound bind a RYR domain of the protein. In some embodiments, the RYR domain is a RY1&2 domain. In some embodiments, the RY1&2 domain has a three-dimensional structure according to TABLE 3. In some embodiments, the synthetic compound has a three-dimensional conformation according to TABLE 4. In some embodiments, the ATP molecule has a three-dimensional conformation according to TABLE 5. In some embodiments, the ATP molecule binds the protein and the synthetic compound. In some embodiments, the synthetic compound binds cooperatively with the ATP molecule in the RY 1&2 domain of RyR2. In some embodiments, the synthetic compound is a ryanodine receptor modulator, e.g., Compound 1.

In some embodiments, the complex further comprises a second ATP molecule, wherein both ATP molecules bind a common RYR domain of the protein.

In some embodiments, the complex further comprises a second binding site for a nucleoside-containing molecule. In some embodiments, the complex further comprises a second nucleoside-containing molecule. In some embodiments, the second nucleoside-containing molecule binds a C-terminal domain of the RyR2 protein. In some embodiments, the second nucleoside-containing molecule is a nucleotide or nucleoside polyphosphate. In some embodiments, the second nucleoside-containing molecule is a second ATP molecule.

In some embodiments, the complex further comprises calmodulin. In some embodiments, the calmodulin is human calmodulin.

In some embodiments, the complex further comprises calstabin (i.e., peptidyl-prolyl cis-trans isomerase FKBP1B). In some embodiments, the calstabin is human calstabin. In some embodiments, the calstabin has a sequence according to SEQ ID NO: 2.

In some embodiments, the RyR2 protein is in a resting (closed) state. In some embodiments, the RyR2 protein is in the primed state. In some embodiments, a primed state comprises a higher distribution of open probability (Pₒ) as compared to a RyR2 in a resting (closed) state. In some embodiments, a primed state RyR2 comprises about 30% to about 60% of the RyR channel in an open state. In some embodiments, a primed state RyR2 comprises about 30%, about 35%, about 40%, about 45%, about 50%, about 55% or about 60% of the RyR channel in an open state.

In some embodiments, the complex further comprises a xanthine alkaloid molecule. In some embodiments, the complex further comprises a xanthine molecule, such as, for example, theobromine, theophylline, caffeine, or xanthine. In some embodiments, the complex further comprises a Ca²⁺ ion.

In some embodiments, the solid medium comprises vitreous ice. In some embodiments, the solid medium is substantially free of crystalline ice.

In some embodiments, the composition is substantially free of cellular membrane. In some embodiments, the RyR2 is a purified RyR2. In some embodiments, the RyR2 is a semi-purified RyR2 that is substantially free of cellular membrane.

In some embodiments, the composition further comprises additional complexes, wherein each of the additional complexes independently comprises the protein and the synthetic compound. In some embodiments, at least about 50%, at least about 60%, at least about 70%, at least about 80%, or at least about 90% of the RyR2 protein in the additional complexes is in a closed state. In some embodiments, at least about 50%, at least about 60%, at least about 70%, at least about 80%, or at least about 90% of the additional complexes is in an open state.

In some embodiments, the RyR2 protein is in a phosphorylated state, wherein the phosphorylated state is prepared by a process comprising contacting RyR2 protein with a phosphorylation reagent. In some embodiments, the phosphorylation reagent comprises protein kinase A. In some embodiments, the phosphorylation reagent further comprises ATP. In some embodiments, the phosphorylation reagent further comprises MgCl₂.

In some embodiments, the protein is in a dephosphorylated state, wherein the dephosphorylated state is prepared by a process comprising contacting RyR2 protein with a dephosphorylation reagent. In some embodiments, the dephosphorylation reagent comprises phosphatase lambda. In some embodiments, the dephosphorylation reagent further comprises MnCl₂.

In some embodiments, the synthetic compound binds a RYR domain of the protein. In some embodiments, the RYR domain is a RY1&2 domain.

In some embodiments, the protein is wild type RyR2. In some embodiments, the protein is mutant RyR2. In some embodiments, the mutant RyR2 is R2474S RyR2. In some embodiments, the protein is human RyR2. In some embodiments, the protein is a tetramer of RyR2 monomers, wherein each RyR2 monomer is SEQ ID NO: 3. In some embodiments, the protein is a tetramer of RyR2 monomers, wherein each RyR2 monomer is SEQ ID NO: 4. In some embodiments, the RyR2 protein is C4-symmetrical. In some embodiments, the protein comprises four RY1&2 domains, each with a three-dimensional conformation according to TABLE 3.

In some embodiments, the RyR2 protein is in a closed state. In some embodiments, the RyR2 protein is in an open state. In some embodiments, the RyR2 protein is in a primed state, wherein the RyR2 in the primed state has an open probability (Pₒ) that is higher an open probability (Pₒ) of the RyR2 in a closed state, and an open probability (Pₒ) that is lower than an open probability (Pₒ) of the RyR2 protein in an open state. In some embodiments, the protein is wild type RyR2. In some embodiments, the protein is a mutant RyR2. In some embodiments, the mutant RyR2 contains at least one mutation that is associated with Catecholaminergic Polymorphic Ventricular Tachycardia (CPVT). In some embodiments, the mutation is R2474S. In some embodiments, the mutation is RyR2-R420Q. In some embodiments, the mutation is RyR2-R420W.

In some embodiments, the protein is a post-translationally modified RyR2 protein. In some embodiments, the post-translationally modified RyR2 protein is a phosphorylated RyR2 protein. In some embodiments, the post-translationally modified RyR2 is an oxidized RyR2 protein. In some embodiments, the post-translationally modified RyR2 is a nitrosylated RyR2. In some embodiments, the post-translationally modified RyR2 protein is associated with a cardiac disease. In some embodiments, the post-translationally modified RyR2 protein is associated with heart failure. In some embodiments, the post-translationally modified RyR2 protein is associated with a cardiac arrhythmia. In some embodiments, the RyR2 is a mutated and post-translationally modified RyR2

In some embodiments, the mutation destabilizes an interaction between NTD and BSol domains of the RyR2 protein. In some embodiments, the mutation destabilizes a cytosolic shell of the RyR2 protein, wherein the cytosolic shell comprises NTD, SPRY, JSol and BSol domains of the RyR2 proteins.

The relative difference in conformational states between a structure (e.g., a protein structure or protein domain structure) and a reference structure (e.g., a reference protein structure or a reference protein domain structure) can be quantified by calculating the average distance between atoms of the structure and the reference structure when the structure and reference structure are superimposed. A nonlimiting example of a measure of a difference in conformational states between a structure and a reference structure is root mean square deviation of atomic positions (RMSD), which can be defined as:

$$RMSD = \sqrt{\frac{1}{N} {\sum }_{i = 1}^{N} {δ}_{i}^{2}}$$

where δᵢ is the distance between atom i and an analogous atom of a reference structure, or the mean position of the N equivalent atoms.

Root mean square deviation of atomic positions (RMSD) of a protein or protein domain structure relative to a reference protein or protein domain structure can be calculated computationally via software such as, for example, UCSF ChimeraX, SuperPose. LGA (Local-Global Alignment), or PDBeFold. In some embodiments, RMSD is calculated on the basis of Ca atomic coordinates of the structure and reference structure.

In some embodiments, the present disclosure provides a composition comprising a complex suspended in a solid medium, wherein the complex comprises a protein. In some embodiments, the present disclosure provides a composition comprising a complex suspended in vitreous ice, wherein the complex comprises a protein. In some embodiments, the protein is a ryanodine receptor protein or a mutant thereof. In some embodiments, the protein is a ryanodine receptor 2 protein (RyR2) or a mutant thereof. The composition can be, for example, a composition suitable for analysis via single-particle cryogenic electron microscopy, where the solid medium is an amorphous material such as vitreous ice.

In some embodiments, the composition is analyzed in a study, wherein the study comprises: (i) determining a structure of the protein (e.g., RyR2 or a mutant thereof) or a domain of the protein by subjecting the complex to single particle cryogenic electron microscopy analysis; and (ii) calculating RMSD of the protein or domain of the protein relative to a reference structure. In some embodiments, the protein is a RyR2 mutant (e.g, R2474S RyR2, RyR2-R420Q, or RyR2-R420W), and the reference structure is a structure of a wild type RyR2 protein. In some embodiments, the complex further comprises a synthetic compound (e.g., a ryanodine receptor channel modulator).

In some embodiments, the reference structure is obtained by a process comprising analyzing a reference composition via single particle cryogenic electron microscopy, wherein the reference composition comprises a reference complex suspended in a solid medium, wherein the reference complex comprises a reference protein. In some embodiments, the reference composition is prepared by a process comprising vitrifying a reference aqueous solution applied to an electron microscopy grid, wherein the reference aqueous solution comprises the reference complex. The reference aqueous solution can be identical to the aqueous solution used to prepare the composition, except that (a) the reference aqueous solution comprises the reference protein in place of the protein, and/or (b) the aqueous solution comprises a synthetic compound, the reference aqueous solution comprises calmodulin, the reference aqueous solution does not comprise the synthetic compound, and the aqueous solution does not comprise calmodulin. In some embodiments, the reference structure is a structure according to Protein Data Bank entry 7U9Q.

In some embodiments, the protein is R2474S RyR2, wherein if a study is conducted, the study comprising:
(i) determining a structure of a BSol2 domain of the protein in the complex by subjecting the complex to single particle cryogenic electron microscopy analysis, and
(ii) calculating root mean square deviation of atomic positions (RMSD) of the BSol2 domain of the protein relative to a BSol2 domain of a reference structure, wherein the reference structure is a structure of a wild type RyR2 protein in a closed state,
then the RMSD is no more than about 4.5, no more than about 4, no more than about 3.5, or no more than about 3.

In some embodiments, the protein is R2474S RyR2, wherein if a study is conducted, the study comprising:
(i) determining a structure of a BSol2 domain of the protein in the complex by subjecting the complex to single particle cryogenic electron microscopy analysis, and
(ii) calculating root mean square deviation of atomic positions (RMSD) of the BSol2 domain of the protein relative to a BSol2 domain of a reference structure, wherein the reference structure is a structure a wild type RyR2 protein in a closed state,
then the RMSD is from about 1 to about 4.5, about 1 to about 4, about 1 to about 3.5, about 1 to about 3, about 2 to 4.5, about 2 to about 4, about 2 to about 3.5, or about 2 to about 3.

In some embodiments, the protein is R2474S RyR2, wherein if a study is conducted, the study comprising:
(i) determining a structure of a BSol domain of the protein in the complex by subjecting the complex to single particle cryogenic electron microscopy analysis, and
(ii) calculating root mean square deviation of atomic positions (RMSD) of the BSol domain of the protein relative to a BSol domain of a reference structure, wherein the reference structure is a structure a wild type RyR2 protein in a closed state,
then the RMSD is no more than about 3, no more than about 2.5, no more than about 2, or no more than about 1.5.

In some embodiments, the protein is R2474S RyR2, wherein if a study is conducted, the study comprising:
(i) determining a structure of a BSol domain of the protein in the complex by subjecting the complex to single particle cryogenic electron microscopy analysis, and
(ii) calculating root mean square deviation of atomic positions (RMSD) of the BSol domain of the protein relative to a BSol domain of a reference structure, wherein the reference structure is a structure a wild type RyR2 protein in a closed state,
then the RMSD is from about 0.5 to about 3, about 0.5 to about 2.5, about 0.5 to about 2, about 0.5 to about 1.5, about 1 to 3, about 1 to about 2.5, about 1 to about 2, or about 1 to about 1.5.

In some embodiments, the protein is R2474S RyR2, wherein if a study is conducted, the study comprising:
(i) determining a structure of a NTD domain of the protein in the complex by subjecting the complex to single particle cryogenic electron microscopy analysis, and
(ii) calculating root mean square deviation of atomic positions (RMSD) of the NTD domain of the protein relative to a NTD domain of a reference structure, wherein the reference structure is a structure a wild type RyR2 protein in a closed state,
then the RMSD is no more than about 1.5, no more than about 1.4, no more than about 1.3, no more than about 1.2, no more than about 1.1, or no more than about 1.

In some embodiments, the protein is R2474S RyR2, wherein if a study is conducted, the study comprising:
(i) determining a structure of a NTD domain of the protein in the complex by subjecting the complex to single particle cryogenic electron microscopy analysis, and
(ii) calculating root mean square deviation of atomic positions (RMSD) of the NTD domain of the protein relative to a NTD domain of a reference structure, wherein the reference structure is a structure a wild type RyR2 protein in a closed state,
then the RMSD is from about 0.5 to about 1.6, about 0.5 to about 1.5, about 0.5 to about 1.4, about 0.5 to about 1.3, or about 0.5 to about 1.2.

In some embodiments, the protein is R2474S RyR2, wherein if a study is conducted, the study comprising:
(i) determining a structure of a SPRY domain of the protein in the complex by subjecting the complex to single particle cryogenic electron microscopy analysis, and
(ii) calculating root mean square deviation of atomic positions (RMSD) of the SPRY domain of the protein relative to a SPRY domain of a reference structure, wherein the reference structure is a structure a wild type RyR2 protein in a closed state,
then the RMSD is less than about 1.2, less than about 1.1, or less than about 1, less than about 0.9, less than about 0.8, or less than about 0.7.

In some embodiments, the protein is R2474S RyR2, wherein if a study is conducted, the study comprising:
(i) determining a structure of a SPRY domain of the protein in the complex by subjecting the complex to single particle cryogenic electron microscopy analysis, and
(ii) calculating root mean square deviation of atomic positions (RMSD) of the SPRY domain of the protein relative to a SPRY domain of a reference structure, wherein the reference structure is a structure a wild type RyR2 protein in a closed state,
   then the RMSD is from about 0.2 to about 1.3, about 0.2 to about 1.2, about 0.2 to about 1.1, about 0.2 to about 1, or about 0.2 to about 0.9.

In some embodiments, the reference protein is the wild type RyR2 protein. In some embodiments, the protein and the reference protein are each independently in a phosphorylated state. In some embodiments, the phosphorylated state of the protein or the reference protein is prepared by a process comprising contacting RyR2 protein or a mutant thereof with a phosphorylation reagent. In some embodiments, the phosphorylation reagent is protein kinase A. In some embodiments, the protein and the reference protein are each independently in a dephosphorylated state. In some embodiments, the dephosphorylated state of the protein or the reference protein is prepared by a process comprising contacting RyR2 protein or a mutant thereof with a dephosphorylation reagent. In some embodiments, the dephosphorylation reagent comprises phosphatase lambda.

In some embodiments, the protein and the reference protein are each independently in an oxidized state. In some embodiments, the protein and the reference protein are each independently in a nitrosylated state.

### Compounds of the Disclosure.

The synthetic compound in the compositions described herein can be a ryanodine receptor modulator compound, such as a benzothiazepane derivative. Some benzothiazepine compounds are voltage-gated Ca²⁺ channel blockers, but ryanodine receptor modulator compounds can be free of any channel blocking activity. The inability of certain ryanodine receptor modulator compounds to block Ca²⁺ channels can be associated with the mechanism of stabilizing the closed state of the RyR without inhibiting the channel. In some embodiments, a ryanodine receptor modulator compounds are modulators of the RyR channel. In some embodiments, ryanodine receptor modulator compounds are allosteric modulators of the RyR channel.

Ryanodine receptor modulator compounds of the disclosure can be used as therapeutics because in some disease states, RyR leaks Ca²⁺ due to destabilization of the closed state of the channel after post-translational modifications such as nitrosylation, oxidation and phosphorylation. In other disease states, Ca²⁺ leak is present due to inherited mutations. The genetic mutations can predispose the RyR channel to post-translational modifications such as oxidation and nitrosylation, further exacerbating the leak. These mutations and post-translational modifications cause the stabilizing subunit, calstabin, to dissociate from the channel, increasing the open probability of the channel, resulting in Ca²⁺ leak. In disease models involving leaky RyR in cells, animals, and patients, treatment with a ryanodine receptor modulator compound can reverse the leak and restore calstabin binding.

In some embodiments, the synthetic compound comprises a benzazepane or benzothiazepane (e.g., 2,3,4,5-tetrahydro-1,4-benzothiazepine) moiety. In some embodiments, the synthetic compound comprises a benzothiazepane moiety. In some embodiments, the synthetic compound comprises a benzothiazepine moiety. In some embodiments, the synthetic compound comprises a 1,4-benzothiazepine moiety.

### Chemical Groups.

The term “alkyl” as used herein refers to a linear or branched, saturated hydrocarbon having from 1 to 6 carbon atoms. Representative alkyl groups include, but are not limited to, methyl, ethyl, propyl, isopropyl, butyl, sec-butyl, tert-butyl, pentyl, isopentyl, neopentyl, hexyl, isohexyl, and neohexyl. The term “C₁-C₄ alkyl” refers to a straight or branched chain alkane (hydrocarbon) radical containing from 1 to 4 carbon atoms, such as methyl, ethyl, propyl, isopropyl, n-butyl, t-butyl, and isobutyl.

The term “alkenyl” as used herein refers to a linear or branched hydrocarbon having from 2 to 6 carbon atoms and having at least one carbon-carbon double bond. In one embodiment, the alkenyl has one or two double bonds. The alkenyl moiety may exist in the E or Z conformation and the compounds of the present invention include both conformations.

The term “alkynyl” as used herein refers to a linear or branched hydrocarbon having from 2 to 6 carbon atoms and having at least one carbon-carbon triple bond.

The term “aryl” as used herein refers to an aromatic group containing 1 to 3 aromatic rings, either fused or linked.

The term “cyclic group” as used herein includes a cycloalkyl group and a heterocyclic group.

The term “cycloalkyl” as used herein refers to a three- to seven-membered saturated or partially unsaturated carbon ring. Any suitable ring position of the cycloalkyl group may be covalently linked to the defined chemical structure. Examples of cycloalkyl groups include cyclopropyl, cyclobutyl, cyclopentyl, cyclohexyl, and cycloheptyl.

The term “halogen” as used herein refers to fluorine, chlorine, bromine, and iodine.

The term “heterocyclic group” or “heterocyclic” or “heterocyclyl” or “heterocyclo” as used herein refers to fully saturated, or partially or fully unsaturated, including aromatic (i.e., “heteroaryl”) cyclic groups (for example, 4 to 7 membered monocyclic, 7 to 11 membered bicyclic, or 10 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached to the remainder of the molecule at any heteroatom or carbon atom of the ring or ring system. Examples of heterocyclic groups include, but are not limited to, azepanyl, azetidinyl, aziridinyl, dioxolanyl, furanyl, furazanyl, homo piperazinyl, imidazolidinyl, imidazolinyl, isothiazolyl, isoxazolyl, morpholinyl, oxadiazolyl, oxazolidinyl, oxazolyl, oxazolidinyl, pyrimidinyl, phenanthridinyl, phenanthrolinyl, piperazinyl, piperidinyl, pyranyl, pyrazinyl, pyrazolidinyl, pyrazolinyl, pyrazolyl, pyridazinyl, pyridooxazolyl, pyridoimidazolyl, pyridothiazolyl, pyridinyl, pyrimidinyl, pyrrolidinyl, pyrrolinyl, quinuclidinyl, tetrahydrofuranyl, thiadiazinyl, thiadiazolyl, thienyl, thienothiazolyl, thienooxazolyl, thienoimidazolyl, thiomorpholinyl, thiophenyl, triazinyl, and triazolyl. Examples of bicyclic heterocyclic groups include indolyl, isoindolyl, benzothiazolyl, benzoxazolyl, benzoxadiazolyl, benzothienyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Examples of tricyclic heterocyclic groups include carbazolyl, benzidolyl, phenanthrolinyl, acridinyl, phenanthridinyl, xanthenyl and the like.

The term “phenyl” as used herein refers to a substituted or unsubstituted phenyl group.

The aforementioned terms “alkyl,” “alkenyl,” “alkynyl,” “aryl,” “phenyl,” “cyclic group,” “cycloalkyl,” “heterocyclyl,” “heterocyclo,” and “heterocycle” can further be optionally substituted with one or more substituents. Examples of substituents include but are not limited to one or more of the following groups: hydrogen, halogen, CF₃, OCF₃, cyano, nitro, N₃, oxo, cycloalkyl, alkenyl, alkynyl, heterocycle, aryl, alkylaryl, heteroaryl, OR^{a}, SR^{a}, S(═O)R^{e}, S(═O)₂R^{e}, P(═O)₂R^{e}, S(═O)₂OR^{a}, P(═O)₂OR^{a}, NR^{b}R^{c}, NR^{b}S(═O)₂R^{e}, NR^{b}P(═O)₂R^{e}, S(═O)₂NR^{b}R^{c}, P(═O)₂NR^{b}R^{c}, C(═O)OR^{a}, C(═O)^{R}ₐ, C(═O)NR^{b}R^{c}, OC(═O)R^{a}, OC(═O)NR^{b}R^{c}, NR C(═O)OR^{a}, NR^{d}C(═O)NR^{b}R^{c}, NR^{d}S(═O)₂NR^{b}R^{c}, NR^{d}P(═O)₂NR^{b}R^{c}, NR^{b}C(═O)R^{a}, or NR^{b}P(═O)₂R^{e}, wherein R^{a} is hydrogen, alkyl, cycloalkyl, alkenyl, alkynyl, alkylaryl, heteroaryl, heterocycle, or aryl; R^{b}, R^{c} and R^{d} are independently hydrogen, alkyl, cycloalkyl, alkylaryl, heteroaryl, heterocycle, aryl, or said R^{b} and R^{c}, together with the N to which R^{b} and R^{c} are bonded optionally form a heterocycle; and R^{c} is alkyl, cycloalkyl, alkenyl, cycloalkenyl, alkynyl, alkylaryl, heteroaryl, heterocycle, or aryl. In the aforementioned examples of substituents, groups such as alkyl, cycloalkyl, alkenyl, alkynyl, cycloalkenyl, alkylaryl; heteroaryl, heterocycle and aryl can themselves be optionally substituted.

Example substituents can further optionally include at least one labeling group, such as a fluorescent, a bioluminescent, a chemiluminescent, a colorimetric and a radioactive labeling group. A fluorescent labeling group can be selected from bodipy, dansyl, fluorescein, rhodamine, Texas red, cyanine dyes, pyrene, coumarins, Cascade Blue™, Pacific Blue, Marina Blue, Oregon Green, 4′,6-Diamidino-2-phenylindole (DAPI), indopyra dyes, lucifer yellow, propidium iodide, porphyrins, arginine, and variants and derivatives thereof. For example, ARM118 of the present invention contains a labeling group BODIPY, which is a family of fluorophores based on the 4,4-difluoro-4-bora-3a,4a-diaza-s-indacene moiety. For further information on fluorescent label moieties and fluorescence techniques, see, e.g., Handbook of Fluorescent Probes and Research Chemicals, by Richard P. Haughland, Sixth Edition, Molecular Probes, (1996), which is hereby incorporated by reference in its entirety. One of skill in the art can readily select a suitable labeling group, and conjugate such a labeling group to any of the compounds of the invention, without undue experimentation.

### Pharmaceutically Acceptable Salts.

The disclosure provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, imidazole, or pyrazine.

In some embodiments, an ammonium salt is a triethyl amine salt, a trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, trifluoroacetic acid, mandelic acid, cinnamic acid, aspartic acid, stearic acid, palmitic acid, glycolic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a trifluoroacetate salt, a mandelate salt, a cinnamate salt, an aspartate salt, a stearate salt, a palmitate salt, a glycolate salt, a propionate salt, a butyrate salt, a fumarate salt, a hemifumarate salt, a succinate salt, a methanesulfonate salt, an ethanesulfonate salt, a benzenesulfonate salt, a p-toluenesulfonate salt, a citrate salt, an oxalate salt, or a maleate salt.

### Compounds.

In some embodiments, a compound capable of binding RyR2 is a compound of Formula I:

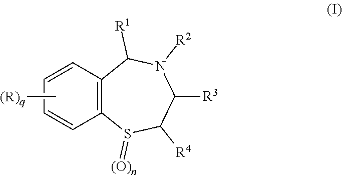

wherein,
n is 0, 1, or 2;
q is 0, 1, 2, 3, or 4;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹; and
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
t is 1, 2, 3, 4, 5, or 6;
m is 1, 2, 3, or 4;
   or a pharmaceutically-acceptable salt thereof.

In some embodiments, R² is unsubstituted alkyl.

In some embodiments, the present disclosure provides compounds of Formula I-a:

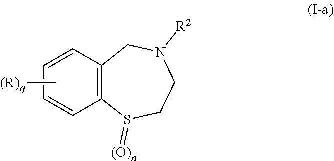

wherein:
n is 0, 1, or 2;
q is 0, 1, 2, 3, or 4;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, N—NR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)R¹², NH(C═O)R¹², —O(C═O)R¹², or —P(═O)R¹³R¹⁴; m is 0, 1, 2, 3, or 4;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is halogen, —CN, —CO₂R¹⁵, —C(═O)NR¹⁵R¹⁶, —NR¹⁵R¹⁶, —OR¹⁵, —SO₂R⁷, or —P(═O)R⁸R⁹; and
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
or a pharmaceutically-acceptable salt thereof.

In some embodiments, the present disclosure provides a compound of formula I-a, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1, or 2.

In some embodiments, the present disclosure provides a compound of formula I-a, wherein R₂ is —C═O(R⁵), —C═S(R⁶), —SO₂R⁷, —P(═O)R⁸R⁹, or —(CH₂)ₘ—R¹⁰.

In some embodiments, the present disclosure provides a compound of formula I-b:

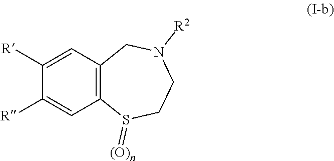

wherein
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰; and
n is 0, 1, or 2;
   or a pharmaceutically-acceptable salt thereof.

In some embodiments, the present disclosure provides a compound of formula I-b, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-b, wherein R₂ is —C═O(R₅), —C═S(R₆), SO₂R₇, P(═O)R₈R₉, or —(CH₂)ₘ—R₁₀.

In some embodiments, the present disclosure provides a compound formula of I-c:

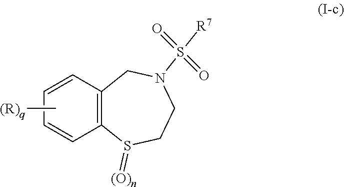

n is 0, 1, or 2;
q is 0, 1, 2, 3, or 4;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
or a pharmaceutically-acceptable salt thereof.

In some embodiments, the present disclosure provides a compound of formula I-c, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1, or 2.

In some embodiments, the present disclosure provides a compound of formula I-c, wherein R⁷ is alkyl, alkenyl, aryl, cycloalkyl, cycloalkylalkyl, heterocyclyl, or heterocyclylalkyl, each of which is independently substituted or unsubstituted; or —OH or —NR¹⁵R¹⁶.

In some embodiments, the present disclosure provides a compound of formula of I-d:

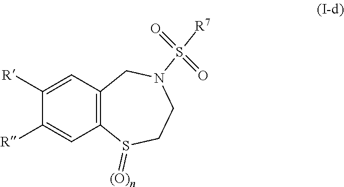

n is 0, 1, or 2;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X,
   or a pharmaceutically-acceptable salt thereof.

In some embodiments, the present disclosure provides a compound of formula wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-d, wherein R₇ is alkyl, alkenyl, aryl, cycloalkyl, cycloalkylalkyl, heterocyclyl, or heterocyclylalkyl, each of which is independently substituted or unsubstituted; or —OH, or —NR¹⁵R¹⁶.

In some embodiments, the present disclosure provides a compound of formula of I-e:

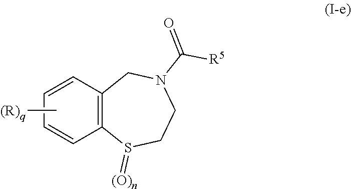

n is 0, 1, or 2;
q is 0, 1, 2, 3, or 4;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃; and
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group,
   or a pharmaceutically-acceptable salt thereof.

In some embodiments, the present disclosure provides a compound of formula I-e, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1, or 2.

In some embodiments, the present disclosure provides a compound of formula I-e, wherein R₅ is alkyl, alkenyl, aryl, cycloalkyl, cycloalkylalkyl, heterocyclyl, or heterocyclylalkyl, each of which is independently substituted or unsubstituted; or —NR¹⁵R¹⁶, —NHOH, —OR¹⁵, or —CH₂X.

In some embodiments, the present disclosure provides a compound of formula of I-f:

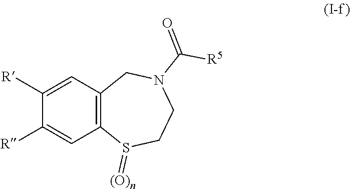

n is 0, 1, or 2;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group,
   or a pharmaceutically-acceptable salt thereof.

In some embodiments, the present disclosure provides a compound of formula I-f, wherein R′ and R″ are each independently H, halogen, —OH, OMe, —NH₂, —NO₂, —CN, —CF₃, —OCF₃, —N₃, —S(═O)₂C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-f, wherein R₅ is alkyl, alkenyl, aryl, cycloalkyl, cycloalkylalkyl, heterocyclyl, or heterocyclylalkyl, each of which is independently substituted or unsubstituted; or —NR¹⁵R¹⁶, —NHOH, —OR¹⁵, or —CH₂X.

In some embodiments, the present disclosure provides a compound of formula of I-g:

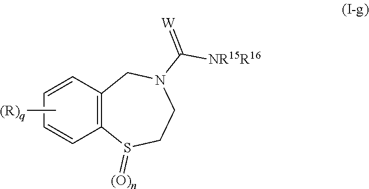

wherein
n is 0, 1, or 2;
q is 0, 1, 2, 3, or 4;
W is S or O;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded may form a heterocycle that is substituted or unsubstituted,
   or a pharmaceutically-acceptable salt thereof.

In some embodiments, the present disclosure provides a compound of formula I-g, wherein each R is independently selected from the group consisting of H, halogen, —OH, OMe, —NH₂, —NO₂, —CN, —CF₃, —OCF₃, —N₃, —S(═O)₂C₁-C₄alkyl, —S(═O)C₁-C₄alkyl, —S-C₁-C₄alkyl, —OS(═O)₂CF₃, Ph, —NHCH₂Ph, —C(═O)Me, —OC(═O)Me, morpholinyl and propenyl; and n is 0, 1, or 2.

In some embodiments, the present disclosure provides a compound of formula I-g, wherein R¹⁵ and R¹⁶ are each independently alkyl, alkylamino, aryl, cycloalkyl, cycloalkylalkyl, heterocyclyl, and heterocyclylalkyl, each of which is independently substituted or unsubstituted; or H, OH, or NH₂; or R¹⁵ and R¹⁶ together with the N to which they are bonded form a heterocycle that is substituted or unsubstituted.

In some embodiments, the present disclosure provides a compound of formula I-g, wherein W is O or S.

In some embodiments, the present disclosure provides a compound of formula of I-h:

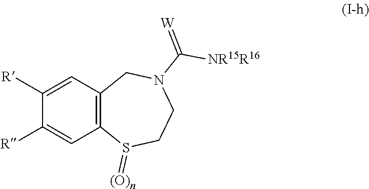

n is 0, 1, or 2;
W is S or O;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃,
   or a pharmaceutically-acceptable salt thereof.

In some embodiments, the present disclosure provides a compound of formula wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-h, wherein R¹⁵ and R¹⁶ are each independently alkyl, alkylamino, aryl, cycloalkyl, cycloalkylalkyl, heterocyclyl, or heterocyclylalkyl, each of which is independently substituted or unsubstituted; or H, OH, NH₂; or R¹⁵ and R¹⁶ together with the N to which R¹⁵ and R¹⁶ are bonded form a heterocycle that is substituted or unsubstituted.

In some embodiments, the present disclosure provides a compound of formula I-g, wherein W is O or S.

In some embodiments, the present disclosure provides a compound of formula of I-i:

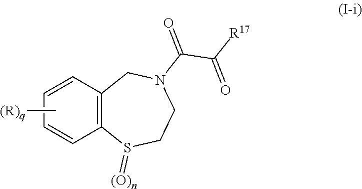

wherein
R¹⁷ is alkenyl, aryl, cycloalkyl, cycloalkylalkyl, heterocyclyl, and heterocyclylalkyl, each of which is independently substituted or unsubstituted; or —NR¹⁵R¹⁶, —NHNR¹⁵R¹⁶, —NHOH, —OR¹⁵, or —CH₂X;
n is 0, 1, or 2;
q is 0, 1, 2, 3, or 4; and
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃,
   or a pharmaceutically-acceptable salt thereof.

In some embodiments, the present disclosure provides a compound of formula I-i, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1, or 2.

In some embodiments, the present disclosure provides a compound of formula I-i, wherein R¹⁷ is —NR¹⁵R¹⁶ or —OR¹⁵. In some embodiments, R¹⁷ is —OH, —OMe, —Net, —NHEt, —NHPh, —NH₂, or —NHCH₂pyridyl.

In some embodiments, the present disclosure provides a compound of formula of I-j:

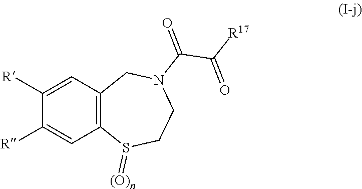

R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
   R₁₇ is selected from the group consisting of —NR₁₅R₁₆, —NHOH, —OR¹⁵, —CH₂X, alkenyl, aryl, cycloalkyl, cycloalkylalkyl, heterocyclyl, and heterocyclylalkyl; wherein each alkenyl, aryl, cycloalkyl, cycloalkylalkyl, heterocyclyl, and heterocyclylalkyl may be substituted or unsubstituted;
   n is 0, 1, or 2,
      or a pharmaceutically-acceptable salt thereof.

In some embodiments, the present disclosure provides a compound of formula I-j, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-j, wherein R¹⁷ is NR¹⁵R¹⁶ or —OR¹⁵. In some embodiments, R¹⁷ is —OH, —OMe, —Net, —NHEt, —NHPh, —NH₂, or —NHCH₂pyridyl.

In some embodiments, the present disclosure provides a compound of formula I-k or I-k-1:

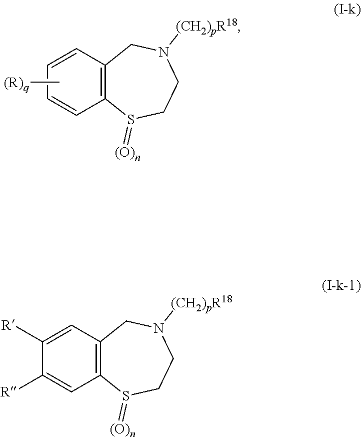

each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
R¹⁸ is alkyl, aryl, cycloalkyl, or heterocyclyl, each of which is independently substituted or unsubstituted; or —NR¹⁵R¹⁶, —C(═O)NR¹⁵R¹⁶, —(C═O)OR¹⁵, or —OR¹⁵;
q is 0, 1, 2, 3, or 4;
p is 1, 2, 3, 4, 5, 6, 7, 8 9, or 10; and
n is 0, 1, or 2,
   or a pharmaceutically-acceptable salt thereof.

In some embodiments, the present disclosure provides a compound of formula I-k, wherein each R is independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S-C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R is OMe at position 7 of the benzothiazepine ring.

In some embodiments, the present disclosure provides a compound of formula I-k-1, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S-C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-k or I-k-1, wherein R₁₈ is —NR¹⁵R¹⁶, —(C═O)OR¹⁵, —OR¹⁵, alkyl that is substituted or unsubstituted, or aryl that is substituted or unsubstituted. In some embodiments, m is 1, and R¹⁸ is Ph, —C(═O)OMe, C(═O)OH, aminoalkyl, NH₂, NHOH, or NHCbz. In other embodiments, m is 0, and R¹⁸ is C₁-C₄ alkyl. In other embodiments, R¹⁸ is Me, Et, propyl, and butyl. In some embodiments, m is 2, and R¹⁸ is pyrrolidine, piperidine, piperazine, or morpholine. In some embodiments, m is 3, 4, 5, 5, 7, or 8, and R¹⁸ is a fluorescent labeling group selected from bodipy, dansyl, fluorescein, rhodamine, Texas red, cyanine dyes, pyrene, coumarins, Cascade Blue™, Pacific Blue, Marina Blue, Oregon Green, 4′,6-Diamidino-2-phenylindole (DAPI), indopyra dyes, lucifer yellow, propidium iodide, porphyrins, arginine, and variants and derivatives thereof.

In some embodiments, the present disclosure provides a compound of formula of I-1 or I-1-1:

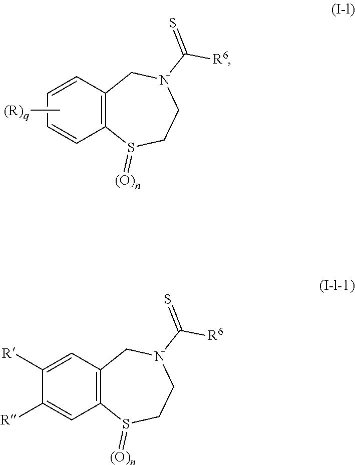

wherein
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
q is 0, 1, 2, 3, or 4; and
n is 0, 1, or 2,
   or a pharmaceutically acceptable salt thereof.

In some embodiments, the present disclosure provides a compound of formula I-1, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R is OMe at position 7 of the benzothiazepine ring.

In some embodiments, the present disclosure provides a compound of formula I-1-1, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-1 or I-1-1, wherein R⁶ is acyl, alkenyl, alkyl, aryl, cycloalkyl, cycloalkylalkyl, heterocyclyl, and heterocyclylalkyl, each of which is independently substituted or unsubstituted; or —NR¹⁵R¹⁶, —NHNR¹⁵R¹⁶, —OR¹⁵, —NHOH, or —CH₂X. In some embodiments, R⁶ is —NR¹⁵R¹⁶. In some embodiments, R⁶ is —NHPh, pyrrolidine, piperidine, piperazine, morpholine. In some embodiments, R⁶ is alkoxyl. In some embodiments, R⁶ is —O-tBu.

In some embodiments, the present disclosure provides a compound of formula I-m or I-m-1:

wherein

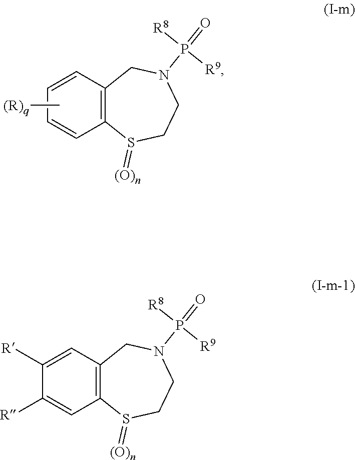

n is 0, 1, or 2;
q is 0, 1, 2, 3, or 4;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃; and
R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH,
   or a pharmaceutically-acceptable salt thereof.

In some embodiments, the present disclosure provides a compound of formula I-m, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R is OMe at position 7 of the benzothiazepine ring.

In some embodiments, the present disclosure provides a compound of formula I-m-1, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-m or I-m-1, wherein R⁸ and R⁹ are each independently alkyl, aryl, OH, alkoxyl, or alkylamino. In some embodiments, R⁸ is C₁-C₄alkyl. In some embodiments, R⁸ is Me, Et, propyl or butyl. In some embodiments, R⁹ is aryl. In some embodiments, R⁹ is phenyl.

In some embodiments, the present disclosure provides a compound of formula I-n,

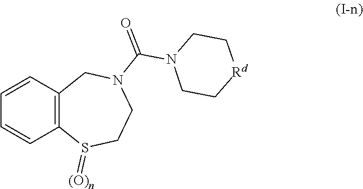

wherein:
R^{d} is CH₂, or NR^{a}; and
R^{a} is H, alkoxy, (C₁-C₆ alkyl)-aryl, wherein the aryl is a disubstituted phenyl or a benzo[1,3]dioxo-5-yl group, or a Boc group.
   or a pharmaceutically-acceptable salt thereof.

In some embodiments, R^{a} is H.

Representative compounds of Formula I-n include without limitation S101, S102, S103, and S114.

In some embodiments, the present disclosure provides a compound of Formula I-o:

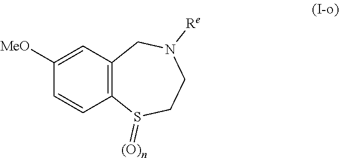

wherein:
R^{e} is (C₁-C₆ alkyl)-phenyl, (C₁-C₆ alkyl)-C(O)R^{b}, or substituted or unsubstituted C₁-C₆ alkyl; and
R^{b} is OH or —O—(C₁-C₆ alkyl),
   wherein the phenyl or the substituted alkyl is substituted with one or more of halogen, hydroxyl, C₁-C₆ alkyl, —O—(C₁-C₆ alkyl), —NH₂, —NH(C₁-C₆ alkyl), —N(C₁-C₆ alkyl)₂, cyano, or dioxolane,
   or a pharmaceutically-acceptable salt thereof.

Representative compounds of Formula I-o include without limitation S107, S110, S111, S120, and S121.

In some embodiments, the present disclosure provides a compound of Formula I-p:

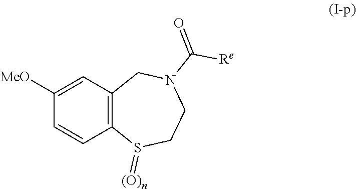

wherein:
R^{c} is —(C₁-C₆ alkyl)-NH₂, —(C₁-C₆ alkyl)-OR^{f}, wherein R_{f} is H or —C(O)—(C₁-C₆)alkyl, or —(C₁-C₆ alkyl)-NHR^{g}, wherein R^{g} is carboxybenzyl.

In some embodiments, the present disclosure provides compounds of Formula II or Formula III:

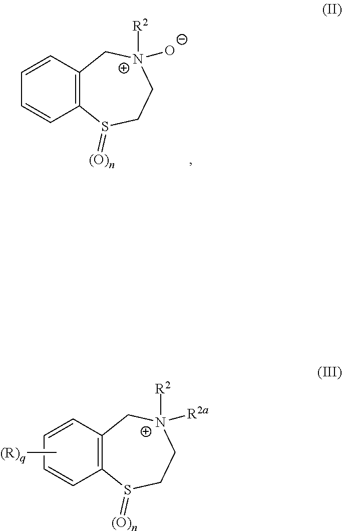

wherein:
n is 0, 1, or 2;
q is 0, 1, 2, 3, or 4;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, ≤N₃, —SO₃H, —S(═O)₂alkyl, —S(═O)alkyl, or —OS(═O)₂CF₃;
each R² and R^{2a} is independently alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R₁₀;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, —C(═O)R¹², NH(C═O)R¹², —O(C═O)R¹², or —P(═O)R¹³R¹⁴;
m is 0, 1, 2, 3, or 4;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is halogen, —CN, —CO₂R¹⁵, —C(═O)NR¹⁵R¹⁶, —NR¹⁵R¹⁶, —OR¹⁵, —SO₂R⁷, or —P(═O)R⁸R⁹; and
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
   or a pharmaceutically acceptable salt thereof.

In some embodiments, the compound of formula (I) is selected from:

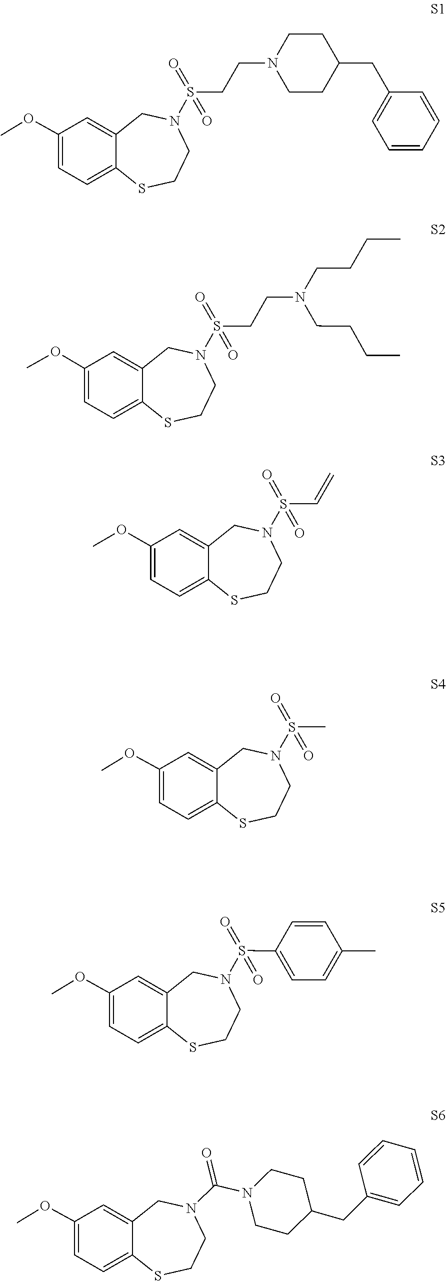

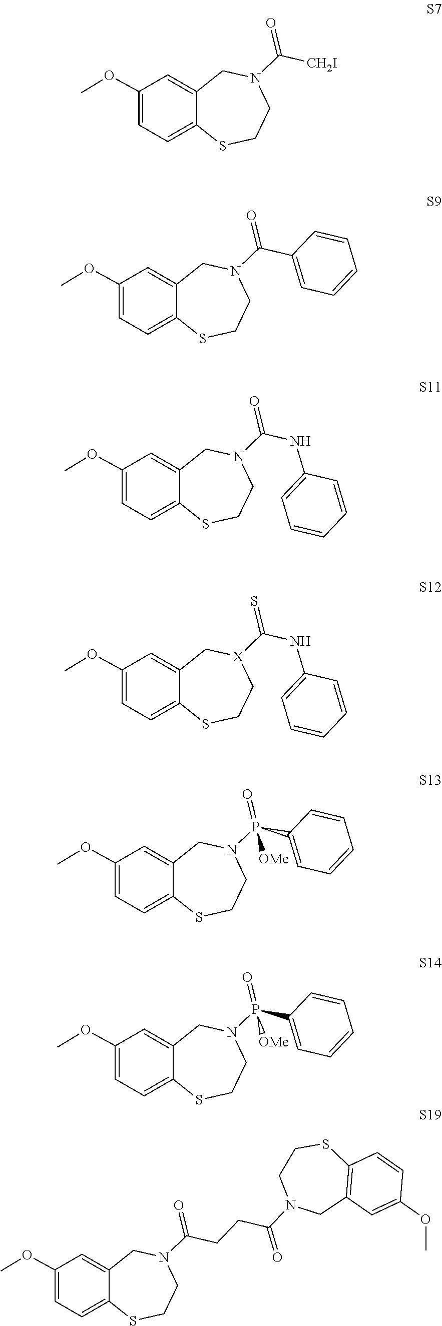

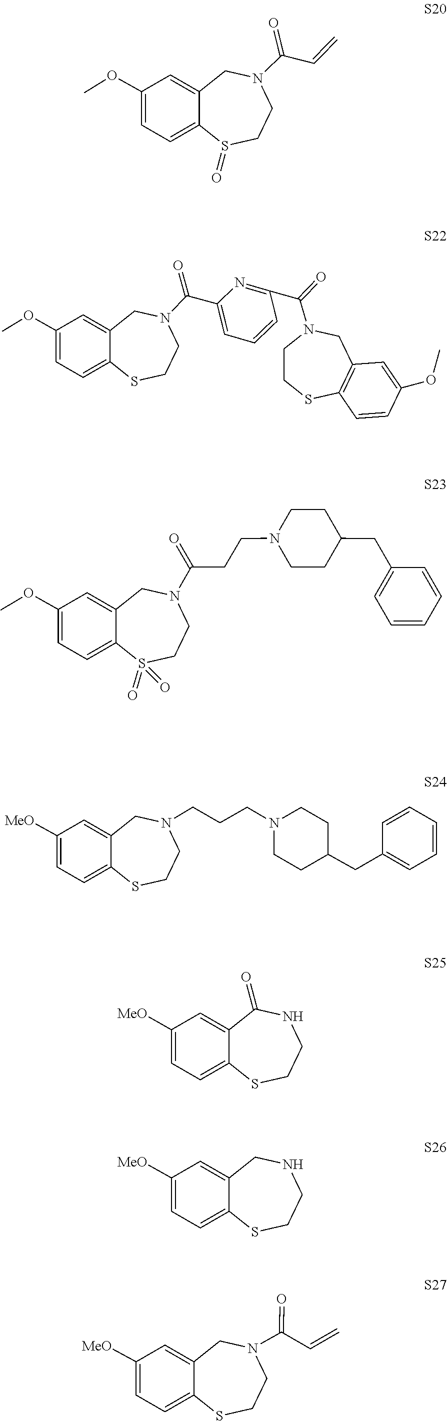

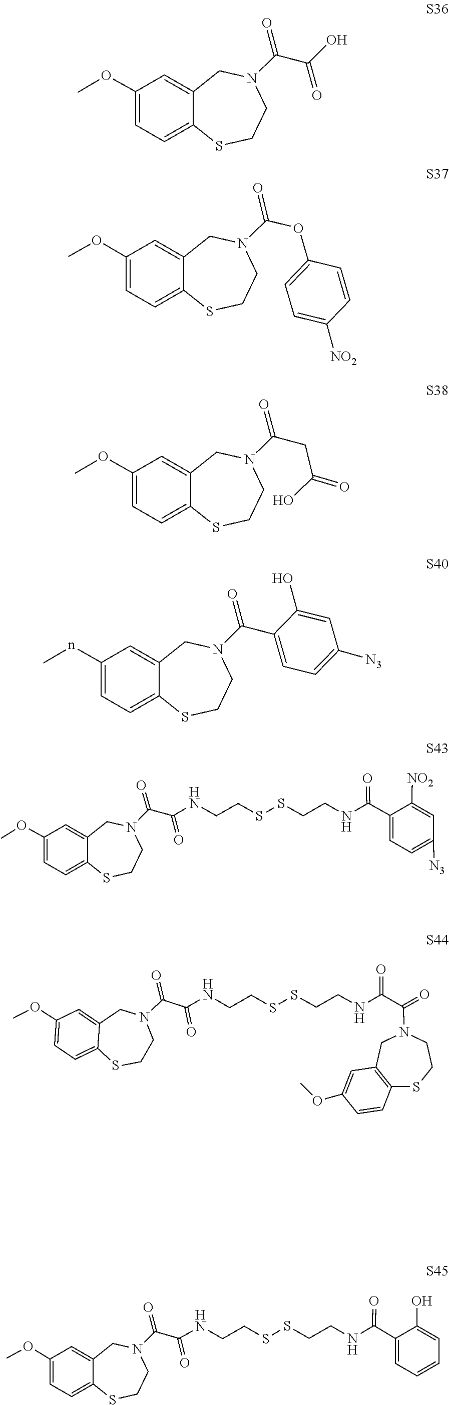

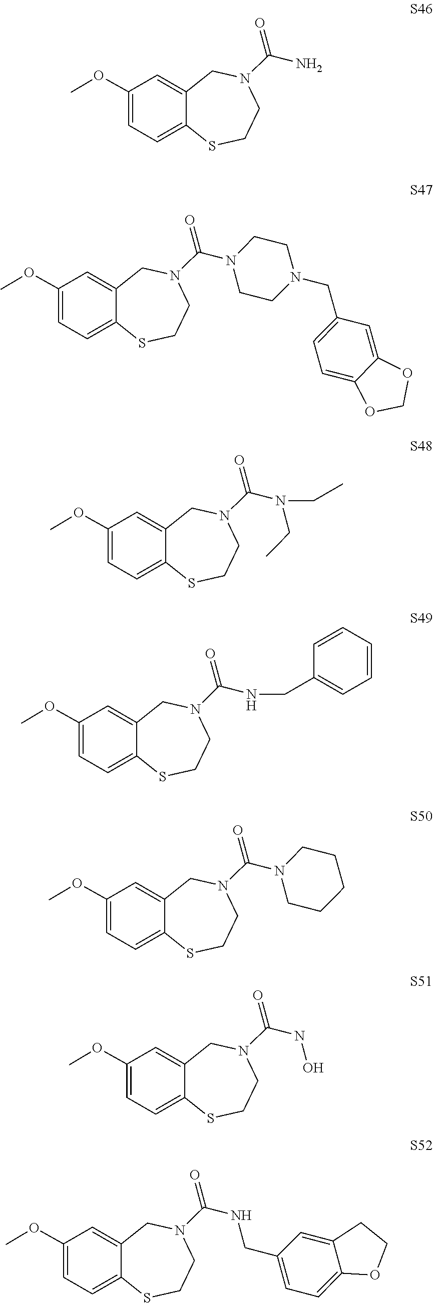

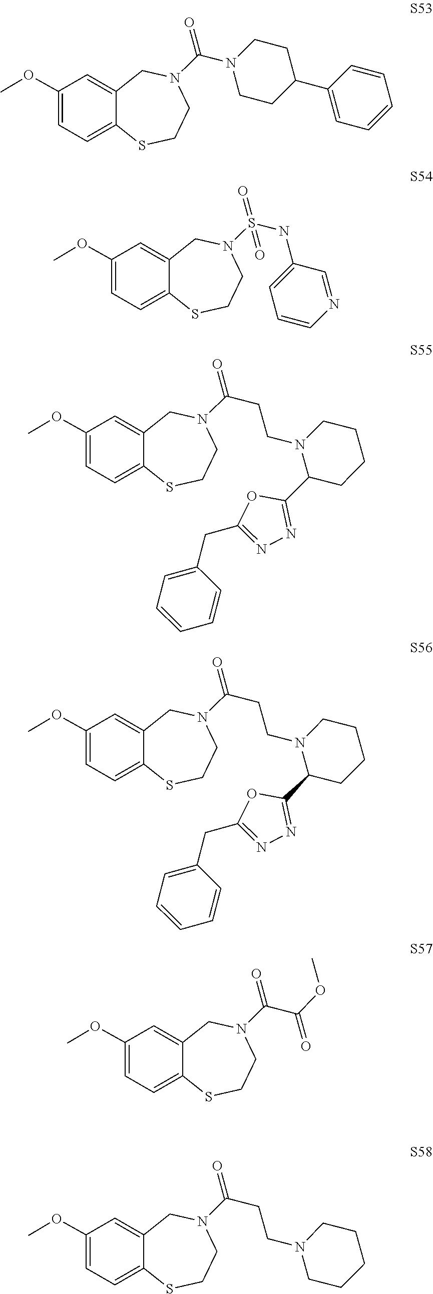

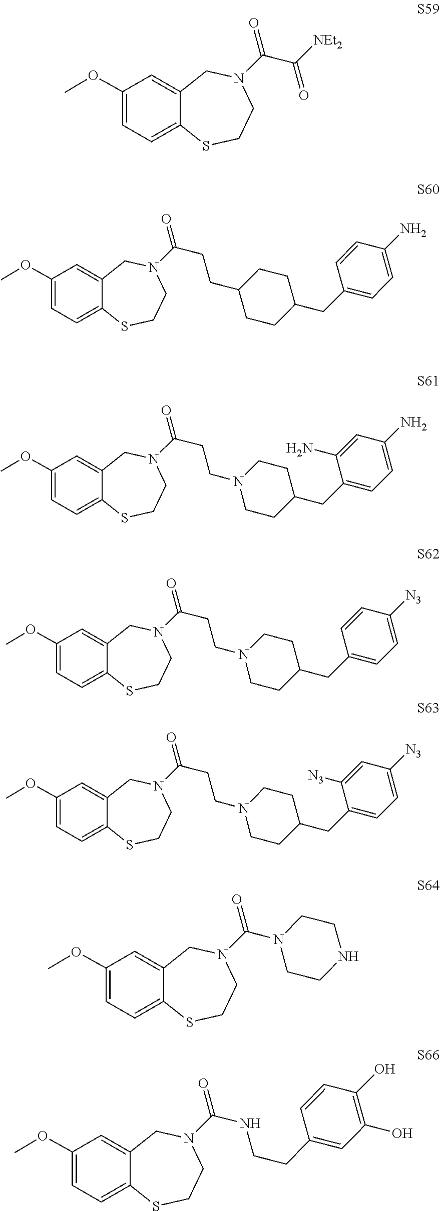

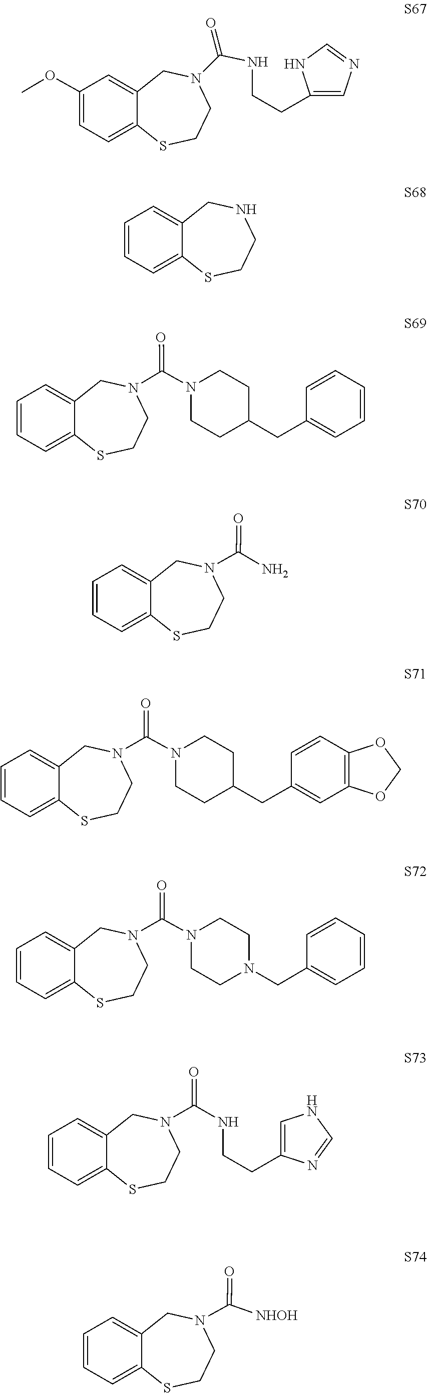

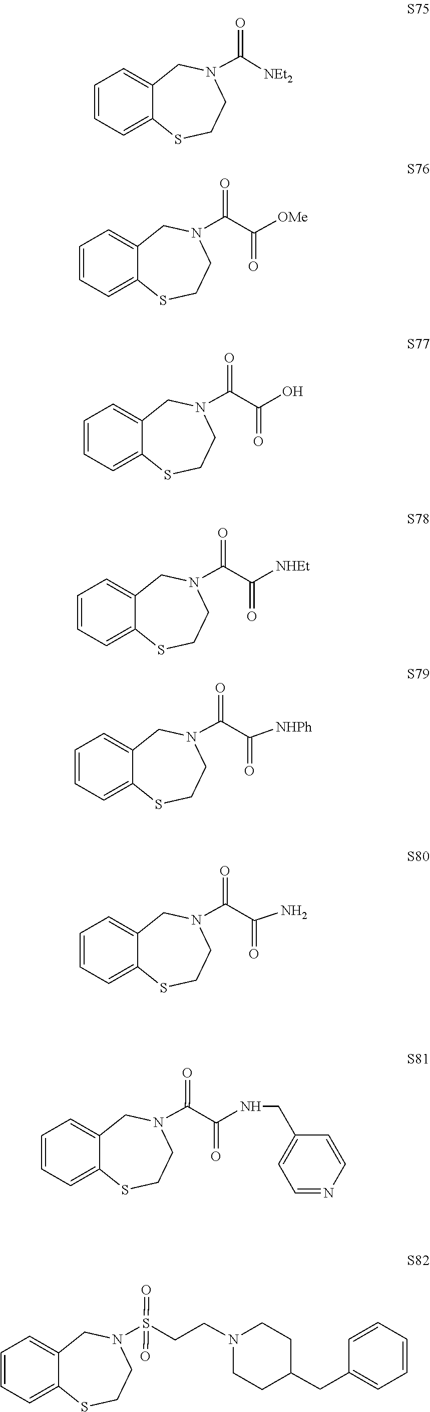

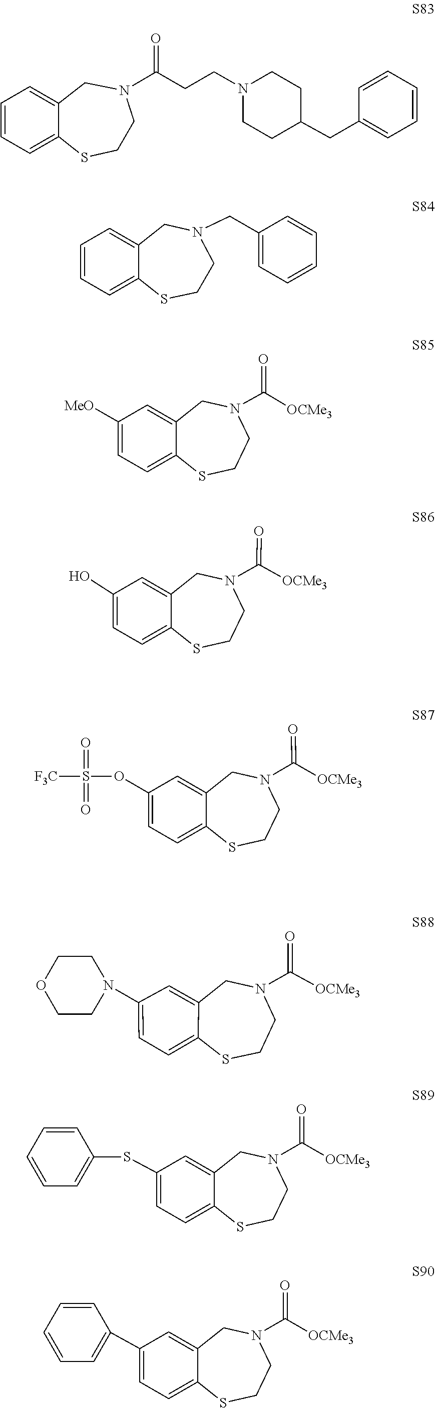

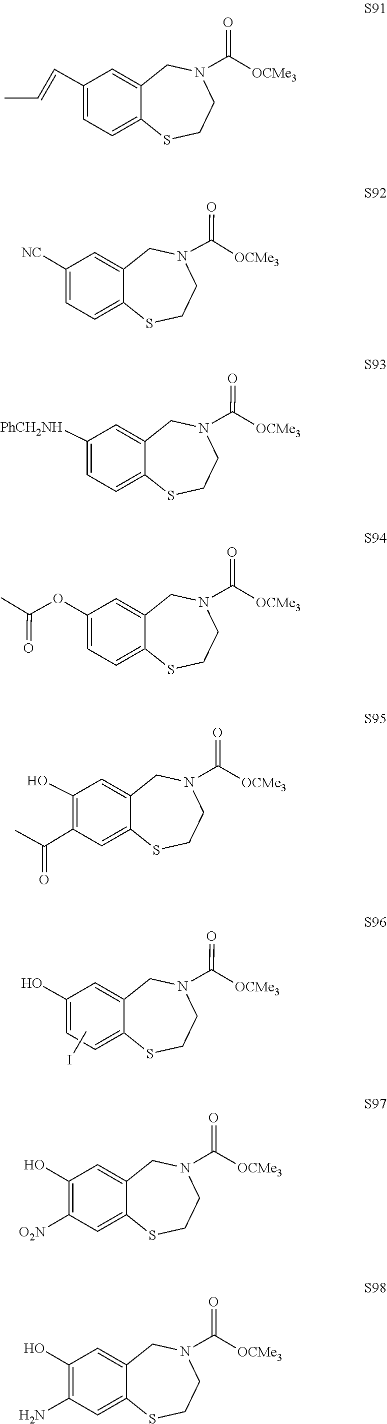

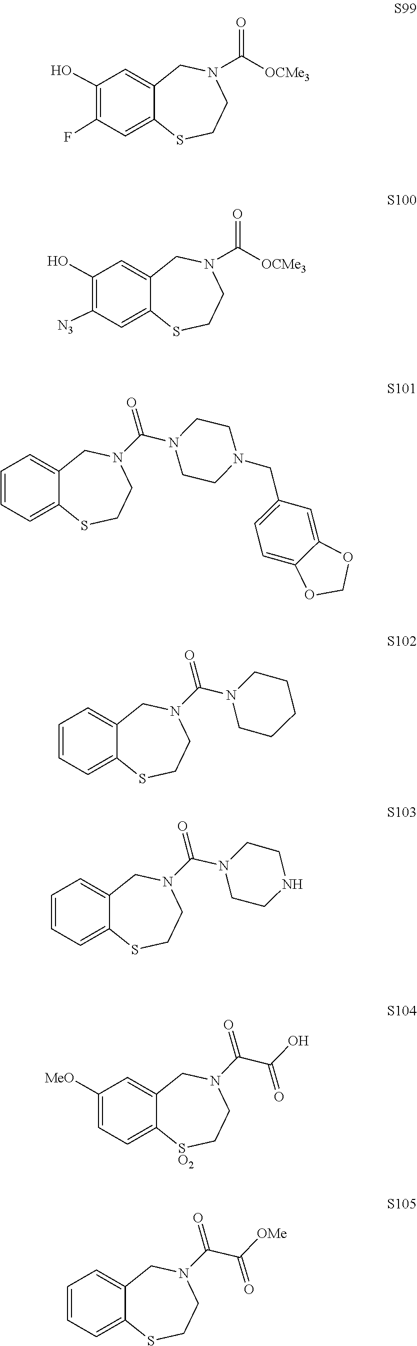

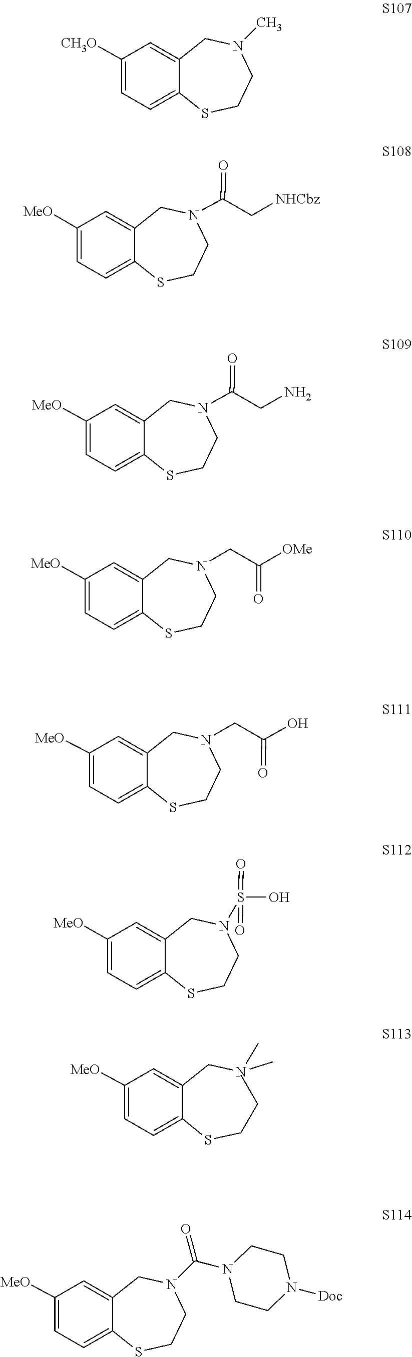

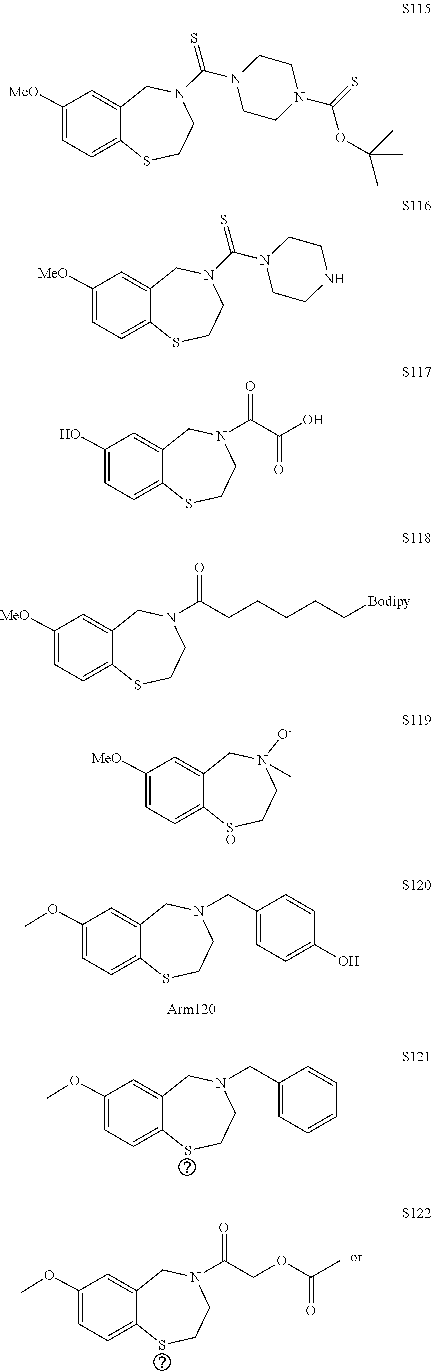

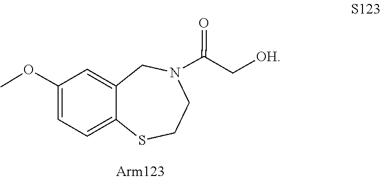 [SMILES: COc1ccc2c(c1)CN(C(=O)CO)CCS2]

In some embodiments, the synthetic compound is a compound of Formula (I), (I-a), (I-b), (I-c), (I-d), (I-e), (I-f), (I-g), (I-h), (I-i), (I-j), (I-k), (I-k-1), (I-1), (I-1-1), (I-m), (I-m-1), (I-n), (I-o), (I-p), (II), or (III). In some embodiments, the synthetic compound is S1, S2, S3, S4, S5, S6, S7, S9, S1, S12, S13, S14, S19, S20, S22, S23, S24, S25, S26, S27, S36, S37, S38, S40, S43, S44, S45, S46, S47, S48, S49, S50, S51, S52, S53, S54, S55, S56, S57, S58, S59, S60, S61, S62, S63, S64, S66, S67, S68, S69, S70, S71, S72, S73, S74, S75, S76, S77, S78, S79, S80, S81, S82, S83, S84, S85, S86, S87, S88, S89, S90, S91, S92, S93, S94, S95, S96, S97, S98, S99, S100, S101, S102, S103, S104, S105, S107, S108, S109, S110, S111, S112, S113, S114, S115, S116, S117, S118, S119, S120, S121, S122, or S123, as herein defined.

In some embodiments, the synthetic compound is:

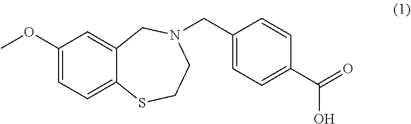 [SMILES: COc1ccc2c(c1)CN(Cc1ccc(C(=O)O)cc1)CCS2]

or a pharmaceutically-acceptable salt thereof or an ionized form thereof.

In some embodiments, the synthetic compound is:

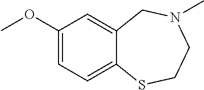

or a pharmaceutically-acceptable salt or an ionized form thereof.

Compounds described herein may exist in their tautomeric form (for example, as an amide or imino ether). All such tautomeric forms are contemplated herein as part of the present disclosure.

All stereoisomers of the compounds of the present disclosure (for example, those which may exist due to asymmetric carbons on various substituents), including enantiomeric forms and diastereomeric forms, are contemplated within the scope of this invention. Individual stereoisomers of the compounds of the disclosure may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the IUPAC 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

### Screening Methods.

The present disclosure provides methods for identifying a compound that binds to a biomolecular target (e.g. RyR2). In some embodiments, the methods described herein can include screening a library of three-dimensional compound structures to identify ligands that fit a binding pocket of the biomolecular target such as RyR2.

In some embodiments, provided is a method comprising:
(a) determining an open probability (Pₒ) of a first RyR2 protein, wherein the first RyR2 protein is treated with a test compound, and
(b) determining an open probability (Pₒ) of a second RyR2 protein, wherein the second RyR2 protein is not treated with the test compound.

In some embodiments, each of the determining the open probability (Pₒ) of the first RyR2 protein and the determining the open probability (Pₒ) of the second RyR2 protein comprises recording a single channel Ca²⁺ current. In some embodiments, the method further comprises determining a difference between the Pₒ of the first RyR2 protein and Pₒ of the second RyR2 protein. In some embodiments, the method further comprises identifying the test compound as a target for further analysis based on the difference between the Pₒ of the first RyR2 protein and Pₒ of the second RyR2 protein. In some embodiments, the Pₒ of the first RyR2 protein is lower than the Pₒ of the second RyR2 protein.

In some embodiments, the method further comprises performing an analogous assay wherein another compound is used in place of the test compound, wherein the analogous assay provides a difference between:
(a) an open probability (Pₒ) of a third RyR2 protein, wherein the third RyR2 protein is treated with the other compound; and
(b) an open probability (Pₒ) of a fourth RyR2 protein, wherein the fourth RyR2 protein is not treated with the other compound, wherein the test compound is prioritized over the other compound for the further analysis based on a comparison of:
(i) the difference between the Pₒ of the first RyR2 protein and Pₒ of the second RyR2 protein; with
(ii) a difference between the Pₒ of the third RyR2 protein and Pₒ of the fourth RyR2 protein.

In some embodiments, the difference of the Pₒ in (b)(ii) is greater than the difference of the Pₒ in (b)(i). In some embodiments, the RyR2 is a mutant RyR2. In some embodiments, the mutant RyR2 contains at least one mutation that is associated with Catecholaminergic Polymorphic Ventricular Tachycardia (CPVT). In some embodiments, the mutation is R2474S. In some embodiments, the RyR2 protein is a post-translationally modified RyR2 protein. In some embodiments, the post-translationally modified RyR2 protein is a phosphorylated, oxidized or nitrosylated RyR2. In some embodiments, the RyR2 is a mutated and post-translationally modified RyR2.

In some embodiments, provided is a method comprising:
(a) contacting a first RyR2 protein with a test compound;
(b) providing a second RyR2 protein;
(c) subsequent to the contacting the first RyR2 protein with the test compound, measuring an open probability (Pₒ) of the first RyR2 protein; and
(d) measuring an open probability (Pₒ) of the second RyR2 protein.

In some embodiments, each of the determining the open probability (Pₒ) of the first RyR2 protein and the determining the open probability (Pₒ) of second RyR2 protein comprises recording a single channel Ca²⁺ current. In some embodiments, the method further comprises determining a difference between the Pₒ of the first RyR2 protein and the Pₒ of the second RyR2 protein. In some embodiments, the method further comprises identifying the test compound as a target for further analysis based on the difference between the Pₒ of the first RyR2 protein and the Pₒ of the second RyR2 protein. In some embodiments, the Pₒ of the first RyR2 protein is lower than the Pₒ of the second RyR2 protein.

In some embodiments, the method further comprises performing an analogous assay wherein another compound is used in place of the test compound, wherein the analogous assay provides a difference between:
(a) an open probability (Pₒ) of a third RyR2 protein, wherein the third RyR2 protein is treated with the other compound; and
(b) an open probability (Pₒ) of a fourth RyR2 protein, wherein the fourth RyR2 protein is not treated with the other compound,
wherein the test compound is prioritized over the other compound for the further analysis based on a comparison of:
(i) the difference between the Pₒ of the first RyR2 protein and Pₒ of the second RyR2 protein; with
(ii) a difference between the Pₒ of the third RyR2 protein and Pₒ of the fourth RyR2 protein.

In some embodiments, the difference of the Pₒ in (b)(ii) is greater than the difference of the Pₒ in (b)(i). In some embodiments, the RyR2 is a mutant RyR2. In some embodiments, the mutant RyR2 contains at least one mutation that is associated with Catecholaminergic Polymorphic Ventricular Tachycardia (CPVT). In some embodiments, the mutation is R2474S. In some embodiments, the RyR2 protein is a post-translationally modified RyR2 protein. In some embodiments, the post-translationally modified RyR2 protein is a phosphorylated, oxidized or nitrosylated RyR2. In some embodiments, the RyR2 is a mutated and post-translationally modified RyR2. In some embodiments, the method further comprises: subsequent to the contacting the first RyR2 protein with the reagent and the test compound, fusing a first microsome containing the first RyR2 protein to a first planar lipid bilayer, and subsequent to the contacting the second RyR2 protein with the reagent, fusing a second microsome containing the second RyR2 protein to a second planar lipid bilayer.

In some embodiments, provided is a method of identifying a compound having RyR2 modulatory activity, the method comprising:
(a) determining an open probability (Pₒ) of a RyR2 protein;
(b) contacting the RyR2 protein with a test compound;
(c) determining an open probability (Pₒ) of the RyR2 protein in the presence of the test compound; and
(d) determining a difference between the Pₒ of the RyR2 protein in the presence and absence of the test compound;
wherein a reduction in the Pₒ of the RyR2 protein in the presence of the test compound relative to the Pₒ of the RyR2 protein in the absence of the test compound is indicative of the compound having RyR2 modulatory activity.

In some embodiments, the RyR2 protein is a mutant RyR2 protein. In some embodiments, the mutant RyR2 contains at least one mutation that is associated with Catecholaminergic Polymorphic Ventricular Tachycardia (CPVT). In some embodiments, the mutation is R2474S. In some embodiments, the RyR2 protein is a post-translationally modified RyR2 protein. In some embodiments, the post-translationally modified RyR2 protein is a phosphorylated, oxidized or nitrosylated RyR2. In some embodiments, the RyR2 protein is a mutated and post-translationally modified RyR2 protein. In some embodiments, the test compound preferentially binds to a mutant RyR2 relative to wild-type RyR2. In some embodiments, the test compound preferentially binds to post-translationally modified RyR2 relative to wild-type RyR2. In some embodiments, the test compound preferentially binds to a mutated and post-translationally modified RyR2 relative to a wild-type RyR2. In some embodiments, determining the open probability (Pₒ) of the RyR2 protein comprises recording a single channel Ca²⁺ current.

In some embodiments, provided is a method for identifying a compound having RyR2 modulatory activity, comprising:
(a) contacting a RyR2 protein with a ligand having known RyR2 modulatory activity to create a mixture, wherein the RyR2 protein is a leaky RyR2, the leaky RyR2 comprising mutant RyR2 protein, post-translationally modified RyR2, or a combination thereof;
(b) contacting the mixture of step (a) with a test compound; and
(c) determining an ability of the test compound to displace the ligand from the RyR2 protein.

In some embodiments, the ligand is radiolabeled. In some embodiments, determining the ability of the test compound to displace the ligand from the RyR2 protein comprises determining a radioactive signal in the RyR2 protein. In some embodiments, the RyR2 protein is a mutant RyR2 protein. In some embodiments, the mutant RyR2 contains at least one mutation that is associated with Catecholaminergic Polymorphic Ventricular Tachycardia (CPVT). In some embodiments, the mutation is R2474S. In some embodiments, the RyR2 protein is a post-translationally modified RyR2 protein. In some embodiments, the RyR2 protein is a mutated and post-translationally modified RyR2 protein. In some embodiments, the test compound preferentially binds to a mutant RyR2 relative to wild-type RyR2. In some embodiments, the test compound preferentially binds to post-translationally modified RyR2 relative to wild-type RyR2. In some embodiments, the test compound preferentially binds to a mutant and post-translationally modified RyR2 relative to a wild-type RyR2.

In some embodiments, provided is a method for identifying a compound that preferentially binds to a mutated, post-translationally modified RyR2 or a combination thereof, comprising:
(a) determining binding affinity of a test compound to a first RyR2 protein, wherein the first RyR2 protein is a wild-type RyR2 protein;
(b) determining binding affinity of a test compound to a second RyR2 protein, wherein second first RyR2 protein is a mutant RyR2 protein, a post-translationally modified RyR2, or a combination thereof, and
(c) selecting a compound having a higher binding affinity to the second RyR2 protein relative to the first RyR2 protein.

In some embodiments, the RyR2 protein is a mutant RyR2 protein. In some embodiments, the mutant RyR2 contains at least one mutation that is associated with Catecholaminergic Polymorphic Ventricular Tachycardia (CPVT). In some embodiments, the mutation is R2474S. In some embodiments, the RyR2 protein is a post-translationally modified RyR2 protein. In some embodiments, the RyR2 protein is a mutated and post-translationally modified RyR2 protein. In some embodiments, the test compound preferentially binds to a mutant RyR2 relative to wild-type RyR2. In some embodiments, the test compound preferentially binds to post-translationally modified RyR2 relative to wild-type RyR2. In some embodiments, the test compound preferentially binds to a mutant and post-translationally modified RyR2 relative to a wild-type RyR2. In some embodiments, the test compound contains a benzothiazepane moiety.

In some embodiments, the test compound is a compound of Formula (I):

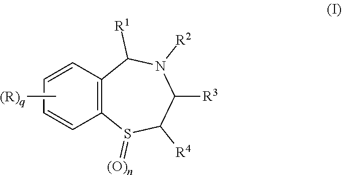

wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, OR, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, —NH₂, —NHNH₂, or —NHOH;
each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
n is 0, 1, or 2;
q is 0, 1, 2, 3, or 4;
t is 1, 2, 3, 4, 5, or 6; and
m is 1, 2, 3, or 4,
   or any other compound herein, or a pharmaceutically acceptable salt thereof.

In some embodiments, the test compound is a compound of Formula (I), (I-a), (I-b), (I-c), (I-d), (I-e), (I-f), (I-g), (I-h), (I-i), (I-j), (I-k), (I-k-1), (I-1), (I-1-1), (I-m), (I-m-1), (I-n), (I-o), (I-p), (II) or (III).

In some embodiments, the test compound is

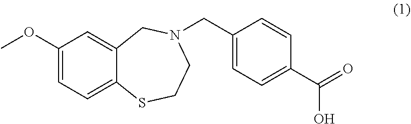 [SMILES: COc1ccc2c(c1)CN(Cc1ccc(C(=O)O)cc1)CCS2]

or an ionized form thereof.

In some embodiments, the test compound is

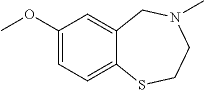

or an ionized form thereof.

### Computational Methods.

In some embodiments, a cryo-EM model disclosed herein can be used as a tool to screen for ryanodine receptor modulator compounds that bind RyR2. In some embodiments, a cryo-EM model disclosed herein can be used as a tool to screen for ryanodine receptor modulator compounds which preferentially bind leaky RyR2 (e.g., mutated RyR2, or post-translationally modified RyR2 (e.g., phosphorylated, dephosphorylated, oxidized and/or nitrosylated RyR2)) and stabilize the closed state of the RyR channel.

Structures of compounds (e.g., Compound 1) and biomolecular targets (e.g. RyR2) provided herein can be used in computational methods for identifying ligands that bind to a biomolecular target (e.g. RyR2). Such methods can include, for example, screening a library of three-dimensional compound structures to identify ligands that fit a binding pocket of the biomolecular target via a molecular docking system (e.g. Glide, DOCK, AutoDock, AutoDock Vina, FRED, and EnzyDock); de-novo generation of a structure of a ligand that binds the biomolecular target via a ligand structure prediction system (e.g., CHARMM, AMBER, or GROMACS); optimization of known ligands (e.g., Compound 1) by evaluating binding of proposed analogs within the binding cavity of the biomolecular target, and combinations of the preceding.

Structures of compounds (e.g., Compound 1) and biomolecular targets (e.g. RyR2) provided herein can be used in computational methods of predicting a docked position of a target ligand in a binding site of a biomolecule, such as the use of a computer to assist in predicting a docked position of a target ligand in a binding site of a biomolecule that is capable of undergoing an induced fit as disclosed in US20210193273A1, which is incorporated herein by reference in its entirety.

In some embodiments, the present disclosure provides a method for predicting a docked position of a target ligand in a binding site of a biomolecule, the method comprising:
receiving a template ligand-biomolecule structure, the template ligand-biomolecule structure comprising a template ligand docked in the binding site of the biomolecule;
comparing a pharmacophore model of the template ligand to a pharmacophore model of the target ligand;
overlapping the pharmacophore model of the target ligand with the pharmacophore model of the template ligand while the template ligand is in the binding site of the biomolecule; and
predicting the docked position of the target ligand in the binding site of the biomolecule based on a position of the pharmacophore model of the target ligand when overlapped with the pharmacophore model of the template ligand, wherein the biomolecule is a RY1&2 domain of RyR2, wherein the template ligand-biomolecule structure is obtained by a process comprising subjecting a complex of the biomolecule and the template ligand to single-particle cryogenic electron microscopy analysis.

In some embodiments, the RY1&2 domain comprises a structure according to TABLE 3. In some embodiments, the template ligand has a three-dimensional conformation according to TABLE 4. In some embodiments, the RY1&2 domain further comprises second binding site. In some embodiments, the second binding site is an ATP-binding site. In some embodiments, the RY1&2 domain further comprises a nucleoside-containing molecule. In some embodiments, the nucleoside-containing molecule is an ATP molecule. In some embodiments the target ligand cooperatively binds the RY1&2 domain with the ATP molecule. In some embodiments, the ATP molecule has a three-dimensional conformation according to TABLE 5. In some embodiments, the target ligand cooperatively binds the RY1&2 domain with the ATP molecule.

In some embodiments, the target ligand is a compound of Formula (I), (I-a), (I-b), (I-c), (I-d), (I-e), (I-f), (I-g), (I-h), (I-i), (I-j), (I-k), (I-k-1), (I-1), (I-1-1), (I-m), (I-m-1), (I-n), (I-o), (I-p), (II) or (III). In some embodiments, the target ligand and the template ligand are each independently a compound of Formula (I), (I-a), (I-b), (I-c), (I-d), (I-e), (I-f), (I-g), (I-h), (I-i), (I-j), (I-k), (I-k-1), (I-1), (I-1-1), (I-m), (I-m-1), (I-n), (I-o), (I-p), (II) or (III).

In some embodiments, the template ligand is

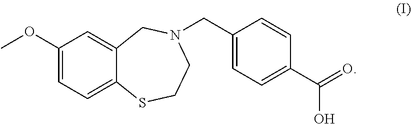 [SMILES: COc1ccc2c(c1)CN(Cc1ccc(C(=O)O)cc1)CCS2]

In some embodiments, the template ligand is

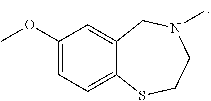

In some embodiments, the template ligand-biomolecule structure obtained by single-particle cryogenic electron microscopy analysis has a resolution from about 2 Å to about 3.5 Å, from about 2 Å to about 3.4 Å, from about 2 Å to about 3.3 Å, from about 2 Å to about 3.2 Å, from about 2 Å to about 3.1 Å, from about 2 Å to about 3 Å, from about 2 Å to about 2.9 Å, from about 2 Å to about 2.8 Å, from about 2 Å to about 2.7 Å, from about 2 Å to about 2.6 Å, from about 2 Å to about 2.5 Å, from about 2.1 Å to about 2.5 Å, from about 2.2 Å to about 2.5 Å, from about 2.3 Å to about 2.5 Å, or from about 2.4 Å to about 2.5 Å.

In some embodiments, the method further comprises selecting the target ligand from a plurality of ligand candidates, each of the ligand candidates being different from the template ligand, and wherein selecting the target ligand comprises comparing the pharmacophore model of the template ligand to a pharmacophore model of each respective one of the plurality of ligand candidates.

In some embodiments, the method further comprises receiving a plurality of template ligand-biomolecule structures, each template ligand-biomolecule structure having a different template ligand docked in the binding site of the biomolecule, and generating the pharmacophore model of the template ligand by combining information from each of the template ligands from the plurality of template ligand-biomolecule structures.

In some embodiments, the target ligand has more than one structural conformation in the unbound state, and the docked position of the target ligand in the binding site of the biomolecule is predicted by enumerating a set of potential target ligand conformations and overlapping a respective pharmacophore model of the target ligand for each of the potential target ligand conformations with the pharmacophore model of the template ligand while the template ligand is in the binding site of the biomolecule.

In some embodiments, predicting the docked position of the target ligand in the binding site of the biomolecule comprises ignoring at least one clash between the target ligand conformation's atomic coordinates and the biomolecule's atomic coordinates.

In some embodiments, the method further comprises, for each target ligand conformation, modifying atomic coordinates of the biomolecule to reduce clashes between the docked target ligand conformation's atomic coordinates and the biomolecule's atomic coordinates, thereby creating an altered ligand-biomolecule structure comprising the docked target ligand and an altered biomolecule.

In some embodiments, the method further comprises predicting a re-docked position of each target ligand conformation by predicting each target ligand conformation's position in the binding site of the altered biomolecule; and for each target ligand conformation, modifying atomic coordinates of the altered biomolecule to reduce clashes between the atomic coordinates of the target ligand conformation's re-docked position and the atomic coordinates of the altered biomolecule, thereby creating are-altered ligand-biomolecule structure comprising a re-docked target ligand and a re-altered biomolecule.

In some embodiments, the method further comprises ranking each altered and re-altered ligand-biomolecule structure using a scoring function.

In some embodiments, the method further comprises identifying a subset of high-ranking target ligands corresponding to target ligands having a threshold value for an empirical activity.

Structures of compounds (e.g., Compound 1) and biomolecular targets (e.g. RyR2) provided herein can be used in systems, devices, and methods that can generate lead compounds on the basis of known structure and activity of a lead compound (e.g., Compound 1) and the structure of a binding site for the lead compound, such as the systems, devices, and methods provided in US20210217500A1, which is incorporated herein by reference in its entirety.

In some embodiments, the present disclosure provides a method of identifying a plurality of potential lead compounds, the method comprising the steps of:
(a) analyzing, using a computer system, an initial lead compound known to bind to a biomolecular target, the analyzing comprising partitioning, by providing a database of known reactions, the initial lead compound into atoms defining partitioned lead compound comprising a lead compound core and atoms defining a lead compound non-core, wherein the initial lead compound is partitioned using a computational retrosynthetic analysis of the initial lead compound;
(b) identifying, using the computer system, a plurality of alternative cores to replace the lead compound core in the initial lead compound, thereby generating a plurality of potential lead compounds each having a respective one of the plurality of alternative cores;
(c) calculating, using the computer system, a difference in binding free energy between the partitioned lead compound and each potential lead compound;
(d) predicting, using the computer system, whether each potential lead compound binds to the biomolecular target and identifying a predicted active set of potential lead compounds based on the prediction;
(e) obtaining a synthesized set of at least some of the potential leads of the predicted active set to establish a first of potential lead compounds; and
(f) determining, empirically, an activity of each of the first set of synthesized potential lead compounds,
wherein the biomolecular target is a RY1&2 domain of RyR2, and the structure of the biomolecular target used in the predicting of (d) is obtained by a process comprising subjecting a complex of the biomolecular target and the initial lead compound to single-particle cryogenic electron microscopy analysis.

In some embodiments, the structure of the biomolecular target obtained by single-particle cryogenic electron microscopy analysis has a resolution from about 2 Å to about 3.5 Å, from about 2 Å to about 3.4 Å, from about 2 Å to about 3.3 Å, from about 2 Å to about 3.2 Å, from about 2 Å to about 3.1 Å, from about 2 Å to about 3 Å, from about 2 Å to about 2.9 Å, from about 2 Å to about 2.8 Å, from about 2 Å to about 2.7 Å, from about 2 Å to about 2.6 Å, from about 2 Å to about 2.5 Å, from about 2.1 Å to about 2.5 Å, from about 2.2 Å to about 2.5 Å, from about 2.3 Å to about 2.5 Å, or from about 2.4 Å to about 2.5 Å.

In some embodiments, the initial lead compound is a compound of Formula (I), (I-a), (I-b), (I-c), (I-d), (I-e), (I-f), (I-g), (I-h), (I-i), (I-j), (I-k), (I-k-1), (I-1), (I-1-1), (I-m), (I-m-1), (I-n), (I-o), (I-p), (II) or (III).

In some embodiments, the initial lead compound is

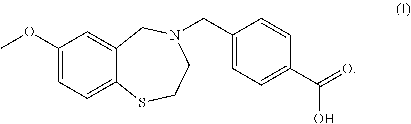 [SMILES: COc1ccc2c(c1)CN(Cc1ccc(C(=O)O)cc1)CCS2]

In some embodiments, the initial lead compound is

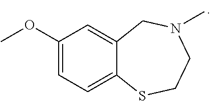

In some embodiments, the RY1&2 domain comprises a structure according to TABLE 3. In some embodiments, the RY1&2 domain contains an ATP molecule. In some embodiments, the ATP molecule has a three-dimensional conformation according to TABLE 5.

In some embodiments, the method further comprises obtaining a synthesized set of at least some of the potential lead compounds predicted to not bind with the biomolecular target to establish a second set of potential lead compounds and empirically determining an activity of each of the second set of synthesized potential lead compounds.

In some embodiments, the method further comprises comparing the empirically determined activity of each of the first set of synthesized potential lead compounds with a threshold activity level.

In some embodiments, the method further comprises comparing the empirically determined activity of each of the second set of synthesized potential lead compounds with a pre-determined activity level.

In some embodiments, the plurality of alternative cores are chosen from a database of synthetically feasible cores.

In some embodiments, the difference in binding free energy is calculated using a free energy perturbation technique.

In some embodiments, the generation of at least one potential lead compound comprises creating an additional covalent bond or annihilating an existing covalent bond, or both creating an additional first covalent bond and annihilating an existing second covalent bond different from the first covalent bond.

In some embodiments, the free energy perturbation technique uses a soft bond potential to calculate a bonded stretch interaction energy of existing covalent bonds for annihilation and additional covalent bonds for creation.

In some embodiments, the present disclosure provides a method for pharmaceutical drug discovery, comprising:
identifying an initial lead compound for binding to a biomolecular target;
using a method to identify a predicted active set of potential lead compounds for binding to the biomolecular target based on the initial lead compound, comprising:
   (a) analyzing, using a computer system, an initial lead compound known to bind to a biomolecular target, the analyzing comprising partitioning, by providing a database of known reactions, the initial lead compound into atoms defining partitioned lead compound comprising a lead compound core and atoms defining a lead compound non-core, wherein the initial lead compound is partitioned using a computational retrosynthetic analysis of the initial lead compound;
   (b) identifying, using the computer system, a plurality of alternative cores to replace the lead compound core in the initial lead compound, thereby generating a plurality of potential lead compounds each having a respective one of the plurality of alternative cores;
   (c) calculating, using the computer system, a difference in binding free energy between the partitioned lead compound and each potential lead compound;
   (d) predicting, using the computer system, whether each potential lead compound binds to the biomolecular target and identifying a predicted active set of potential lead compounds based on the prediction;
   (e) obtaining a synthesized set of at least some of the potential leads of the predicted active set to establish a first of potential lead compounds; and
   (f) determining, empirically, an activity of each of the first set of synthesized potential lead compounds,
   (g) selecting one or more of the predicted active set of potential lead compounds for synthesis; and
   (h) assaying the one or more synthesized selected compounds to assess each synthesized selected compounds suitability for in vivo use as a pharmaceutical compound,
wherein the biomolecular target is a RY1&2 domain of RyR2, and the structure of the biomolecular target used in the predicting of (d) is obtained by a process comprising subjecting a complex of the biomolecular target and the initial lead compound to single-particle cryogenic electron microscopy analysis.

In some embodiments, the structure of the biomolecular target obtained by single-particle cryogenic electron microscopy analysis has a resolution from about 2 Å to about 3.5 Å, from about 2 Å to about 3.4 Å, from about 2 Å to about 3.3 Å, from about 2 Å to about 3.2 Å, from about 2 Å to about 3.1 Å, from about 2 Å to about 3 Å, from about 2 Å to about 2.9 Å, from about 2 Å to about 2.8 Å, from about 2 Å to about 2.7 Å, from about 2 Å to about 2.6 Å, from about 2 Å to about 2.5 Å, from about 2.1 Å to about 2.5 Å, from about 2.2 Å to about 2.5 Å, from about 2.3 Å to about 2.5 Å, or from about 2.4 Å to about 2.5 Å.

In some embodiments, the initial lead compound is a compound of Formula (I), (I-a), (I-b), (I-c), (I-d), (I-e), (I-f), (I-g), (I-h), (I-i), (I-j), (I-k), (I-k-1), (I-1), (I-1-1), (I-m), (I-m-1), (I-n), (I-o), (I-p), (II) or (III).

In some embodiments, the initial lead compound is

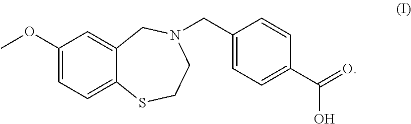 [SMILES: COc1ccc2c(c1)CN(Cc1ccc(C(=O)O)cc1)CCS2]

In some embodiments, the initial lead compound is

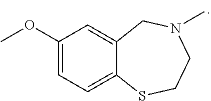

In some embodiments, the RY1&2 domain comprises a structure according to TABLE 3. In some embodiments, the RY1&2 domain contains an ATP molecule. In some embodiments, the ATP molecule has a three-dimensional conformation according to TABLE 5.

Structures of compounds (e.g., Compound 1) and biomolecular targets (e.g. RyR2) provided herein can be used in methods that estimate binding affinity between a ligand and a receptor molecule, including the systems and methods disclosed in U.S. Pat. No. 8,160,820B2, which is incorporated by reference herein in its entirety.

In some embodiments, the present disclosure provides a computer-implemented method of quantifying binding affinity between a ligand and a receptor molecule domain, the method comprising:
receiving by one or more computers, data representing a ligand molecule,
receiving by one or more computers, data representing a receptor molecule domain,
using the data representing the ligand molecule and the data representing the receptor molecule domain in computer analysis to identify ring structure within the ligand, the ring structure being an entire ring or a fused ring;
using the data representative of the identified ligand ring structure to designate a first ring face and a second ring face opposite to the first ring face, and classifying the ring structure by:
a) determining proximity of receptor atoms to atoms on the first face of the ligand ring; and
b) determining proximity of receptor atoms to atoms on the second face of the ligand ring;
c) determining solvation of the first face of the ligand ring and solvation of the second face of the ligand ring;
classifying the identified ligand ring structure as buried, solvent exposed, or having a single face exposed to solvent based on receptor atom proximity to and solvation of the first ring face and receptor atom proximity to and solvation of the second ring face; quantifying the binding affinity between the ligand and the receptor molecule domain based at least in part on the classification of the ring structure; and
displaying, via computer, information related to the classification of the ring structure, wherein the receptor molecule domain is a RY1&2 domain of RyR2, wherein the data representing a ligand molecule and the data representing a receptor molecule domain are obtained by a process comprising subjecting a complex comprising the ligand molecule and the receptor molecule domain to single-particle cryogenic electron microscopy analysis.

In some embodiments, the structure of the receptor molecule domain obtained by single-particle cryogenic electron microscopy analysis has a resolution from about 2 Å to about 3.5 Å, from about 2 Å to about 3.4 Å, from about 2 Å to about 3.3 Å, from about 2 Å to about 3.2 Å, from about 2 Å to about 3.1 Å, from about 2 Å to about 3 Å, from about 2 Å to about 2.9 Å, from about 2 Å to about 2.8 Å, from about 2 Å to about 2.7 Å, from about 2 Å to about 2.6 Å, from about 2 Å to about 2.5 Å, from about 2.1 Å to about 2.5 Å, from about 2.2 Å to about 2.5 Å, from about 2.3 Å to about 2.5 Å, or from about 2.4 Å to about 2.5 Å.

In some embodiments, ligand molecule is a compound of Formula (I), (I-a), (I-b), (I-c), (I-d), (I-e), (I-f), (I-g), (I-h), (I-i), (I-j), (I-k), (I-k-1), (I-1), (I-1-1), (I-m), (I-m-1), (I-n), (I-o), (I-p), (II) or

In some embodiments, the ligand molecule is

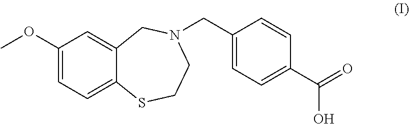 [SMILES: COc1ccc2c(c1)CN(Cc1ccc(C(=O)O)cc1)CCS2]

or a pharmaceutically-acceptable salt or ionized form thereof.

In some embodiments, the ligand molecule is

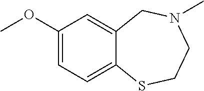

or a pharmaceutically-acceptable salt or ionized form thereof.

In some embodiments, the complex further comprises a RyR2 protein, wherein the RY1&2 domain is a domain of the RyR2 protein.

In some embodiments, the data representing the receptor molecule domain represents a three-dimensional structure of the receptor molecule according to TABLE 3. In some embodiments, the data representing a ligand molecule represents a three-dimensional structure of the ligand molecule according to TABLE 4.

In some embodiments, the receptor molecule domain contains an ATP molecule. In some embodiments, the data representing the receptor molecule domain further comprises data representing a three-dimensional structure of the ATP molecule according to TABLE 5.

In some embodiments, quantifying the binding affinity includes a step that scores hydrophobic interactions between one or more ligand atoms and one or more receptor atoms by awarding a bonus for the presence of hydrophobic enclosure of one or more atoms of said ligand by the receptor molecule domain, said bonus being indicative of enhanced binding affinity between said ligand and said receptor molecule domain.

In some embodiments, the method further comprises calculating an initial binding affinity and then adjusting the initial binding affinity based on the classification of the ring structure as buried, solvent exposed, or solvent exposed on one face.

In some embodiments, the classification of a ring structure as buried, solvent exposed, or solvent exposed on one surface, includes using a parameter substantially correlated with the number of close contacts on both sides of the ring structure or part thereof with the receptor molecule domain.

In some embodiments, the number of close contacts at different distances between receptor atoms and the two ring faces are determined, an initial classification of the ring is made based on the numbers of these contacts, and this initial classification is then followed by calculation of a scoring function, said scoring function comprising identifying a first ring shell and a second ring shell, and calculating the number of water molecules in the first shell and in the second shell, or calculating the number of water molecules in the first and second shell combined.

In some embodiments, the scoring function for classification of the ring structure as buried, solvent exposed, or solvent exposed on one surface, includes using a parameter substantially correlated with the lipophilic-lipophilic pair score between the ring structure or part thereof and the receptor molecule domain.

In some embodiments, the scoring function used to classify a ring structure as buried, solvent exposed, or solvent exposed on one surface, includes calculating the degree of enclosure of each atom of the ring structure by atoms of the receptor.

In some embodiments, the scoring function used to classify a ring structure as buried, solvent exposed, or solvent exposed on one surface, includes using a parameter that is substantially correlated with the degree of enclosure of each atom of the ring structure by atoms of the receptor.

In some embodiments, the scoring function enabling classification of the ring structure as buried, solvent exposed, or solvent exposed on one surface, includes the use of a parameter corresponding to a hydrophobic interaction of the ring structure or part thereof with the receptor molecule domain.

In some embodiments, the information displayed by computer includes a depiction of at least one of the degree to which the ring structure is enclosed by atoms of the receptor molecule domain; water molecules surrounding the ring structure in a first shell or a second shell or both the first and the second shell of the ligand; a value of a lipophilic-lipophilic pair score of the ring structure; and a number of close contacts of a face of the ring structure with the receptor molecule domain.

In some embodiments, solvent exposed ring structures in the ligand, if any, are substantially ignored in quantifying the component of the binding affinity between the ligand and the receptor molecule domains, other than to recognize hydrogen bonds and other parameters that are independent of the classification of ring structure.

In some embodiments, hydrophobic contribution to binding affinity from ring structures classified as solvent exposed, if any, is substantially ignored in quantifying the component of the binding affinity.

In some embodiments, a ring structure is classified as buried, and the method further comprises identifying a quantity representative of a strain energy induced in the ligand-receptor complex by the buried ring structure, in which the quantification of the component of binding affinity is further based in part on strain energy.

In some embodiments, the method further comprises identifying a quantity representative of a strain energy induced in the ligand-receptor complex by the aggregate of the ring structures identified as buried; identifying a quantity representative of a total neutral-neutral hydrogen bond energy; and quantifying the component of binding affinity between the ligand and the receptor molecule domain based at least in part on the quantity representative of the strain energy induced in the receptor by the aggregate of the buried ring structures, and on the quantity representative of the total neutral-neutral hydrogen bond energy.

In some embodiments, quantifying the component of binding affinity further comprises identifying a hydrogen bond capping energy associated with the entire ligand, and the component of binding affinity is quantified based on a greater of the hydrogen bond capping energy and the quantity representative of the strain energy induced in the receptor by the aggregate of the identified structures.

In some embodiments, the method further comprises identifying a binding motif of the receptor molecule domain with respect to the ligand; identifying a reorganization energy of the receptor molecule domain based on the binding motif, and identifying a first ring structure as contributing to the reorganization energy, the quantity representative of strain energy being identified independently of the classification of the first ring structure.

In some embodiments, the component of binding affinity attributable to strain is quantified using at least one of: molecular dynamics, molecule mechanics, conformational searching and minimization.

In some embodiments, the information displayed by computer includes a depiction of solvent exposure, if any, of the ring structure.

In some embodiments, the information displayed by computer includes a depiction of burial, if any, of the ring structure.

In some embodiments, the information displayed by computer includes a depiction of at least one of: the degree to which the ring structure is enclosed by atoms of the receptor molecule domain; water molecules surrounding the ring structure in a first shell or a second shell or both the first and the second shell of the ligand; a value of a lipophilic-lipophilic pair score of the ring structure; and a number of close contacts of a face of the ring structure with the receptor molecule domain.

In some embodiments, the method further comprises performing a test on a physical sample that includes the ligand and the receptor molecule domain, test components being selected based at least in part on the binding affinity between the ligand or part thereof and the receptor molecule, or on the component of such binding affinity.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively, or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which is also referred to or described as a program, software, a software application, an app, a module, a software module, a script, or code, can be written in any form of programming language, including compiled or interpreted languages, or declarative or procedural languages; and can be deployed in any form, including as a stand-alone program or as a module, component, subroutine, or other unit suitable for use in a computing environment. A program can, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit receives instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer can also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

### EMBODIMENTS

The following non-limiting embodiments provide illustrative examples of the invention, but do not limit the scope of the invention.
Embodiment 1. A composition comprising a mixture of water and a protein, wherein the protein is a ryanodine receptor 2 protein (RyR2) or a mutant thereof.
Embodiment 2. The composition of embodiment 1, further comprising a synthetic compound.
Embodiment 3. The composition of embodiment 1 or embodiment 2, further comprising calmodulin.
Embodiment 4. The composition of embodiments 3, wherein the calmodulin is human calmodulin.
Embodiment 5. The composition of embodiment 3 or embodiment 4, wherein calmodulin is present in the mixture at a concentration from about 1 μM to about 200 μM.
Embodiment 6. The composition of embodiment 3 or embodiment 4, wherein calmodulin is present in the mixture at a concentration from about 1 μM to about 60 μM.
Embodiment 7. The composition of embodiment 3 or embodiment 4, wherein calmodulin is present in the mixture at a concentration of about 20 μM.
Embodiment 8. The composition of embodiment 3 or embodiment 4, wherein calmodulin is present in the mixture at a concentration of about 40 μM.
Embodiment 9. The composition of any one of embodiments 1-8, further comprising a buffering agent in the mixture.
Embodiment 10. The composition of embodiment 9, wherein the buffering agent is HEPES.
Embodiment 11. The composition of embodiment 9, wherein the buffering agent is EGTA.
Embodiment 12. The composition of any one of embodiments 1-11, further comprising a phospholipid in the mixture.
Embodiment 13. The composition of embodiment 12, wherein the phospholipid is dioleoylphosphatidylcholine (DOPC).
Embodiment 14. The composition of embodiment 12, wherein the phospholipid is 1,2-dioleoyl-sn-glycero-3-phospho-L-serine sodium salt (DOPS).
Embodiment 15. The composition of any one of embodiments 1-14, further comprising a zwitterionic surfactant in the mixture.
Embodiment 16. The composition of embodiment 15, wherein the zwitterionic surfactant is CHAPS.
Embodiment 17. The composition of any one of embodiments 1-16, further comprising a disulfide-reducing agent in the mixture.
Embodiment 18. The composition of embodiment 17, wherein the disulfide-reducing agent is TCEP.
Embodiment 19. The composition of embodiment 17, wherein the disulfide-reducing agent is dithiothreitol.
Embodiment 20. The composition of any one of embodiments 1-18, further comprising a protease inhibitor in the mixture.
Embodiment 21. The composition of embodiment 20, wherein the protease inhibitor is AEBSF.
Embodiment 22. The composition of embodiment 20, wherein the protease inhibitor is benzamidine or a salt thereof.
Embodiment 23. The composition of any one of embodiments 1-22, further comprising xanthine in the mixture.
Embodiment 24. The composition of embodiment 23, wherein xanthine is present in the mixture at a concentration from about 300 μM to about 700 μM.
Embodiment 25. The composition of embodiment 23, wherein xanthine is present in the mixture at a concentration of about 500 μM.
Embodiment 26. The composition of any one of embodiments 1-25, further comprising dissolved Ca²⁺ in the mixture.
Embodiment 27. The composition of embodiment 26, wherein dissolved Ca²⁺ is present in the mixture at a concentration from about 15 nM to about 500 nM.
Embodiment 28. The composition of embodiment 26, wherein dissolved Ca²⁺ is present in the mixture at a concentration from about 100 nM to about 300 nM.
Embodiment 29. The composition of embodiment 26, wherein dissolved Ca²⁺ is present in the mixture at a concentration of about 150 nM.
Embodiment 30. The composition of any one of embodiments 1-29, wherein the protein is present in the mixture at a concentration from about 0.1 mg/mL to about 50 mg/mL.
Embodiment 31. The composition of any one of embodiments 1-29, wherein the protein is present in the mixture at a concentration from about 0.1 mg/mL to about 20 mg/mL.
Embodiment 32. The composition of any one of embodiments 1-29, wherein the protein is present in the mixture at a concentration from about 1 mg/mL to about 10 mg/mL.
Embodiment 33. The composition of any one of embodiments 1-32, further comprising sodium adenosine triphosphate (NaATP) in the mixture.
Embodiment 34. The composition of embodiment 33, wherein the NaATP is present in the mixture at a concentration from about 3 mM to about 15 nM.
Embodiment 35. The composition of embodiment 33, wherein the NaATP is present in the mixture at a concentration from about 10 mM.
Embodiment 36. The composition of any one of embodiments 1-35, further comprising cyclic adenosine monophosphate (cAMP) in the mixture.
Embodiment 37. The composition of embodiment 36, wherein the cAMP is present in the mixture at a concentration from about 50 μM to about 500 μM.
Embodiment 38. The composition of embodiment 36, wherein the cAMP is present in the mixture at a concentration from about 200 μM.
Embodiment 39. The composition of any one of embodiments 1-38, wherein the protein is human RyR2.
Embodiment 40. The composition of any one of embodiments 1-39, wherein the protein is mutant RyR2.
Embodiment 41. The composition of embodiment 40, wherein the mutant RyR2 is R2474S-RyR2.
Embodiment 42. The composition of any one of embodiments 1-41, wherein the protein is in a phosphorylated state, wherein the phosphorylated state is prepared by a process comprising contacting the RyR2 protein with a phosphorylation reagent.
Embodiment 43. The composition of embodiment 42, wherein the phosphorylation reagent comprises protein kinase A.
Embodiment 44. The composition of embodiment 43, wherein the phosphorylation reagent further comprises ATP.
Embodiment 45. The composition of embodiment 43 or embodiment 44, wherein the phosphorylation reagent further comprises MgCl₂.
Embodiment 46. The composition of any one of embodiments 1-41, wherein the protein is in a dephosphorylated state, wherein the dephosphorylated state is prepared by a process comprising contacting the RyR2 protein with a dephosphorylation reagent.
Embodiment 47. The composition of embodiment 46, wherein the dephosphorylation reagent comprises phosphatase lambda.
Embodiment 48. The composition of embodiment 47, wherein the dephosphorylation reagent further comprises MnCl₂.
Embodiment 49. The composition of any one of embodiments 1-48, wherein the composition is substantially free of cellular membrane.
Embodiment 50. A composition comprising a complex suspended in a solid medium, wherein the complex comprises a protein, wherein the protein is a ryanodine receptor 2 protein (RyR2) or mutant thereof.
Embodiment 51. The composition of embodiment 50, wherein the composition is prepared by a process, the process comprising vitrifying an aqueous solution that is applied to an electron microscopy grid, wherein the aqueous solution comprises the complex.
Embodiment 52. The composition of embodiment 51, wherein, prior to the vitrifying, the aqueous solution is applied to the electron microscopy grid, and excess aqueous solution is removed from the electron microscopy grid by blotting the excess aqueous solution.
Embodiment 53. The composition of embodiment 51 or embodiment 52, wherein the vitrifying comprises plunge freezing the aqueous solution applied to the electron microscopy grid into liquid ethane chilled with liquid nitrogen.
Embodiment 54. The composition of any one of embodiments 51-53, wherein the aqueous solution further comprises a buffering agent.
Embodiment 55. The composition of embodiment 54, wherein the buffering agent is HEPES.
Embodiment 56. The composition of embodiment 54, wherein the buffering agent is EGTA.
Embodiment 57. The composition of any one of embodiments 51-56, wherein the aqueous solution further comprises a phospholipid.
Embodiment 58. The composition of embodiment 57, wherein the phospholipid is DOPS.
Embodiment 59. The composition of embodiment 57, wherein the phospholipid is DOPC.
Embodiment 60. The composition of any one of embodiments 51-59, wherein the aqueous solution further comprises a zwitterionic surfactant.
Embodiment 61. The composition of embodiment 60, wherein the zwitterionic surfactant is CHAPS.
Embodiment 62. The composition of any one of embodiments 51-61, wherein the aqueous solution further comprises a disulfide-reducing agent.
Embodiment 63. The composition of embodiment 62, wherein the disulfide-reducing agent is TCEP.
Embodiment 64. The composition of embodiment 62, wherein the disulfide-reducing agent is dithiothreitol.
Embodiment 65. The composition of any one of embodiments 51-64, wherein the aqueous solution further comprises a protease inhibitor.
Embodiment 66. The composition of embodiment 65, wherein the protease inhibitor is AEBSF.
Embodiment 67. The composition of embodiment 65, wherein the protease inhibitor is benzamidine or a salt thereof.
Embodiment 68. The composition of any one of embodiments 51-67, wherein the aqueous solution further comprises xanthine.
Embodiment 69. The composition of embodiment 68, wherein the xanthine is present in the aqueous solution at a concentration from about 300 μM to about 700 μM.
Embodiment 70. The composition of embodiment 68, wherein the xanthine is present in the aqueous solution at a concentration of about 500 μM.
Embodiment 71. The composition of any one of embodiments 51-70, wherein the aqueous solution further comprises dissolved Ca²⁺.
Embodiment 72. The composition of embodiment 71, wherein the dissolved Ca²⁺ is present in the aqueous solution at a concentration from about 15 μM to about 500 μM.
Embodiment 73. The composition of embodiment 71, wherein the dissolved Ca²⁺ is present in the aqueous solution at a concentration from about 100 nM to about 300 nM.
Embodiment 74. The composition of embodiment 71, wherein the dissolved Ca²⁺ is present in the aqueous solution at a concentration of about 150 nM.
Embodiment 75. The composition of any one of embodiments 50-74, wherein the protein is present in the aqueous solution at a concentration from about 0.1 mg/mL to about 50 mg/mL.
Embodiment 76. The composition of any one of embodiments 50-74, wherein the protein is present in the aqueous solution at a concentration from about 0.1 mg/mL to about 20 mg/mL.
Embodiment 77. The composition of any one of embodiments 50-74, wherein the protein is present in the aqueous solution at a concentration from about 0.1 mg/mL to about 10 mg/mL.
Embodiment 78. The composition of any one of embodiments 51-77, wherein the aqueous solution further comprises sodium adenosine triphosphate (NaATP).
Embodiment 79. The composition of embodiment 78, wherein the NaATP is present at a concentration from about 3 mM to about 15 nM.
Embodiment 80. The composition of embodiment 78, wherein the NaATP is present at a concentration of about 10 mM.
Embodiment 81. The composition of any one of embodiments 51-80, further comprising cyclic adenosine monophosphate (cAMP).
Embodiment 82. The composition of embodiment 81, wherein the cAMP is present in the aqueous solution at a concentration from about 50 μM to about 500 μM.
Embodiment 83. The composition of embodiment 81, wherein the cAMP is present in the aqueous solution at a concentration from about 200 μM.
Embodiment 84. The composition of any one of embodiments 51-83, wherein the aqueous solution further comprises calmodulin.
Embodiment 85. The composition of embodiment 84, wherein the calmodulin is human calmodulin.
Embodiment 86. The composition of embodiment 84 or embodiment 85, wherein calmodulin is present in the aqueous solution at a concentration from about 1 μM to about 200 μM.
Embodiment 87. The composition of embodiment 84 or embodiment 85, wherein calmodulin is present in the aqueous solution at a concentration from about 1 μM to about 60 μM.
Embodiment 88. The composition of embodiment 84 or embodiment 85, wherein calmodulin is present in the aqueous solution at a concentration of about 20 μM.
Embodiment 89. The composition of embodiment 84 or embodiment 85, wherein calmodulin is present in the aqueous solution at a concentration of about 40 μM.
Embodiment 90. The composition of any one of embodiments 50-77, wherein the complex further comprises calmodulin.
Embodiment 91. The composition of embodiment 90, wherein the calmodulin is human calmodulin.
Embodiment 92. The composition of any one of embodiments 50-91, wherein the complex further comprises calstabin.
Embodiment 93. The composition of embodiment 92, wherein the calstabin is calstabin-2.
Embodiment 94. The composition of embodiment 92, wherein the calstabin is human calstabin.
Embodiment 95. The composition of any one of embodiments 50-94, wherein the complex further comprises a xanthine molecule.
Embodiment 96. The composition of any one of embodiments 50-95, wherein the complex further comprises a Ca²⁺ ion.
Embodiment 97. The composition of any one of embodiments 50-96, wherein the RyR2 protein is in the closed state.
Embodiment 98. The composition of any one of embodiments 50-97, wherein the composition is substantially free of cellular membrane.
Embodiment 99. The composition of any one of embodiments 50-98, wherein the solid medium comprises vitreous ice.
Embodiment 100. The composition of embodiment 99, wherein the solid medium is substantially free of crystalline ice.
Embodiment 101. The composition of any one of embodiments 50-100, wherein the RyR2 protein is in a closed state.
Embodiment 102. The composition of any one of embodiments 50-100, wherein the RyR2 protein is in an open state.
Embodiment 103. The composition of any one of embodiments 50-100, wherein the RyR2 protein is in a primed state, wherein if the RyR2 protein is placed in a physiological medium, then the RyR2 protein in the primed state has an open probability (Pₒ) that is higher than an open probability (Pₒ) of the RyR2 in a closed state, and an open probability (Pₒ) that is lower than an open probability (Pₒ) of the RyR2 protein in an open state.
Embodiment 104. The composition of any one of embodiments 50-100, wherein the composition further comprises additional complexes, wherein each of the additional complexes independently comprises the protein.
Embodiment 105. The composition of embodiment 104, wherein at least about 50%, at least about 60%, at least about 70%, at least about 80%, or at least about 90% of the additional complexes are in the closed state.
Embodiment 106. The composition of any one of embodiments 50-100, wherein the protein is R2474S-RyR2, wherein if a study is conducted, the study comprising:
   (i) determining a structure of a BSol2 domain of the protein in the complex by subjecting the complex to single particle cryogenic electron microscopy analysis, and
   (ii) calculating root mean square deviation of atomic positions (RMSD) of the BSol2 domain of the protein relative to a BSol2 domain of a reference structure, wherein the reference structure is a structure of a wild type RyR2 protein in a closed state,
then the RMSD is no more than about 4.5, no more than about 4, no more than about 3.5, or no more than about 3.
Embodiment 107. The composition of any one of embodiments 50-100, wherein the protein is RyR2-R2474S, wherein if a study is conducted, the study comprising:
   (i) determining a structure of a BSol2 domain of the protein in the complex by subjecting the complex to single particle cryogenic electron microscopy analysis, and
   (ii) calculating root mean square deviation of atomic positions (RMSD) of the BSol2 domain of the protein relative to a BSol2 domain of a reference structure, wherein the reference structure is a structure of a wild type RyR2 protein in a closed state,
   then the RMSD is from about 1 to about 4.5, about 1 to about 4, about 1 to about 3.5, about 1 to about 3, about 2 to 4.5, about 2 to about 4, about 2 to about 3.5, or about 2 to about 3. 103981 Embodiment 108. The composition of any one of embodiments 50-100, wherein the protein is RyR2-R2474S, wherein if a study is conducted, the study comprising:
   (i) determining a structure of a BSol domain of the protein in the complex by subjecting the complex to single particle cryogenic electron microscopy analysis, and
   (ii) calculating root mean square deviation of atomic positions (RMSD) of the BSol domain of the protein relative to a BSol domain of a reference structure, wherein the reference structure is a structure of a wild type RyR2 protein in a closed state,
   then the RMSD is no more than about 3, no more than about 2.5, no more than about 2, or no more than about 1.5.
Embodiment 109. The composition of any one of embodiments 50-100, wherein the protein is RyR2-R2474S, wherein if a study is conducted, the study comprising:
   (i) determining a structure of a BSol domain of the protein in the complex by subjecting the complex to single particle cryogenic electron microscopy analysis, and
   (ii) calculating root mean square deviation of atomic positions (RMSD) of the BSol domain of the protein relative to a BSol domain of a reference structure, wherein the reference structure is a structure of a wild type RyR2 protein in a closed state,
   then the RMSD is from about 0.5 to about 3, about 0.5 to about 2.5, about 0.5 to about 2, about 0.5 to about 1.5, about 1 to 3, about 1 to about 2.5, about 1 to about 2, or about 1 to about 1.5.
Embodiment 110. The composition of any one of embodiments 50-100, wherein the protein is RyR2-R2474S, wherein if a study is conducted, the study comprising:
   (i) determining a structure of a NTD domain of the protein in the complex by subjecting the complex to single particle cryogenic electron microscopy analysis, and
   (ii) calculating root mean square deviation of atomic positions (RMSD) of the NTD domain of the protein relative to a NTD domain of a reference structure, wherein the reference structure is a structure of a wild type RyR2 protein in a closed state,
   then the RMSD is no more than about 1.5, no more than about 1.4, no more than about 1.3, no more than about 1.2, no more than about 1.1, or no more than about 1.
Embodiment 111. The composition of any one of embodiments 50-100, wherein the protein is RyR2-R2474S, wherein if a study is conducted, the study comprising:
   (i) determining a structure of a NTD domain of the protein in the complex by subjecting the complex to single particle cryogenic electron microscopy analysis, and
   (ii) calculating root mean square deviation of atomic positions (RMSD) of the NTD domain of the protein relative to a NTD domain of a reference structure, wherein the reference structure is a structure of a wild type RyR2 protein in a closed state,
   then the RMSD is from about 0.5 to about 1.6, about 0.5 to about 1.5, about 0.5 to about 1.4, about 0.5 to about 1.3, or about 0.5 to about 1.2.
Embodiment 112. The composition of any one of embodiments 50-100, wherein the protein is RyR2-R2474S, wherein if a study is conducted, the study comprising:
   (i) determining a structure of a SPRY domain of the protein in the complex by subjecting the complex to single particle cryogenic electron microscopy analysis, and
   (ii) calculating root mean square deviation of atomic positions (RMSD) of the SPRY domain of the protein relative to a SPRY domain of a reference structure, wherein the reference structure is a structure of a wild type RyR2 protein in a closed state,
   then the RMSD is less than about 1.2, less than about 1.1, or less than about 1, less than about 0.9, less than about 0.8, or less than about 0.7.
Embodiment 113. The composition of any one of embodiments 50-100, wherein the protein is RyR2-R2474S, wherein if a study is conducted, the study comprising:
   (i) determining a structure of a SPRY domain of the protein in the complex by subjecting the complex to single particle cryogenic electron microscopy analysis, and
   (ii) calculating root mean square deviation of atomic positions (RMSD) of the SPRY domain of the protein relative to a SPRY domain of a reference structure, wherein the reference structure is a structure of a wild type RyR2 protein in a closed state,
   then the RMSD is from about 0.2 to about 1.3, about 0.2 to about 1.2, about 0.2 to about 1.1, about 0.2 to about 1, or about 0.2 to about 0.9.
Embodiment 114. The composition of any one of embodiments 106-113, wherein the reference structure is a structure according to Protein Data Bank entry 7U9Q.
Embodiment 115. The composition of any one of embodiments 106-113, wherein the RMSD is calculated via a process, the process comprising calculating a difference in atomic positions between a structure of the protein and a structure of the wild type RyR2 protein in the closed state, wherein the structure of the protein is determined via cryogenic electronic microscopy analysis of the complex.
Embodiment 116. The composition of any one of embodiments 106-115, wherein the wild type RyR2 protein in the closed state has a structure according to Protein Data Bank entry 7U9Q.
Embodiment 117. The composition of any one of embodiments 50-105, wherein the protein is wild type RyR2.
Embodiment 118. The composition of any one of embodiments 50-105, wherein the protein is a mutant RyR2.
Embodiment 119. The composition of embodiment 118, wherein the mutant RyR2 contains at least one mutation that is associated with Catecholaminergic Polymorphic Ventricular Tachycardia (CPVT).
Embodiment 120. The composition of embodiment 119, wherein the mutation is RyR2-R2474S.
Embodiment 121. The composition of embodiment 119, wherein the mutation is RyR2-R420Q.
Embodiment 122. The composition of embodiment 119, wherein the mutation is RyR2-R420W.
Embodiment 123. The composition of any one of embodiments 119-122, wherein the mutation destabilizes an interaction between NTD and BSol domains of the RyR2 protein.
Embodiment 124. The composition of any one of embodiments 119-122, wherein the mutation destabilizes a cytosolic shell of the RyR2 protein, wherein the cytosolic shell comprises NTD, SPRY, JSol and BSol domains of the RyR2 proteins.
Embodiment 125. The composition of any one of embodiments 50-124, wherein the protein is a post-translationally modified RyR2 protein.
Embodiment 126. The composition of embodiment 125, wherein the post-translationally modified RyR2 protein is a phosphorylated, oxidized or nitrosylated RyR2.
Embodiment 127. The composition of embodiment 125 or 126, wherein the post-translationally modified RyR2 protein is a phosphorylated RyR2.
Embodiment 128. The composition of any one of embodiments 125-127, wherein the post-translationally modified RyR2 protein is associated with heart failure.
Embodiment 129. The composition of any one of embodiments 125-128, wherein the post-translational modification destabilizes an interaction between NTD and BSol domains of the RyR2 protein.
Embodiment 130. The composition of any one of embodiments 125-128, wherein the post-translational modification destabilizes a cytosolic shell of the RyR2 protein, wherein the cytosolic shell comprises NTD, SPRY, JSol and BSol domains of the RyR2 proteins.
Embodiment 131. The composition of any one of embodiments 105-130, wherein the RyR2 is a mutated and post-translationally modified RyR2.
Embodiment 132. The composition of any one of embodiments 50-131, wherein the protein is human RyR2.
Embodiment 133. The composition of any one of embodiments 50-132, wherein the protein is in a phosphorylated state, wherein the phosphorylated state is prepared by a process comprising contacting RyR2 protein with a phosphorylation reagent.
Embodiment 134. The composition of embodiment 133, wherein the phosphorylation reagent comprises protein kinase A.
Embodiment 135. The composition of embodiment 134, wherein the phosphorylation reagent further comprises ATP.
Embodiment 136. The composition of embodiment 133 or embodiment 134, wherein the phosphorylation reagent further comprises MgCl₂.
Embodiment 137. The composition of any one of embodiments 50-132, wherein the protein is in a dephosphorylated state, wherein the dephosphorylated state is prepared by a process comprising contacting RyR2 protein with a dephosphorylation reagent.
Embodiment 138. The composition of embodiment 137, wherein the dephosphorylation reagent comprises phosphatase lambda.
Embodiment 139. The composition of embodiment 138, wherein the dephosphorylation reagent further comprises MnCl₂.
Embodiment 140. The composition of any one of embodiments 1-105, wherein the protein is a tetramer of RyR2 monomers, wherein each RyR2 monomer is SEQ ID NO: 3.
Embodiment 141. The composition of any one of embodiments 1-105, wherein the protein is a tetramer of RyR2 monomers, wherein each RyR2 monomer is SEQ ID NO: 4.
Embodiment 142. The composition of any one of embodiments 50-141, wherein the complex further comprises a nucleoside-containing molecule.
Embodiment 143. The composition of embodiment 142, wherein the nucleoside-containing molecule is a purine nucleoside-containing molecule.
Embodiment 144. The composition of embodiment 142 or embodiment 143, wherein the nucleoside-containing molecule is a nucleotide or nucleoside polyphosphate.
Embodiment 145. The composition of any one of embodiments 142-144, wherein the nucleoside-containing molecule is an adenosine triphosphate (ATP) molecule.
Embodiment 146. The composition of embodiment 145, wherein the ATP molecule has a three-dimensional conformation according to TABLE 5.
Embodiment 147. The composition of embodiment 142 or embodiment 143, wherein the nucleoside-containing molecule is an adenosine diphosphate (ATP) molecule.
Embodiment 148. The composition of embodiment 147, wherein the complex further comprises a second ATP molecule, wherein both ATP molecules bind a common RYR domain of the protein.
Embodiment 149. The composition of embodiment 142, wherein the complex further comprises a second nucleoside-containing molecule.
Embodiment 150. The composition of embodiment 149, wherein the second nucleoside-containing molecule binds a C-terminal domain of the RyR2 protein.
Embodiment 151. The composition of embodiment 149 or embodiment 150, wherein the second nucleoside-containing molecule is a nucleotide or nucleoside polyphosphate.
Embodiment 152. The composition of any one of embodiments 149-151, wherein the second nucleoside-containing molecule is a second ATP molecule.
Embodiment 153. The composition of any one of embodiments 50-140, further comprising a synthetic compound.
Embodiment 154. The composition of embodiment 153, wherein the complex further comprises a nucleoside-containing molecule.
Embodiment 155. The composition of embodiment 154, wherein the nucleoside-containing molecule and the synthetic compound bind a RYR domain of the protein.
Embodiment 156. The composition of embodiment 155, wherein the RYR domain is a RY1&2 domain.
Embodiment 157. The composition of embodiment 156, wherein the RY1&2 domain has a three-dimensional structure according to TABLE 3.
Embodiment 158. The composition of any one of embodiments 154-157, wherein the nucleoside-containing molecule is a purine nucleoside-containing molecule.
Embodiment 159. The composition of any one of embodiments 154-158, wherein the nucleoside-containing molecule is a nucleotide or nucleoside polyphosphate.
Embodiment 160. The composition of any one of embodiments 154-159, wherein the nucleoside-containing molecule is an adenosine triphosphate (ATP) molecule.
Embodiment 161. The composition of embodiment 160, wherein the ATP molecule has a three-dimensional conformation according to TABLE 5.
Embodiment 162. The composition of embodiment 160 or embodiment 161, wherein the ATP molecule is cooperatively bound to the protein with the synthetic compound.
Embodiment 163. The composition of any one of embodiments 160-162, wherein the complex further comprises a second ATP molecule, wherein both ATP molecules bind a common RYR domain of the protein.
Embodiment 164. The composition of any one of embodiments 154-159, wherein the complex further comprises a second nucleoside-containing molecule.
Embodiment 165. The composition of embodiment 164, wherein the second nucleoside-containing molecule binds a C-terminal domain of the RyR2 protein.
Embodiment 166. The composition of embodiment 164, wherein the second nucleoside-containing molecule is a nucleotide or nucleoside polyphosphate.
Embodiment 167. The composition of any one of embodiments 164-166, wherein the second nucleoside-containing molecule is a second ATP molecule.
Embodiment 168. The composition of any one of embodiments 1-49 and 153, wherein the synthetic compound binds a RYR domain of the protein.
Embodiment 169. The composition of embodiment 168, wherein the RYR domain is a RY1&2 domain.
Embodiment 170. The composition of any one of embodiments 1-49 and 153-169, wherein the synthetic compound comprises a benzazepane, benzothiazepane, or benzodiazepane moiety.
Embodiment 171. The composition of any one of embodiments 1-49 and 153-169, wherein the synthetic compound comprises a benzothiazepane moiety.
Embodiment 172. The composition of any one of embodiments 1-49 and 153-169, wherein the synthetic compound is a compound of Formula (I):

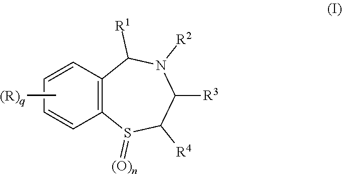

wherein:
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
   R¹ is alkyl, alkenyl, aryl, alkylaryl, cycloalkyl, heteroaryl, or heterocyclyl, each of which is independently substituted or unsubstituted; or H;
   R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R₁₀;
   R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
   Y is alkyl, aryl, alkylaryl, cycloalkyl, heteroaryl, or heterocyclyl, each of which is independently substituted or unsubstituted; or H;
   R⁴ is alkyl, alkenyl, aryl, alkylaryl, cycloalkyl, heteroaryl, or heterocyclyl, each of which is independently substituted or unsubstituted; or H;
   each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
   each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
   each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
   each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
   each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
   each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
   each X is halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
   each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
   n is 0, 1, or 2;
   q is 0, 1, 2, 3, or 4;
   t is 1, 2, 3, 4, 5, or 6; and
   m is 1, 2, 3, or 4,
or a pharmaceutically-acceptable salt thereof.
Embodiment 173. The composition of any one of embodiments 1-49 and 153-172, wherein the synthetic compound is a compound of Formula (I-k):

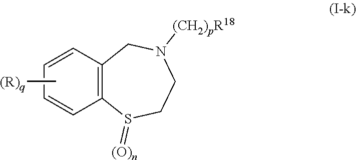

wherein:
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
   R¹⁸ is alkyl, aryl, cycloalkyl, or heterocyclyl, each of which is independently substituted or unsubstituted; or —NR¹⁵R¹⁶, —C(═O)NR¹⁵R¹⁶, —(C═O)OR¹⁵, or —OR¹⁵;
   q is 0, 1, 2, 3, or 4;
   p is 1, 2, 3, 4, 5, 6, 7, 8 9, or 10; and
   n is 0, 1, or 2,
or a pharmaceutically-acceptable salt thereof.
Embodiment 174. The composition of embodiment 172 or embodiment 173, wherein each R is independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2.
Embodiment 175. The composition of any one of embodiments 172-174, wherein R¹⁸ is —NR¹⁵R¹⁶, —(C═O)OR¹⁵, —OR¹⁵, alkyl that is substituted or unsubstituted, or aryl that is substituted or unsubstituted
Embodiment 176. The composition of any one of embodiments 1-49 and 153-172, wherein the synthetic compound is a compound of Formula (I-0):

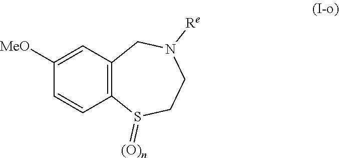

wherein:
   R^{e} is (C₁-C₆ alkyl)-phenyl, (C₁-C₆ alkyl)-C(O)R^{b}, or substituted or unsubstituted —C₁-C₆ alkyl; and
   R^{b} is OH or —O—(C₁-C₆ alkyl), wherein the phenyl or the substituted alkyl is substituted with one or more of halogen, hydroxyl, C₁-C₆ alkyl, —O—(C₁-C₆ alkyl), —NH₂, —NH(C₁-C₆ alkyl), —N(C₁-C₆ alkyl)₂, cyano, or dioxolane,
or a pharmaceutically-acceptable salt thereof.
Embodiment 177. The composition of any one of embodiments 1-49 and 153-176, wherein the synthetic compound is:

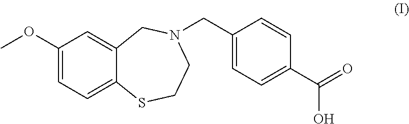 [SMILES: COc1ccc2c(c1)CN(Cc1ccc(C(=O)O)cc1)CCS2]

or an ionized form thereof.
Embodiment 178. The composition of any one of embodiments 1-49 and 153-176, wherein the synthetic compound is:

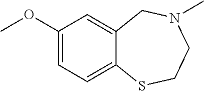

or an ionized form thereof.
Embodiment 179. The composition of embodiment 177, wherein the synthetic compound has a three-dimensional conformation according to TABLE 4.
Embodiment 180. A vessel containing the composition of any one of embodiments 1-49.
Embodiment 181. The vessel of embodiment 181, wherein the vessel is a vial, ampule, test tube, or microwell plate.
Embodiment 182. A method of determining a binding site of a synthetic compound in a protein, the method comprising subjecting a composition of any one of embodiments 153-179 to single-particle cryogenic electron microscopy analysis.
Embodiment 183. The method of embodiment 182, wherein the structure of the of protein obtained by single-particle cryogenic electron microscopy analysis has a resolution from about 2 Å to about 3.5 Å, from about 2 Å to about 3.4 Å, from about 2 Å to about 3.3 Å, from about 2 Å to about 3.2 Å, from about 2 Å to about 3.1 Å, from about 2 Å to about 3 Å, from about 2 Å to about 2.9 Å, from about 2 Å to about 2.8 Å, from about 2 Å to about 2.7 Å, from about 2 Å to about 2.6 Å, from about 2 Å to about 2.5 Å, from about 2.1 Å to about 2.5 Å, from about 2.2 Å to about 2.5 Å, from about 2.3 Å to about 2.5 Å, or from about 2.4 Å to about 2.5 Å.
Embodiment 184. A method for predicting a docked position of a target ligand in a binding site of a biomolecule, the method comprising:
   receiving a template ligand-biomolecule structure, the template ligand-biomolecule structure comprising a template ligand docked in the binding site of the biomolecule;
   comparing a pharmacophore model of the template ligand to a pharmacophore model of the target ligand;
   overlapping the pharmacophore model of the target ligand with the pharmacophore model of the template ligand while the template ligand is in the binding site of the biomolecule; and
   predicting the docked position of the target ligand in the binding site of the biomolecule based on a position of the pharmacophore model of the target ligand when overlapped with the pharmacophore model of the template ligand,
   wherein the biomolecule is a RY1&2 domain of RyR2, wherein the template ligand-biomolecule structure is obtained by a process comprising subjecting a complex of the biomolecule and the template ligand to single-particle cryogenic electron microscopy analysis.
Embodiment 185. The method of embodiment 184, wherein the RY1&2 domain comprises a structure according to TABLE 3.
Embodiment 186. The method of embodiment 184, wherein the template ligand has a three-dimensional conformation according to TABLE 4.
Embodiment 187. The method of embodiment 184, wherein the RY1&2 domain further comprises a nucleoside-containing molecule.
Embodiment 188. The method of embodiment 187, wherein the nucleoside-containing molecule is an ATP molecule.
Embodiment 189. The method of embodiment 188, wherein the ATP molecule has a three-dimensional conformation according to TABLE 5.
Embodiment 190. The method of embodiment 187 or embodiment 188, wherein the target ligand cooperatively binds the RY1&2 domain with the ATP molecule.
Embodiment 191. The method of embodiment 184, wherein the target ligand and the template ligand are each independently a compound of Formula (I):

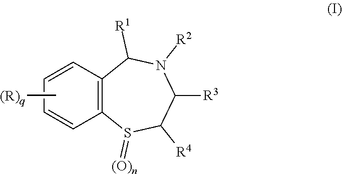

wherein:
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
   R¹ is alkyl, alkenyl, aryl, alkylaryl, cycloalkyl, heteroaryl, or heterocyclyl, each of which is independently substituted or unsubstituted; or H;
   R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
   R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
   Y is alkyl, aryl, alkylaryl, cycloalkyl, heteroaryl, or heterocyclyl, each of which is independently substituted or unsubstituted; or H;
   R⁴ is alkyl, alkenyl, aryl, alkylaryl, cycloalkyl, heteroaryl, or heterocyclyl, each of which is independently substituted or unsubstituted; or H;
   each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, OR, —C(═O)NHNR¹⁵R¹⁶, —CO₂R¹⁵, —C(═O)NR¹⁵R¹⁶, or —CH₂X;
   each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
   each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
   each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
   each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
   each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
   each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
   each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
   n is 0, 1, or 2;
   q is 0, 1, 2, 3, or 4;
   t is 1, 2, 3, 4, 5, or 6; and
   m is 1, 2, 3, or 4.
Embodiment 192. The method of any one of embodiments 184-192, wherein the template ligand is

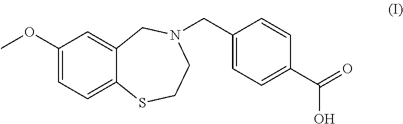 [SMILES: COc1ccc2c(c1)CN(Cc1ccc(C(=O)O)cc1)CCS2]

or an ionized form thereof.
Embodiment 193. The method of any one of embodiments 184-192, wherein the template ligand is

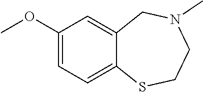

or an ionized form thereof.
Embodiment 194. The method of embodiment 184, further comprising selecting the target ligand from a plurality of ligand candidates, each of the ligand candidates being different from the template ligand, and wherein selecting the target ligand comprises comparing the pharmacophore model of the template ligand to a pharmacophore model of each respective one of the plurality of ligand candidates.
Embodiment 195. The method of embodiment 184, further comprising receiving a plurality of template ligand-biomolecule structures, each template ligand-biomolecule structure having a different template ligand docked in the binding site of the biomolecule, and generating the pharmacophore model of the template ligand by combining information from each of the template ligands from the plurality of template ligand-biomolecule structures.
Embodiment 196. The method of embodiment 184, wherein the target ligand has more than one structural conformation in an unbound state, and the docked position of the target ligand in the binding site of the biomolecule is predicted by enumerating a set of potential target ligand conformations and overlapping a respective pharmacophore model of the target ligand for each of the potential target ligand conformations with the pharmacophore model of the template ligand while the template ligand is in the binding site of the biomolecule.
Embodiment 197. The method of embodiment 196, wherein predicting the docked position of the target ligand in the binding site of the biomolecule comprises ignoring at least one clash between the target ligand conformation's atomic coordinates and the biomolecule's atomic coordinates.
Embodiment 198. The method of embodiment 197, further comprising, for each target ligand conformation, modifying atomic coordinates of the biomolecule to reduce clashes between the docked target ligand conformation's atomic coordinates and the biomolecule's atomic coordinates, thereby creating an altered ligand-biomolecule structure comprising the docked target ligand and an altered biomolecule.
Embodiment 199. The method of embodiment 198, further comprising, predicting a re-docked position of each target ligand conformation by predicting each target ligand conformation's position in the binding site of the altered biomolecule; and
   for each target ligand conformation, modifying atomic coordinates of the altered biomolecule to reduce clashes between the atomic coordinates of the target ligand conformation's re-docked position and the atomic coordinates of the altered biomolecule,
   thereby creating a re-altered ligand-biomolecule structure comprising a re-docked target ligand and a re-altered biomolecule.
Embodiment 200. The method of embodiment 199, further comprising ranking each altered and re-altered ligand-biomolecule structure using a scoring function.
Embodiment 201. The method of embodiment 200, further comprising identifying a subset of high-ranking target ligands corresponding to target ligands having a threshold value for an empirical activity.
Embodiment 202. A method of identifying a plurality of potential lead compounds, the method comprising the steps of:
   (a) analyzing, using a computer system, an initial lead compound known to bind to a biomolecular target, the analyzing comprising partitioning, by providing a database of known reactions, the initial lead compound into atoms defining partitioned lead compound comprising a lead compound core and atoms defining a lead compound non-core, wherein the initial lead compound is partitioned using a computational retrosynthetic analysis of the initial lead compound;
   (b) identifying, using the computer system, a plurality of alternative cores to replace the lead compound core in the initial lead compound, thereby generating a plurality of potential lead compounds each having a respective one of the plurality of alternative cores;
   (c) calculating, using the computer system, a difference in binding free energy between the partitioned lead compound and each potential lead compound;
   (d) predicting, using the computer system, whether each potential lead compound will bind to the biomolecular target and identifying a predicted active set of potential lead compounds based on the prediction;
   (e) obtaining a synthesized set of at least some of the potential leads of the predicted active set to establish a first of potential lead compounds; and
   (f) determining, empirically, an activity of each of the first set of synthesized potential lead compounds,
   wherein the biomolecular target is a RY1&2 domain of RyR2, and the structure of the biomolecular target used in the predicting of (d) is obtained by a process comprising subjecting a complex of the biomolecular target and the initial lead compound to single-particle cryogenic electron microscopy analysis.
Embodiment 203. The method of embodiment 202, wherein the structure of the of the biomolecular target obtained by single-particle cryogenic electron microscopy analysis has a resolution from about 2 Å to about 3.5 Å, from about 2 Å to about 3.4 Å, from about 2 Å to about 3.3 Å, from about 2 Å to about 3.2 Å, from about 2 Å to about 3.1 Å, from about 2 Å to about 3 Å, from about 2 Å to about 2.9 Å, from about 2 Å to about 2.8 Å, from about 2 Å to about 2.7 Å, from about 2 Å to about 2.6 Å, from about 2 Å to about 2.5 Å, from about 2.1 Å to about 2.5 Å, from about 2.2 Å to about 2.5 Å, from about 2.3 Å to about 2.5 Å, or from about 2.4 Å to about 2.5 Å.
Embodiment 204. The method of embodiment 202, wherein the initial lead compound is a compound of Formula (I):

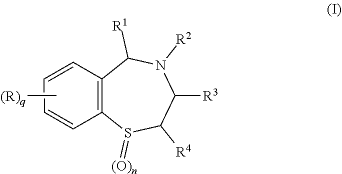

wherein.
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
   R¹ is alkyl, alkenyl, aryl, alkylaryl, cycloalkyl, heteroaryl, or heterocyclyl, each of which is independently substituted or unsubstituted; or H;
   R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, —(CH₂)ₘ—R¹⁰;
   R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
   Y is alkyl, aryl, alkylaryl, cycloalkyl, heteroaryl, or heterocyclyl, each of which is independently substituted or unsubstituted; or H;
   R⁴ is alkyl, alkenyl, aryl, alkylaryl, cycloalkyl, heteroaryl, or heterocyclyl, each of which is independently substituted or unsubstituted; or H;
   each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, OR, —C(═O)NHNR¹⁵R¹⁶, —CO₂R¹⁵, —C(═O)NR¹⁵R¹⁶, or —CH₂X;
   each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
   each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
   each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
   each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
   each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
   each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
   each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
   n is 0, 1, or 2;
   q is 0, 1, 2, 3, or 4;
   t is 1, 2, 3, 4, 5, or 6; and
   m is 1, 2, 3, or 4.
Embodiment 205. The method of embodiment 202, wherein the initial lead compound is

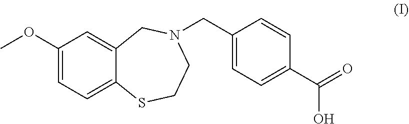 [SMILES: COc1ccc2c(c1)CN(Cc1ccc(C(=O)O)cc1)CCS2]

or an ionized form thereof.
Embodiment 206. The method of embodiment 202, wherein the initial lead compound is

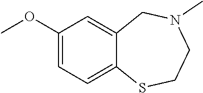

or an ionized form thereof.
Embodiment 207. The method of embodiment 202, wherein the RY1&2 domain comprises a structure according to TABLE 3.
Embodiment 208. The method of embodiment 202, wherein the RY1&2 domain contains an ATP molecule.
Embodiment 209. The method of embodiment 208, wherein the ATP molecule has a three-dimensional conformation according to TABLE 5.
Embodiment 210. The method of embodiment 202, further comprising obtaining a synthesized set of at least some of the potential lead compounds predicted to not bind with the biomolecular target to establish a second set of potential lead compounds and empirically determining an activity of each of the second set of synthesized potential lead compounds.
Embodiment 211. The method of embodiment 202, further comprising comparing the empirically determined activity of each of the first set of synthesized potential lead compounds with a threshold activity level.
Embodiment 212. The method of embodiment 203, further comprising comparing the empirically determined activity of each of the second set of synthesized potential lead compounds with a pre-determined activity level.
Embodiment 213. The method of embodiment 202, wherein the plurality of alternative cores are chosen from a database of synthetically feasible cores.
Embodiment 214. The method of embodiment 202, wherein the difference in binding free energy is calculated using a free energy perturbation technique.
Embodiment 215. The method of embodiment 210, wherein the generation of at least one potential lead compound comprises creating an additional covalent bond or annihilating an existing covalent bond, or both creating an additional first covalent bond and annihilating an existing second covalent bond different from the first covalent bond.
Embodiment 216. The method of embodiment 211, wherein the free energy perturbation technique uses a soft bond potential to calculate a bonded stretch interaction energy of existing covalent bonds for annihilation and additional covalent bonds for creation.
Embodiment 217. A method for pharmaceutical drug discovery, comprising:
   identifying an initial lead compound for binding to a biomolecular target; using the method of embodiment 202 to identify a predicted active set of potential lead compounds for binding to the biomolecular target based on the initial lead compound; selecting one or more of the predicted active set of potential lead compounds for synthesis; and
   assaying the one or more synthesized selected compounds to assess each synthesized selected compounds suitability for in vivo use as a pharmaceutical compound,
   wherein the biomolecular target is a RY1&2 domain of RyR2, and the structure of the biomolecular target used in the predicting of (d) is obtained by a process comprising subjecting a complex of the biomolecular target and the initial lead compound to single-particle cryogenic electron microscopy analysis.
Embodiment 218. The method of embodiment 217, wherein the initial lead compound is compound of Formula (I):

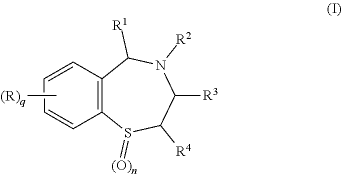

wherein:
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
   R¹ is alkyl, alkenyl, aryl, alkylaryl, cycloalkyl, heteroaryl, or heterocyclyl, each of which is independently substituted or unsubstituted; or H;
   R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, —(CH₂)ₘ—R¹⁰;
   R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
   Y is alkyl, aryl, alkylaryl, cycloalkyl, heteroaryl, or heterocyclyl, each of which is independently substituted or unsubstituted; or H;
   R⁴ is alkyl, alkenyl, aryl, alkylaryl, cycloalkyl, heteroaryl, or heterocyclyl, each of which is independently substituted or unsubstituted; or H;
   each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, OR, —C(═O)NHNR¹⁵R¹⁶, —CO₂R¹⁵, —C(═O)NR¹⁵R¹⁶, or —CH₂X;
   each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
   each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
   each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
   each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
   each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
   each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
   each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
   n is 0, 1, or 2;
   q is 0, 1, 2, 3, or 4;
   t is 1, 2, 3, 4, 5, or 6; and
   m is 1, 2, 3, or 4.
Embodiment 219. The method of embodiment 217, wherein the initial lead compound is

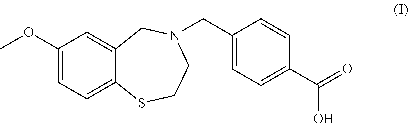 [SMILES: COc1ccc2c(c1)CN(Cc1ccc(C(=O)O)cc1)CCS2]

or an ionized form thereof.
Embodiment 220. The method of embodiment 217, wherein the initial lead compound is

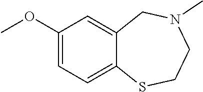

or an ionized form thereof.
Embodiment 221. The method of embodiment 217, wherein the RY1&2 domain comprises a structure according to TABLE 3.
Embodiment 222. The method of embodiment 217, wherein the RY1&2 domain contains an ATP molecule.
Embodiment 223. The method of embodiment 221, wherein the ATP molecule has a three-dimensional conformation according to TABLE 5.
Embodiment 224. A computer-implemented method of quantifying binding affinity between a ligand and a receptor molecule, the method comprising:
   receiving by one or more computers, data representing a ligand molecule,
   receiving by one or more computers, data representing a receptor molecule domain,
   using the data representing the ligand molecule and the data representing the receptor molecule domain in computer analysis to identify ring structure within the ligand, the ring structure being an entire ring or a fused ring;
   using the data representative of the identified ligand ring structure to designate a first ring face and a second ring face opposite to the first ring face, and classifying the ring structure by:
   a) determining proximity of receptor atoms to atoms on the first face of the ligand ring; and
   b) determining proximity of receptor atoms to atoms on the second face of the ligand ring;
   c) determining solvation of the first face of the ligand ring and solvation of the second face of the ligand ring;
   classifying the identified ligand ring structure as buried, solvent exposed or having a single face exposed to solvent based on receptor atom proximity to and solvation of the first ring face and receptor atom proximity to and solvation of the second ring face;
   quantifying the binding affinity between the ligand and the receptor molecule domain based at least in part on the classification of the ring structure; and
   displaying, via computer, information related to the classification of the ring structure, wherein the receptor molecule domain is a RY1&2 domain of RyR2, wherein the data representing a ligand molecule and the data representing a receptor molecule domain are obtained by a process comprising subjecting a complex comprising the ligand molecule and the receptor molecule domain to single-particle cryogenic electron microscopy analysis.
Embodiment 225. The method of embodiment 224, wherein the initial lead compound is compound of Formula (I):

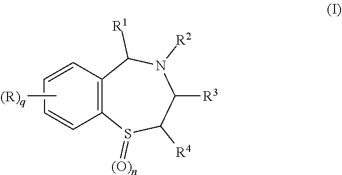

wherein:
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
   R¹ is alkyl, alkenyl, aryl, alkylaryl, cycloalkyl, heteroaryl, or heterocyclyl, each of which is independently substituted or unsubstituted; or H;
   R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
   R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
   Y is alkyl, aryl, alkylaryl, cycloalkyl, heteroaryl, or heterocyclyl, each of which is independently substituted or unsubstituted; or H;
   R⁴ is alkyl, alkenyl, aryl, alkylaryl, cycloalkyl, heteroaryl, or heterocyclyl, each of which is independently substituted or unsubstituted; or H;
   each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
   each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
   each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
   each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
   each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
   each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, —NH₂, —NHNH₂, or —NHOH;
   each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
   each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
   n is 0, 1, or 2;
   q is 0, 1, 2, 3, or 4;
   t is 1, 2, 3, 4, 5, or 6; and
   m is 1, 2, 3, or 4.
Embodiment 226. The method of embodiment 224, wherein the ligand molecule is

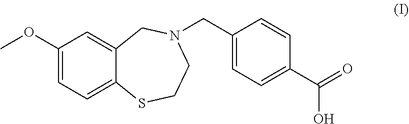 [SMILES: COc1ccc2c(c1)CN(Cc1ccc(C(=O)O)cc1)CCS2]

or an ionized form thereof.
Embodiment 227. The method of embodiment 224, wherein the ligand molecule is

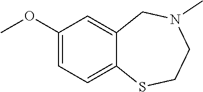

or an ionized form thereof.
Embodiment 228. The method of embodiment 224, wherein the complex further comprises a RyR2 protein, wherein the RY1&2 domain is a domain of the RyR2 protein.
Embodiment 229. The method of embodiment 224, wherein the data representing the receptor molecule domain represents a three-dimensional structure of the receptor molecule according to TABLE 3.
Embodiment 230. The method of embodiment 224, wherein the data representing a ligand molecule represents a three-dimensional structure of the ligand molecule according to TABLE 4.
Embodiment 231. The method of embodiment 224, wherein the receptor molecule domain contains an ATP molecule.
Embodiment 232. The method of embodiment 231, wherein the data representing the receptor molecule domain further comprises data representing a three-dimensional structure of the ATP molecule according to TABLE 5.
Embodiment 233. The method of embodiment 224, wherein quantifying the binding affinity includes a step that scores hydrophobic interactions between one or more ligand atoms and one or more receptor atoms by awarding a bonus for the presence of hydrophobic enclosure of one or more atoms of said ligand by the receptor molecule domain, said bonus being indicative of enhanced binding affinity between said ligand and said receptor molecule domain.
Embodiment 234. The method of embodiment 224, further comprising calculating an initial binding affinity and then adjusting the initial binding affinity based on the classification of the ring structure as buried, solvent exposed or solvent exposed on one face.
Embodiment 235. The method of embodiment 224, wherein the classification of a ring structure as buried, solvent exposed, or solvent exposed on one surface, includes using a parameter substantially correlated with the number of close contacts on both sides of the ring structure or part thereof with the receptor molecule domain.
Embodiment 236. The method of embodiment 224, wherein the number of close contacts at different distances between receptor atoms and the two ring faces are determined, an initial classification of the ring is made based on the numbers of these contacts, and this initial classification is then followed by calculation of a scoring function, said scoring function comprising identifying a first ring shell and a second ring shell, and calculating the number of water molecules in the first shell and in the second shell, or calculating the number of water molecules in the first and second shell combined.
Embodiment 237. The method of embodiment 236, wherein the scoring function allowing classification of the ring structure as buried, solvent exposed, or solvent exposed on one surface, includes using a parameter substantially correlated with the lipophilic-lipophilic pair score between the ring structure or part thereof and the receptor molecule domain.
Embodiment 238. The method of embodiment 236, wherein the scoring function used to classify a ring structure as buried, solvent exposed, or solvent exposed on one surface, includes calculating the degree of enclosure of each atom of the ring structure by atoms of the receptor.
Embodiment 239. The method of embodiment 236, wherein the scoring function used to classify a ring structure as buried, solvent exposed, or solvent exposed on one surface, includes using a parameter that is substantially correlated with the degree of enclosure of each atom of the ring structure by atoms of the receptor.
Embodiment 240. The method of embodiment 224 or embodiment 236, wherein the scoring function allowing classification of the ring structure as buried, solvent exposed, or solvent exposed on one surface, includes the use of a parameter corresponding to a hydrophobic interaction of the ring structure or part thereof with the receptor molecule domain.
Embodiment 241. The method of embodiment 240, wherein the information displayed by computer includes a depiction of at least one of
   the degree to which the ring structure is enclosed by atoms of the receptor molecule domain;
   water molecules surrounding the ring structure in a first shell or a second shell or both the first and the second shell of the ligand;
   a value of a lipophilic-lipophilic pair score of the ring structure; and
   a number of close contacts of a face of the ring structure with the receptor molecule domain.
Embodiment 242. The method of embodiment 224, wherein solvent exposed ring structures in the ligand, if any, are substantially ignored in quantifying the component of the binding affinity between the ligand and the receptor molecule domains, other than to recognize hydrogen bonds and other parameters that are independent of the classification of ring structure.
Embodiment 243. The method of embodiment 224, wherein hydrophobic contribution to binding affinity from ring structures classified as solvent exposed, if any, is substantially ignored in quantifying the component of the binding affinity.
Embodiment 244. The method of embodiment 224, wherein a ring structure is classified as buried, and the method further comprises:
   identifying a quantity representative of a strain energy induced in the ligand-receptor complex by the buried ring structure, in which the quantification of the component of binding affinity is further based in part on strain energy.
Embodiment 245. The method of embodiment 244, further comprising
   identifying a quantity representative of a strain energy induced in the ligand-receptor complex by the aggregate of the ring structures identified as buried;
   identifying a quantity representative of a total neutral-neutral hydrogen bond energy; and
   quantifying the component of binding affinity between the ligand and the receptor molecule domain based at least in part on the quantity representative of the strain energy induced in the receptor by the aggregate of the buried ring structures, and on the quantity representative of the total neutral-neutral hydrogen bond energy.
Embodiment 246. The method of embodiment 245, wherein
   quantifying the component of binding affinity further comprises identifying a hydrogen bond capping energy associated with the entire ligand, and
   the component of binding affinity is quantified based on a greater of the hydrogen bond capping energy and the quantity representative of the strain energy induced in the receptor by the aggregate of the identified structures.
Embodiment 247. The method of embodiment 245, further comprising:
   identifying a binding motif of the receptor molecule domain with respect to the ligand;
   identifying a reorganization energy of the receptor molecule domain based on the binding motif; and
   identifying a first ring structure as contributing to the reorganization energy,
   the quantity representative of strain energy being identified independently of the classification of the first ring structure.
Embodiment 248. The method of embodiment 244, wherein the component of binding affinity attributable to strain is quantified using at least one of: molecular dynamics, molecule mechanics, conformational searching and minimization.
Embodiment 249. The method of embodiment 224, wherein the information displayed by computer includes a depiction of solvent exposure, if any, of the ring structure.
Embodiment 250. The method of embodiment 224, wherein the information displayed by computer includes a depiction of burial, if any, of the ring structure.
Embodiment 251. The method of embodiment 224, wherein the information displayed by computer includes a depiction of at least one of
   the degree to which the ring structure is enclosed by atoms of the receptor molecule domain;
   water molecules surrounding the ring structure in a first shell or a second shell or both the first and the second shell of the ligand;
   a value of a lipophilic-lipophilic pair score of the ring structure; and
   a number of close contacts of a face of the ring structure with the receptor molecule domain.
Embodiment 252. The method of embodiment 224, further comprising,
   performing a test on a physical sample that includes the ligand and the receptor molecule domain, test components being selected based at least in part on the binding affinity between the ligand or part thereof and the receptor molecule, or on the component of such binding affinity.
Embodiment 253. A method comprising:
   (a) determining an open probability (Pₒ) of a first RyR2 protein, wherein the first RyR2 protein is treated with a test compound, and
   (b) determining an open probability (Pₒ) of a second RyR2 protein, wherein the second RyR2 protein is not treated with the test compound.
Embodiment 254. The method of embodiment 253, wherein each of the determining the open probability (Pₒ) of the first RyR2 protein and the determining the open probability (Pₒ) of the second RyR2 protein comprises recording a single channel Ca²⁺ current.
Embodiment 255. The method of embodiments 253-254, further comprising determining a difference between the Pₒ of the first RyR2 protein and Pₒ of the second RyR2 protein.
Embodiment 256. The method of embodiment 255, further comprising identifying the test compound as a target for further analysis based on the difference between the Pₒ of the first RyR2 protein and Pₒ of the second RyR2 protein.
Embodiment 257. The method of embodiment 256, wherein the Pₒ of the first RyR2 protein is lower than the Pₒ of the second RyR2 protein.
Embodiment 258. The method of embodiment 255, further comprising performing an analogous assay wherein another compound is used in place of the test compound, wherein the analogous assay provides a difference between:
   (a) an open probability (Pₒ) of a third RyR2 protein, wherein the third RyR2 protein is treated with the other compound; and
   (b) an open probability (Pₒ) of a fourth RyR2 protein, wherein the fourth RyR2 protein is not treated with the other compound,
   wherein the test compound is prioritized over the other compound for the further analysis based on a comparison of:
   (i) the difference between the Pₒ of the first RyR2 protein and Pₒ of the second RyR2 protein; with
   (ii) a difference between the Pₒ of the third RyR2 protein and Pₒ of the fourth RyR2 protein.
Embodiment 259. The method of any one of embodiments 255-258, wherein the difference of the Pₒ in (b)(ii) is greater than the difference of the Pₒ in (b)(i).
Embodiment 260. The methods of any one of embodiments 255-259, wherein the RyR2 is a mutant RyR2.
Embodiment 261. The method of embodiment 260, wherein the mutant RyR2 contains at least one mutation that is associated with Catecholaminergic Polymorphic Ventricular Tachycardia (CPVT).
Embodiment 262. The composition of embodiment 261, wherein the mutation is RyR2-R2474S.
Embodiment 263. The composition of embodiment 261, wherein the mutation is RyR2-R420Q.
Embodiment 264. The composition of embodiment 261, wherein the mutation is RyR2-R420W.
Embodiment 265. The composition of any one of embodiments 260-264, wherein the mutation destabilizes an interaction between NTD and BSol domains of the RyR2 protein.
Embodiment 266. The composition of any one of embodiments 260-265, wherein the mutation destabilizes a cytosolic shell of the RyR2 protein, wherein the cytosolic shell comprises NTD, SPRY, JSol and BSol domains of the RyR2 proteins.
Embodiment 267. The method of any one of embodiments 255-266, wherein the RyR2 protein is a post-translationally modified RyR2 protein.
Embodiment 268. The method of embodiment 268, wherein the post-translationally modified RyR2 protein is a phosphorylated, oxidized or nitrosylated RyR2.
Embodiment 269. The composition of embodiment 267 or embodiment 268, wherein the post-translationally modified RyR2 protein is a phosphorylated RyR2.
Embodiment 270. The composition of any one of embodiments 267-269, wherein the post-translationally modified RyR2 protein is associated with heart failure.
Embodiment 271. The composition of any one of embodiments 267-270, wherein the post-translational modification destabilizes an interaction between NTD and BSol domains of the RyR2 protein.
Embodiment 272. The composition of any one of embodiments 267-271, wherein the post-translational modification destabilizes a cytosolic shell of the RyR2 protein, wherein the cytosolic shell comprises NTD, SPRY, JSol and BSol domains of the RyR2 proteins.
Embodiment 273. The method of any one of embodiments 255-272, wherein the RyR2 is a mutated and post-translationally modified RyR2.
Embodiment 274. A method comprising:
   (a) contacting a first RyR2 protein with a test compound;
   (b) providing a second RyR2 protein;
   (c) subsequent to the contacting the first RyR2 protein with the test compound, measuring an open probability (Pₒ) of the first RyR2 protein; and
   (d) measuring an open probability (Pₒ) of the second RyR2 protein.
Embodiment 275. The method of embodiment 274, wherein each of the determining the open probability (Pₒ) of the first RyR2 protein and the determining the open probability (Pₒ) of second RyR2 protein comprises recording a single channel Ca²⁺ current.
Embodiment 276. The method of any one of embodiments 274-275, further comprising determining a difference between the Pₒ of the first RyR2 protein and the Pₒ of the second RyR2 protein.
Embodiment 277. The method of embodiment 276, further comprising identifying the test compound as a target for further analysis based on the difference between the Pₒ of the first RyR2 protein and the Pₒ of the second RyR2 protein.
Embodiment 278. The method of embodiment 277, wherein the Pₒ of the first RyR2 protein is lower than the Pₒ of the second RyR2 protein.
Embodiment 279. The method of embodiment 276, further comprising performing an analogous assay wherein another compound is used in place of the test compound, wherein the analogous assay provides a difference between:
   (a) an open probability (Pₒ) of a third RyR2 protein, wherein the third RyR2 protein is treated with the other compound; and
   (b) an open probability (Pₒ) of a fourth RyR2 protein, wherein the fourth RyR2 protein is not treated with the other compound,
   wherein the test compound is prioritized over the other compound for the further analysis based on a comparison of:
   (i) the difference between the Pₒ of the first RyR2 protein and Pₒ of the second RyR2 protein; with
   (ii) a difference between the Pₒ of the third RyR2 protein and Pₒ of the fourth RyR2 protein.
Embodiment 280. The method of any one of embodiments 276-279, wherein the difference of the Pₒ in (b)(ii) is greater than the difference of the Pₒ in (b)(i).
Embodiment 281. The methods of any one of embodiments 274-280, wherein the RyR2 is a mutant RyR2.
Embodiment 282. The method of embodiment 281, wherein the mutant RyR2 contains at least one mutation that is associated with Catecholaminergic Polymorphic Ventricular Tachycardia (CPVT).
Embodiment 283. The composition of embodiment 282, wherein the mutation is RyR2-R2474S.
Embodiment 284. The composition of embodiment 282, wherein the mutation is RyR2-R420Q.
Embodiment 285. The composition of embodiment 282, wherein the mutation is RyR2-R420W.
Embodiment 286. The composition of any one of embodiments 282-285, wherein the mutation destabilizes an interaction between NTD and BSol domains of the RyR2 protein.
Embodiment 287. The composition of any one of embodiments 282-286, wherein the mutation destabilizes a cytosolic shell of the RyR2 protein, wherein the cytosolic shell comprises NTD, SPRY, JSol and BSol domains of the RyR2 proteins.
Embodiment 288. The method of any one of embodiments 274-287, wherein the RyR2 protein is a post-translationally modified RyR2 protein.
Embodiment 289. The method of embodiment 288, wherein the post-translationally modified RyR2 protein is a phosphorylated, oxidized or nitrosylated RyR2.
Embodiment 290. The method of any one of embodiments 274-289, wherein the RyR2 is a mutated and post-translationally modified RyR2.
Embodiment 291. The method of any one of embodiments 253-290, further comprising: subsequent to the contacting the first RyR2 protein with the reagent and the test compound, fusing a first microsome containing the first RyR2 protein to a first planar lipid bilayer, and subsequent to the contacting the second RyR2 protein with the reagent, fusing a second microsome containing the second RyR2 protein to a second planar lipid bilayer.
Embodiment 292. A method of identifying a compound having RyR2 modulatory activity, the method comprising:
   (a) determining an open probability (Pₒ) of a RyR2 protein;
   (b) contacting the RyR2 protein with a test compound;
   (c) determining an open probability (Pₒ) of the RyR2 protein in the presence of the test compound; and
   (d) determining a difference between the Pₒ of the RyR2 protein in the presence and absence of the test compound;
   wherein a reduction in the Pₒ of the RyR2 protein in the presence of the test compound relative to the Pₒ of the RyR2 protein in the absence of the test compound is indicative of the compound having RyR2 modulatory activity.
Embodiment 293. The method of embodiment 292, wherein the RyR2 protein is a mutant RyR2 protein.
Embodiment 294. The method of embodiment 293, wherein the mutant RyR2 contains at least one mutation that is associated with Catecholaminergic Polymorphic Ventricular Tachycardia (CPVT).
Embodiment 295. The composition of embodiment 294, wherein the mutation is RyR2-R2474S.
Embodiment 296. The composition of embodiment 294, wherein the mutation is RyR2-R420Q.
Embodiment 297. The composition of embodiment 294, wherein the mutation is RyR2-R420W.
Embodiment 298. The composition of any one of embodiments 294-297, wherein the mutation destabilizes an interaction between NTD and BSol domains of the RyR2 protein.
Embodiment 299. The composition of any one of embodiments 294-297, wherein the mutation destabilizes a cytosolic shell of the RyR2 protein, wherein the cytosolic shell comprises NTD, SPRY, JSol and BSol domains of the RyR2 proteins.
Embodiment 300. The method of any one of embodiments 292-299, wherein the RyR2 protein is a post-translationally modified RyR2 protein.
Embodiment 301. The method of embodiment 300, wherein the post-translationally modified RyR2 protein is a phosphorylated, oxidized or nitrosylated RyR2.
Embodiment 302. The method of any one of embodiments 292-301, wherein the RyR2 protein is a mutated and post-translationally modified RyR2 protein.
Embodiment 303. The method of any one of embodiments 292-302, wherein the test compound preferentially binds to a mutant RyR2 relative to wild-type RyR2.
Embodiment 304. The method of any one of embodiments 292-303, wherein the test compound preferentially binds to post-translationally modified RyR2 relative to wild-type RyR2.
Embodiment 305. The method of any one of embodiments 292-305, wherein the test compound preferentially binds to a mutated and post-translationally modified RyR2 relative to a wild-type RyR2.
Embodiment 306. The method of any one of embodiments 292-305, wherein determining the open probability (Pₒ) of the RyR2 protein comprises recording a single channel Ca²⁺ current.
Embodiment 307. A method for identifying a compound having RyR2 modulatory activity, comprising:
   (a) contacting a RyR2 protein with a ligand having known RyR2 modulatory activity to create a mixture, wherein the RyR2 protein is a leaky RyR2, the leaky RyR2 comprising mutant RyR2 protein, post-translationally modified RyR2, or a combination thereof;
   (b) contacting the mixture of step (a) with a test compound; and
   (c) determining an ability of the test compound to displace the ligand from the RyR2 protein.
Embodiment 308. The method of embodiment 307, wherein the ligand is radiolabeled.
Embodiment 309. The method of embodiment 307 or embodiment 308, wherein determining the ability of the test compound to displace the ligand from the RyR2 protein comprises determining a radioactive signal in the RyR2 protein.
Embodiment 310. The method of any one of embodiments 307-309, wherein the RyR2 protein is a mutant RyR2 protein.
Embodiment 311. The method of embodiment 309 or 310, wherein the mutant RyR2 contains at least one mutation that is associated with Catecholaminergic Polymorphic Ventricular Tachycardia (CPVT).
Embodiment 312. The composition of embodiment 311, wherein the mutation is RyR2-R2474S.
Embodiment 313. The composition of embodiment 311, wherein the mutation is RyR2-R420Q.
Embodiment 314. The composition of embodiment 311, wherein the mutation is RyR2-R420W.
Embodiment 315. The composition of any one of embodiments 311-314, wherein the mutation destabilizes an interaction between NTD and BSol domains of the RyR2 protein.
Embodiment 316. The composition of any one of embodiments 311-315, wherein the mutation destabilizes a cytosolic shell of the RyR2 protein, wherein the cytosolic shell comprises NTD, SPRY, JSol and BSol domains of the RyR2 proteins.
Embodiment 317. The method of any one of embodiments 307-316, wherein the RyR2 protein is a post-translationally modified RyR2 protein.
Embodiment 318. The method of any one of embodiments 307-316, wherein the RyR2 protein is a mutated and post-translationally modified RyR2 protein.
Embodiment 319. The method of any one of embodiments 307-318, wherein the test compound preferentially binds to a mutant RyR2 relative to wild-type RyR2.
Embodiment 320. The method of any one of embodiments 307-318, wherein the test compound preferentially binds to post-translationally modified RyR2 relative to wild-type RyR2.
Embodiment 321. The method of any one of embodiments 307-318, wherein the test compound preferentially binds to a mutant and post-translationally modified RyR2 relative to a wild-type RyR2.
Embodiment 322. A method for identifying a compound that preferentially binds to a mutated, post-translationally modified RyR2 or a combination thereof, comprising:
   (a) determining binding affinity of a test compound to a first RyR2 protein, wherein the first RyR2 protein is a wild-type RyR2 protein;
   (b) determining binding affinity of a test compound to a second RyR2 protein, wherein second first RyR2 protein is a mutant RyR2 protein, a post-translationally modified RyR2, or a combination thereof, and
   (c) selecting a compound having a higher binding affinity to the second RyR2 protein relative to the first RyR2 protein.
Embodiment 323. The method of embodiment 322, wherein the RyR2 protein is a mutant RyR2 protein.
Embodiment 324. The method of embodiment 322 or 323, wherein the mutant RyR2 contains at least one mutation that is associated with Catecholaminergic Polymorphic Ventricular Tachycardia (CPVT).
Embodiment 325. The composition of embodiment 324, wherein the mutation is RyR2-R2474S.
Embodiment 326. The composition of embodiment 324, wherein the mutation is RyR2-R420Q.
Embodiment 327. The composition of embodiment 324, wherein the mutation is RyR2-R420W.
Embodiment 328. The composition of any one of embodiments 324-327, wherein the mutation destabilizes an interaction between NTD and BSol domains of the RyR2 protein.
Embodiment 329. The composition of any one of embodiments 324-328, wherein the mutation destabilizes a cytosolic shell of the RyR2 protein, wherein the cytosolic shell comprises NTD, SPRY, JSol and BSol domains of the RyR2 proteins.
Embodiment 330. The method of any one of embodiments 322-329, wherein the RyR2 protein is a post-translationally modified RyR2 protein.
Embodiment 331. The method of any one of embodiments 322-329, wherein the RyR2 protein is a mutated and post-translationally modified RyR2 protein.
Embodiment 332. The method of any one of embodiments 253-331, wherein the test compound preferentially binds to a mutant RyR2 relative to wild-type RyR2.
Embodiment 333. The method of any one of embodiments 253-331, wherein the test compound preferentially binds to post-translationally modified RyR2 relative to wild-type RyR2.
Embodiment 334. The method of any one of embodiments 253-331, wherein the test compound preferentially binds to a mutant and post-translationally modified RyR2 relative to a wild-type RyR2.
Embodiment 335. The method of any one of embodiments 253-334, wherein the test compound contains a benzothiazepane moiety.
Embodiment 336. The method of any one of embodiments 253-335, wherein the test compound is a compound of Formula (I):

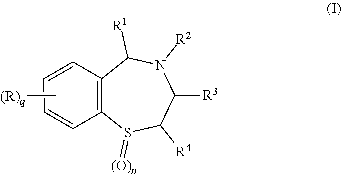

wherein:
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
   R¹ is alkyl, alkenyl, aryl, alkylaryl, cycloalkyl, heteroaryl, or heterocyclyl, each of which is independently substituted or unsubstituted; or H;
   R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
   R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
   Y is alkyl, aryl, alkylaryl, cycloalkyl, heteroaryl, or heterocyclyl, each of which is independently substituted or unsubstituted; or H;
   R⁴ is alkyl, alkenyl, aryl, alkylaryl, cycloalkyl, heteroaryl, or heterocyclyl, each of which is independently substituted or unsubstituted; or H;
   each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, OR, —C(═O)NHNR¹⁵R¹⁶, —CO₂R¹⁵, —C(═O)NR¹⁵R¹⁶, or —CH₂X;
   each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
   each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
   each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
   each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
   each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
   each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
   each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
   n is 0, 1, or 2;
   q is 0, 1, 2, 3, or 4;
   t is 1, 2, 3, 4, 5, or 6; and
   m is 1, 2, 3, or 4,
      or any other compound herein, or a pharmaceutically acceptable salt thereof.

### EXAMPLES

### Example 1: Generation of Stable Cell Lines

Complementary DNA (cDNA) for human RyR2 was subcloned into the A1.2 vector in two steps, using the Nhe I-Xho I fragment and then the Not I-Nhe I fragment of human RYR2. Human RYR2 was inserted 3′ of a cytomegalovirus (CMV) promoter and followed in 5′ by internal ribosomal entry site (IRES)-green fluorescent protein (GFP). The sequence of the construct was confirmed by Sanger sequencing and restriction digestion with Afl III enzyme. The resulting vector (G418-resistant) was cotransfected into HEK293T cells with a plasmid carrying a puromycin resistance gene using calcium chloride. Cells were maintained in a G418- and puromycin-containing medium for approximately 3 weeks and then underwent two cycles of clonal selection, where the top 0.1% of the most highly fluorescent cells were propagated.

### Example 2: Generation of Human RYR2-R2474S DNA and HEK293 Transfection

Constructs expressing RyR2-R2474S were formed by introducing the respective mutation into fragments of human RYR2 using the QuikChange II XL Site-Directed Mutagenesis Kit (Agilent). Three nucleotide changes were introduced (WT sequence: AGGGTCTAT to R2474S mutant: AGCGTATAC). The first was the mutation R2474S, and the other two were silent mutations that introduced a BstZ171 restriction site (GTATAC) to facilitate screening for mutant clones. Each fragment was subcloned into a full-length human RYR2 construct in pCMV5 vector, confirmed by sequencing, and expressed in 293T/17 cells using Lipofectamine 2000 (Thermo Fisher Scientific). For final expression, HEK293 cells grown in 150-mm dishes with Dulbecco's Modified Eagle Medium (DMEM) supplemented with 10% (v/v) fetal bovine serum (Invitrogen), penicillin (100 U/ml), streptomycin (100 μg/ml), and 2 mM 1-glutamine were cotransfected with 25 mg per dish of human RYR2-R2474S cDNA using PEI MAX at a 1:5 ratio (Polysciences). Cells were collected 48 hours after transfection.

### Example 3: Purification of Recombinant GST-Calstabin-2

Recombinant human GST-calstabin-2 was expressed in BL21 (DE3) Escherichia co/i cells with a thrombin protease cleavage site between GST and calstabin-2. Protein expression was induced with 0.8 mM isopropyl-o-d-thiogalactopyranoside (IPTG) added to E. coli at an OD₆₀₀ (optical density at 600 nm) of 0.8 with overnight incubation at 18° C. before centrifugation for 10 min at 6500 g. The pellets were resuspended in buffer A (phosphate-buffered saline+0.5 mM AEBSF) and lysed using an emulsiflex (Avestin EmulsiFlex-C3). The lysate was pelleted by centrifugation for 10 min at 100,000 g. The supernatant was then loaded into a 5-ml GSTrap HP column (Cytiva) and washed with 5 column volume (CV) of buffer A to remove contaminants before elution with buffer B [tris (pH 8), 2 mM DTT, and 20 mM glutathione]. Fractions containing GST-calstabin-2 were pooled, concentrated, and dialyzed overnight at 4° C. into buffer A. final concentration was determined by spectroscopy using NanoDrop 1000 (Thermo Fisher Scientific) with absorbance at 280 nm and an extinction coefficient of 46,200 M⁻¹ cm⁻¹. GST-calstabin-2 was stored at −80° C.

### Example 4: Purification of Recombinant Calmodulin (CaM) and TEV Protease

Recombinant human calmodulin (CaM) was expressed in BL21 (DE3) E. coli cells with an N-terminal 6-histidine tag and a tobacco etch virus (TEV) protease cleavage site. Protein expression was induced with 0.8 mM IPTG added to E. coli at an OD₆₀₀ of 0.8 with overnight incubation at 18° C. before centrifugation for 10 min at 6500 g and storage at −80° C. CaM was purified using a two-step 5-ml HisTrap HP column (Cytiva) purification. In brief, the pellets were resuspended in buffer A [20 mM Hepes (pH 7.5), 150 mM NaCl, 20 mM imidazole, 5 mM 2-Mercaptoethanol, and 0.5 mM AEBSF] and lysed using an emulsiflex (Avestin EmulsiFlex-C3). The lysate was pelleted by centrifugation for 10 min at 100,000 g. The supernatant was then loaded over a HisTrap column and washed with 5 CV of buffer A to remove contaminants before elution using a linear gradient from buffer A to buffer B (buffer A containing 500 mM imidazole). Fractions containing CaM were pooled, 1 to 2 mg of purified TEV protease was added, and the mixture was dialyzed overnight at 4° C. into buffer C (buffer A with no imidazole). CaM was then loaded onto a HisTrap column with the flowthrough collected and the wash fractionated to retain fractions containing CaM before elution of TEV and any remaining contaminants with a linear gradient from buffer C to buffer B. The flowthrough and any fractions containing CaM were pooled, concentrated to >2 mM, and determined by spectroscopy using NanoDrop 1000 (Thermo Fisher Scientific) with absorbance at 280 nm and the extinction coefficient of CaM (3000 M⁻¹ cm⁻¹). CaM was stored at −20° C. TEV protease was purified in the same manner except for using an uncleavable his-tag and thus ending after the first HisTrap column wherein the purified protease was stored at −80° C. in buffer C with 10% glycerol.

### Example 5: Purification and Treatment of Recombinant Human RyR2

To obtain high-resolution structures of WT and mutant human RyR2, the purification of channels expressed recombinantly in HEK293 cells was optimized. All purification steps were performed on ice unless otherwise stated. HEK293 cells (25 to 50 dishes) expressing human RyR2 or RyR2-R2474S (prepared according to EXAMPLE 1 and EXAMPLE 2) were harvested by centrifugation for 10 min at 1500 g. The pellet fraction was resuspended in tris malate buffer [10 mM tris malate (pH 6.8), 1 mM EGTA, 1 mM dithiothreitol (DTT), 1 mM benzamidine, 0.5 mM 4-benzenesulfonyl fluoride hydrochloride (AEBSF), and protease inhibitor cocktail] and was sonicated with six pulses of 20 s at 35% amplitude. The membrane fraction was precipitated by centrifugation at 100,000 g for 30 min and was resuspended with a glass homogenizer in CHAPS buffer [10 mM Hepes (pH 7.4), 1 M NaCl, 1.5% CHAPS, 0.5% phosphatidylcholine (PC), 1 mM EGTA, 2 mM DTT, 0.5 mM AEBSF, 1 mM benzamidine, and protease inhibitor cocktail]. The sample was diluted 1:4 with the same buffer without NaCl. To achieve stabilization of peripherical domains, 100 nmol of GST-calstabin-2 was added to both tris malate buffer and CHAPS buffer. The remaining insoluble material was separated with a second centrifugation at 100,000 g for 30 min.

The supernatant-containing detergent-solubilized human RyR2—was filtered and loaded into a 5-ml HiTrap Q HP column (Cytiva) previously equilibrated with buffer A [10 mM Hepes (pH 7.4), 0.4% CHAPS, 1 mM EGTA, 0.001% dioleoylphosphatidylcholine (DOPC), 250 mM NaCl, and 0.5 mM TCEP (tris(2-carboxyethyl)phosphine)]. The HiTrap Q HP column was eluted with a linear gradient between 300 and 600 mM NaCl. The fractions containing human RyR2 (300 to 350 mM NaCl) were pooled, and 100 nmol of GST-calstabin-2 (prepared according to EXAMPLE 3) was added. The pooled fractions were loaded into a 1-ml GSTrap HP column (Cytiva), which was left recirculating overnight. The GSTrap HP column was washed with buffer A and eluted with glutathione buffer {10 mM Hepes (pH 8), 0.4% CHAPS, 1 mM EGTA, 0.001% DOPC, 200 mM NaCl, 10 mM GSH [glutathione (reduced form)], and 1 mM DTT}. Immediately after, a second 1-ml HiTrap Q HP column (Cytiva) binding/elution step was applied to separate human RyR2 from the excess of unbound GST-calstabin-2 and GSH.

Six preparations of RyR2 and RyR2-R2474S were prepared:
(1) Dephosphorylated RyR2 (DeP-RyR2);
(2) PKA phosphorylated RyR2 (P-RyR2);
(3) PKA phosphorylated RyR2+20 μM CaM (P-RyR2+CaM);
(4) PKA phosphorylated RyR2-R2474S (P-RyR2-R2474S);
(5) PKA phosphorylated RyR2-R2474S+20M Compound 1 (P-RyR2-R2474S+Cpd1); and
(6) PKA phosphorylated RyR2-R2474S+40 μM CaM (P-RyR2-R2474S+CaM).

For phosphorylated samples (P-RyR2, P-RyR2+CaM, P-RyR2-R2474S, P-RyR2-R2474S+Cpd1, and P-RyR2-R2474S+CaM), simultaneous cleavage of GST tag and PKA phosphorylation was performed by addition of 50 U of thrombin and 100 U of PKA (+10 mM EGTA, 8 mM MgCl₂, and 100 μM ATP for activity), respectively, for 30 min on ice. For samples requiring dephosphorylation treatment (DeP-RyR2), PKA was replaced by 2000 U of phosphatase lambda (PX from NEB, +1×Protein MetalloPhosphatases (PMP) buffer, and 1 mM MnCl₂). Each sample was concentrated to 0.5 ml, and a gel filtration step was run with TSKgel G4SW_{XL} (TOSOH Biosciences) with buffer A. RyR2 fractions were pooled and concentrated to a concentration of 4 to 8 mg/ml (with centrifugal filters of 100-kDa cutoff) and were filtered (with centrifugal filters of 0.22-μm cutoff) to eliminate aggregates.

To resemble exercise diastolic conditions, 10 mM NaATP, 500 μM xanthine, 150 nM Ca²⁺ free (650 μM total Ca²⁺), and 200 μM cAMP were added to all samples. 20 μM CaM (prepared according to EXAMPLE 4) was added to PKA-phosphorylated RyR2 (P-RyR2+CaM), 40 μM CaM was added to PKA-phosphorylated RyR2-R2474S (P-RyR2-R2474S+CaM), and 500 μM Compound 1 was added to PKA-phosphorylated RyR2-R2474S (P-RyR2-R2474S+Cpd1). MaxChelator webserver was used to calculate total/free Ca²⁺ concentrations.

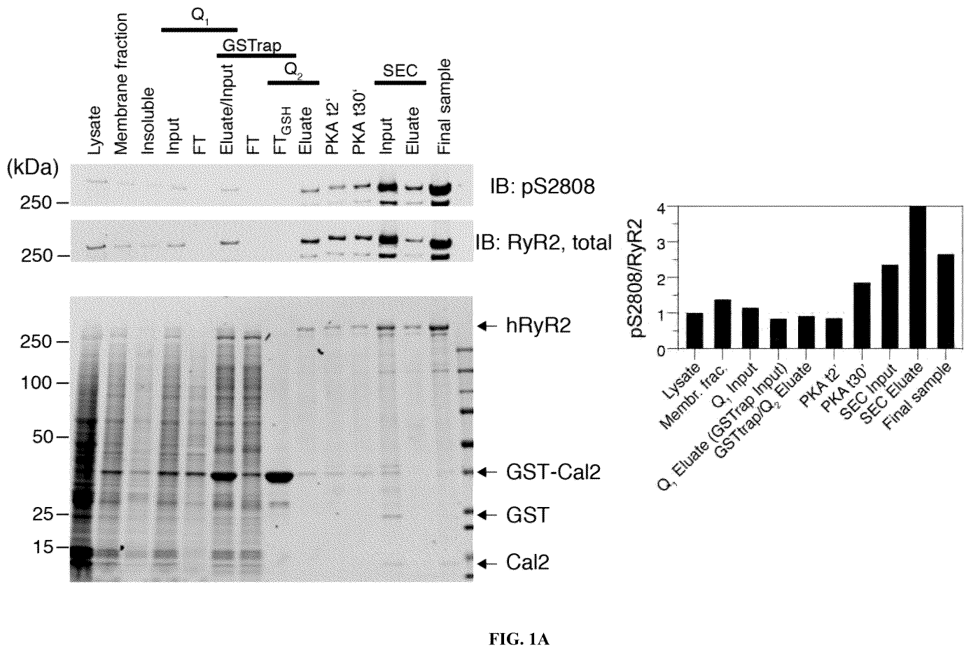

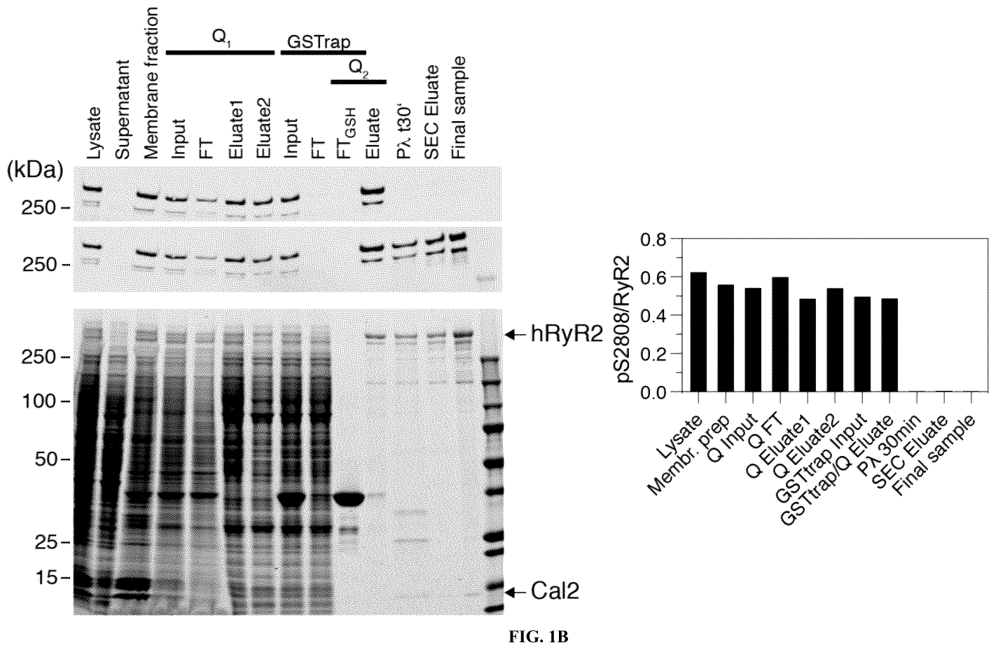

Quality control was assessed by SDS-polyacrylamide gel electrophoresis (SDS-PAGE) and immunoblots using anti-RyR-5029 and anti-RyR2-pS2809 antibodies for total and phosphorylated RyR2, respectively. FIG. 1A depicts an immunoblot (left, top) and SDS-PAGE (left, middle) of purification of human recombinant RyR2 expressed in HEK293 cells. Ratio of normalized intensities of the pS2808 and total RyR2 bands (right). After PKA phosphorylation, the intensity increases ˜3-fold, reaching a saturation point. RyR2 expressed in HEK293 cells show a basal phosphorylation of S2808 as confirmed by mass spectrometry. FIG. 1B depicts an immunoblot (left, top) and SDS-PAGE (left, bottom) of purification of dephosphorylated (with phosphatase lambda) human recombinant RyR2 expressed in HEK293 cells.

### Example 6: Cryo-EM Analysis

### Methods.

### Sample Preparation and Data Collection.

Each of the final samples (3 μl each) prepared in EXAMPLE 5 was applied to UltrAuFoil holey gold grids (Quantifoil R 0.6/1.0, Au 300) previously cleaned with easiGlow (PELCO). Grids were blotted with ashless filter paper (Whatman) using blot force 10 and blot time 8 s before vitrification by plunge-freezing into liquid ethane chilled with liquid nitrogen using Vitrobot Mark IV (Thermo Fisher Scientific) operated at 4° C. with 100% relative humidity.

Prepared grids were screened in-house on a Glacios Cryo-TEM (Thermo Fisher Scientific) microscope with a 200-kV x-FEG source and a Falcon 3EC direct electron detector (Thermo Fisher Scientific). Microscope operations and data collection were carried out using EPU software (Thermo Fisher Scientific). High-resolution data collection was performed at Columbia University on a Titan Krios 300-kV (Thermo Fisher Scientific) microscope equipped with an energy filter (slit width 20 eV) and a K3 direct electron detector (Gatan). Data were collected using Leginon and at a nominal magnification of ×105,000 in electron counting mode, corresponding to a pixel size of 0.83 Å. The electron dose rate was set to 16 e⁻/pixel per second with 2.5-s exposures for a total dose of 50 to 60 e/A2.

### Data Processing and Model Building.

Cryo-EM data processing was performed in cryoSPARC with image stacks aligned using Patch motion, defocus value estimation by Patch CTF estimation. Particle picking was performed using the template picker with templates created from preexisting cryo-EM maps. Particles were subjected to 2D classification in cryoSPARC with 100 classes. Particles from the highest-resolution classes were pooled for ab initio 3D reconstruction with a single class followed by homogeneous refinement with C4 symmetry imposed. 3D variability and further clustering were performed using a mask comprising the TM domain to separate those particles in the closed and open states followed by heterogeneous refinement with four classes to further select the best particles. C4 symmetry expansion was performed before local refinements. The masks used were TaF+TM+CTD domains (residues 4131 to 4967), calstabin-2+NTD+SPRY domains (residues 1 to 1646), JSol+CSol domains (residues 1700 to 2476, 3590 to 4130), and BSol domain (residues 2400 to 3344). Only the TaF+TM+CTD mask used C4 symmetry. Smaller masks, for a second round of 3D variability analysis, clustering, and local refinement, were RY1&2 (residues 862 to 1076), RY3&4 (residues 2685 to 2909), and BSol2 (residues 3042 to 3344). Local refinements used a dynamical mask with a far distance of 10, 50, and 150 Å for the initial masks, small masks, and expanded RY3&4 mask for comparing the effect of RY3&4 stabilization, respectively. The resulting maps were combined in ChimeraX to generate a composite map before calibration of the pixel size using correlation coefficients with a map generated from the crystal structure of the NTD of RyR2 (PDB ID: 4JKQ). The pixel size was altered by 0.001 Å per step, up to 20 steps in each direction.

An initial model of RYR2 was generated with Phenix tool sculptor from a 2.45-Å structure of RyR1 (PDB ID: 7TZC). Truncated residues were manually corrected to obtain the full side chains. Only domains RY1&2 and RY3&4, which show weak cryo-EM density and confidence in structure, were based on crystallographic structures. The initial model of RyR2 RY1&2 domain was generated with Phenix tool sculptor from the RY1&2 domain of RyR3 with ATP (PDB ID: 6UHIH). The model of RyR2 RY3&4 domain was obtained as is (PDB ID: 4ETV). Calstabin-2 and CaM models were obtained as is (PDB ID: 6JI8). Model building was performed in Coot and refined with Phenix tool RealSpaceRefine. Figures of the final structure were created using ChimeraX. cryo-EM statistics are summarized in TABLE 1A and TABLE 1B.

TABLE 1A

| | Sample | | | | |
|---|---|---|---|---|---|
| | Dephosphorylated | | PKA phosphorylated | | PKA phosphorylated |
| | hRyR2 | | hRyR2 | | hRyR2 + CaM |
| In vitro treatment | Pλ dephosphorylation | | PKA phosphorylation | | PKA phosphorylation |
| State | closed | open | closed | open | closed |
| PDB ID | 7UA5 | 7UA9 | 7U9Q | 7U9R | 7U9T |
| EMDB ID | EMD-26415 | EMD-26416 | EMD-26405 | EMD-26407 | EMD-26408 |
| Data collection | | | | | |
| Microscope | FEI Titan | FEI Titan | FEI Titan | FEI Titan | FEI Titan |
| | Krios | Krios | Krios | Krios | Krios |
| Detector | Gatan K3 | Gatan K3 | Gatan K3 | Gatan K3 | Gatan K3 |
| Voltage (kV) | 300 | 300 | 300 | 300 | 300 |
| Magnification | 105,000 | 105,000 | 105,000 | 105,000 | 105,000 |
| Exposure (e⁻/Å²) | 58 | 58 | 50.54 | 58 | 58 |
| Defocus range (μm) | −0.4 to −1.2 | −0.4 to −1.2 | −0.4 to −1.2 | −0.4 to −1.2 | −0.4 to −1.2 |
| Pixel size (Å) | 0.83 | 0.83 | 0.844 | 0.83 | 0.83 |
| Processing | | | | | |
| Software | cryoSPARC | cryoSPARC | cryoSPARC | cryoSPARC | cryoSPARC |
| Symmetry | C4 | C4 | C4 | C4 | C4 |
| Initial particles (N) | 148,746 | 148,746 | 94,476 | 148,830 | 148,830 |
| Final particles (N) | 90,375 | 36,491 | 94,476 | 20,156 | 93,881 |
| Global map res. (Å)* | 2.83 | 3.59 | 3.04 | 3.54 | 2.68 |
| Local maps res. range | 2.52-4.06 | 2.92-5.45 | 2.76-4.13 | 3.13-3.71 | 2.41-3.49 |
| (Å)^{‡} | | | | | |
| Model Composition | | | | | |
| Peptide chains | 8 | 8 | 8 | 8 | 12 |
| Nonhydrogen | 138,608 | 138,656 | 138,608 | 138,656 | 143,236 |
| Protein residues | 17,324 | 17,324 | 17,324 | 17,324 | 17,912 |
| Ligands | 12 | 20 | 12 | 20 | 12 |
| Mean B factors (Å²) | | | | | |
| Protein | 107.18 | 135.07 | 120.33 | 111.46 | 109.79 |
| Ligands | 149.67 | 187.73 | 203.75 | 116.42 | 176.02 |
| R.m.s. deviations | | | | | |
| Bond length (Å) | 0.003 | 0.010 | 0.003 | 0.003 | 0.004 |
| Bond angles (°) | 0.779 | 0.877 | 0.848 | 0.848 | 0.81 |
| Ramachandran | | | | | |
| Favored (%) | 96.84 | 96.83 | 96.55 | 97.48 | 96.4 |
| Allowed (%) | 3.07 | 3.10 | 3.36 | 2.43 | 3.53 |
| Disallowed (%) | 0.09 | 0.07 | 0.09 | 0.09 | 0.07 |
| Validation | | | | | |
| MolProbity score | 1.86 | 2.06 | 1.82 | 2.02 | 1.99 |
| Clashscore | 10.84 | 14.72 | 12.18 | 12.81 | 10.53 |
| Rotamer outliers (%) | 1.41 | 1.82 | 0.66 | 2.45 | 1.89 |
| FSC model 0.5 (Å)^{#} | 2.88 | 3.38 | 3.20 | 3.88 | 2.92 |

*Map resolution of the non-uniform refinement determined by CryoSPARC before local masks.
^{‡}Map resolution range represents the range determined by local refinements in cryoSPARC using the masks described in Methods.
^{#}Value obtained from Phenix validation tool.

TABLE 1B

| | Model | | | | |
|---|---|---|---|---|---|
| | PKA phosphorylated | | PKA phosphorylated | PKA phosphorylated | |
| | hRyR2-R2474S | | hRyR2-R2474S + Cpd1 | hRyR2-R2474S + CaM | |
| In vitro treatment | PKA phosphorylation | | PKA phosphorylation | PKA phosphorylation | |
| State | primed | open | closed | closed | open |
| PDB ID | 7U9X | 7U9Z | 7UA1 | 7UA3 | 7UA4 |
| EMDB ID | EMD-26409 | EMD-26410 | EMD-26412 | EMD-26413 | EMD-26414 |
| Data collection | | | | | |
| Microscope | FEI Titan | FEI Titan | FEI Titan | FEI Titan | FEI Titan |
| | Krios | Krios | Krios | Krios | Krios |
| Detector | Gatan K3 | Gatan K3 | Gatan K3 | Gatan K3 | Gatan K3 |
| Voltage (kV) | 300 | 300 | 300 | 300 | 300 |
| Magnification | 105,000 | 105,000 | 105,000 | 105,000 | 105,000 |
| Exposure (e⁻/Å²) | 58.2 | 58.2 | 58.2 | 58.2 | 58.2 |
| Defocus range (μm) | −0.4 to −1.2 | −0.4 to −1.2 | −0.4 to −1.2 | −0.4 to −1.2 | −0.4 to −1.2 |
| Pixel size (Å) | 0.83 | 0.83 | 0.83 | 0.83 | 0.83 |
| Processing | | | | | |
| Software | cryoSPARC | cryoSPARC | cryoSPARC | cryoSPARC | cryoSPARC |
| Symmetry | C4 | C4 | C4 | C4 | C4 |
| Initial particles (N) | 271,481 | 271,481 + | 133,548 | 205,393 | 205,393 |
| | | 133,548 | | | |
| Final particles (N) | 212,141 | 42,295 | 113,172 | 73,052 | 102,257 |
| Global map res. (Å)* | 2.58 | 3.23 | 2.93 | 2.87 | 2.93 |
| Local maps res. range | 2.25-2.97 | 2.79-3.76 | 2.53-3.56 | 2.52-3.46 | 2.46-3.32 |
| (Å)^{‡} | | | | | |
| Model Composition | | | | | |
| Peptide chains | 8 | 8 | 8 | 12 | 12 |
| Nonhydrogen | 138,588 | 138,688 | 138,680 | 150,252 | 147,856 |
| Protein residues | 17,324 | 17,332 | 17,324 | 18,768 | 18,472 |
| Ligands | 12 | 20 | 16 | 12 | 20 |
| Mean B factors (Å²) | | | | | |
| Protein | 85.11 | 112.14 | 96.72 | 101.58 | 105.11 |
| Ligands | 110.48 | 180.94 | 135.79 | 136.45 | 199.47 |
| R.m.s. deviations | | | | | |
| Bond length (Å) | 0.003 | 0.003 | 0.003 | 0.003 | 0.006 |
| Bond angles (°) | 0.614 | 0.752 | 0.618 | 0.687 | 0.729 |
| Ramachandran | | | | | |
| Favored (%) | 97.7 | 95.26 | 96.56 | 95.93 | 96.36 |
| Allowed (%) | 2.28 | 4.6 | 3.32 | 3.92 | 3.55 |
| Disallowed (%) | 0.02 | 0.14 | 0.12 | 0.15 | 0.09 |
| Validation | | | | | |
| MolProbity score | 1.55 | 1.96 | 1.69 | 1.78 | 1.84 |
| Clashscore | 9.1 | 11.55 | 8.72 | 9.46 | 10.59 |
| Rotamer outliers (%) | 0.11 | 1.19 | 0.29 | 0.52 | 1.19 |
| FSC model 0.5 (Å)# | 2.65 | 3.16 | 3.03 | 2.97 | 2.90 |

*Map resolution of the non-uniform refinement determined by CryoSPARC before local masks.
^{‡}Map resolution range represents the range determined by local refinements in cryoSPARC using the masks described in Methods.
#Value obtained from Phenix validation tool.

### Normalized Difference in RMSD Analyses: Pairwise 1-Domain Comparison.

This analysis determined the conformation of a certain query domain from a model X relative to reference closed and open states of RyR2. To this end, the RMSD of Ca of the query domain was measured between model X and the closed state (RMSD_{X-closed}), and between model X and the open state (RMSD_{X-open}). The normalized difference in RMSD was calculated as RMSD_{X-closed}/(RMSD_{X-closed}+RMSD_{X-open}). A value of 0 means that the conformation of the query domain is identical to the same domain in the closed state. A value of 1 means that the conformation of the query domain is identical to the same domain in the open state. A value of 0.5 means that the conformation of the query domain is equally distant to the closed and open states. The error, considered as the intrinsic variability between atomic models, was calculated as the RMSD between aligned domains of the respective models. Propagation of error was calculated using the webserver uncertaintycalculator.com. Bar graphs were made with GraphPad Prism software. RMSD was measured in ChimeraX. When needed, previous alignment of the atomic models centered on the control domains was performed.

### Results.

### CPVT-Linked Mutation RyR2-R2474S Induction of Primed State, and Compound I and CaM Induction of Closed State.

For cryo-EM experiments, conditions that resemble those found during the resting phase of the cardiac cycle known as diastole (150 nM free Ca¹² and 10 mM ATP) were used. Under these conditions, wild-type RyR2 channels are tightly closed, but CPVT RyR2 variants are leaky. Wild-type RyR2 and RyR2-R2474S were pretreated with PKA to induce hyperphosphorylation and to mimic exercise-induced β-adrenergic signaling, which can be a trigger for fatal cardiac arrhythmias in patients with CPVT (FIG. 1A). Xanthine, a physiologically relevant analog of the RyR2 agonist caffeine, was added (500 μM). Xanthine activates RyRs and increases to micromolar range during exercise and can be endogenous activator of RyR2 during exercise.

Across the six preparations of RyR1 subjected to cryo-EM analysis, 10 structures of human RyR2 were obtained:

### Dephosphorylated RyR2 (DeP-RyR2)

(i) Open state (DeP-RyR2-0)
(ii) Closed state (DeP-RyR2-C)

### PKA Phosphorylated RyR2 (P-RyR2)

(iii) Open state (P-RyR2-0)
(iv) Closed state (P-RyR2-C)

### PKA Phosphorylated RyR2+20 μM CaM (P-RyR2+CaM)

(v) Closed state (P-RyR2+CaM-C)

### PKA Phosphorylated RyR2-R2474S (P-RyR2-R2474S)

(vi) Open state (P-RyR2-R2474S-O)
(vii) Primed state (P-RyR2-R2474S-Pr)

### PKA Phosphorylated RyR2-R2474S+20M Compound I (P-RyR2-R2474S+Cpd1)

(viii) Closed state (P-RyR2-R2474S+Cpd1-C). Compound 1 shifts the equilibrium of the RyR2-R2474S channel pore from a primed state to a closed state. In this case, the cytosolic shell is in a primed state, but less open in the presence than the absence of Compound 1. In other words, in the presence of Compound 1, more RyR2-R2474S particles are in a closed state than in the absence of the compound.

### PKA Phosphorylated RyR2-R2474S+40 μM CaM (P-RyR2-R2474S+CaM)

(ix) Open state (P-RyR2-R2474S+CaM-O)
(x) Closed state (P-RyR2-R2474S+CaM-C)

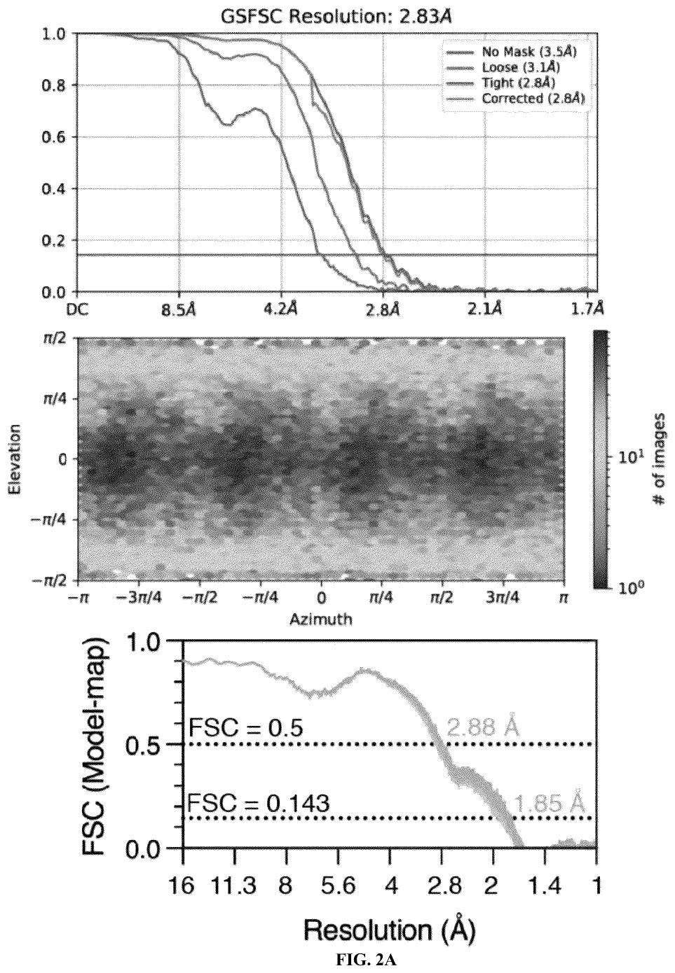

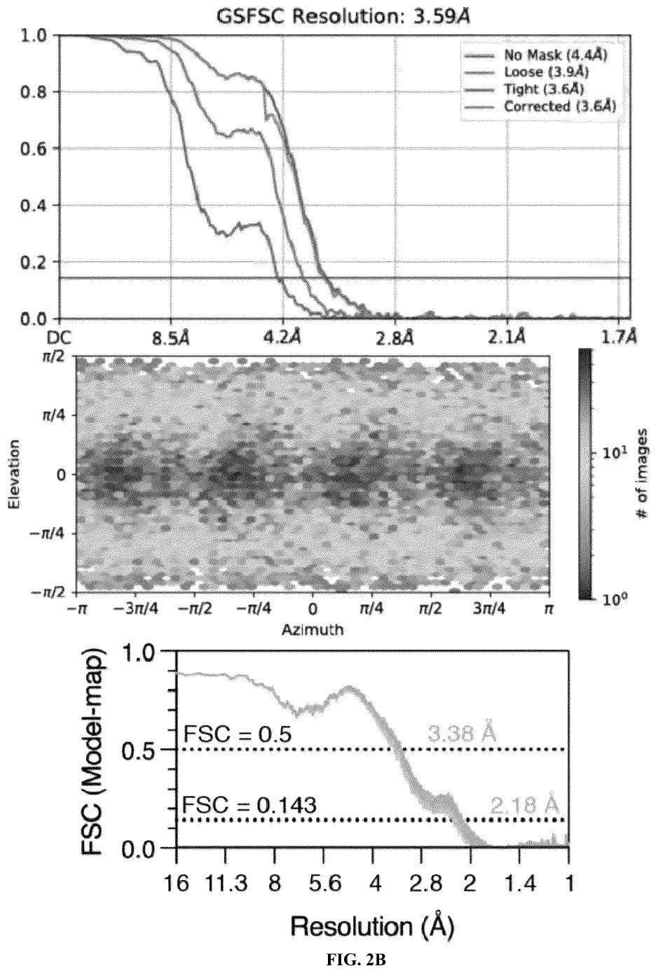

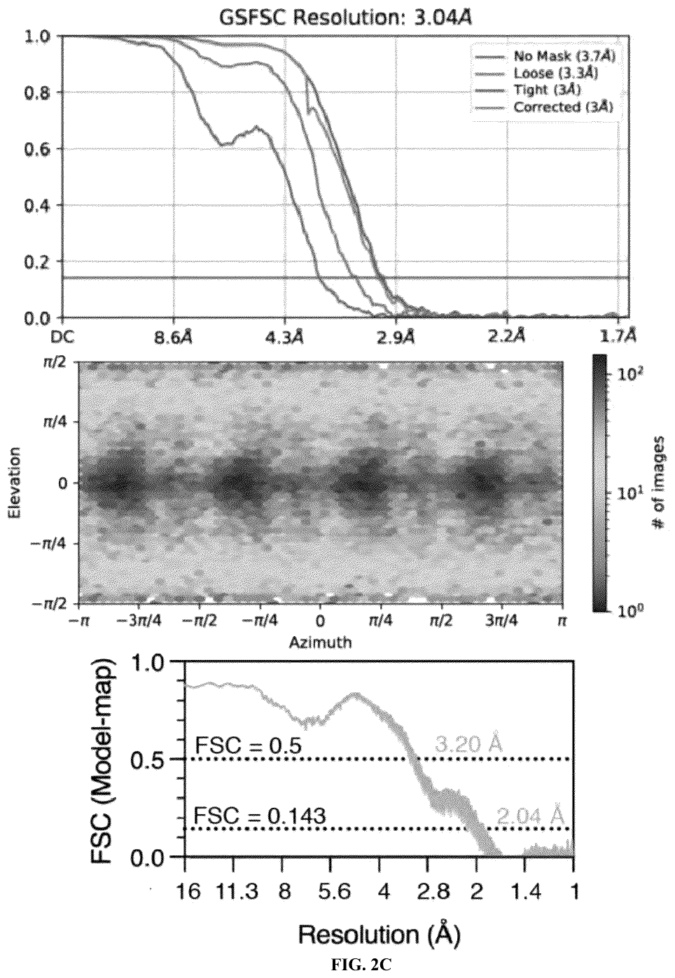

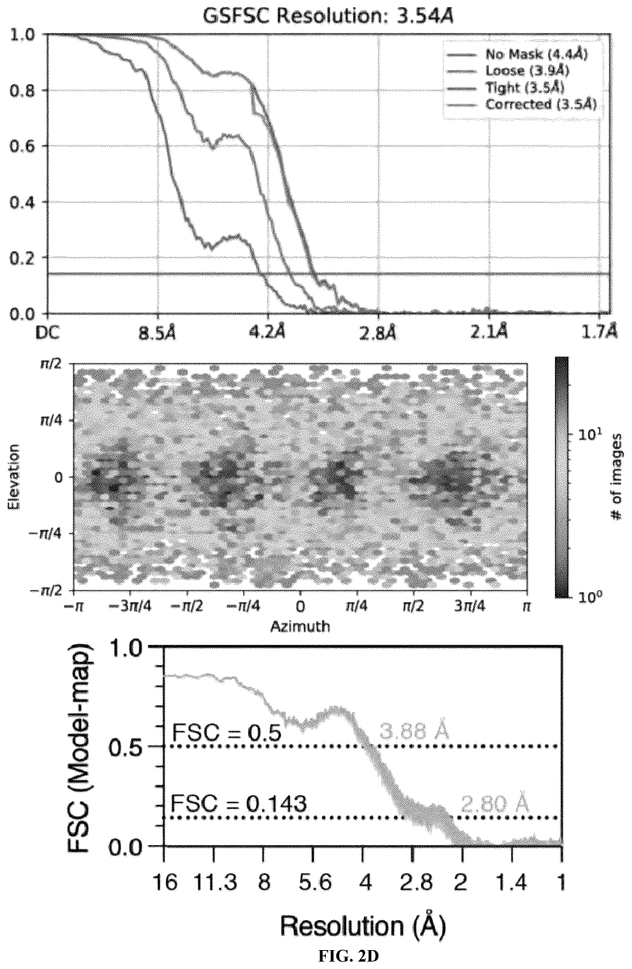

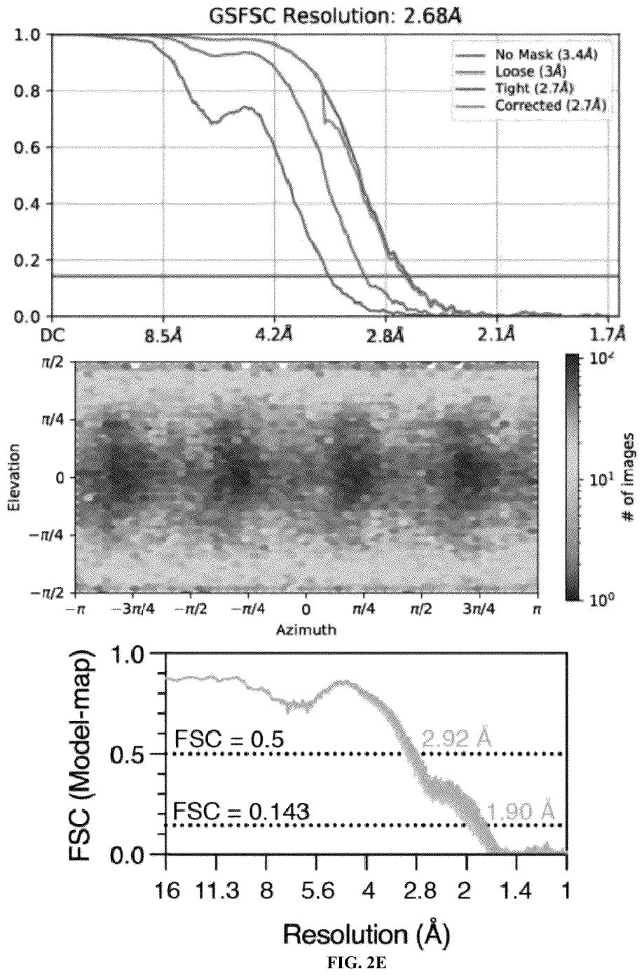

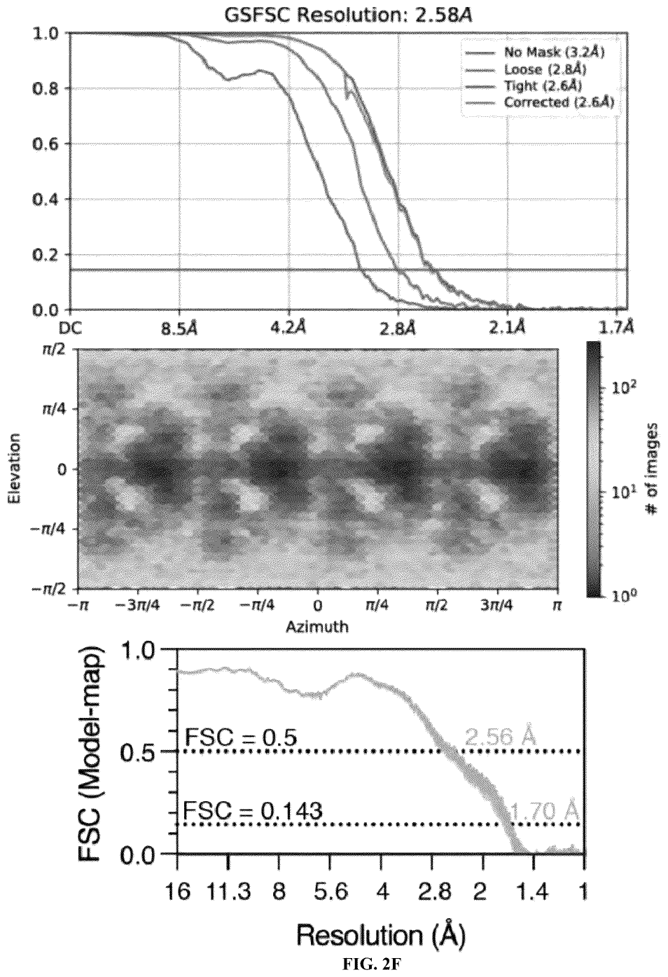

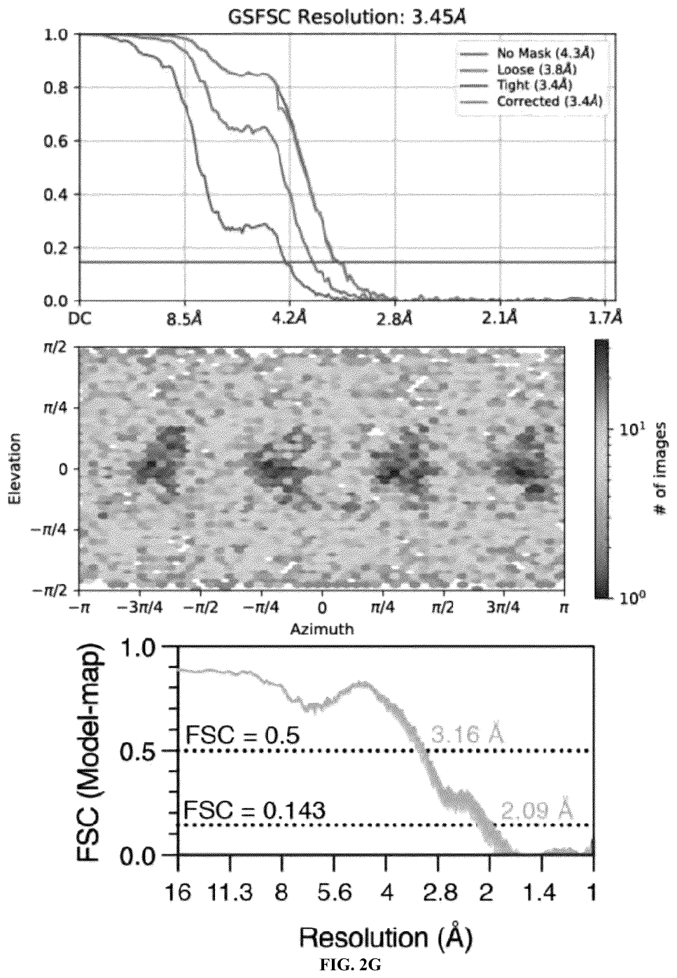

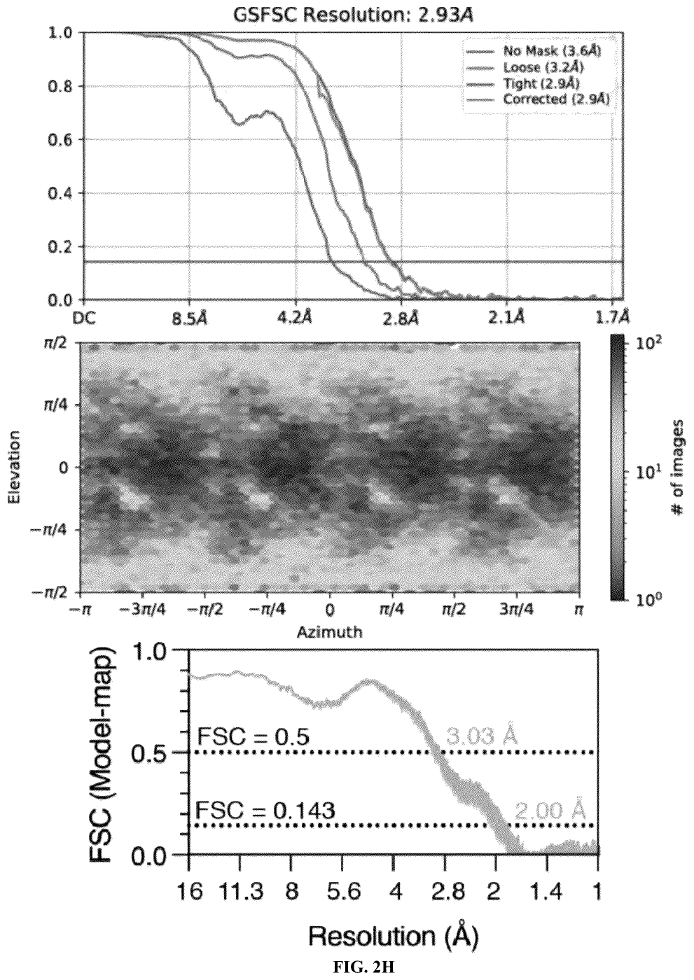

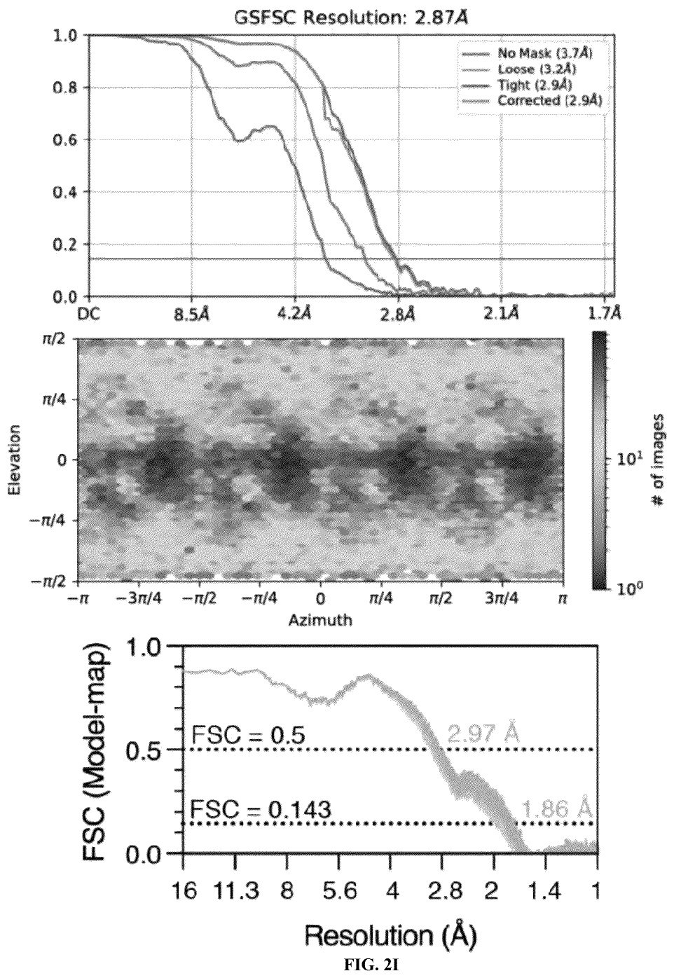

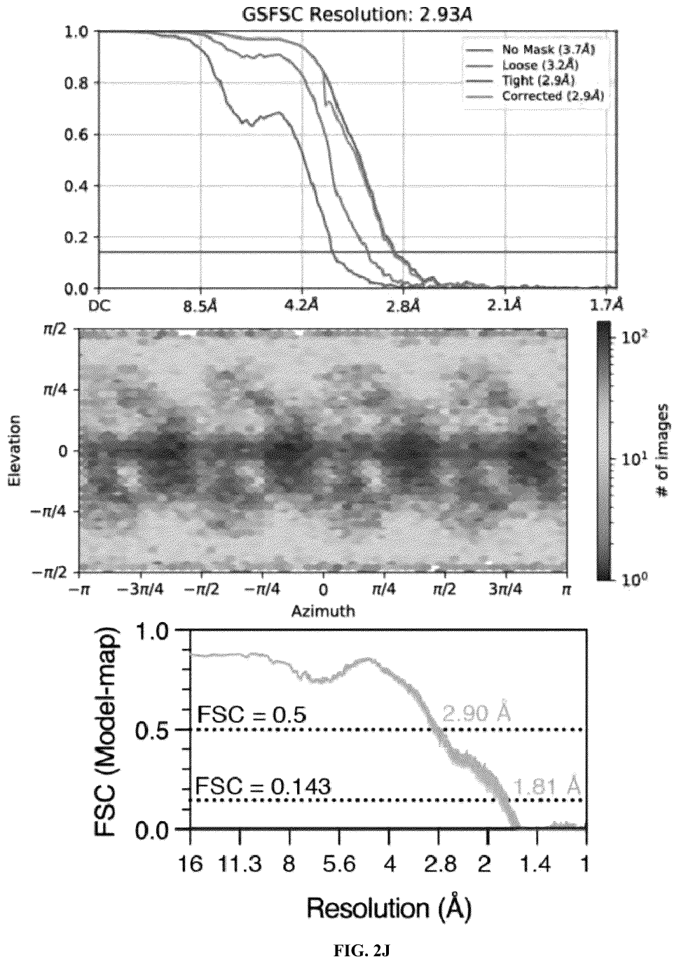

˜3-Å resolution was obtained for most structures. FIGS. 2A-2J provide GSFSCs (top) and viewing angle distributions (middle) of the global nonuniform refinements performed in cryoSPARC before any local refinement for each structure, and FSC model-map performed in PHENIX (bottom) for DeP-RyR2-C (FIG. 2A), DeP-RyR2-0 (FIG. 2B), P-RyR2-C (FIG. 2C), P-RyR2-0 (FIG. 2D), P-RyR2+CaM-C (FIG. 2E), P-RyR2-R2474S-Pr, (FIG. 2F), P-RyR2-R2474S-0 (FIG. 2G), P-RyR2-R2474S+Cpd1-C (FIG. 211), P-RyR2-R2474S+CaM-C (FIG. 2I), and P-RyR2-R2474S+CaM-O (FIG. 2J).

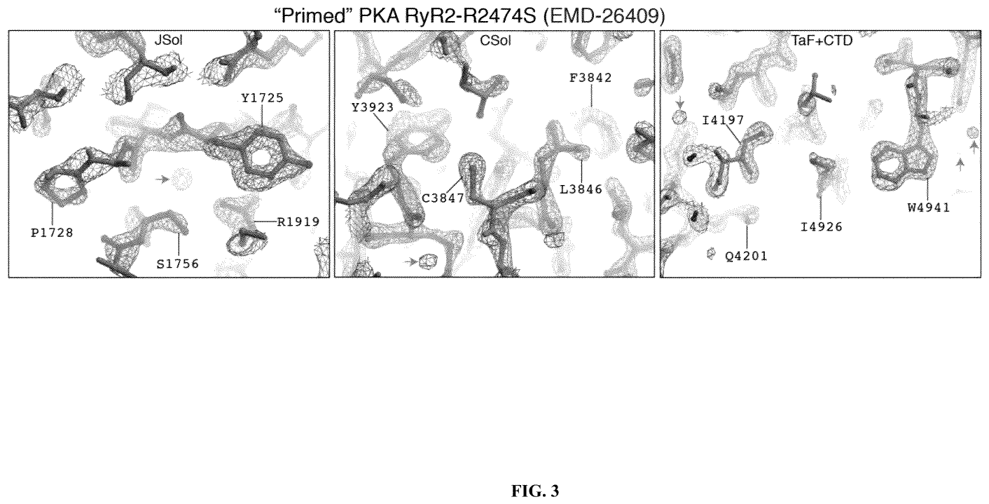

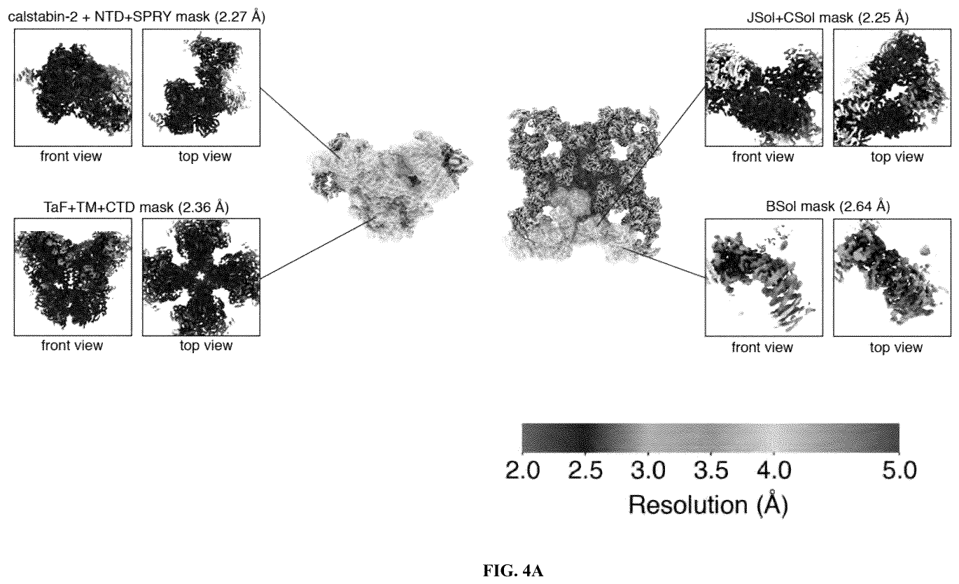

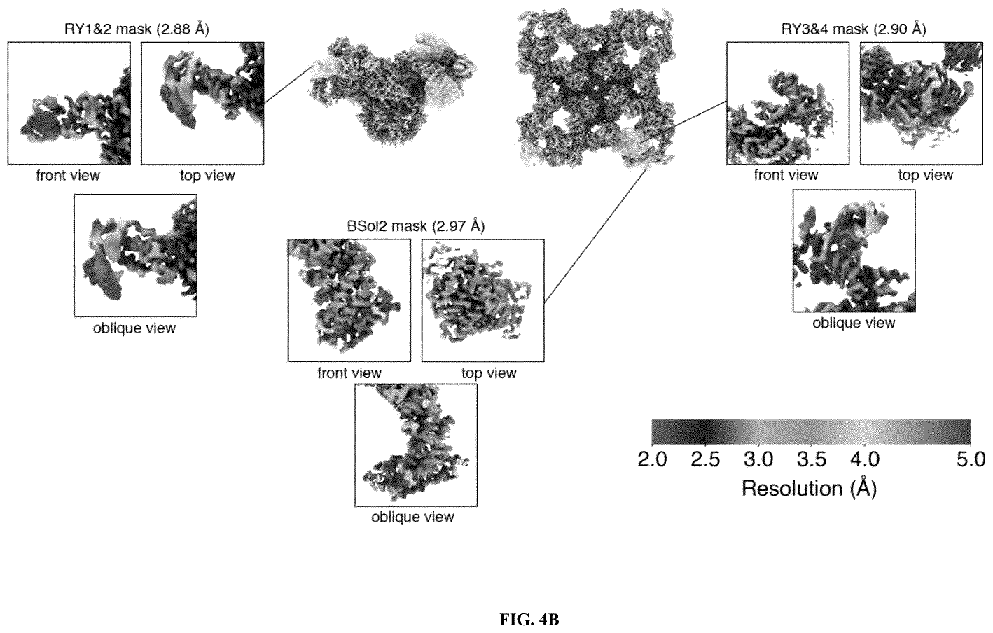

The highest-resolution cryo-EM coulombic potential maps (cryo-EM maps) show well-defined densities of most domains, including the NTD, SPRY1-3, JSol, BSol1, and activation core and channel domains, where near-atomic details including “holes” in aromatic residues were observed. FIG. 3 depicts representative high-resolution details of cryo-EM maps showing the holes in proline and aromatic residues, precise side-chain conformations, and stabilized water molecules (blue arrows). Respective regions of the JSol (left), CSol (center), and TaF+CTD domains (right) show that these details are distributed over the structure. FIG. 4A and FIG. 4B depict local refinement cryo-EM maps colored by local resolution shown for the “primed” PKA RyR2-R2474S, which showed the highest resolution and was used for building the initial atomic model.

For the remaining dynamic domains usually missing in previously published structures (RY1&2, RY3&4, and BSol2), the local resolution was reduced but sufficient to follow the backbone and detect bulky side chains. The cryo-EM map quality was sufficient to build atomic models with a high level of confidence for the previously less well-resolved domains of RyR2 and the accessory proteins calstabin-2 (known as PPIase FKBP1B or, alternatively, as FKBP12.6) and CaM. Using this high-resolution model of the human RyR2 isoform, the residue span and nomenclature of domains and subdomains for human RyR2 were updated as shown in TABLE 2.

TABLE 2
Nomenclature for Human RyR2

| Identification | Symbol | Residue Span |
|---|---|---|
| Cytosolic shell | | (1-3633) |
| N-terminal domain | NTD | (1-639) |
| N-terminal domain A | NTD-A | (1-219) |
| N-terminal domain B | NTD-B | (220-408) |
| N-terminal solenoid | NSol | (409-639) |
| SPRY domain | SPRY | (640-1646) |
| SP1a/ryanodine receptor domain 1 | SPRY1 | (640-861, 1463-1483, |
| | | 1595-1646) |
| SP1a/ryanodine receptor domain 2 | SPRY2 | (1076-1255) |
| SP1a/ryanodine receptor domain 3 | SPRY3 | (1256-1462, 1484-1594) |
| RYR repeats 1 & 2 | RY1&2 | (862-1076) |
| Junctional solenoid | JSol | (1647-2108) |
| Bridging solenoid | BSol | (2109-3564) |
| Bridging solenoid 1 | BSo11 | (2109-2681, 2916-3042) |
| Bridging solenoid 2 | BSo12 | (3042-3344) |
| Bridging solenoid 3 | BSo13 | (3345-3564) |
| RYR repeats 3 & 4 | RY3&4 | (2682-2915) |
| Shell-core linker peptide | SCLP | (3565-3633) |
| Activation core and channel | | (3634-4967) |
| Core solenoid | CSol | (3634-4130) |
| EF-hand pair | EF1&2 | (4016-4090) |
| Thumb and forefingers domain | TaF | (4131-4209) |
| Transmembrane domain | TM | (4237-4886) |
| Auxiliary intramembrane helices | Sx | (4237-4310) |
| Pseudo voltage sensor domain | pVSD | (4480-4750) |
| Channel pore domain | Pore | (4751-4886) |
| C-terminal domain | CTD | (4887-4967) |
| Zn-finger domain | ZnF | (4887-4914) |

To separate the particles in the closed state from those in the open states in the same dataset, local three-dimensional (3D) variability, clustering, and heterogeneous refinement were performed, which rendered better results than 3D classification or heterogeneous refinement alone. This improvement was attributed to the dynamic cytosolic shell of RyR2, which, being almost 90% of the mass of the protein, can predominate and result in mixed populations.

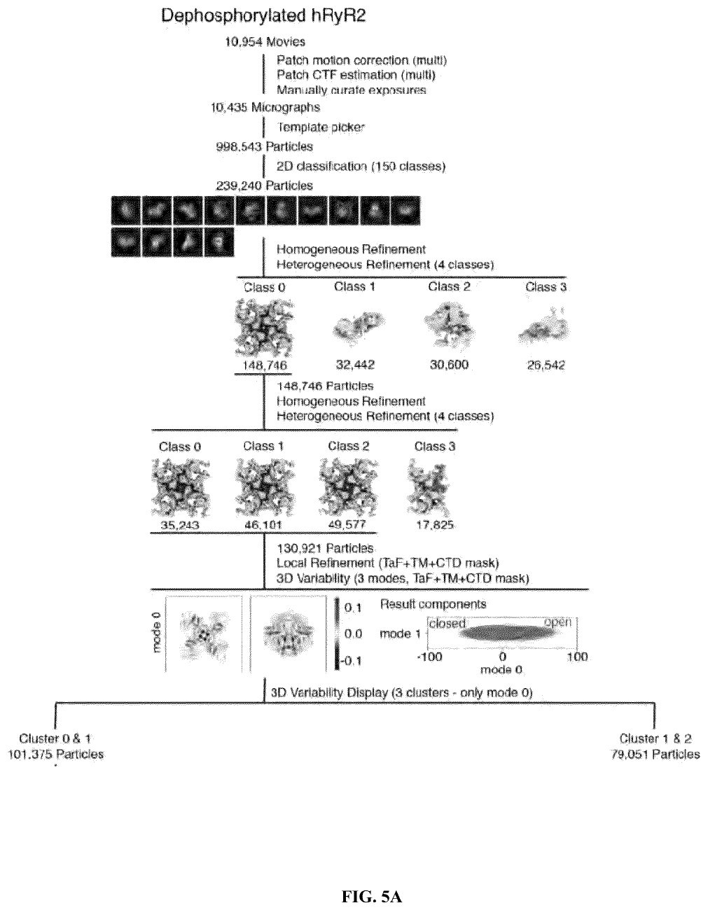

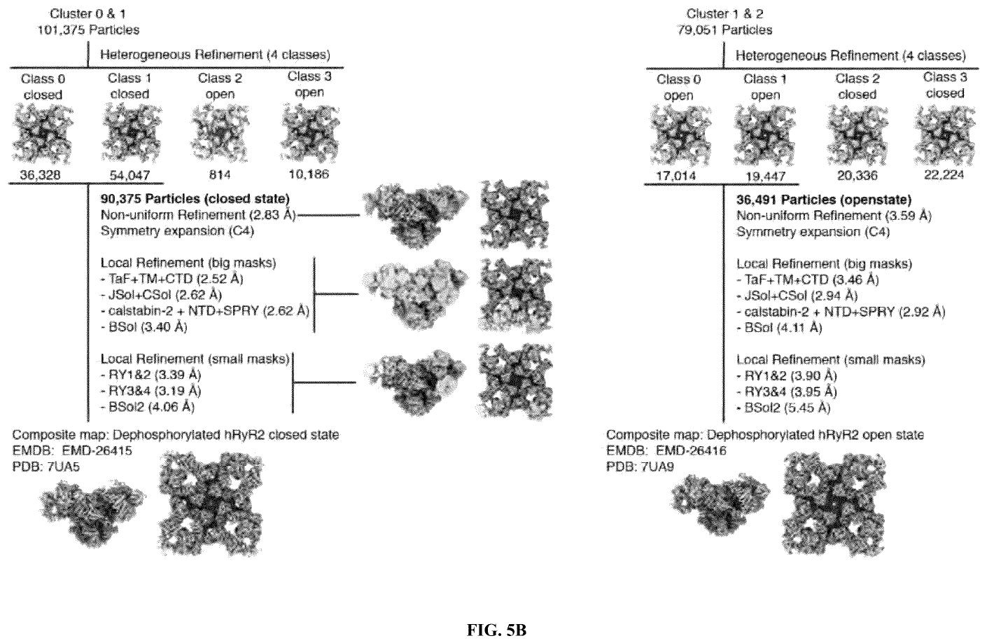

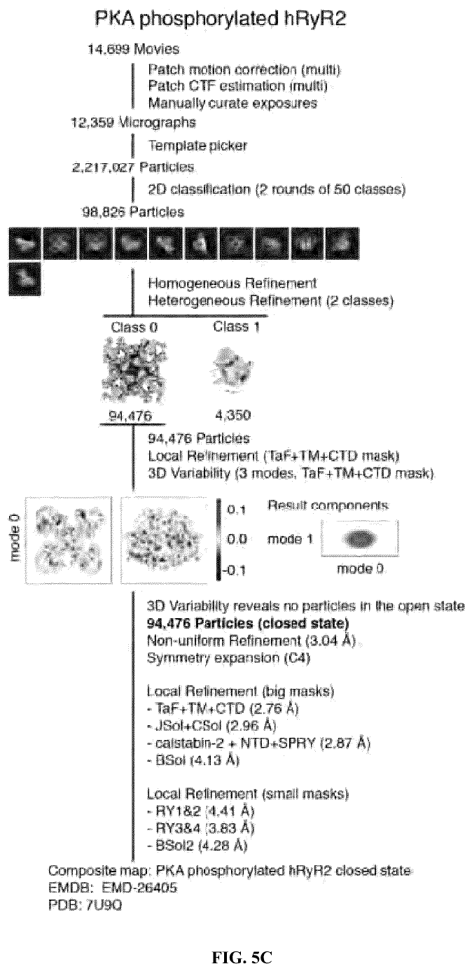

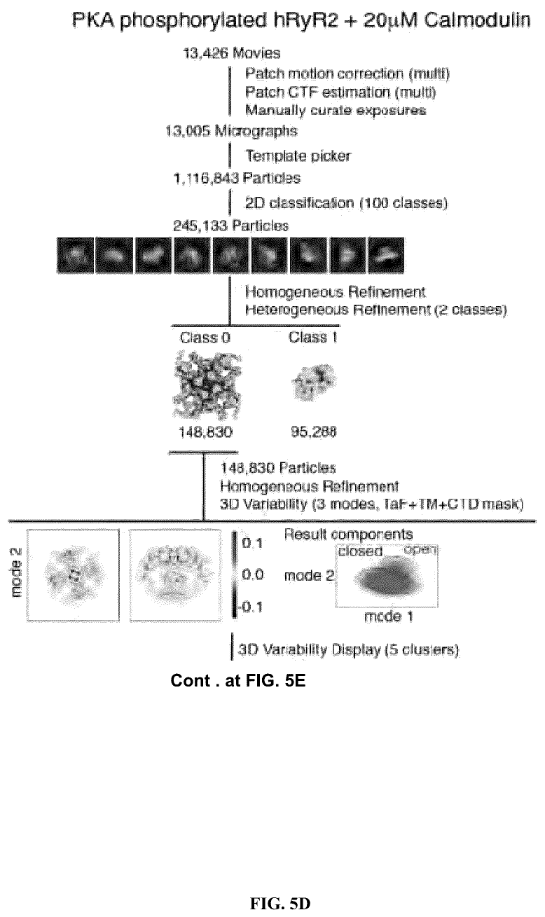

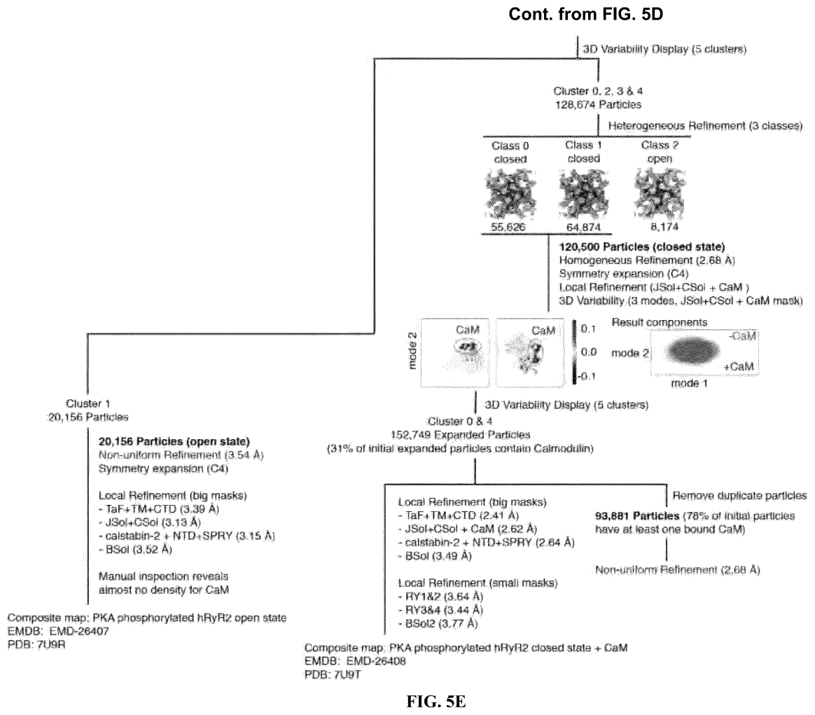

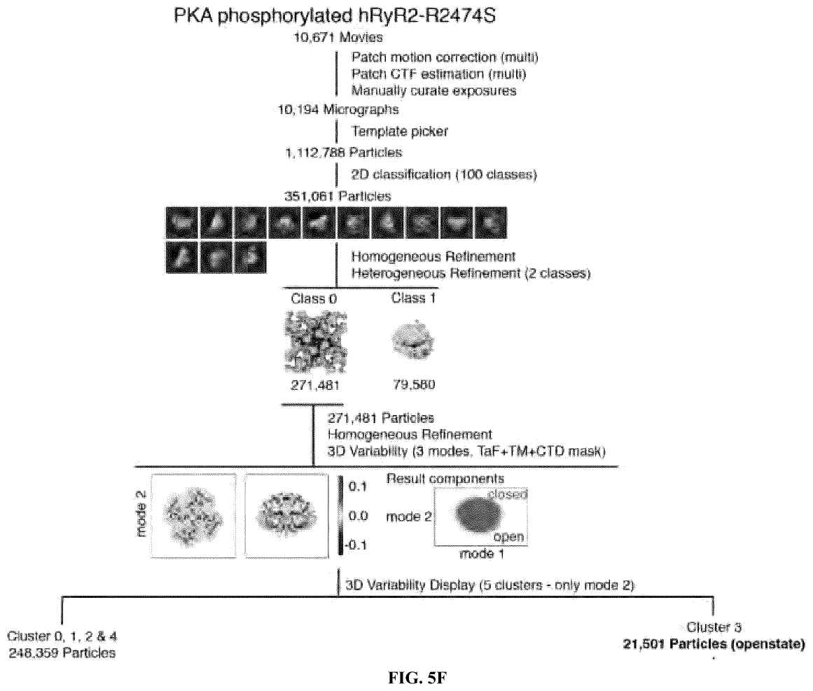

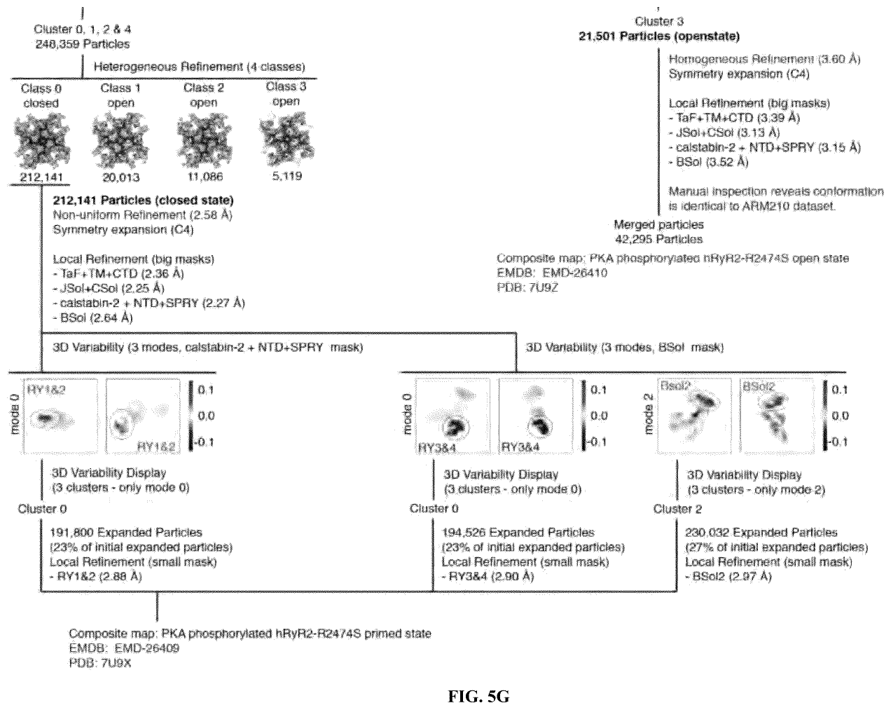

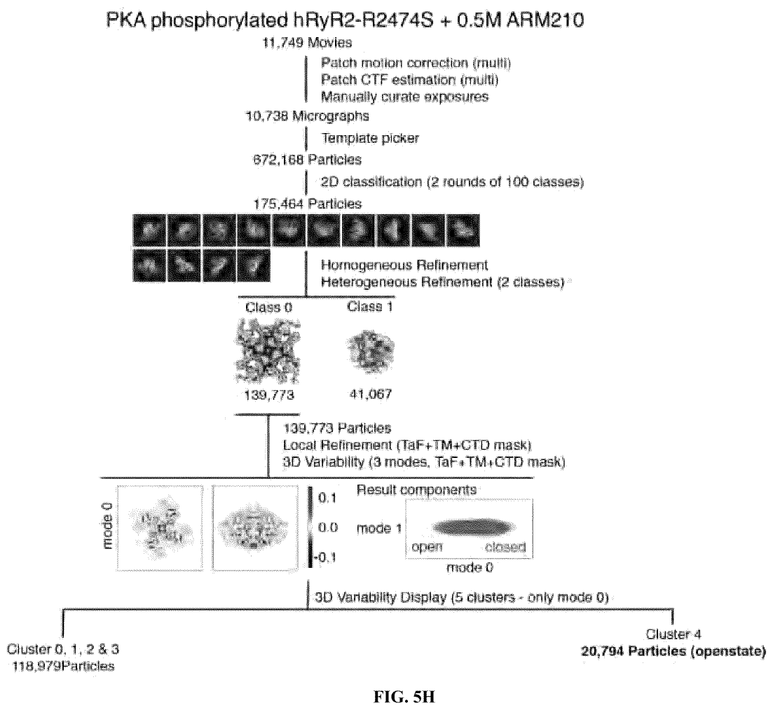

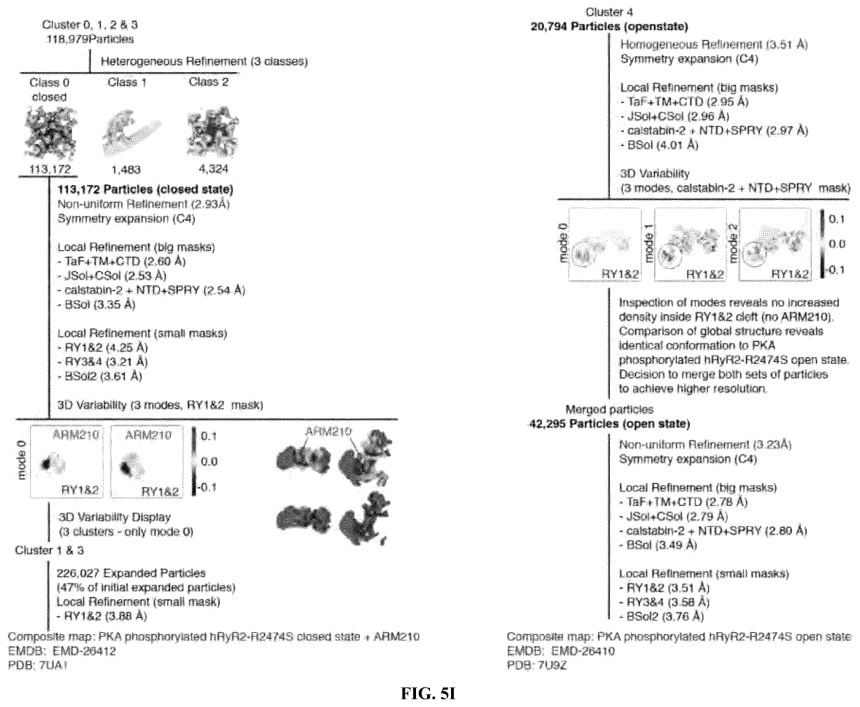

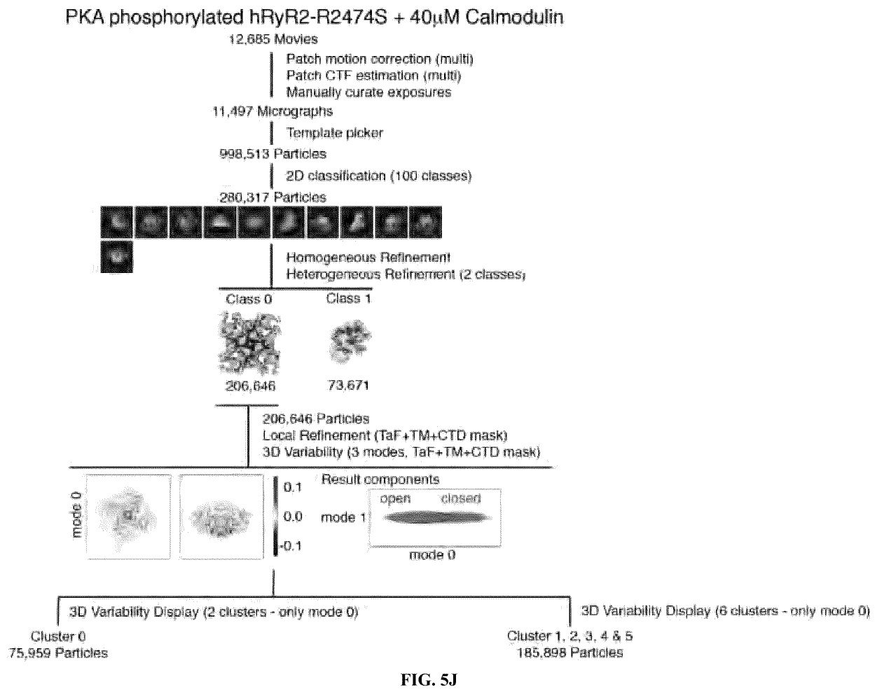

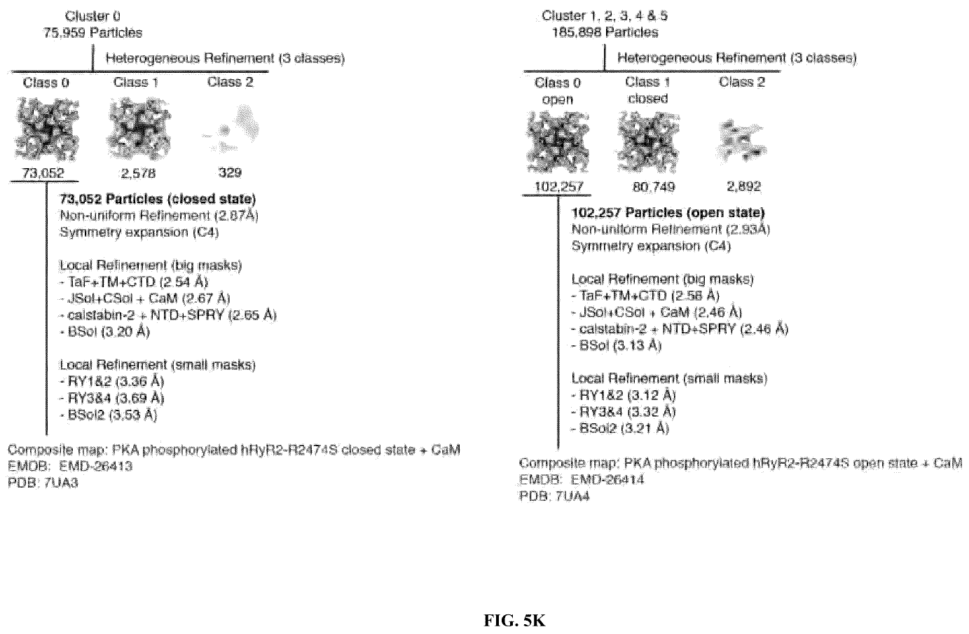

FIGS. 5A-5K are flowcharts that summarize the entire cryoSPARC processing of the cryo-EM datasets to obtain final composite maps of DeP-RyR2 (FIG. 5A and FIG. 5B), P-RyR2 (FIG. 5C), P-RyR2+CaM (FIG. 5D and FIG. 5E), P-RyR2-R2474S (FIG. 5F and FIG. 5G), P-RyR2-R2474S+Cpd1 (FIG. 5H and FIG. 5I), and P-RyR2-R2474S+CaM (FIG. 5J and FIG. 5K). Number of particles and resolution achieved for each refinement are shown for each step. Masks used are shown only in (FIG. 5B) but are identical in all processing procedures depicted in FIGS. 5A-5K. For better clarity, cryo-EM maps of the 3D variability of Compound 1 are shown in FIG. 5I.

The three-dimensional atomic coordinates as determined by cryo-EM for the RY1&2 domain in PKA phosphorylated RyR2-R2474S treated with 20M Compound 1 (P-RyR2-R2474S+Cpd1-C, residues 862-1076), are provided in TABLE 3. The three-dimensional atomic coordinates for Compound 1 and ATP bound to the RY1&2 domain of the P-RyR2-R2474S+Cpd1-C structure are provided in TABLE 4 and TABLE 5, respectively.

TABLE 3
Three-dimensional atomic coordinates of RY1&2 domain.

| Id¹ | type_symbol² | label_atom_id³ | label_comp_id⁴ | label_seq_id⁵ | Cartn_x⁶ | Cartn_y⁷ | Cartn_z⁸ | B_iso_or_equiv⁹ |
|---|---|---|---|---|---|---|---|---|
| 6450 | N | N | PHE | 862 | 74.748 | 182.682 | 181.942 | 121.08 |
| 6451 | C | CA | PHE | 862 | 74.218 | 181.323 | 181.964 | 121.08 |
| 6452 | C | C | PHE | 862 | 74.061 | 180.829 | 180.533 | 121.08 |
| 6453 | O | O | PHE | 862 | 73.314 | 181.419 | 179.745 | 121.08 |
| 6454 | C | CB | PHE | 862 | 72.885 | 181.267 | 182.706 | 121.08 |
| 6455 | C | CG | PHE | 862 | 72.197 | 179.935 | 182.614 | 121.08 |
| 6456 | C | CD1 | PHE | 862 | 72.898 | 178.761 | 182.827 | 121.08 |
| 6457 | C | CD2 | PHE | 862 | 70.849 | 179.858 | 182.308 | 121.08 |
| 6458 | C | CE1 | PHE | 862 | 72.268 | 177.535 | 182.739 | 121.08 |
| 6459 | C | CE2 | PHE | 862 | 70.213 | 178.635 | 182.22 | 121.08 |
| 6460 | C | CZ | PHE | 862 | 70.924 | 177.472 | 182.435 | 121.08 |
| 6461 | N | N | THR | 863 | 74.763 | 179.752 | 180.205 | 117.62 |
| 6462 | C | CA | THR | 863 | 74.615 | 179.081 | 178.918 | 117.62 |
| 6463 | C | C | THR | 863 | 74.3 | 177.614 | 179.172 | 117.62 |
| 6464 | O | O | THR | 863 | 75.19 | 176.857 | 179.603 | 117.62 |
| 6465 | C | CB | THR | 863 | 75.876 | 179.226 | 178.067 | 117.62 |
| 6466 | O | OG1 | THR | 863 | 76.027 | 180.592 | 177.665 | 117.62 |
| 6467 | C | CG2 | THR | 863 | 75.783 | 178.351 | 176.825 | 117.62 |
| 6468 | N | N | PRO | 864 | 73.066 | 177.169 | 178.935 | 121.81 |
| 6469 | C | CA | PRO | 864 | 72.74 | 175.752 | 179.141 | 121.81 |
| 6470 | C | C | PRO | 864 | 73.41 | 174.899 | 178.075 | 121.81 |
| 6471 | O | O | PRO | 864 | 73.23 | 175.123 | 176.875 | 121.81 |
| 6472 | C | CB | PRO | 864 | 71.213 | 175.715 | 179.022 | 121.81 |
| 6473 | C | CG | PRO | 864 | 70.86 | 176.897 | 178.186 | 121.81 |
| 6474 | C | CD | PRO | 864 | 71.96 | 177.919 | 178.316 | 121.81 |
| 6475 | N | N | ILE | 865 | 74.184 | 173.918 | 178.52 | 129.57 |
| 6476 | C | CA | ILE | 865 | 75.048 | 173.139 | 177.643 | 129.57 |
| 6477 | C | C | ILE | 865 | 74.427 | 171.76 | 177.436 | 129.57 |
| 6478 | O | O | ILE | 865 | 74.069 | 171.074 | 178.398 | 129.57 |
| 6479 | C | CB | ILE | 865 | 76.483 | 173.049 | 178.198 | 129.57 |
| 6480 | C | CG1 | ILE | 865 | 77.44 | 172.506 | 177.134 | 129.57 |
| 6481 | C | CG2 | ILE | 865 | 76.55 | 172.252 | 179.502 | 129.57 |
| 6482 | C | CD1 | ILE | 865 | 77.617 | 173.431 | 175.947 | 129.57 |
| 6483 | N | N | PRO | 866 | 74.226 | 171.348 | 176.189 | 138.28 |
| 6484 | C | CA | PRO | 866 | 73.634 | 170.033 | 175.925 | 138.28 |
| 6485 | C | C | PRO | 866 | 74.641 | 168.911 | 176.121 | 138.28 |
| 6486 | O | O | PRO | 866 | 75.85 | 169.13 | 176.223 | 138.28 |
| 6487 | C | CB | PRO | 866 | 73.204 | 170.125 | 174.454 | 138.28 |
| 6488 | C | CG | PRO | 866 | 73.318 | 171.584 | 174.092 | 138.28 |
| 6489 | C | CD | PRO | 866 | 74.408 | 172.118 | 174.951 | 138.28 |
| 6490 | N | N | VAL | 867 | 74.118 | 167.687 | 176.171 | 145.84 |
| 6491 | C | CA | VAL | 867 | 74.967 | 166.508 | 176.085 | 145.84 |
| 6492 | C | C | VAL | 867 | 75.276 | 166.226 | 174.622 | 145.84 |
| 6493 | O | O | VAL | 867 | 74.513 | 166.599 | 173.72 | 145.84 |
| 6494 | C | CB | VAL | 867 | 74.297 | 165.296 | 176.756 | 145.84 |
| 6495 | C | CG1 | VAL | 867 | 74.14 | 165.542 | 178.245 | 145.84 |
| 6496 | C | CG2 | VAL | 867 | 72.949 | 165.01 | 176.111 | 145.84 |
| 6497 | N | N | ASP | 868 | 76.409 | 165.574 | 174.378 | 164.98 |
| 6498 | C | CA | ASP | 868 | 76.797 | 165.25 | 173.014 | 164.98 |
| 6499 | C | C | ASP | 868 | 75.807 | 164.266 | 172.403 | 164.98 |
| 6500 | O | O | ASP | 868 | 75.265 | 163.397 | 173.091 | 164.98 |
| 6501 | C | CB | ASP | 868 | 78.21 | 164.668 | 172.987 | 164.98 |
| 6502 | C | CG | ASP | 868 | 78.729 | 164.459 | 171.578 | 164.98 |
| 6503 | O | OD1 | ASP | 868 | 78.217 | 165.119 | 170.649 | 164.98 |
| 6504 | O | OD2 | ASP | 868 | 79.652 | 163.636 | 171.398 | 164.98 |
| 6505 | N | N | THR | 869 | 75.558 | 164.419 | 171.102 | 166.07 |
| 6506 | C | CA | THR | 869 | 74.63 | 163.557 | 170.388 | 166.07 |
| 6507 | C | C | THR | 869 | 75.235 | 162.888 | 169.162 | 166.07 |
| 6508 | O | O | THR | 869 | 74.484 | 162.373 | 168.328 | 166.07 |
| 6509 | C | CB | THR | 869 | 73.379 | 164.343 | 169.964 | 166.07 |
| 6510 | O | OG1 | THR | 869 | 73.77 | 165.526 | 169.257 | 166.07 |
| 6511 | C | CG2 | THR | 869 | 72.554 | 164.729 | 171.182 | 166.07 |
| 6512 | N | N | SER | 870 | 76.559 | 162.91 | 169.006 | 171.46 |
| 6513 | C | CA | SER | 870 | 77.182 | 162.13 | 167.942 | 171.46 |
| 6514 | C | C | SER | 870 | 77.225 | 160.65 | 168.301 | 171.46 |
| 6515 | O | O | SER | 870 | 77.075 | 159.787 | 167.429 | 171.46 |
| 6516 | C | CB | SER | 870 | 78.589 | 162.657 | 167.66 | 171.46 |
| 6517 | O | OG | SER | 870 | 79.429 | 162.501 | 168.791 | 171.46 |
| 6518 | N | N | GLN | 871 | 77.43 | 160.342 | 169.584 | 173.01 |
| 6519 | C | CA | GLN | 871 | 77.625 | 158.961 | 170.01 | 173.01 |
| 6520 | C | C | GLN | 871 | 76.309 | 158.212 | 170.179 | 173.01 |
| 6521 | O | O | GLN | 871 | 76.319 | 156.989 | 170.357 | 173.01 |
| 6522 | C | CB | GLN | 871 | 78.401 | 158.933 | 171.326 | 173.01 |
| 6523 | C | CG | GLN | 871 | 77.523 | 159.153 | 172.551 | 173.01 |
| 6524 | C | CD | GLN | 871 | 77.232 | 160.62 | 172.811 | 173.01 |
| 6525 | O | OE1 | GLN | 871 | 76.725 | 161.327 | 171.941 | 173.01 |
| 6526 | N | NE2 | GLN | 871 | 77.546 | 161.081 | 174.016 | 173.01 |
| 6527 | N | N | ILE | 872 | 75.178 | 158.913 | 170.133 | 166.17 |
| 6528 | C | CA | ILE | 872 | 73.904 | 158.318 | 170.522 | 166.17 |
| 6529 | C | C | ILE | 872 | 73.5 | 157.244 | 169.521 | 166.17 |
| 6530 | O | O | ILE | 872 | 73.535 | 157.443 | 168.301 | 166.17 |
| 6531 | C | CB | ILE | 872 | 72.813 | 159.398 | 170.663 | 166.17 |
| 6532 | C | CG1 | ILE | 872 | 72.644 | 160.176 | 169.358 | 166.17 |
| 6533 | C | CG2 | ILE | 872 | 73.145 | 160.344 | 171.807 | 166.17 |
| 6534 | C | CD1 | ILE | 872 | 71.528 | 161.195 | 169.389 | 166.17 |
| 6535 | N | N | VAL | 873 | 73.134 | 156.079 | 170.05 | 173.29 |
| 6536 | C | CA | VAL | 873 | 72.581 | 154.981 | 169.27 | 173.29 |
| 6537 | C | C | VAL | 873 | 71.38 | 154.43 | 170.023 | 173.29 |
| 6538 | O | O | VAL | 873 | 71.28 | 154.577 | 171.246 | 173.29 |
| 6539 | C | CB | VAL | 873 | 73.618 | 153.866 | 169.011 | 173.29 |
| 6540 | C | CG1 | VAL | 873 | 74.755 | 154.379 | 168.139 | 173.29 |
| 6541 | C | CG2 | VAL | 873 | 74.151 | 153.317 | 170.326 | 173.29 |
| 6542 | N | N | LEU | 874 | 70.47 | 153.807 | 169.294 | 176.69 |
| 6543 | C | CA | LEU | 874 | 69.303 | 153.216 | 169.932 | 176.69 |
| 6544 | C | C | LEU | 874 | 69.679 | 151.859 | 170.523 | 176.69 |
| 6545 | O | O | LEU | 874 | 70.272 | 151.029 | 169.827 | 176.69 |
| 6546 | C | CB | LEU | 874 | 68.155 | 153.09 | 168.926 | 176.69 |
| 6547 | C | CG | LEU | 874 | 66.831 | 152.423 | 169.31 | 176.69 |
| 6548 | C | CD1 | LEU | 874 | 65.703 | 153.034 | 168.499 | 176.69 |
| 6549 | C | CD2 | LEU | 874 | 66.875 | 150.932 | 169.056 | 176.69 |
| 6550 | N | N | PRO | 875 | 69.365 | 151.609 | 171.797 | 174.54 |
| 6551 | C | CA | PRO | 875 | 69.841 | 150.384 | 172.441 | 174.54 |
| 6552 | C | C | PRO | 875 | 69.26 | 149.149 | 171.781 | 174.54 |
| 6553 | O | O | PRO | 875 | 68.117 | 149.165 | 171.292 | 174.54 |
| 6554 | C | CB | PRO | 875 | 69.354 | 150.538 | 173.893 | 174.54 |
| 6555 | C | CG | PRO | 875 | 68.181 | 151.452 | 173.793 | 174.54 |
| 6556 | C | CD | PRO | 875 | 68.491 | 152.402 | 172.677 | 174.54 |
| 6557 | N | N | PRO | 876 | 70.001 | 148.036 | 171.769 | 176.91 |
| 6558 | C | CA | PRO | 876 | 69.699 | 146.963 | 170.805 | 176.91 |
| 6559 | C | C | PRO | 876 | 68.48 | 146.119 | 171.14 | 176.91 |
| 6560 | O | O | PRO | 876 | 68.011 | 145.386 | 170.262 | 176.91 |
| 6561 | C | CB | PRO | 876 | 70.977 | 146.109 | 170.803 | 176.91 |
| 6562 | C | CG | PRO | 876 | 71.805 | 146.567 | 171.957 | 176.91 |
| 6563 | C | CD | PRO | 876 | 71.15 | 147.725 | 172.635 | 176.91 |
| 6564 | N | N | HIS | 877 | 67.974 | 146.144 | 172.375 | 173.58 |
| 6565 | C | CA | HIS | 877 | 66.801 | 145.324 | 172.669 | 173.58 |
| 6566 | C | C | HIS | 877 | 65.531 | 146.15 | 172.838 | 173.58 |
| 6567 | O | O | HIS | 877 | 64.49 | 145.594 | 173.202 | 173.58 |
| 6568 | C | CB | HIS | 877 | 67.043 | 144.455 | 173.907 | 173.58 |
| 6569 | C | CG | HIS | 877 | 67.253 | 145.227 | 175.173 | 173.58 |
| 6570 | N | ND1 | HIS | 877 | 68.464 | 145.79 | 175.511 | 173.58 |
| 6571 | C | CD2 | HIS | 877 | 66.411 | 145.5 | 176.198 | 173.58 |
| 6572 | C | CE1 | HIS | 877 | 68.356 | 146.389 | 176.684 | 173.58 |
| 6573 | N | NE2 | HIS | 877 | 67.12 | 146.229 | 177.122 | 173.58 |
| 6574 | N | N | LEU | 878 | 65.588 | 147.461 | 172.592 | 174.63 |
| 6575 | C | CA | LEU | 878 | 64.427 | 148.312 | 172.832 | 174.63 |
| 6576 | C | C | LEU | 878 | 63.662 | 148.689 | 171.567 | 174.63 |
| 6577 | O | O | LEU | 878 | 62.798 | 149.571 | 171.63 | 174.63 |
| 6578 | C | CB | LEU | 878 | 64.836 | 149.59 | 173.571 | 174.63 |
| 6579 | C | CG | LEU | 878 | 65.149 | 149.484 | 175.067 | 174.63 |
| 6580 | C | CD1 | LEU | 878 | 66.489 | 148.834 | 175.323 | 174.63 |
| 6581 | C | CD2 | LEU | 878 | 65.088 | 150.86 | 175.715 | 174.63 |
| 6582 | N | N | GLU | 879 | 63.95 | 148.061 | 170.424 | 183.39 |
| 6583 | C | CA | GLU | 879 | 63.233 | 148.416 | 169.2 | 183.39 |
| 6584 | C | C | GLU | 879 | 61.745 | 148.104 | 169.313 | 183.39 |
| 6585 | O | O | GLU | 879 | 60.902 | 148.997 | 169.168 | 183.39 |
| 6586 | C | CB | GLU | 879 | 63.83 | 147.686 | 167.996 | 183.39 |
| 6587 | C | CG | GLU | 879 | 65.239 | 148.107 | 167.639 | 183.39 |
| 6588 | C | CD | GLU | 879 | 66.27 | 147.395 | 168.481 | 183.39 |
| 6589 | O | OE1 | GLU | 879 | 65.864 | 146.537 | 169.29 | 183.39 |
| 6590 | O | OE2 | GLU | 879 | 67.476 | 147.69 | 168.336 | 183.39 |
| 6591 | N | N | ARG | 880 | 61.403 | 146.839 | 169.572 | 185.93 |
| 6592 | C | CA | ARG | 880 | 60.001 | 146.44 | 169.617 | 185.93 |
| 6593 | C | C | ARG | 880 | 59.271 | 147.022 | 170.821 | 185.93 |
| 6594 | O | O | ARG | 880 | 58.036 | 147.056 | 170.826 | 185.93 |
| 6595 | C | CB | ARG | 880 | 59.89 | 144.915 | 169.63 | 185.93 |
| 6596 | C | CG | ARG | 880 | 60.425 | 144.267 | 170.896 | 185.93 |
| 6597 | C | CD | ARG | 880 | 60.086 | 142.786 | 170.95 | 185.93 |
| 6598 | N | NE | ARG | 880 | 60.823 | 142.019 | 169.953 | 185.93 |
| 6599 | C | CZ | ARG | 880 | 61.944 | 141.356 | 170.2 | 185.93 |
| 6600 | N | NH1 | ARG | 880 | 62.487 | 141.341 | 171.407 | 185.93 |
| 6601 | N | NH2 | ARG | 880 | 62.536 | 140.691 | 169.212 | 185.93 |
| 6602 | N | N | ILE | 881 | 60.002 | 147.48 | 171.837 | 184.38 |
| 6603 | C | CA | ILE | 881 | 59.395 | 147.982 | 173.064 | 184.38 |
| 6604 | C | C | ILE | 881 | 59.237 | 149.498 | 173.041 | 184.38 |
| 6605 | O | O | ILE | 881 | 58.747 | 150.081 | 174.02 | 184.38 |
| 6606 | C | CB | ILE | 881 | 60.207 | 147.537 | 174.294 | 184.38 |
| 6607 | C | CG1 | ILE | 881 | 59.359 | 147.587 | 175.56 | 184.38 |
| 6608 | C | CG2 | ILE | 881 | 61.411 | 148.429 | 174.489 | 184.38 |
| 6609 | C | CD1 | ILE | 881 | 60.04 | 146.974 | 176.745 | 184.38 |
| 6610 | N | N | ARG | 882 | 59.629 | 150.154 | 171.945 | 191.08 |
| 6611 | C | CA | ARG | 882 | 59.509 | 151.606 | 171.873 | 191.08 |
| 6612 | C | C | ARG | 882 | 58.056 | 152.046 | 171.982 | 191.08 |
| 6613 | O | O | ARG | 882 | 57.749 | 153.027 | 172.668 | 191.08 |
| 6614 | C | CB | ARG | 882 | 60.133 | 152.127 | 170.579 | 191.08 |
| 6615 | C | CG | ARG | 882 | 60.023 | 153.634 | 170.422 | 191.08 |
| 6616 | C | CD | ARG | 882 | 60.829 | 154.367 | 171.481 | 191.08 |
| 6617 | N | NE | ARG | 882 | 60.748 | 155.813 | 171.318 | 191.08 |
| 6618 | C | CZ | ARG | 882 | 59.785 | 156.57 | 171.826 | 191.08 |
| 6619 | N | NH1 | ARG | 882 | 58.799 | 156.048 | 172.537 | 191.08 |
| 6620 | N | NH2 | ARG | 882 | 59.811 | 157.883 | 171.615 | 191.08 |
| 6621 | N | N | GLU | 883 | 57.142 | 151.322 | 171.332 | 195.18 |
| 6622 | C | CA | GLU | 883 | 55.729 | 151.666 | 171.451 | 195.18 |
| 6623 | C | C | GLU | 883 | 55.254 | 151.546 | 172.893 | 195.18 |
| 6624 | O | O | GLU | 883 | 54.762 | 152.525 | 173.47 | 195.18 |
| 6625 | C | CB | GLU | 883 | 54.888 | 150.778 | 170.533 | 195.18 |
| 6626 | C | CG | GLU | 883 | 54.872 | 151.22 | 169.079 | 195.18 |
| 6627 | C | CD | GLU | 883 | 54.298 | 152.615 | 168.899 | 195.18 |
| 6628 | O | OE1 | GLU | 883 | 53.367 | 152.981 | 169.649 | 195.18 |
| 6629 | O | OE2 | GLU | 883 | 54.776 | 153.346 | 168.005 | 195.18 |
| 6630 | N | N | LYS | 884 | 55.476 | 150.385 | 173.517 | 193.06 |
| 6631 | C | CA | LYS | 884 | 54.968 | 150.179 | 174.869 | 193.06 |
| 6632 | C | C | LYS | 884 | 55.541 | 151.221 | 175.814 | 193.06 |
| 6633 | O | O | LYS | 884 | 54.833 | 151.728 | 176.689 | 193.06 |
| 6634 | C | CB | LYS | 884 | 55.293 | 148.769 | 175.368 | 193.06 |
| 6635 | C | CG | LYS | 884 | 54.702 | 148.458 | 176.751 | 193.06 |
| 6636 | C | CD | LYS | 884 | 55.659 | 148.736 | 177.908 | 193.06 |
| 6637 | C | CE | LYS | 884 | 56.787 | 147.732 | 177.97 | 193.06 |
| 6638 | N | NZ | LYS | 884 | 57.707 | 148.022 | 179.106 | 193.06 |
| 6639 | N | N | LEU | 885 | 56.815 | 151.576 | 175.634 | 189.96 |
| 6640 | C | CA | LEU | 885 | 57.387 | 152.659 | 176.423 | 189.96 |
| 6641 | C | C | LEU | 885 | 56.725 | 153.993 | 176.103 | 189.96 |
| 6642 | O | O | LEU | 885 | 56.601 | 154.844 | 176.986 | 189.96 |
| 6643 | C | CB | LEU | 885 | 58.895 | 152.737 | 176.194 | 189.96 |
| 6644 | C | CG | LEU | 885 | 59.693 | 151.539 | 176.713 | 189.96 |
| 6645 | C | CD1 | LEU | 885 | 61.157 | 151.651 | 176.323 | 189.96 |
| 6646 | C | CD2 | LEU | 885 | 59.541 | 151.4 | 178.219 | 189.96 |
| 6647 | N | N | ALA | 886 | 56.28 | 154.191 | 174.86 | 196.35 |
| 6648 | C | CA | ALA | 886 | 55.6 | 155.435 | 174.507 | 196.35 |
| 6649 | C | C | ALA | 886 | 54.281 | 155.585 | 175.261 | 196.35 |
| 6650 | O | O | ALA | 886 | 54.041 | 156.604 | 175.923 | 196.35 |
| 6651 | C | CB | ALA | 886 | 55.366 | 155.489 | 172.997 | 196.35 |
| 6652 | N | N | GLU | 887 | 53.413 | 154.57 | 175.183 | 200.88 |
| 6653 | C | CA | GLU | 887 | 52.174 | 154.671 | 175.962 | 200.88 |
| 6654 | C | C | GLU | 887 | 52.436 | 154.604 | 177.462 | 200.88 |
| 6655 | O | O | GLU | 887 | 51.659 | 155.163 | 178.245 | 200.88 |
| 6656 | C | CB | GLU | 887 | 51.114 | 153.628 | 175.573 | 200.88 |
| 6657 | C | CG | GLU | 887 | 50.495 | 153.769 | 174.174 | 200.88 |
| 6658 | C | CD | GLU | 887 | 51.31 | 153.163 | 173.067 | 200.88 |
| 6659 | O | OE1 | GLU | 887 | 52.344 | 152.561 | 173.373 | 200.88 |
| 6660 | O | OE2 | GLU | 887 | 50.911 | 153.286 | 171.89 | 200.88 |
| 6661 | N | N | ASN | 888 | 53.529 | 153.969 | 177.888 | 197.83 |
| 6662 | C | CA | ASN | 888 | 53.86 | 153.965 | 179.307 | 197.83 |
| 6663 | C | C | ASN | 888 | 54.207 | 155.371 | 179.781 | 197.83 |
| 6664 | O | O | ASN | 888 | 53.744 | 155.813 | 180.837 | 197.83 |
| 6665 | C | CB | ASN | 888 | 55.02 | 153.003 | 179.564 | 197.83 |
| 6666 | C | CG | ASN | 888 | 55.287 | 152.792 | 181.038 | 197.83 |
| 6667 | O | OD1 | ASN | 888 | 54.481 | 153.169 | 181.885 | 197.83 |
| 6668 | N | ND2 | ASN | 888 | 56.43 | 152.195 | 181.353 | 197.83 |
| 6669 | N | N | ILE | 889 | 55.005 | 156.097 | 178.996 | 199.93 |
| 6670 | C | CA | ILE | 889 | 55.34 | 157.476 | 179.333 | 199.93 |
| 6671 | C | C | ILE | 889 | 54.099 | 158.353 | 179.274 | 199.93 |
| 6672 | O | O | ILE | 889 | 53.953 | 159.294 | 180.059 | 199.93 |
| 6673 | C | CB | ILE | 889 | 56.457 | 157.999 | 178.412 | 199.93 |
| 6674 | C | CG1 | ILE | 889 | 57.747 | 157.213 | 178.647 | 199.93 |
| 6675 | C | CG2 | ILE | 889 | 56.689 | 159.485 | 178.641 | 199.93 |
| 6676 | C | CD1 | ILE | 889 | 58.293 | 157.342 | 180.052 | 199.93 |
| 6677 | N | N | HIS | 890 | 53.182 | 158.06 | 178.349 | 207.04 |
| 6678 | C | CA | HIS | 890 | 51.899 | 158.759 | 178.347 | 207.04 |
| 6679 | C | C | HIS | 890 | 51.161 | 158.537 | 179.665 | 207.04 |
| 6680 | O | O | HIS | 890 | 50.592 | 159.474 | 180.245 | 207.04 |
| 6681 | C | CB | HIS | 890 | 51.058 | 158.29 | 177.155 | 207.04 |
| 6682 | C | CG | HIS | 890 | 49.668 | 158.852 | 177.119 | 207.04 |
| 6683 | N | ND1 | HIS | 890 | 48.712 | 158.552 | 178.066 | 207.04 |
| 6684 | C | CD2 | HIS | 890 | 49.073 | 159.694 | 176.241 | 207.04 |
| 6685 | C | CE1 | HIS | 890 | 47.591 | 159.187 | 177.776 | 207.04 |
| 6686 | N | NE2 | HIS | 890 | 47.783 | 159.887 | 176.673 | 207.04 |
| 6687 | N | N | GLU | 891 | 51.167 | 157.294 | 180.153 | 208.01 |
| 6688 | C | CA | GLU | 891 | 50.507 | 156.981 | 181.416 | 208.01 |
| 6689 | C | C | GLU | 891 | 51.155 | 157.721 | 182.581 | 208.01 |
| 6690 | O | O | GLU | 891 | 50.458 | 158.301 | 183.422 | 208.01 |
| 6691 | C | CB | GLU | 891 | 50.533 | 155.472 | 181.655 | 208.01 |
| 6692 | C | CG | GLU | 891 | 49.534 | 154.695 | 180.815 | 208.01 |
| 6693 | C | CD | GLU | 891 | 50.094 | 153.377 | 180.318 | 208.01 |
| 6694 | O | OE1 | GLU | 891 | 50.853 | 152.731 | 181.071 | 208.01 |
| 6695 | O | OE2 | GLU | 891 | 49.776 | 152.987 | 179.175 | 208.01 |
| 6696 | N | N | LEU | 892 | 52.49 | 157.707 | 182.651 | 203.06 |
| 6697 | C | CA | LEU | 892 | 53.17 | 158.47 | 183.698 | 203.06 |
| 6698 | C | C | LEU | 892 | 52.871 | 159.956 | 183.592 | 203.06 |
| 6699 | O | O | LEU | 892 | 52.673 | 160.623 | 184.61 | 203.06 |
| 6700 | C | CB | LEU | 892 | 54.684 | 158.24 | 183.688 | 203.06 |
| 6701 | C | CG | LEU | 892 | 55.304 | 156.969 | 184.278 | 203.06 |
| 6702 | C | CD1 | LEU | 892 | 55.059 | 155.738 | 183.465 | 203.06 |
| 6703 | C | CD2 | LEU | 892 | 56.799 | 157.182 | 184.472 | 203.06 |
| 6704 | N | N | TRP | 893 | 52.837 | 160.497 | 182.375 | 209.79 |
| 6705 | C | CA | TRP | 893 | 52.554 | 161.917 | 182.208 | 209.79 |
| 6706 | C | C | TRP | 893 | 51.174 | 162.266 | 182.748 | 209.79 |
| 6707 | O | O | TRP | 893 | 51.022 | 163.219 | 183.523 | 209.79 |
| 6708 | C | CB | TRP | 893 | 52.683 | 162.297 | 180.732 | 209.79 |
| 6709 | C | CG | TRP | 893 | 52.582 | 163.767 | 180.461 | 209.79 |
| 6710 | C | CD1 | TRP | 893 | 53.571 | 164.695 | 180.607 | 209.79 |
| 6711 | C | CD2 | TRP | 893 | 51.441 | 164.473 | 179.958 | 209.79 |
| 6712 | N | NE1 | TRP | 893 | 53.111 | 165.939 | 180.246 | 209.79 |
| 6713 | C | CE2 | TRP | 893 | 51.807 | 165.829 | 179.842 | 209.79 |
| 6714 | C | CE3 | TRP | 893 | 50.143 | 164.092 | 179.603 | 209.79 |
| 6715 | C | CZ2 | TRP | 893 | 50.922 | 166.805 | 179.386 | 209.79 |
| 6716 | C | CZ3 | TRP | 893 | 49.266 | 165.062 | 179.151 | 209.79 |
| 6717 | C | CH2 | TRP | 893 | 49.66 | 166.402 | 179.047 | 209.79 |
| 6718 | N | N | VAL | 894 | 50.159 | 161.477 | 182.386 | 212.83 |
| 6719 | C | CA | VAL | 894 | 48.805 | 161.806 | 182.824 | 212.83 |
| 6720 | C | C | VAL | 894 | 48.652 | 161.6 | 184.331 | 212.83 |
| 6721 | O | O | VAL | 894 | 48.038 | 162.427 | 185.019 | 212.83 |
| 6722 | C | CB | VAL | 894 | 47.755 | 161.019 | 182.013 | 212.83 |
| 6723 | C | CG1 | VAL | 894 | 47.885 | 161.341 | 180.532 | 212.83 |
| 6724 | C | CG2 | VAL | 894 | 47.874 | 159.522 | 182.24 | 212.83 |
| 6725 | N | N | MET | 895 | 49.229 | 160.522 | 184.881 | 215.43 |
| 6726 | C | CA | MET | 895 | 49.084 | 160.301 | 186.318 | 215.43 |
| 6727 | C | C | MET | 895 | 49.857 | 161.345 | 187.115 | 215.43 |
| 6728 | O | O | MET | 895 | 49.384 | 161.811 | 188.155 | 215.43 |
| 6729 | C | CB | MET | 895 | 49.523 | 158.887 | 186.714 | 215.43 |
| 6730 | C | CG | MET | 895 | 50.991 | 158.561 | 186.505 | 215.43 |
| 6731 | S | SD | MET | 895 | 51.393 | 156.846 | 186.907 | 215.43 |
| 6732 | C | CE | MET | 895 | 50.666 | 155.964 | 185.529 | 215.43 |
| 6733 | N | N | ASN | 896 | 51.043 | 161.731 | 186.646 | 214.43 |
| 6734 | C | CA | ASN | 896 | 51.798 | 162.779 | 187.314 | 214.43 |
| 6735 | C | C | ASN | 896 | 51.031 | 164.092 | 187.283 | 214.43 |
| 6736 | O | O | ASN | 896 | 50.93 | 164.792 | 188.297 | 214.43 |
| 6737 | C | CB | ASN | 896 | 53.164 | 162.927 | 186.645 | 214.43 |
| 6738 | C | CG | ASN | 896 | 54.117 | 163.786 | 187.441 | 214.43 |
| 6739 | O | OD1 | ASN | 896 | 53.779 | 164.292 | 188.511 | 214.43 |
| 6740 | N | ND2 | ASN | 896 | 55.322 | 163.958 | 186.919 | 214.43 |
| 6741 | N | N | LYS | 897 | 50.449 | 164.425 | 186.128 | 216.99 |
| 6742 | C | CA | LYS | 897 | 49.693 | 165.665 | 186.016 | 216.99 |
| 6743 | C | C | LYS | 897 | 48.476 | 165.661 | 186.933 | 216.99 |
| 6744 | O | O | LYS | 897 | 48.125 | 166.702 | 187.5 | 216.99 |
| 6745 | C | CB | LYS | 897 | 49.271 | 165.886 | 184.562 | 216.99 |
| 6746 | C | CG | LYS | 897 | 48.513 | 167.181 | 184.316 | 216.99 |
| 6747 | C | CD | LYS | 897 | 49.39 | 168.391 | 184.616 | 216.99 |
| 6748 | C | CE | LYS | 897 | 50.505 | 168.529 | 183.584 | 216.99 |
| 6749 | N | NZ | LYS | 897 | 51.357 | 169.737 | 183.8 | 216.99 |
| 6750 | N | N | ILE | 898 | 47.825 | 164.508 | 187.1 | 222.49 |
| 6751 | C | CA | ILE | 898 | 46.595 | 164.478 | 187.887 | 222.49 |
| 6752 | C | C | ILE | 898 | 46.879 | 164.37 | 189.387 | 222.49 |
| 6753 | O | O | ILE | 898 | 46.108 | 164.888 | 190.202 | 222.49 |
| 6754 | C | CB | ILE | 898 | 45.667 | 163.35 | 187.395 | 222.49 |
| 6755 | C | CG1 | ILE | 898 | 44.258 | 163.532 | 187.964 | 222.49 |
| 6756 | C | CG2 | ILE | 898 | 46.215 | 161.989 | 187.762 | 222.49 |
| 6757 | C | CD1 | ILE | 898 | 43.499 | 164.69 | 187.355 | 222.49 |
| 6758 | N | N | GLU | 899 | 47.971 | 163.716 | 189.789 | 222.71 |
| 6759 | C | CA | GLU | 899 | 48.315 | 163.636 | 191.206 | 222.71 |
| 6760 | C | C | GLU | 899 | 49.36 | 164.657 | 191.643 | 222.71 |
| 6761 | O | O | GLU | 899 | 49.868 | 164.552 | 192.764 | 222.71 |
| 6762 | C | CB | GLU | 899 | 48.784 | 162.225 | 191.596 | 222.71 |
| 6763 | C | CG | GLU | 899 | 47.652 | 161.236 | 191.908 | 222.71 |
| 6764 | C | CD | GLU | 899 | 47.263 | 160.34 | 190.75 | 222.71 |
| 6765 | O | OE1 | GLU | 899 | 47.985 | 160.316 | 189.738 | 222.71 |
| 6766 | O | OE2 | GLU | 899 | 46.225 | 159.653 | 190.855 | 222.71 |
| 6767 | N | N | LEU | 900 | 49.699 | 165.635 | 190.801 | 223.05 |
| 6768 | C | CA | LEU | 900 | 50.414 | 166.802 | 191.306 | 223.05 |
| 6769 | C | C | LEU | 900 | 49.497 | 167.783 | 192.023 | 223.05 |
| 6770 | O | O | LEU | 900 | 49.971 | 168.829 | 192.48 | 223.05 |
| 6771 | C | CB | LEU | 900 | 51.148 | 167.529 | 190.175 | 223.05 |
| 6772 | C | CG | LEU | 900 | 52.518 | 166.988 | 189.763 | 223.05 |
| 6773 | C | CD1 | LEU | 900 | 53.037 | 167.72 | 188.535 | 223.05 |
| 6774 | C | CD2 | LEU | 900 | 53.506 | 167.097 | 190.914 | 223.05 |
| 6775 | N | N | GLY | 901 | 48.206 | 167.473 | 192.133 | 227.61 |
| 6776 | C | CA | GLY | 901 | 47.242 | 168.346 | 192.765 | 227.61 |
| 6777 | C | C | GLY | 901 | 46.22 | 168.948 | 191.827 | 227.61 |
| 6778 | O | O | GLY | 901 | 45.286 | 169.607 | 192.299 | 227.61 |
| 6779 | N | N | TRP | 902 | 46.362 | 168.743 | 190.522 | 230.04 |
| 6780 | C | CA | TRP | 902 | 45.421 | 169.285 | 189.556 | 230.04 |
| 6781 | C | C | TRP | 902 | 44.301 | 168.285 | 189.281 | 230.04 |
| 6782 | O | O | TRP | 902 | 44.419 | 167.087 | 189.552 | 230.04 |
| 6783 | C | CB | TRP | 902 | 46.132 | 169.656 | 188.254 | 230.04 |
| 6784 | C | CG | TRP | 902 | 47.177 | 170.725 | 188.413 | 230.04 |
| 6785 | C | CD1 | TRP | 902 | 48.254 | 170.704 | 189.252 | 230.04 |
| 6786 | C | CD2 | TRP | 902 | 47.233 | 171.978 | 187.721 | 230.04 |
| 6787 | N | NE1 | TRP | 902 | 48.98 | 171.863 | 189.12 | 230.04 |
| 6788 | C | CE2 | TRP | 902 | 48.374 | 172.662 | 188.187 | 230.04 |
| 6789 | C | CE3 | TRP | 902 | 46.432 | 172.587 | 186.75 | 230.04 |
| 6790 | C | CZ2 | TRP | 902 | 48.732 | 173.923 | 187.716 | 230.04 |
| 6791 | C | CZ3 | TRP | 902 | 46.789 | 173.839 | 186.285 | 230.04 |
| 6792 | C | CH2 | TRP | 902 | 47.929 | 174.493 | 186.767 | 230.04 |
| 6793 | N | N | GLN | 903 | 43.201 | 168.796 | 188.732 | 235.31 |
| 6794 | C | CA | GLN | 903 | 42.032 | 167.975 | 188.457 | 235.31 |
| 6795 | C | C | GLN | 903 | 41.333 | 168.498 | 187.211 | 235.31 |
| 6796 | O | O | GLN | 903 | 41.534 | 169.641 | 186.792 | 235.31 |
| 6797 | C | CB | GLN | 903 | 41.072 | 167.95 | 189.652 | 235.31 |
| 6798 | C | CG | GLN | 903 | 40.422 | 169.29 | 189.954 | 235.31 |
| 6799 | C | CD | GLN | 903 | 41.228 | 170.119 | 190.936 | 235.31 |
| 6800 | O | OE1 | GLN | 903 | 42.405 | 169.848 | 191.175 | 235.31 |
| 6801 | N | NE2 | GLN | 903 | 40.597 | 171.136 | 191.51 | 235.31 |
| 6802 | N | N | TYR | 904 | 40.508 | 167.636 | 186.62 | 235.11 |
| 6803 | C | CA | TYR | 904 | 39.791 | 167.988 | 185.401 | 235.11 |
| 6804 | C | C | TYR | 904 | 38.795 | 169.111 | 185.666 | 235.11 |
| 6805 | O | O | TYR | 904 | 38.073 | 169.1 | 186.667 | 235.11 |
| 6806 | C | CB | TYR | 904 | 39.07 | 166.757 | 184.85 | 235.11 |
| 6807 | C | CG | TYR | 904 | 38.151 | 167.042 | 183.686 | 235.11 |
| 6808 | C | CD1 | TYR | 904 | 38.642 | 167.571 | 182.5 | 235.11 |
| 6809 | C | CD2 | TYR | 904 | 36.79 | 166.778 | 183.772 | 235.11 |
| 6810 | C | CE1 | TYR | 904 | 37.804 | 167.83 | 181.433 | 235.11 |
| 6811 | C | CE2 | TYR | 904 | 35.944 | 167.034 | 182.711 | 235.11 |
| 6812 | C | CZ | TYR | 904 | 36.456 | 167.56 | 181.544 | 235.11 |
| 6813 | O | OH | TYR | 904 | 35.616 | 167.817 | 180.484 | 235.11 |
| 6814 | N | N | GLY | 905 | 38.751 | 170.08 | 184.753 | 235.87 |
| 6815 | C | CA | GLY | 905 | 37.875 | 171.219 | 184.896 | 235.87 |
| 6816 | C | C | GLY | 905 | 37.31 | 171.703 | 183.576 | 235.87 |
| 6817 | O | O | GLY | 905 | 37.768 | 171.317 | 182.497 | 235.87 |
| 6818 | N | N | PRO | 906 | 36.276 | 172.548 | 183.641 | 235.54 |
| 6819 | C | CA | PRO | 906 | 35.668 | 173.063 | 182.404 | 235.54 |
| 6820 | C | C | PRO | 906 | 36.564 | 174.016 | 181.628 | 235.54 |
| 6821 | O | O | PRO | 906 | 36.775 | 173.828 | 180.425 | 235.54 |
| 6822 | C | CB | PRO | 906 | 34.403 | 173.775 | 182.907 | 235.54 |
| 6823 | C | CG | PRO | 906 | 34.17 | 173.238 | 184.291 | 235.54 |
| 6824 | C | CD | PRO | 906 | 35.533 | 172.97 | 184.837 | 235.54 |
| 6825 | N | N | VAL | 907 | 37.1 | 175.038 | 182.295 | 233.89 |
| 6826 | C | CA | VAL | 907 | 37.833 | 176.112 | 181.637 | 233.89 |
| 6827 | C | C | VAL | 907 | 39.171 | 176.297 | 182.344 | 233.89 |
| 6828 | O | O | VAL | 907 | 39.31 | 176.005 | 183.535 | 233.89 |
| 6829 | C | CB | VAL | 907 | 37.018 | 177.428 | 181.626 | 233.89 |
| 6830 | C | CG1 | VAL | 907 | 36.736 | 177.904 | 183.046 | 233.89 |
| 6831 | C | CG2 | VAL | 907 | 37.704 | 178.511 | 180.796 | 233.89 |
| 6832 | N | N | ARG | 908 | 40.16 | 176.782 | 181.595 | 230.2 |
| 6833 | C | CA | ARG | 908 | 41.526 | 176.928 | 182.088 | 230.2 |
| 6834 | C | C | ARG | 908 | 41.692 | 178.304 | 182.723 | 230.2 |
| 6835 | O | O | ARG | 908 | 41.503 | 179.329 | 182.061 | 230.2 |
| 6836 | C | CB | ARG | 908 | 42.526 | 176.719 | 180.951 | 230.2 |
| 6837 | C | CG | ARG | 908 | 43.994 | 176.7 | 181.369 | 230.2 |
| 6838 | C | CD | ARG | 908 | 44.632 | 178.082 | 181.28 | 230.2 |
| 6839 | N | NE | ARG | 908 | 46.068 | 178.042 | 181.531 | 230.2 |
| 6840 | C | CZ | ARG | 908 | 46.831 | 179.115 | 181.687 | 230.2 |
| 6841 | N | NH1 | ARG | 908 | 46.327 | 180.337 | 181.622 | 230.2 |
| 6842 | N | NH2 | ARG | 908 | 48.132 | 178.959 | 181.913 | 230.2 |
| 6843 | N | N | ASP | 909 | 42.053 | 178.32 | 184.005 | 233.34 |
| 6844 | C | CA | ASP | 909 | 42.372 | 179.543 | 184.725 | 233.34 |
| 6845 | C | C | ASF | 909 | 43.584 | 179.297 | 185.61 | 233.34 |
| 6846 | O | O | ASF | 909 | 43.808 | 178.182 | 186.088 | 233.34 |
| 6847 | C | CB | ASP | 909 | 41.189 | 180.032 | 185.574 | 233.34 |
| 6848 | C | CG | ASP | 909 | 40.093 | 180.666 | 184.741 | 233.34 |
| 6849 | O | OD1 | ASF | 909 | 40.415 | 181.309 | 183.719 | 233.34 |
| 6850 | O | OD2 | ASP | 909 | 38.907 | 180.524 | 185.108 | 233.34 |
| 6851 | N | N | ASP | 910 | 44.371 | 180.355 | 185.823 | 233.95 |
| 6852 | C | CA | ASP | 910 | 45.572 | 180.224 | 186.641 | 233.95 |
| 6853 | C | C | ASP | 910 | 45.226 | 180.019 | 188.111 | 233.95 |
| 6854 | O | O | ASP | 910 | 45.833 | 179.181 | 188.789 | 233.95 |
| 6855 | C | CB | ASP | 910 | 46.467 | 181.453 | 186.461 | 233.95 |
| 6856 | C | CG | ASP | 910 | 45.744 | 182.754 | 186.76 | 233.95 |
| 6857 | O | OD1 | ASP | 910 | 44.517 | 182.72 | 186.991 | 233.95 |
| 6858 | O | OD2 | ASP | 910 | 46.406 | 183.813 | 186.764 | 233.95 |
| 6859 | N | N | ASN | 911 | 44.252 | 180.774 | 188.623 | 234.55 |
| 6860 | C | CA | ASN | 911 | 43.873 | 180.647 | 190.027 | 234.55 |
| 6861 | C | C | ASN | 911 | 43.117 | 179.353 | 190.293 | 234.55 |
| 6862 | O | O | ASN | 911 | 43.117 | 178.858 | 191.426 | 234.55 |
| 6863 | C | CB | ASN | 911 | 43.028 | 181.847 | 190.454 | 234.55 |
| 6864 | C | CG | ASN | 911 | 41.801 | 182.034 | 189.584 | 234.55 |
| 6865 | O | OD1 | ASN | 911 | 40.746 | 181.458 | 189.846 | 234.55 |
| 6866 | N | ND2 | ASN | 911 | 41.935 | 182.842 | 188.539 | 234.55 |
| 6867 | N | N | LYS | 912 | 42.467 | 178.795 | 189.269 | 233.92 |
| 6868 | C | CA | LYS | 912 | 41.677 | 177.584 | 189.463 | 233.92 |
| 6869 | C | C | LYS | 912 | 42.553 | 176.378 | 189.775 | 233.92 |
| 6870 | O | O | LYS | 912 | 42.105 | 175.455 | 190.465 | 233.92 |
| 6871 | C | CB | LYS | 912 | 40.823 | 177.314 | 188.225 | 233.92 |
| 6872 | C | CG | LYS | 912 | 39.411 | 177.867 | 188.312 | 233.92 |
| 6873 | C | CD | LYS | 912 | 38.652 | 177.644 | 187.014 | 233.92 |
| 6874 | C | CE | LYS | 912 | 38.12 | 176.224 | 186.927 | 233.92 |
| 6875 | N | NZ | LYS | 912 | 37.586 | 175.908 | 185.574 | 233.92 |
| 6876 | N | N | ARG | 913 | 43.794 | 176.369 | 189.281 | 232.94 |
| 6877 | C | CA | ARG | 913 | 44.713 | 175.243 | 189.459 | 232.94 |
| 6878 | C | C | ARG | 913 | 44.093 | 173.941 | 188.958 | 232.94 |
| 6879 | O | O | ARG | 913 | 44.263 | 172.878 | 189.558 | 232.94 |
| 6880 | C | CB | ARG | 913 | 45.153 | 175.108 | 190.919 | 232.94 |
| 6881 | C | CG | ARG | 913 | 45.959 | 176.286 | 191.447 | 232.94 |
| 6882 | C | CD | ARG | 913 | 47.363 | 176.318 | 190.864 | 232.94 |
| 6883 | N | NE | ARG | 913 | 47.455 | 177.187 | 189.697 | 232.94 |
| 6884 | C | CZ | ARG | 913 | 48.551 | 177.349 | 188.968 | 232.94 |
| 6885 | N | NH1 | ARG | 913 | 49.674 | 176.713 | 189.258 | 232.94 |
| 6886 | N | NH2 | ARG | 913 | 48.52 | 178.171 | 187.923 | 232.94 |
| 6887 | N | N | GLN | 914 | 43.367 | 174.026 | 187.846 | 235.52 |
| 6888 | C | CA | GLN | 914 | 42.646 | 172.897 | 187.279 | 235.52 |
| 6889 | C | C | GLN | 914 | 43.173 | 172.618 | 185.879 | 235.52 |
| 6890 | O | O | GLN | 914 | 43.654 | 173.527 | 185.195 | 235.52 |
| 6891 | C | CB | GLN | 914 | 41.14 | 173.176 | 187.24 | 235.52 |
| 6892 | C | CG | GLN | 914 | 40.447 | 172.967 | 188.579 | 235.52 |
| 6893 | C | CD | GLN | 914 | 38.99 | 172.579 | 188.436 | 235.52 |
| 6894 | O | OE1 | GLN | 914 | 38.671 | 171.47 | 188.013 | 235.52 |
| 6895 | N | NE2 | GLN | 914 | 38.096 | 173.493 | 188.794 | 235.52 |
| 6896 | N | N | HIS | 915 | 43.088 | 171.359 | 185.459 | 235.61 |
| 6897 | C | CA | HIS | 915 | 43.644 | 170.927 | 184.178 | 235.61 |
| 6898 | C | C | HIS | 915 | 42.519 | 170.492 | 183.25 | 235.61 |
| 6899 | O | O | HIS | 915 | 42.106 | 169.318 | 183.286 | 235.61 |
| 6900 | C | CB | HIS | 915 | 44.641 | 169.788 | 184.388 | 235.61 |
| 6901 | C | CG | HIS | 915 | 45.301 | 169.318 | 183.13 | 235.61 |
| 6902 | N | ND1 | HIS | 915 | 46.278 | 170.047 | 182.486 | 235.61 |
| 6903 | C | CD2 | HIS | 915 | 45.134 | 168.189 | 182.402 | 235.61 |
| 6904 | C | CE1 | HIS | 915 | 46.68 | 169.39 | 181.413 | 235.61 |
| 6905 | N | NE2 | HIS | 915 | 46.002 | 168.258 | 181.34 | 235.61 |
| 6906 | N | N | PRO | 916 | 41.991 | 171.387 | 182.412 | 234.91 |
| 6907 | C | CA | PRO | 916 | 40.875 | 171.003 | 181.533 | 234.91 |
| 6908 | C | C | PRO | 916 | 41.28 | 170.113 | 180.373 | 234.91 |
| 6909 | O | O | PRO | 916 | 40.4 | 169.579 | 179.687 | 234.91 |
| 6910 | C | CB | PRO | 916 | 40.342 | 172.353 | 181.028 | 234.91 |
| 6911 | C | CG | PRO | 916 | 40.909 | 173.378 | 181.952 | 234.91 |
| 6912 | C | CD | PRO | 916 | 42.22 | 172.839 | 182.416 | 234.91 |
| 6913 | N | N | CYS | 917 | 42.577 | 169.931 | 180.129 | 233.71 |
| 6914 | C | CA | CYS | 917 | 43.041 | 169.258 | 178.922 | 233.71 |
| 6915 | C | C | CYS | 917 | 42.78 | 167.756 | 178.927 | 233.71 |
| 6916 | O | O | CYS | 917 | 43.107 | 167.089 | 177.939 | 233.71 |
| 6917 | C | CB | CYS | 917 | 44.533 | 169.524 | 178.713 | 233.71 |
| 6918 | S | SG | CYS | 917 | 45.001 | 171.266 | 178.842 | 233.71 |
| 6919 | N | N | LEU | 918 | 42.206 | 167.205 | 179.997 | 230.57 |
| 6920 | C | CA | LEU | 918 | 41.789 | 165.803 | 180.008 | 230.57 |
| 6921 | C | C | LEU | 918 | 40.485 | 165.67 | 179.22 | 230.57 |
| 6922 | O | O | LEU | 918 | 39.4 | 165.453 | 179.762 | 230.57 |
| 6923 | C | CB | LEU | 918 | 41.641 | 165.292 | 181.435 | 230.57 |
| 6924 | C | CG | LEU | 918 | 42.922 | 164.777 | 182.094 | 230.57 |
| 6925 | C | CD1 | LEU | 918 | 42.815 | 164.841 | 183.609 | 230.57 |
| 6926 | C | CD2 | LEU | 918 | 43.231 | 163.36 | 181.631 | 230.57 |
| 6927 | N | N | VAL | 919 | 40.616 | 165.806 | 177.902 | 231.33 |
| 6928 | C | CA | VAL | 919 | 39.484 | 165.813 | 176.985 | 231.33 |
| 6929 | C | C | VAL | 919 | 39.901 | 165.04 | 175.738 | 231.33 |
| 6930 | O | O | VAL | 919 | 41.029 | 164.542 | 175.655 | 231.33 |
| 6931 | C | CB | VAL | 919 | 39.045 | 167.255 | 176.651 | 231.33 |
| 6932 | C | CG1 | VAL | 919 | 40.035 | 167.92 | 175.698 | 231.33 |
| 6933 | C | CG2 | VAL | 919 | 37.606 | 167.307 | 176.126 | 231.33 |
| 6934 | N | N | GLU | 920 | 38.993 | 164.921 | 174.772 | 229.76 |
| 6935 | C | CA | GLU | 920 | 39.283 | 164.206 | 173.538 | 229.76 |
| 6936 | C | C | GLU | 920 | 40.49 | 164.816 | 172.827 | 229.76 |
| 6937 | O | O | GLU | 920 | 40.868 | 165.969 | 173.055 | 229.76 |
| 6938 | C | CB | GLU | 920 | 38.064 | 164.217 | 172.616 | 229.76 |
| 6939 | C | CG | GLU | 920 | 37.089 | 163.078 | 172.865 | 229.76 |
| 6940 | C | CD | GLU | 920 | 36.013 | 163.441 | 173.871 | 229.76 |
| 6941 | O | OE1 | GLU | 920 | 35.931 | 164.626 | 174.254 | 229.76 |
| 6942 | O | OE2 | GLU | 920 | 35.25 | 162.54 | 174.277 | 229.76 |
| 6943 | N | N | PHE | 921 | 41.092 | 164.019 | 171.941 | 228.6 |
| 6944 | C | CA | PHE | 921 | 42.341 | 164.415 | 171.298 | 228.6 |
| 6945 | C | C | PHE | 921 | 42.148 | 165.524 | 170.271 | 228.6 |
| 6946 | O | O | PHE | 921 | 43.121 | 165.937 | 169.63 | 228.6 |
| 6947 | C | CB | PHE | 921 | 42.996 | 163.196 | 170.642 | 228.6 |
| 6948 | C | CG | PHE | 921 | 42.176 | 162.584 | 169.538 | 228.6 |
| 6949 | C | CD1 | PHE | 921 | 41.252 | 161.589 | 169.814 | 228.6 |
| 6950 | C | CD2 | PHE | 921 | 42.34 | 162.991 | 168.223 | 228.6 |
| 6951 | C | CE1 | PHE | 921 | 40.499 | 161.021 | 168.803 | 228.6 |
| 6952 | C | CE2 | PHE | 921 | 41.59 | 162.427 | 167.208 | 228.6 |
| 6953 | C | CZ | PHE | 921 | 40.669 | 161.44 | 167.498 | 228.6 |
| 6954 | N | N | SER | 922 | 40.916 | 166.005 | 170.087 | 230.33 |
| 6955 | C | CA | SER | 922 | 40.666 | 167.048 | 169.096 | 230.33 |
| 6956 | C | C | SER | 922 | 41.44 | 168.32 | 169.42 | 230.33 |
| 6957 | O | O | SER | 922 | 42.179 | 168.842 | 168.576 | 230.33 |
| 6958 | C | CB | SER | 922 | 39.167 | 167.339 | 169.012 | 230.33 |
| 6959 | O | OG | SER | 922 | 38.704 | 167.963 | 170.197 | 230.33 |
| 6960 | N | N | LYS | 923 | 41.287 | 168.834 | 170.64 | 226.95 |
| 6961 | C | CA | LYS | 923 | 41.992 | 170.041 | 171.052 | 226.95 |
| 6962 | C | C | LYS | 923 | 43.325 | 169.735 | 171.724 | 226.95 |
| 6963 | O | O | LYS | 923 | 44.217 | 170.591 | 171.713 | 226.95 |
| 6964 | C | CB | LYS | 923 | 41.105 | 170.872 | 171.993 | 226.95 |
| 6965 | C | CG | LYS | 923 | 41.692 | 172.216 | 172.444 | 226.95 |
| 6966 | C | CD | LYS | 923 | 42.425 | 172.136 | 173.78 | 226.95 |
| 6967 | C | CE | LYS | 923 | 41.471 | 171.932 | 174.942 | 226.95 |
| 6968 | N | NZ | LYS | 923 | 42.204 | 171.87 | 176.237 | 226.95 |
| 6969 | N | N | LEU | 924 | 43.482 | 168.535 | 172.282 | 220.74 |
| 6970 | C | CA | LEU | 924 | 44.664 | 168.213 | 173.066 | 220.74 |
| 6971 | C | C | LEU | 924 | 45.923 | 168.412 | 172.224 | 220.74 |
| 6972 | O | O | LEU | 924 | 46.016 | 167.902 | 171.103 | 220.74 |
| 6973 | C | CB | LEU | 924 | 44.589 | 166.775 | 173.578 | 220.74 |
| 6974 | C | CG | LEU | 924 | 45.496 | 166.367 | 174.744 | 220.74 |
| 6975 | C | CD1 | LEU | 924 | 44.856 | 165.23 | 175.527 | 220.74 |
| 6976 | C | CD2 | LEU | 924 | 46.888 | 165.968 | 174.276 | 220.74 |
| 6977 | N | N | PRO | 925 | 46.898 | 169.163 | 172.735 | 213.7 |
| 6978 | C | CA | PRO | 925 | 48.038 | 169.569 | 171.902 | 213.7 |
| 6979 | C | C | PRO | 925 | 48.844 | 168.385 | 171.388 | 213.7 |
| 6980 | O | O | PRO | 925 | 49.502 | 167.668 | 172.152 | 213.7 |
| 6981 | C | CB | PRO | 925 | 48.866 | 170.444 | 172.848 | 213.7 |
| 6982 | C | CG | PRO | 925 | 47.902 | 170.94 | 173.865 | 213.7 |
| 6983 | C | CD | PRO | 925 | 46.867 | 169.866 | 174.029 | 213.7 |
| 6984 | N | N | GLU | 926 | 48.8 | 168.192 | 170.068 | 209.72 |
| 6985 | C | CA | GLU | 926 | 49.663 | 167.2 | 169.441 | 209.72 |
| 6986 | C | C | GLU | 926 | 51.127 | 167.583 | 169.595 | 209.72 |
| 6987 | O | O | GLU | 926 | 51.997 | 166.71 | 169.684 | 209.72 |
| 6988 | C | CB | GLU | 926 | 49.298 | 167.038 | 167.966 | 209.72 |
| 6989 | C | CG | GLU | 926 | 47.983 | 166.311 | 167.73 | 209.72 |
| 6990 | C | CD | GLU | 926 | 48.009 | 164.874 | 168.216 | 209.72 |
| 6991 | O | OE1 | GLU | 926 | 49.089 | 164.247 | 168.171 | 209.72 |
| 6992 | O | OE2 | GLU | 926 | 46.95 | 164.372 | 168.645 | 209.72 |
| 6993 | N | N | GLN | 927 | 51.419 | 168.886 | 169.64 | 205.13 |
| 6994 | C | CA | GLN | 927 | 52.781 | 169.315 | 169.93 | 205.13 |
| 6995 | C | C | GLN | 927 | 53.181 | 168.933 | 171.35 | 205.13 |
| 6996 | O | O | GLN | 927 | 54.337 | 168.583 | 171.599 | 205.13 |
| 6997 | C | CB | GLN | 927 | 52.931 | 170.822 | 169.7 | 205.13 |
| 6998 | C | CG | GLN | 927 | 52.268 | 171.714 | 170.738 | 205.13 |
| 6999 | C | CD | GLN | 927 | 50.794 | 171.939 | 170.466 | 205.13 |
| 7000 | O | OE1 | GLN | 927 | 50.169 | 171.198 | 169.708 | 205.13 |
| 7001 | N | NE2 | GLN | 927 | 50.23 | 172.968 | 171.087 | 205.13 |
| 7002 | N | N | GLU | 928 | 52.234 | 168.968 | 172.293 | 201.87 |
| 7003 | C | CA | GLU | 928 | 52.537 | 168.507 | 173.645 | 201.87 |
| 7004 | C | C | GLU | 928 | 52.755 | 167 | 173.68 | 201.87 |
| 7005 | O | O | GLU | 928 | 53.627 | 166.512 | 174.407 | 201.87 |
| 7006 | C | CB | GLU | 928 | 51.426 | 168.91 | 174.614 | 201.87 |
| 7007 | C | CG | GLU | 928 | 51.602 | 168.359 | 176.023 | 201.87 |
| 7008 | C | CD | GLU | 928 | 52.903 | 168.792 | 176.676 | 201.87 |
| 7009 | O | OE1 | GLU | 928 | 53.428 | 169.87 | 176.325 | 201.87 |
| 7010 | O | OE2 | GLU | 928 | 53.409 | 168.042 | 177.537 | 201.87 |
| 7011 | N | N | ARG | 929 | 51.975 | 166.245 | 172.902 | 202.57 |
| 7012 | C | CA | ARG | 929 | 52.229 | 164.809 | 172.799 | 202.57 |
| 7013 | C | C | ARG | 929 | 53.623 | 164.541 | 172.242 | 202.57 |
| 7014 | O | O | ARG | 929 | 54.364 | 163.695 | 172.764 | 202.57 |
| 7015 | C | CB | ARG | 929 | 51.173 | 164.147 | 171.913 | 202.57 |
| 7016 | C | CG | ARG | 929 | 49.765 | 164.139 | 172.48 | 202.57 |
| 7017 | C | CD | ARG | 929 | 48.78 | 163.639 | 171.433 | 202.57 |
| 7018 | N | NE | ARG | 929 | 47.394 | 163.733 | 171.875 | 202.57 |
| 7019 | C | CZ | ARG | 929 | 46.793 | 162.842 | 172.652 | 202.57 |
| 7020 | N | NH1 | ARG | 929 | 47.43 | 161.771 | 173.096 | 202.57 |
| 7021 | N | NH2 | ARG | 929 | 45.521 | 163.03 | 172.99 | 202.57 |
| 7022 | N | N | ASN | 930 | 53.997 | 165.264 | 171.185 | 198.21 |
| 7023 | C | CA | ASN | 930 | 55.319 | 165.094 | 170.593 | 198.21 |
| 7024 | C | C | ASN | 930 | 56.41 | 165.481 | 171.58 | 198.21 |
| 7025 | O | O | ASN | 930 | 57.456 | 164.829 | 171.647 | 198.21 |
| 7026 | C | CB | ASN | 930 | 55.429 | 165.918 | 169.311 | 198.21 |
| 7027 | C | CG | ASN | 930 | 54.574 | 165.366 | 168.188 | 198.21 |
| 7028 | O | OD1 | ASN | 930 | 54.508 | 164.154 | 167.981 | 198.21 |
| 7029 | N | ND2 | ASN | 930 | 53.913 | 166.254 | 167.455 | 198.21 |
| 7030 | N | N | TYR | 931 | 56.183 | 166.543 | 172.357 | 193.04 |
| 7031 | C | CA | TYR | 931 | 57.146 | 166.944 | 173.377 | 193.04 |
| 7032 | C | C | TYR | 931 | 57.293 | 165.87 | 174.445 | 193.04 |
| 7033 | O | O | TYR | 931 | 58.405 | 165.593 | 174.907 | 193.04 |
| 7034 | C | CB | TYR | 931 | 56.727 | 168.273 | 174.007 | 193.04 |
| 7035 | C | CG | TYR | 931 | 56.819 | 169.458 | 173.072 | 193.04 |
| 7036 | C | CD1 | TYR | 931 | 57.525 | 169.375 | 171.878 | 193.04 |
| 7037 | C | CD2 | TYR | 931 | 56.197 | 170.66 | 173.382 | 193.04 |
| 7038 | C | CE1 | TYR | 931 | 57.609 | 170.457 | 171.021 | 193.04 |
| 7039 | C | CE2 | TYR | 931 | 56.276 | 171.746 | 172.532 | 193.04 |
| 7040 | C | CZ | TYR | 931 | 56.983 | 171.639 | 171.353 | 193.04 |
| 7041 | O | OH | TYR | 931 | 57.064 | 172.719 | 170.504 | 193.04 |
| 7042 | N | N | ASN | 932 | 56.181 | 165.254 | 174.848 | 191.16 |
| 7043 | C | CA | ASN | 932 | 56.241 | 164.183 | 175.835 | 191.16 |
| 7044 | C | C | ASN | 932 | 57.069 | 163.015 | 175.314 | 191.16 |
| 7045 | O | O | ASN | 932 | 57.976 | 162.519 | 176 | 191.16 |
| 7046 | C | CB | ASN | 932 | 54.823 | 163.72 | 176.175 | 191.16 |
| 7047 | C | CG | ASN | 932 | 54.041 | 164.757 | 176.957 | 191.16 |
| 7048 | O | OD | ASN | 932 | 54.558 | 165.377 | 177.882 | 191.16 |
| 7049 | N | ND2 | ASN | 932 | 52.783 | 164.954 | 176.581 | 191.16 |
| 7050 | N | N | LEU | 933 | 56.787 | 162.585 | 174.082 | 180.56 |
| 7051 | C | CA | LEU | 933 | 57.53 | 161.471 | 173.498 | 180.56 |
| 7052 | C | C | LEU | 933 | 59.006 | 161.808 | 173.33 | 180.56 |
| 7053 | O | O | LEU | 933 | 59.882 | 160.981 | 173.623 | 180.56 |
| 7054 | C | CB | LEU | 933 | 56.913 | 161.067 | 172.158 | 180.56 |
| 7055 | C | CG | LEU | 933 | 55.706 | 160.126 | 172.18 | 180.56 |
| 7056 | C | CD1 | LEU | 933 | 56.168 | 158.816 | 172.759 | 180.56 |
| 7057 | C | CD2 | LEU | 933 | 54.519 | 160.65 | 172.968 | 180.56 |
| 7058 | N | N | GLN | 934 | 59.308 | 163.024 | 172.868 | 179.21 |
| 7059 | C | CA | GLN | 934 | 60.7 | 163.37 | 172.635 | 179.21 |
| 7060 | C | C | GLN | 934 | 61.462 | 163.544 | 173.937 | 179.21 |
| 7061 | O | O | GLN | 934 | 62.645 | 163.205 | 173.989 | 179.21 |
| 7062 | C | CB | GLN | 934 | 60.825 | 164.629 | 171.767 | 179.21 |
| 7063 | C | CG | GLN | 934 | 60.288 | 165.909 | 172.371 | 179.21 |
| 7064 | C | CD | GLN | 934 | 61.29 | 166.602 | 173.274 | 179.21 |
| 7065 | | OE1 | GLN | 934 | 62.501 | 166.494 | 173.076 | 179.21 |
| 7066 | N | NE2 | GLN | 934 | 60.79 | 167.313 | 174.278 | 179.21 |
| 7067 | N | N | MET | 935 | 60.82 | 164.031 | 175.004 | 175.62 |
| 7068 | C | CA | MET | 935 | 61.551 | 164.126 | 176.261 | 175.62 |
| 7069 | C | C | MET | 935 | 61.738 | 162.749 | 176.876 | 175.62 |
| 7070 | O | O | MET | 935 | 62.745 | 162.505 | 177.541 | 175.62 |
| 7071 | C | CB | MET | 935 | 60.859 | 165.051 | 177.264 | 175.62 |
| 7072 | C | CG | MET | 935 | 59.511 | 164.591 | 177.775 | 175.62 |
| 7073 | S | SD | MET | 935 | 58.81 | 165.808 | 178.905 | 175.62 |
| 7074 | C | CE | MET | 935 | 57.258 | 165.028 | 179.327 | 175.62 |
| 7075 | N | N | SER | 936 | 60.8 | 161.825 | 176.646 | 166.44 |
| 7076 | C | CA | SER | 936 | 61.014 | 160.454 | 177.104 | 166.44 |
| 7077 | C | C | SER | 936 | 62.206 | 159.82 | 176.395 | 166.44 |
| 7078 | O | O | SER | 936 | 63.08 | 159.212 | 177.034 | 166.44 |
| 7079 | C | CB | SER | 936 | 59.755 | 159.622 | 176.876 | 166.44 |
| 7080 | O | OG | SER | 936 | 59.358 | 159.662 | 175.517 | 166.44 |
| 7081 | N | N | LEU | 937 | 62.261 | 159.954 | 175.067 | 164.45 |
| 7082 | C | CA | LEU | 937 | 63.375 | 159.361 | 174.334 | 164.45 |
| 7083 | C | C | LEU | 937 | 64.684 | 160.072 | 174.656 | 164.45 |
| 7084 | O | O | LEU | 937 | 65.739 | 159.433 | 174.727 | 164.45 |
| 7085 | C | CB | LEU | 937 | 63.094 | 159.362 | 172.828 | 164.45 |
| 7086 | C | CG | LEU | 937 | 62.884 | 160.648 | 172.027 | 164.45 |
| 7087 | C | CD1 | LEU | 937 | 64.201 | 161.279 | 171.593 | 164.45 |
| 7088 | C | CD2 | LEU | 937 | 61.997 | 160.372 | 170.821 | 164.45 |
| 7089 | N | N | GLU | 938 | 64.636 | 161.386 | 174.883 | 162.4 |
| 7090 | C | CA | GLU | 938 | 65.834 | 162.11 | 175.29 | 162.4 |
| 7091 | C | C | GLU | 938 | 66.281 | 161.678 | 176.677 | 162.4 |
| 7092 | O | O | GLU | 938 | 67.481 | 161.586 | 176.948 | 162.4 |
| 7093 | C | CB | GLU | 938 | 65.576 | 163.616 | 175.252 | 162.4 |
| 7094 | C | CG | GLU | 938 | 66.817 | 164.471 | 175.456 | 162.4 |
| 7095 | C | CD | GLU | 938 | 67.719 | 164.486 | 174.238 | 162.4 |
| 7096 | O | OE1 | GLU | 938 | 67.209 | 164.268 | 173.119 | 162.4 |
| 7097 | O | OE2 | GLU | 938 | 68.935 | 164.724 | 174.397 | 162.4 |
| 7098 | N | N | THR | 939 | 65.33 | 161.387 | 177.564 | 153.44 |
| 7099 | C | CA | THR | 939 | 65.675 | 160.889 | 178.887 | 153.44 |
| 7100 | C | C | THR | 939 | 66.402 | 159.556 | 178.789 | 153.44 |
| 7101 | O | O | THR | 939 | 67.472 | 159.375 | 179.382 | 153.44 |
| 7102 | C | CB | THR | 939 | 64.41 | 160.753 | 179.735 | 153.44 |
| 7103 | O | OG1 | THR | 939 | 63.783 | 162.035 | 179.874 | 153.44 |
| 7104 | C | CG2 | THR | 939 | 64.747 | 160.203 | 181.114 | 153.44 |
| 7105 | N | N | LEU | 940 | 65.847 | 158.617 | 178.017 | 151.97 |
| 7106 | C | CA | LEU | 940 | 66.495 | 157.311 | 177.896 | 151.97 |
| 7107 | C | C | LEU | 940 | 67.848 | 157.431 | 177.199 | 151.97 |
| 7108 | O | O | LEU | 940 | 68.812 | 156.741 | 177.565 | 151.97 |
| 7109 | C | CB | LEU | 940 | 65.574 | 156.321 | 177.178 | 151.97 |
| 7110 | C | CG | LEU | 940 | 65.088 | 156.531 | 175.741 | 151.97 |
| 7111 | C | CD1 | LEU | 940 | 66.103 | 156.041 | 174.709 | 151.97 |
| 7112 | C | CD2 | LEU | 940 | 63.741 | 155.854 | 175.534 | 151.97 |
| 7113 | N | N | LYS | 941 | 67.949 | 158.325 | 176.213 | 150.11 |
| 7114 | C | CA | LYS | 941 | 69.225 | 158.537 | 175.544 | 150.11 |
| 7115 | C | C | LYS | 941 | 70.244 | 159.137 | 176.505 | 150.11 |
| 7116 | O | O | LYS | 941 | 71.435 | 158.822 | 176.438 | 150.11 |
| 7117 | C | CB | LYS | 941 | 69.024 | 159.427 | 174.318 | 150.11 |
| 7118 | C | CG | LYS | 941 | 68.431 | 158.67 | 173.133 | 150.11 |
| 7119 | C | CD | LYS | 941 | 68.604 | 159.391 | 171.805 | 150.11 |
| 7120 | C | CE | LYS | 941 | 68.539 | 158.397 | 170.655 | 150.11 |
| 7121 | N | NZ | LYS | 941 | 69.379 | 158.785 | 169.493 | 150.11 |
| 7122 | N | N | THR | 942 | 69.789 | 159.986 | 177.427 | 148.53 |
| 7123 | C | CA | THR | 942 | 70.706 | 160.557 | 178.405 | 148.53 |
| 7124 | C | C | THR | 942 | 71.146 | 159.52 | 179.432 | 148.53 |
| 7125 | O | O | THR | 942 | 72.307 | 159.529 | 179.855 | 148.53 |
| 7126 | C | CB | THR | 942 | 70.07 | 161.764 | 179.092 | 148.53 |
| 7127 | O | OG1 | THR | 942 | 68.736 | 161.441 | 179.496 | 148.53 |
| 7128 | C | CG2 | THR | 942 | 70.044 | 162.955 | 178.148 | 148.53 |
| 7129 | N | N | LEU | 943 | 70.248 | 158.617 | 179.852 | 146.13 |
| 7130 | C | CA | LEU | 943 | 70.712 | 157.55 | 180.74 | 146.13 |
| 7131 | C | C | LEU | 943 | 71.731 | 156.66 | 180.04 | 146.13 |
| 7132 | O | O | LEU | 943 | 72.731 | 156.268 | 180.649 | 146.13 |
| 7133 | C | CB | LEU | 943 | 69.586 | 156.683 | 181.33 | 146.13 |
| 7134 | C | CG | LEU | 943 | 68.343 | 157.119 | 182.127 | 146.13 |
| 7135 | C | CD1 | LEU | 943 | 67.074 | 157.251 | 181.308 | 146.13 |
| 7136 | C | CD2 | LEU | 943 | 68.108 | 156.174 | 183.3 | 146.13 |
| 7137 | N | N | LEU | 944 | 71.506 | 156.326 | 178.764 | 149.98 |
| 7138 | C | CA | LEU | 944 | 72.514 | 155.516 | 178.079 | 149.98 |
| 7139 | C | C | LEU | 944 | 73.812 | 156.298 | 177.9 | 149.98 |
| 7140 | O | O | LEU | 944 | 74.902 | 155.716 | 177.929 | 149.98 |
| 7141 | C | CB | LEU | 944 | 72.002 | 154.996 | 176.731 | 149.98 |
| 7142 | C | CG | LEU | 944 | 71.582 | 155.887 | 175.561 | 149.98 |
| 7143 | C | CD1 | LEU | 944 | 72.779 | 156.319 | 174.722 | 149.98 |
| 7144 | C | CD2 | LEU | 944 | 70.564 | 155.176 | 174.689 | 149.98 |
| 7145 | N | N | ALA | 945 | 73.716 | 157.619 | 177.718 | 144.56 |
| 7146 | C | CA | ALA | 945 | 74.915 | 158.444 | 177.611 | 144.56 |
| 7147 | C | C | ALA | 945 | 75.675 | 158.502 | 178.928 | 144.56 |
| 7148 | O | O | ALA | 945 | 76.904 | 158.635 | 178.93 | 144.56 |
| 7149 | C | CB | ALA | 945 | 74.546 | 159.853 | 177.149 | 144.56 |
| 7150 | N | N | LEU | 946 | 74.967 | 158.412 | 180.055 | 142.66 |
| 7151 | C | CA | LEU | 946 | 75.631 | 158.369 | 181.353 | 142.66 |
| 7152 | C | C | LEU | 946 | 76.406 | 157.076 | 181.558 | 142.66 |
| 7153 | O | O | LEU | 946 | 77.169 | 156.971 | 182.525 | 142.66 |
| 7154 | C | CB | LEU | 946 | 74.611 | 158.544 | 182.48 | 142.66 |
| 7155 | C | CG | LEU | 946 | 73.894 | 159.891 | 182.583 | 142.66 |
| 7156 | C | CD1 | LEU | 946 | 72.822 | 159.846 | 183.662 | 142.66 |
| 7157 | C | CD2 | LEU | 946 | 74.888 | 161.008 | 182.859 | 142.66 |
| 7158 | N | N | GLY | 947 | 76.225 | 156.095 | 180.68 | 145.9 |
| 7159 | C | CA | GLY | 947 | 76.848 | 154.798 | 180.823 | 145.9 |
| 7160 | C | C | GLY | 947 | 75.933 | 153.707 | 181.324 | 145.9 |
| 7161 | O | O | GLY | 947 | 76.407 | 152.595 | 181.581 | 145.9 |
| 7162 | N | N | CYS | 948 | 74.639 | 153.986 | 181.467 | 150.29 |
| 7163 | C | CA | CYS | 948 | 73.682 | 153.02 | 181.997 | 150.29 |
| 7164 | C | C | CYS | 948 | 73.35 | 152.002 | 180.914 | 150.29 |
| 7165 | O | O | CYS | 948 | 72.482 | 152.229 | 180.068 | 150.29 |
| 7166 | C | CB | CYS | 948 | 72.418 | 153.726 | 182.477 | 150.29 |
| 7167 | S | SG | CYS | 948 | 72.668 | 154.946 | 183.782 | 150.29 |
| 7168 | N | N | HIS | 949 | 74.045 | 150.865 | 180.933 | 150.01 |
| 7169 | C | CA | HIS | 949 | 73.691 | 149.758 | 180.044 | 150.01 |
| 7170 | C | C | HIS | 949 | 72.556 | 148.957 | 180.689 | 150.01 |
| 7171 | O | O | HIS | 949 | 72.731 | 147.88 | 181.262 | 150.01 |
| 7172 | C | CB | HIS | 949 | 74.923 | 148.925 | 179.699 | 150.01 |
| 7173 | C | CG | HIS | 949 | 75.55 | 148.217 | 180.861 | 150.01 |
| 7174 | N | ND1 | HIS | 949 | 76.4 | 148.847 | 181.744 | 150.01 |
| 7175 | C | CD2 | HIS | 949 | 75.501 | 146.92 | 181.249 | 150.01 |
| 7176 | C | CE1 | HIS | 949 | 76.82 | 147.978 | 182.645 | 150.01 |
| 7177 | N | NE2 | HIS | 949 | 76.29 | 146.8 | 182.367 | 150.01 |
| 7178 | N | N | VAL | 950 | 71.357 | 149.539 | 180.582 | 154.38 |
| 7179 | C | CA | VAL | 950 | 70.18 | 149.02 | 181.267 | 154.38 |
| 7180 | C | C | VAL | 950 | 69.908 | 147.582 | 180.844 | 154.38 |
| 7181 | O | O | VAL | 950 | 70.033 | 147.218 | 179.668 | 154.38 |
| 7182 | C | CB | VAL | 950 | 68.968 | 149.921 | 180.973 | 154.38 |
| 7183 | C | CG1 | VAL | 950 | 67.704 | 149.365 | 181.613 | 154.38 |
| 7184 | C | CG2 | VAL | 950 | 69.238 | 151.342 | 181.438 | 154.38 |
| 7185 | N | N | GLY | 951 | 69.528 | 146.753 | 181.816 | 156.18 |
| 7186 | C | CA | GLY | 951 | 69.304 | 145.346 | 181.563 | 156.18 |
| 7187 | C | C | GLY | 951 | 67.87 | 144.943 | 181.847 | 156.18 |
| 7188 | O | O | GLY | 951 | 67.223 | 145.451 | 182.767 | 156.18 |
| 7189 | N | N | ILE | 952 | 67.381 | 144.014 | 181.033 | 165.46 |
| 7190 | C | CA | ILE | 952 | 66.071 | 143.408 | 181.244 | 165.46 |
| 7191 | C | C | ILE | 952 | 66.207 | 142.302 | 182.284 | 165.46 |
| 7192 | O | O | ILE | 952 | 67.027 | 141.388 | 182.137 | 165.46 |
| 7193 | C | CB | ILE | 952 | 65.486 | 142.89 | 179.916 | 165.46 |
| 7194 | C | CG1 | ILE | 952 | 64.035 | 142.435 | 180.096 | 165.46 |
| 7195 | C | CG2 | ILE | 952 | 66.367 | 141.812 | 179.275 | 165.46 |
| 7196 | C | CD1 | ILE | 952 | 63.223 | 142.473 | 178.818 | 165.46 |
| 7197 | N | N | SER | 953 | 65.44 | 142.41 | 183.37 | 173.51 |
| 7198 | C | CA | SER | 953 | 65.581 | 141.436 | 184.447 | 173.51 |
| 7199 | C | C | SER | 953 | 64.59 | 140.286 | 184.299 | 173.51 |
| 7200 | O | O | SER | 953 | 64.984 | 139.149 | 184.026 | 173.51 |
| 7201 | C | CB | SER | 953 | 65.404 | 142.129 | 185.8 | 173.51 |
| 7202 | O | OG | SER | 953 | 64.165 | 142.815 | 185.862 | 173.51 |
| 7203 | N | N | ASP | 954 | 63.295 | 140.585 | 184.403 | 186.74 |
| 7204 | C | CA | ASF | 954 | 62.238 | 139.576 | 184.433 | 186.74 |
| 7205 | C | C | ASP | 954 | 60.941 | 140.191 | 183.933 | 186.74 |
| 7206 | O | O | ASP | 954 | 60.655 | 141.36 | 184.209 | 186.74 |
| 7207 | C | CB | ASP | 954 | 62.028 | 139.009 | 185.848 | 186.74 |
| 7208 | C | CG | ASF | 954 | 63.162 | 138.106 | 186.31 | 186.74 |
| 7209 | O | OD1 | ASP | 954 | 63.788 | 137.436 | 185.466 | 186.74 |
| 7210 | O | OD2 | ASP | 954 | 63.429 | 138.07 | 187.529 | 186.74 |
| 7211 | N | N | GLU | 955 | 60.156 | 139.396 | 183.202 | 201.71 |
| 7212 | C | CA | GLU | 955 | 58.81 | 139.803 | 182.815 | 201.71 |
| 7213 | C | C | GLU | 955 | 57.856 | 139.846 | 184.004 | 201.71 |
| 7214 | C | O | GLU | 955 | 56.788 | 140.462 | 183.91 | 201.71 |
| 7215 | C | CB | GLU | 955 | 58.27 | 138.853 | 181.741 | 201.71 |
| 7216 | C | CG | GLU | 955 | 57.007 | 139.326 | 181.032 | 201.71 |
| 7217 | C | CD | GLU | 955 | 55.742 | 138.769 | 181.659 | 201.71 |
| 7218 | O | OE1 | GLU | 955 | 55.756 | 137.597 | 182.089 | 201.71 |
| 7219 | C | OE2 | GLU | 955 | 54.735 | 139.505 | 181.725 | 201.71 |
| 7220 | N | N | HIS | 956 | 58.231 | 139.231 | 185.128 | 209.87 |
| 7221 | C | CA | HIS | 956 | 57.313 | 139.029 | 186.243 | 209.87 |
| 7222 | C | C | HIS | 956 | 56.89 | 140.322 | 186.932 | 209.87 |
| 7223 | O | O | HIS | 956 | 56.158 | 140.255 | 187.923 | 209.87 |
| 7224 | C | CB | HIS | 956 | 57.943 | 138.089 | 187.272 | 209.87 |
| 7225 | C | CG | HIS | 956 | 58.347 | 136.762 | 186.71 | 209.87 |
| 7226 | N | ND1 | HIS | 956 | 57.435 | 135.778 | 186.397 | 209.87 |
| 7227 | C | CD2 | HIS | 956 | 59.565 | 136.258 | 186.402 | 209.87 |
| 7228 | C | CE1 | HIS | 956 | 58.074 | 134.723 | 185.922 | 209.87 |
| 7229 | N | NE2 | HIS | 956 | 59.367 | 134.989 | 185.914 | 209.87 |
| 7230 | N | N | ALA | 957 | 57.339 | 141.486 | 186.457 | 211.98 |
| 7231 | C | CA | ALA | 957 | 56.913 | 142.74 | 187.07 | 211.98 |
| 7232 | C | C | ALA | 957 | 55.409 | 142.944 | 186.925 | 211.98 |
| 7233 | O | O | ALA | 957 | 54.739 | 143.391 | 187.865 | 211.98 |
| 7234 | C | CB | ALA | 957 | 57.676 | 143.913 | 186.455 | 211.98 |
| 7235 | N | N | GLU | 958 | 54.862 | 142.622 | 185.749 | 217.11 |
| 7236 | C | CA | GLU | 958 | 53.426 | 142.759 | 185.528 | 217.11 |
| 7237 | C | C | GLU | 958 | 52.638 | 141.812 | 186.424 | 217.11 |
| 7238 | O | O | GLU | 958 | 51.535 | 142.146 | 186.873 | 217.11 |
| 7239 | C | CB | GLU | 958 | 53.101 | 142.506 | 184.055 | 217.11 |
| 7240 | C | CG | GLU | 958 | 51.635 | 142.683 | 183.694 | 217.11 |
| 7241 | C | CD | GLU | 958 | 51.446 | 143.286 | 182.315 | 217.11 |
| 7242 | O | OE1 | GLU | 958 | 52.428 | 143.331 | 181.544 | 217.11 |
| 7243 | O | OE2 | GLU | 958 | 50.315 | 143.713 | 182.001 | 217.11 |
| 7244 | N | N | ASP | 959 | 53.18 | 140.622 | 186.685 | 218.73 |
| 7245 | C | CA | ASP | 959 | 52.516 | 139.695 | 187.595 | 218.73 |
| 7246 | C | C | ASP | 959 | 52.599 | 140.18 | 189.038 | 218.73 |
| 7247 | O | O | ASP | 959 | 51.626 | 140.071 | 189.793 | 218.73 |
| 7248 | C | CB | ASP | 959 | 53.131 | 138.302 | 187.462 | 218.73 |
| 7249 | C | CG | ASP | 959 | 52.888 | 137.686 | 186.098 | 218.73 |
| 7250 | O | OD1 | ASP | 959 | 51.857 | 138.01 | 185.472 | 218.73 |
| 7251 | O | OD2 | ASP | 959 | 53.73 | 136.878 | 185.651 | 218.73 |
| 7252 | N | N | LYS | 960 | 53.754 | 140.717 | 189.438 | 217.59 |
| 7253 | C | CA | LYS | 960 | 53.946 | 141.118 | 190.828 | 217.59 |
| 7254 | C | C | LYS | 960 | 53.134 | 142.36 | 191.172 | 217.59 |
| 7255 | O | O | LYS | 960 | 52.626 | 142.483 | 192.293 | 217.59 |
| 7256 | C | CB | LYS | 960 | 55.43 | 141.352 | 191.118 | 217.59 |
| 7257 | C | CG | LYS | 960 | 56.283 | 140.094 | 191.095 | 217.59 |
| 7258 | C | CD | LYS | 960 | 57.681 | 140.371 | 191.627 | 217.59 |
| 7259 | C | CE | LYS | 960 | 58.478 | 141.257 | 190.684 | 217.59 |
| 7260 | N | NZ | LYS | 960 | 58.781 | 140.575 | 189.398 | 217.59 |
| 7261 | N | N | VAL | 961 | 53.008 | 143.3 | 190.232 | 217.24 |
| 7262 | C | CA | VAL | 961 | 52.268 | 144.525 | 190.512 | 217.24 |
| 7263 | C | C | VAL | 961 | 50.8 | 144.188 | 190.746 | 217.24 |
| 7264 | O | O | VAL | 961 | 50.181 | 143.433 | 189.983 | 217.24 |
| 7265 | C | CB | VAL | 961 | 52.452 | 145.555 | 189.38 | 217.24 |
| 7266 | C | CG1 | VAL | 961 | 51.896 | 145.044 | 188.06 | 217.24 |
| 7267 | C | CG2 | VAL | 961 | 51.801 | 146.88 | 189.759 | 217.24 |
| 7268 | N | N | LYS | 962 | 50.247 | 144.722 | 191.832 | 214.27 |
| 7269 | C | CA | LYS | 962 | 48.89 | 144.414 | 192.257 | 214.27 |
| 7270 | C | C | LYS | 962 | 48.207 | 145.697 | 192.706 | 214.27 |
| 7271 | O | O | LYS | 962 | 48.843 | 146.583 | 193.283 | 214.27 |
| 7272 | C | CB | LYS | 962 | 48.893 | 143.364 | 193.384 | 214.27 |
| 7273 | C | CG | LYS | 962 | 47.517 | 142.912 | 193.857 | 214.27 |
| 7274 | C | CD | LYS | 962 | 47.104 | 143.619 | 195.138 | 214.27 |
| 7275 | C | CE | LYS | 962 | 47.949 | 143.157 | 196.315 | 214.27 |
| 7276 | N | NZ | LYS | 962 | 47.568 | 143.842 | 197.581 | 214.27 |
| 7277 | N | N | LYS | 963 | 46.911 | 145.794 | 192.425 | 211.36 |
| 7278 | C | CA | LYS | 963 | 46.162 | 147.006 | 192.725 | 211.36 |
| 7279 | C | C | LYS | 963 | 45.867 | 147.116 | 194.216 | 211.36 |
| 7280 | O | O | LYS | 963 | 45.227 | 146.24 | 194.806 | 211.36 |
| 7281 | C | CB | LYS | 963 | 44.855 | 147.023 | 191.932 | 211.36 |
| 7282 | C | CG | LYS | 963 | 44.017 | 148.274 | 192.13 | 211.36 |
| 7283 | C | CD | LYS | 963 | 42.794 | 148.259 | 191.227 | 211.36 |
| 7284 | C | CE | LYS | 963 | 41.87 | 149.428 | 191.522 | 211.36 |
| 7285 | N | NZ | LYS | 963 | 42.504 | 150.733 | 191.184 | 211.36 |
| 7286 | N | N | MET | 964 | 46.334 | 148.203 | 194.827 | 208.65 |
| 7287 | C | CA | MET | 964 | 45.974 | 148.501 | 196.205 | 208.65 |
| 7288 | C | C | MET | 964 | 44.638 | 149.24 | 196.259 | 208.65 |
| 7289 | O | O | MET | 964 | 43.922 | 149.37 | 195.261 | 208.65 |
| 7290 | C | CB | MET | 964 | 47.064 | 149.323 | 196.894 | 208.65 |
| 7291 | C | CG | MET | 964 | 48.295 | 148.532 | 197.318 | 208.65 |
| 7292 | S | SD | MET | 964 | 49.371 | 148.037 | 195.963 | 208.65 |
| 7293 | C | CE | MET | 964 | 50.142 | 149.603 | 195.565 | 208.65 |
| 7294 | N | N | LYS | 965 | 44.306 | 149.735 | 197.449 | 210.93 |
| 7295 | C | CA | LYS | 965 | 43.057 | 150.458 | 197.655 | 210.93 |
| 7296 | C | C | LYS | 965 | 43.199 | 151.882 | 197.133 | 210.93 |
| 7297 | O | O | LYS | 965 | 44.052 | 152.647 | 197.591 | 210.93 |
| 7298 | C | CB | LYS | 965 | 42.675 | 150.465 | 199.135 | 210.93 |
| 7299 | C | CG | LYS | 965 | 42.043 | 149.171 | 199.633 | 210.93 |
| 7300 | C | CD | LYS | 965 | 43.075 | 148.07 | 199.825 | 210.93 |
| 7301 | C | CE | LYS | 965 | 42.448 | 146.817 | 200.41 | 210.93 |
| 7302 | N | NZ | LYS | 965 | 43.418 | 145.689 | 200.456 | 210.93 |
| 7303 | N | N | LEU | 966 | 42.359 | 152.235 | 196.163 | 219.26 |
| 7304 | C | CA | LEU | 966 | 42.447 | 153.523 | 195.505 | 219.26 |
| 7305 | C | C | LEU | 966 | 41.093 | 154.222 | 195.535 | 219.26 |
| 7306 | O | O | LEU | 966 | 40.082 | 153.63 | 195.132 | 219.26 |
| 7307 | C | CB | LEU | 966 | 42.939 | 153.337 | 194.057 | 219.26 |
| 7308 | C | CG | LEU | 966 | 43.291 | 154.533 | 193.174 | 219.26 |
| 7309 | C | CD1 | LEU | 966 | 44.312 | 154.116 | 192.136 | 219.26 |
| 7310 | C | CD2 | LEU | 966 | 42.062 | 155.04 | 192.478 | 219.26 |
| 7311 | N | N | PRO | 967 | 41.032 | 155.467 | 196.004 | 227.55 |
| 7312 | C | CA | PRO | 967 | 39.737 | 156.141 | 196.162 | 227.55 |
| 7313 | C | C | PRO | 967 | 39.095 | 156.486 | 194.825 | 227.55 |
| 7314 | O | O | PRO | 967 | 39.755 | 156.607 | 193.79 | 227.55 |
| 7315 | C | CB | PRO | 967 | 40.088 | 157.41 | 196.95 | 227.55 |
| 7316 | C | CG | PRO | 967 | 41.438 | 157.14 | 197.551 | 227.55 |
| 7317 | C | CD | PRO | 967 | 42.137 | 156.262 | 196.562 | 227.55 |
| 7318 | N | N | LYS | 968 | 37.771 | 156.662 | 194.871 | 230.22 |
| 7319 | C | CA | LYS | 968 | 37.012 | 156.97 | 193.663 | 230.22 |
| 7320 | C | C | LYS | 968 | 37.441 | 158.295 | 193.044 | 230.22 |
| 7321 | O | O | LYS | 968 | 37.536 | 158.4 | 191.816 | 230.22 |
| 7322 | C | CB | LYS | 968 | 35.514 | 156.998 | 193.975 | 230.22 |
| 7323 | C | CG | LYS | 968 | 34.939 | 155.688 | 194.51 | 230.22 |
| 7324 | C | CD | LYS | 968 | 34.847 | 154.611 | 193.435 | 230.22 |
| 7325 | C | CE | LYS | 968 | 35.971 | 153.591 | 193.556 | 230.22 |
| 7326 | N | NZ | LYS | 968 | 35.825 | 152.489 | 192.566 | 230.22 |
| 7327 | N | N | ASN | 969 | 37.695 | 159.314 | 193.869 | 230.88 |
| 7328 | C | CA | ASN | 969 | 38.126 | 160.603 | 193.335 | 230.88 |
| 7329 | C | C | ASN | 969 | 39.492 | 160.498 | 192.665 | 230.88 |
| 7330 | O | O | ASA | 969 | 39.717 | 161.09 | 191.603 | 230.88 |
| 7331 | C | CB | ASN | 969 | 38.139 | 161.657 | 194.445 | 230.88 |
| 7332 | C | CG | ASN | 969 | 39.046 | 161.282 | 195.606 | 230.88 |
| 7333 | O | OD1 | ASN | 969 | 39.761 | 160.282 | 195.559 | 230.88 |
| 7334 | N | ND2 | ASN | 969 | 39.017 | 162.09 | 196.659 | 230.88 |
| 7335 | N | N | TYR | 970 | 40.416 | 159.747 | 193.268 | 230.04 |
| 7336 | C | CA | TYR | 970 | 41.729 | 159.555 | 192.666 | 230.04 |
| 7337 | C | C | TYR | 970 | 41.674 | 158.663 | 191.433 | 230.04 |
| 7338 | O | O | TYR | 970 | 42.592 | 158.708 | 190.607 | 230.04 |
| 7339 | C | CB | TYR | 970 | 42.706 | 158.972 | 193.69 | 230.04 |
| 7340 | C | CG | TYR | 970 | 43.122 | 159.948 | 194.769 | 230.04 |
| 7341 | C | CD1 | TYR | 970 | 42.917 | 161.314 | 194.615 | 230.04 |
| 7342 | C | CD2 | TYR | 970 | 43.728 | 159.506 | 195.938 | 230.04 |
| 7343 | C | CE1 | TYR | 970 | 43.298 | 162.21 | 195.596 | 230.04 |
| 7344 | C | CE2 | TYR | 970 | 44.113 | 160.395 | 196.925 | 230.04 |
| 7345 | C | CZ | TYR | 970 | 43.895 | 161.745 | 196.748 | 230.04 |
| 7346 | O | OH | TYR | 970 | 44.276 | 162.634 | 197.727 | 230.04 |
| 7347 | N | N | GLN | 971 | 40.63 | 157.849 | 191.294 | 228.26 |
| 7348 | C | CA | GLN | 971 | 40.477 | 157.03 | 190.098 | 228.26 |
| 7349 | C | C | GLN | 971 | 40.222 | 157.925 | 188.892 | 228.26 |
| 7350 | O | O | GLN | 971 | 39.365 | 158.813 | 188.934 | 228.26 |
| 7351 | C | CB | GLN | 971 | 39.336 | 156.028 | 190.279 | 228.26 |
| 7352 | C | CG | GLN | 971 | 39.244 | 154.976 | 189.183 | 228.26 |
| 7353 | C | CD | GLN | 971 | 38.318 | 155.378 | 188.053 | 228.26 |
| 7354 | O | OE1 | GLN | 971 | 37.386 | 156.158 | 188.246 | 228.26 |
| 7355 | N | NE2 | GLN | 971 | 38.57 | 154.842 | 186.864 | 228.26 |
| 7356 | N | N | LEU | 972 | 40.969 | 157.69 | 187.817 | 223.62 |
| 7357 | C | CA | LEU | 972 | 40.879 | 158.547 | 186.644 | 223.62 |
| 7358 | C | C | LEU | 972 | 39.544 | 158.361 | 185.936 | 223.62 |
| 7359 | O | O | LEU | 972 | 39.109 | 157.235 | 185.676 | 223.62 |
| 7360 | C | CB | LEU | 972 | 42.026 | 158.255 | 185.677 | 223.62 |
| 7361 | C | CG | LEU | 972 | 43.412 | 158.07 | 186.296 | 223.62 |
| 7362 | C | CD1 | LEU | 972 | 44.407 | 157.577 | 185.257 | 223.62 |
| 7363 | C | CD2 | LEU | 972 | 43.888 | 159.365 | 186.929 | 223.62 |
| 7364 | N | N | THR | 973 | 38.894 | 159.482 | 185.622 | 222.81 |
| 7365 | C | CA | THR | 973 | 37.655 | 159.455 | 184.857 | 222.81 |
| 7366 | C | C | THR | 973 | 37.898 | 159.301 | 183.362 | 222.81 |
| 7367 | O | O | THR | 973 | 36.933 | 159.136 | 182.607 | 222.81 |
| 7368 | C | CB | THR | 973 | 36.845 | 160.727 | 185.117 | 222.81 |
| 7369 | O | OG1 | THR | 973 | 37.682 | 161.876 | 184.93 | 222.81 |
| 7370 | C | CG2 | THR | 973 | 36.302 | 160.73 | 186.539 | 222.81 |
| 7371 | N | N | SER | 974 | 39.158 | 159.355 | 182.921 | 220.23 |
| 7372 | C | CA | SER | 974 | 39.467 | 159.213 | 181.502 | 220.23 |
| 7373 | C | C | SER | 974 | 39.129 | 157.825 | 180.975 | 220.23 |
| 7374 | O | O | SER | 974 | 38.943 | 157.657 | 179.765 | 220.23 |
| 7375 | C | CB | SER | 974 | 40.945 | 159.521 | 181.257 | 220.23 |
| 7376 | O | OG | SER | 974 | 41.745 | 158.367 | 181.45 | 220.23 |
| 7377 | N | N | GLY | 975 | 39.048 | 156.826 | 181.853 | 215.94 |
| 7378 | C | CA | GLY | 975 | 38.723 | 155.466 | 181.476 | 215.94 |
| 7379 | C | C | GLY | 975 | 39.871 | 154.49 | 181.608 | 215.94 |
| 7380 | O | O | GLY | 975 | 39.628 | 153.282 | 181.723 | 215.94 |
| 7381 | N | N | TYR | 976 | 41.111 | 154.971 | 181.596 | 211.6 |
| 7382 | C | CA | TYR | 976 | 42.252 | 154.091 | 181.8 | 211.6 |
| 7383 | C | C | TYR | 976 | 42.359 | 153.705 | 183.27 | 211.6 |
| 7384 | O | O | TYR | 976 | 41.993 | 154.476 | 184.162 | 211.6 |
| 7385 | C | CB | TYR | 976 | 43.541 | 154.764 | 181.33 | 211.6 |
| 7386 | C | CG | TYR | 976 | 44.739 | 153.84 | 181.306 | 211.6 |
| 7387 | C | CD1 | TYR | 976 | 44.948 | 152.971 | 180.243 | 211.6 |
| 7388 | C | CD2 | TYR | 976 | 45.662 | 153.839 | 182.343 | 211.6 |
| 7389 | C | CE1 | TYR | 976 | 46.04 | 152.124 | 180.215 | 211.6 |
| 7390 | C | CE2 | TYR | 976 | 46.758 | 152.995 | 182.324 | 211.6 |
| 7391 | C | CZ | TYR | 976 | 46.941 | 152.141 | 181.258 | 211.6 |
| 7392 | O | OH | TYR | 976 | 48.03 | 151.299 | 181.235 | 211.6 |
| 7393 | N | N | LYS | 977 | 42.867 | 152.501 | 183.52 | 208.77 |
| 7394 | C | CA | LYS | 977 | 42.982 | 151.986 | 184.878 | 208.77 |
| 7395 | C | C | LYS | 977 | 44.318 | 152.408 | 185.472 | 208.77 |
| 7396 | O | O | LYS | 977 | 45.373 | 152.026 | 184.942 | 208.77 |
| 7397 | C | CB | LYS | 977 | 42.85 | 150.463 | 184.892 | 208.77 |
| 7398 | C | CG | LYS | 977 | 42.848 | 149.858 | 186.286 | 208.77 |
| 7399 | C | CD | LYS | 977 | 42.627 | 148.356 | 186.236 | 208.77 |
| 7400 | C | CE | LYS | 977 | 42.63 | 147.754 | 187.631 | 208.77 |
| 7401 | N | NZ | LYS | 977 | 42.413 | 146.282 | 187.605 | 208.77 |
| 7402 | N | N | PRO | 978 | 44.331 | 153.185 | 186.553 | 207.52 |
| 7403 | C | CA | PRO | 978 | 45.603 | 153.584 | 187.162 | 207.52 |
| 7404 | C | C | PRO | 978 | 46.368 | 152.376 | 187.678 | 207.52 |
| 7405 | O | O | PRO | 978 | 45.788 | 151.367 | 188.086 | 207.52 |
| 7406 | C | CB | PRO | 978 | 45.176 | 154.5 | 188.316 | 207.52 |
| 7407 | C | CG | PRO | 978 | 43.789 | 154.934 | 187.971 | 207.52 |
| 7408 | C | CD | PRO | 978 | 43.177 | 153.777 | 187.248 | 207.52 |
| 7409 | N | N | ALA | 979 | 47.694 | 152.492 | 187.657 | 194.87 |
| 7410 | C | CA | ALA | 979 | 48.582 | 151.445 | 188.161 | 194.87 |
| 7411 | C | C | ALA | 979 | 49.579 | 152.054 | 189.137 | 194.87 |
| 7412 | O | O | ALA | 979 | 50.778 | 152.15 | 188.846 | 194.87 |
| 7413 | C | CB | ALA | 979 | 49.3 | 150.736 | 187.013 | 194.87 |
| 7414 | N | N | PRO | 980 | 49.117 | 152.478 | 190.318 | 189.41 |
| 7415 | C | CA | PRO | 980 | 50.058 | 152.931 | 191.35 | 189.41 |
| 7416 | C | C | PRO | 980 | 50.625 | 151.741 | 192.108 | 189.41 |
| 7417 | O | O | PRO | 980 | 49.886 | 150.976 | 192.733 | 189.41 |
| 7418 | C | CB | PRO | 980 | 49.188 | 153.822 | 192.243 | 189.41 |
| 7419 | C | CG | PRO | 980 | 47.83 | 153.205 | 192.15 | 189.41 |
| 7420 | C | CD | PRO | 980 | 47.718 | 152.568 | 190.778 | 189.41 |
| 7421 | N | N | MET | 981 | 51.94 | 151.564 | 192.031 | 176.42 |
| 7422 | C | CA | MET | 981 | 52.582 | 150.398 | 192.616 | 176.42 |
| 7423 | C | C | MET | 981 | 53.214 | 150.74 | 193.965 | 176.42 |
| 7424 | O | O | MET | 981 | 53.339 | 151.906 | 194.35 | 176.42 |
| 7425 | C | CB | MET | 981 | 53.604 | 149.818 | 191.634 | 176.42 |
| 7426 | C | CG | MET | 981 | 54.752 | 150.748 | 191.238 | 176.42 |
| 7427 | S | SD | MET | 981 | 56.084 | 150.887 | 192.442 | 176.42 |
| 7428 | C | CE | MET | 981 | 56.822 | 149.264 | 192.28 | 176.42 |
| 7429 | N | N | ASP | 982 | 53.615 | 149.694 | 194.687 | 167.59 |
| 7430 | C | CA | ASF | 982 | 54.168 | 149.83 | 196.032 | 167.59 |
| 7431 | C | C | ASP | 982 | 55.602 | 150.342 | 195.937 | 167.59 |
| 7432 | O | O | ASF | 982 | 56.566 | 149.575 | 195.98 | 167.59 |
| 7433 | C | CB | ASP | 982 | 54.103 | 148.495 | 196.771 | 167.59 |
| 7434 | C | CG | ASP | 982 | 54.458 | 148.62 | 198.239 | 167.59 |
| 7435 | O | OD1 | ASP | 982 | 53.912 | 149.524 | 198.907 | 167.59 |
| 7436 | O | OD2 | ASP | 982 | 55.28 | 147.816 | 198.727 | 167.59 |
| 7437 | N | N | LEU | 983 | 55.745 | 151.659 | 195.807 | 163.5 |
| 7438 | C | CA | LEU | 983 | 57.04 | 152.282 | 195.565 | 163.5 |
| 7439 | C | C | LEU | 983 | 57.842 | 152.527 | 196.838 | 163.5 |
| 7440 | O | O | LEU | 983 | 58.963 | 153.04 | 196.756 | 163.5 |
| 7441 | C | CB | LEU | 983 | 56.858 | 153.601 | 194.805 | 163.5 |
| 7442 | C | CG | LEU | 983 | 56.294 | 154.833 | 195.523 | 163.5 |
| 7443 | C | CD1 | LEU | 983 | 56.575 | 156.078 | 194.696 | 163.5 |
| 7444 | C | CD2 | LEU | 983 | 54.801 | 154.708 | 195.79 | 163.5 |
| 7445 | N | N | SER | 984 | 57.302 | 152.18 | 198.008 | 162.16 |
| 7446 | C | CA | SER | 984 | 58.019 | 152.451 | 199.25 | 162.16 |
| 7447 | C | C | SER | 984 | 59.183 | 151.487 | 199.453 | 162.16 |
| 7448 | O | O | SER | 984 | 60.276 | 151.907 | 199.853 | 162.16 |
| 7449 | C | CB | SER | 984 | 57.06 | 152.386 | 200.439 | 162.16 |
| 7450 | O | OG | SER | 984 | 56.57 | 151.071 | 200.633 | 162.16 |
| 7451 | N | N | PHE | 985 | 58.977 | 150.195 | 199.182 | 157 |
| 7452 | C | CA | PHE | 985 | 59.991 | 149.21 | 199.543 | 157 |
| 7453 | C | C | PHE | 985 | 61.216 | 149.268 | 198.639 | 157 |
| 7454 | O | O | PHE | 985 | 62.27 | 148.747 | 199.019 | 157 |
| 7455 | C | CB | PHE | 985 | 59.401 | 147.796 | 199.539 | 157 |
| 7456 | C | CG | PHE | 985 | 59.283 | 147.178 | 198.174 | 157 |
| 7457 | C | CD1 | PHE | 985 | 58.194 | 147.449 | 197.366 | 157 |
| 7458 | C | CD2 | PHE | 985 | 60.257 | 146.309 | 197.706 | 157 |
| 7459 | C | CE1 | PHE | 985 | 58.081 | 146.872 | 196.114 | 157 |
| 7460 | C | CE2 | PHE | 985 | 60.152 | 145.734 | 196.455 | 157 |
| 7461 | C | CZ | PHE | 985 | 59.063 | 146.017 | 195.657 | 157 |
| 7462 | N | N | ILE | 986 | 61.108 | 149.879 | 197.459 | 150.75 |
| 7463 | C | CA | ILE | 986 | 62.274 | 150.017 | 196.595 | 150.75 |
| 7464 | C | C | ILE | 986 | 63.249 | 150.994 | 197.236 | 150.75 |
| 7465 | O | O | ILE | 986 | 62.926 | 152.171 | 197.445 | 150.75 |
| 7466 | C | CB | ILE | 986 | 61.872 | 150.485 | 195.189 | 150.75 |
| 7467 | C | CG1 | ILE | 986 | 61.014 | 149.436 | 194.481 | 150.75 |
| 7468 | C | CG2 | ILE | 986 | 63.113 | 150.786 | 194.364 | 150.75 |
| 7469 | C | CD1 | ILE | 986 | 59.531 | 149.65 | 194.652 | 150.75 |
| 7470 | N | N | LYS | 987 | 64.448 | 150.514 | 197.547 | 139.36 |
| 7471 | C | CA | LYS | 987 | 65.479 | 151.334 | 198.167 | 139.36 |
| 7472 | C | C | LYS | 987 | 66.317 | 151.978 | 197.07 | 139.36 |
| 7473 | O | O | LYS | 987 | 66.966 | 151.277 | 196.285 | 139.36 |
| 7474 | C | CB | LYS | 987 | 66.343 | 150.493 | 199.11 | 139.36 |
| 7475 | C | CG | LYS | 987 | 67.57 | 151.197 | 199.685 | 139.36 |
| 7476 | C | CD | LYS | 987 | 68.845 | 150.864 | 198.921 | 139.36 |
| 7477 | C | CE | LYS | 987 | 70.03 | 151.649 | 199.464 | 139.36 |
| 7478 | N | NZ | LYS | 987 | 71.245 | 151.497 | 198.618 | 139.36 |
| 7479 | N | N | LEU | 988 | 66.286 | 153.307 | 197.009 | 132.98 |
| 7480 | C | CA | LEU | 988 | 67.048 | 154.047 | 196.013 | 132.98 |
| 7481 | C | C | LEU | 988 | 68.505 | 154.122 | 196.443 | 132.98 |
| 7482 | O | O | LEU | 988 | 68.847 | 154.834 | 197.394 | 132.98 |
| 7483 | C | CB | LEU | 988 | 66.471 | 155.448 | 195.826 | 132.98 |
| 7484 | C | CG | LEU | 988 | 66.932 | 156.213 | 194.585 | 132.98 |
| 7485 | C | CD1 | LEU | 988 | 66.519 | 155.49 | 193.316 | 132.98 |
| 7486 | C | CD2 | LEU | 988 | 66.371 | 157.621 | 194.611 | 132.98 |
| 7487 | N | N | THR | 989 | 69.356 | 153.381 | 195.747 | 130.98 |
| 7488 | C | CA | THR | 989 | 70.777 | 153.406 | 196.044 | 130.98 |
| 7489 | C | C | THR | 989 | 71.346 | 154.789 | 195.709 | 130.98 |
| 7490 | O | O | THR | 989 | 70.915 | 155.43 | 194.735 | 130.98 |
| 7491 | C | CB | THR | 989 | 71.481 | 152.286 | 195.262 | 130.98 |
| 7492 | O | OG1 | THR | 989 | 70.831 | 151.04 | 195.542 | 130.98 |
| 7493 | C | CG2 | THR | 989 | 72.95 | 152.146 | 195.634 | 130.98 |
| 7494 | N | N | PRO | 990 | 72.278 | 155.304 | 196.521 | 133.56 |
| 7495 | C | CA | PRO | 990 | 72.781 | 156.667 | 196.276 | 133.56 |
| 7496 | C | C | PRO | 990 | 73.356 | 156.872 | 194.887 | 133.56 |
| 7497 | O | O | PRO | 990 | 73.39 | 158.012 | 194.408 | 133.56 |
| 7498 | C | CB | PRO | 990 | 73.845 | 156.865 | 197.367 | 133.56 |
| 7499 | C | CG | PRO | 990 | 74.045 | 155.548 | 198.014 | 133.56 |
| 7500 | C | CD | PRO | 990 | 72.852 | 154.704 | 197.738 | 133.56 |
| 7501 | N | N | SER | 991 | 73.807 | 155.812 | 194.214 | 133.78 |
| 7502 | C | CA | SER | 991 | 74.237 | 155.969 | 192.829 | 133.78 |
| 7503 | C | C | SER | 991 | 73.086 | 156.443 | 191.947 | 133.78 |
| 7504 | O | O | SER | 991 | 73.229 | 157.422 | 191.202 | 133.78 |
| 7505 | C | CB | SER | 991 | 74.817 | 154.656 | 192.303 | 133.78 |
| 7506 | O | OG | SER | 991 | 73.823 | 153.65 | 192.222 | 133.78 |
| 7507 | N | N | GLN | 992 | 71.932 | 155.77 | 192.019 | 133.67 |
| 7508 | C | CA | GLN | 992 | 70.776 | 156.245 | 191.263 | 133.67 |
| 7509 | C | C | GLN | 992 | 70.304 | 157.598 | 191.763 | 133.67 |
| 7510 | O | O | GLN | 992 | 69.742 | 158.377 | 190.993 | 133.67 |
| 7511 | C | CB | GLN | 992 | 69.624 | 155.239 | 191.284 | 133.67 |
| 7512 | C | CG | GLN | 992 | 69.915 | 153.948 | 190.554 | 133.67 |
| 7513 | C | CD | GLN | 992 | 70.697 | 153.003 | 191.404 | 133.67 |
| 7514 | O | OE1 | GLN | 992 | 70.748 | 153.17 | 192.612 | 133.67 |
| 7515 | N | NE2 | GLN | 992 | 71.317 | 152.007 | 190.787 | 133.67 |
| 7516 | N | N | GLU | 993 | 70.517 | 157.908 | 193.043 | 138.41 |
| 7517 | C | CA | GLU | 993 | 70.239 | 159.279 | 193.474 | 138.41 |
| 7518 | C | C | GLU | 993 | 71.119 | 160.276 | 192.719 | 138.41 |
| 7519 | O | O | GLU | 993 | 70.661 | 161.355 | 192.32 | 138.41 |
| 7520 | C | CB | GLU | 993 | 70.427 | 159.418 | 194.984 | 138.41 |
| 7521 | C | CG | GLU | 993 | 70.186 | 160.834 | 195.491 | 138.41 |
| 7522 | C | CD | GLU | 993 | 68.723 | 161.242 | 195.442 | 138.41 |
| 7523 | O | OE1 | GLU | 993 | 67.849 | 160.355 | 195.533 | 138.41 |
| 7524 | O | OE2 | GLU | 993 | 68.451 | 162.453 | 195.298 | 138.41 |
| 7525 | N | N | ALA | 994 | 72.388 | 159.924 | 192.508 | 138.7 |
| 7526 | C | CA | ALA | 994 | 73.288 | 160.795 | 191.755 | 138.7 |
| 7527 | C | C | ALA | 994 | 72.854 | 160.921 | 190.297 | 138.7 |
| 7528 | O | O | ALA | 994 | 72.868 | 162.022 | 189.727 | 138.7 |
| 7529 | C | CB | ALA | 994 | 74.722 | 160.275 | 191.845 | 138.7 |
| 7530 | N | N | MET | 995 | 72.482 | 159.803 | 189.666 | 144.61 |
| 7531 | C | CA | MET | 995 | 71.959 | 159.904 | 188.304 | 144.61 |
| 7532 | C | C | MET | 995 | 70.653 | 160.689 | 188.263 | 144.61 |
| 7533 | O | O | MET | 995 | 70.356 | 161.343 | 187.263 | 144.61 |
| 7534 | C | CB | MET | 995 | 71.773 | 158.535 | 187.635 | 144.61 |
| 7535 | C | CG | MET | 995 | 73.056 | 157.862 | 187.123 | 144.61 |
| 7536 | S | SD | MET | 995 | 74.226 | 157.138 | 188.28 | 144.61 |
| 7537 | C | CE | MET | 995 | 73.322 | 155.66 | 188.735 | 144.61 |
| 7538 | N | N | VAL | 996 | 69.865 | 160.643 | 189.336 | 139.77 |
| 7539 | C | CA | VAL | 996 | 68.663 | 161.468 | 189.425 | 139.77 |
| 7540 | C | C | VAL | 996 | 69.033 | 162.947 | 189.459 | 139.77 |
| 7541 | O | O | VAL | 996 | 68.381 | 163.784 | 188.82 | 139.77 |
| 7542 | C | CB | VAL | 996 | 67.832 | 161.045 | 190.652 | 139.77 |
| 7543 | C | CG1 | VAL | 996 | 66.859 | 162.12 | 191.044 | 139.77 |
| 7544 | C | CG2 | VAL | 996 | 67.07 | 159.762 | 190.351 | 139.77 |
| 7545 | N | N | ASP | 997 | 70.088 | 163.289 | 190.201 | 140.79 |
| 7546 | C | CA | ASP | 997 | 70.562 | 164.67 | 190.214 | 140.79 |
| 7547 | C | C | ASP | 997 | 71.003 | 165.109 | 188.823 | 140.79 |
| 7548 | O | O | ASP | 997 | 70.666 | 166.212 | 188.368 | 140.79 |
| 7549 | C | CB | ASP | 997 | 71.717 | 164.821 | 191.204 | 140.79 |
| 7550 | C | CG | ASP | 997 | 71.276 | 164.671 | 192.646 | 140.79 |
| 7551 | O | OD1 | ASP | 997 | 70.111 | 164.996 | 192.951 | 140.79 |
| 7552 | O | OD2 | ASP | 997 | 72.1 | 164.226 | 193.474 | 140.79 |
| 7553 | N | N | LYS | 998 | 71.757 | 164.253 | 188.131 | 141.72 |
| 7554 | C | CA | LYS | 998 | 72.156 | 164.566 | 186.761 | 141.72 |
| 7555 | C | C | LYS | 998 | 70.957 | 164.673 | 185.829 | 141.72 |
| 7556 | O | O | LYS | 998 | 70.949 | 165.515 | 184.927 | 141.72 |
| 7557 | C | CB | LYS | 998 | 73.159 | 163.539 | 186.238 | 141.72 |
| 7558 | C | CG | LYS | 998 | 74.445 | 163.467 | 187.036 | 141.72 |
| 7559 | C | CD | LYS | 998 | 75.195 | 164.788 | 186.88 | 141.72 |
| 7560 | C | CE | LYS | 998 | 76.578 | 164.755 | 187.506 | 141.72 |
| 7561 | N | NZ | LYS | 998 | 76.527 | 164.685 | 188.989 | 141.72 |
| 7562 | N | N | LEU | 999 | 69.943 | 163.826 | 186.012 | 143.25 |
| 7563 | C | CA | LEU | 999 | 68.747 | 163.922 | 185.179 | 143.25 |
| 7564 | C | C | LEU | 999 | 68.036 | 165.25 | 185.395 | 143.25 |
| 7565 | O | O | LEU | 999 | 67.581 | 165.878 | 184.434 | 143.25 |
| 7566 | C | CB | LEU | 999 | 67.8 | 162.755 | 185.465 | 143.25 |
| 7567 | C | CG | LEU | 999 | 68.212 | 161.349 | 185.013 | 143.25 |
| 7568 | C | CD1 | LEU | 999 | 66.988 | 160.463 | 184.812 | 143.25 |
| 7569 | C | CD2 | LEU | 999 | 69.069 | 161.387 | 183.754 | 143.25 |
| 7570 | N | N | ALA | 1000 | 67.929 | 165.69 | 186.65 | 142.71 |
| 7571 | C | CA | ALA | 1000 | 67.324 | 166.989 | 186.923 | 142.71 |
| 7572 | C | C | ALA | 1000 | 68.119 | 168.11 | 186.271 | 142.71 |
| 7573 | O | O | ALA | 1000 | 67.549 | 168.977 | 185.594 | 142.71 |
| 7574 | C | CB | ALA | 1000 | 67.217 | 167.213 | 188.431 | 142.71 |
| 7575 | N | N | GLU | 1001 | 69.442 | 168.094 | 186.45 | 143.2 |
| 7576 | C | CA | GLU | 1001 | 70.289 | 169.123 | 185.857 | 143.2 |
| 7577 | C | C | GLU | 1001 | 70.133 | 169.147 | 184.341 | 143.2 |
| 7578 | O | O | GLU | 1001 | 69.97 | 170.212 | 183.735 | 143.2 |
| 7579 | C | CB | GLU | 1001 | 71.749 | 168.876 | 186.242 | 143.2 |
| 7580 | C | CG | GLU | 1001 | 72.736 | 169.871 | 185.653 | 143.2 |
| 7581 | C | CD | GLU | 1001 | 72.739 | 171.195 | 186.388 | 143.2 |
| 7582 | O | OE1 | GLU | 1001 | 72.108 | 171.276 | 187.462 | 143.2 |
| 7583 | O | OE2 | GLU | 1001 | 73.368 | 172.154 | 185.89 | 143.2 |
| 7584 | N | N | ASN | 1002 | 70.152 | 167.969 | 183.716 | 141.05 |
| 7585 | C | CA | ASN | 1002 | 70.122 | 167.884 | 182.262 | 141.05 |
| 7586 | C | C | ASN | 1002 | 68.763 | 168.287 | 181.707 | 141.05 |
| 7587 | O | O | ASN | 1002 | 68.688 | 168.97 | 180.681 | 141.05 |
| 7588 | C | CB | ASN | 1002 | 70.488 | 166.467 | 181.825 | 141.05 |
| 7589 | C | CG | ASN | 1002 | 70.875 | 166.385 | 180.365 | 141.05 |
| 7590 | O | OD1 | ASN | 1002 | 70.718 | 167.343 | 179.608 | 141.05 |
| 7591 | N | ND2 | ASN | 1002 | 71.396 | 165.235 | 179.962 | 141.05 |
| 7592 | N | N | ALA | 1003 | 67.676 | 167.872 | 182.362 | 137.21 |
| 7593 | C | CA | ALA | 1003 | 66.351 | 168.281 | 181.914 | 137.21 |
| 7594 | C | C | ALA | 1003 | 66.177 | 169.787 | 182.034 | 137.21 |
| 7595 | O | O | ALA | 1003 | 65.616 | 170.43 | 181.137 | 137.21 |
| 7596 | C | CB | ALA | 1003 | 65.275 | 167.547 | 182.714 | 137.21 |
| 7597 | N | N | HIS | 1004 | 66.661 | 170.372 | 183.134 | 139.95 |
| 7598 | C | CA | HIS | 1004 | 66.576 | 171.819 | 183.285 | 139.95 |
| 7599 | C | C | HIS | 1004 | 67.396 | 172.528 | 182.214 | 139.95 |
| 7600 | O | O | HIS | 1004 | 66.961 | 173.543 | 181.658 | 139.95 |
| 7601 | C | CB | HIS | 1004 | 67.034 | 172.219 | 184.688 | 139.95 |
| 7602 | C | CG | HIS | 1004 | 67.221 | 173.693 | 184.87 | 139.95 |
| 7603 | N | ND1 | HIS | 1004 | 66.267 | 174.491 | 185.463 | 139.95 |
| 7604 | C | CD2 | HIS | 1004 | 68.255 | 174.51 | 184.561 | 139.95 |
| 7605 | C | CE1 | HIS | 1004 | 66.699 | 175.738 | 185.499 | 139.95 |
| 7606 | N | NEZ | HIS | 1004 | 67.903 | 175.777 | 184.958 | 139.95 |
| 7607 | N | N | ASN | 1005 | 68.586 | 172.002 | 181.907 | 138.12 |
| 7608 | C | CA | ASN | 1005 | 69.408 | 172.594 | 180.856 | 138.12 |
| 7609 | C | C | ASN | 1005 | 68.729 | 172.498 | 179.495 | 138.12 |
| 7610 | O | O | ASN | 1005 | 68.789 | 173.439 | 178.698 | 138.12 |
| 7611 | C | CB | ASN | 1005 | 70.781 | 171.923 | 180.817 | 138.12 |
| 7612 | C | CG | ASN | 1005 | 71.556 | 172.107 | 182.106 | 138.12 |
| 7613 | O | OD1 | ASN | 1005 | 71.243 | 172.983 | 182.912 | 138.12 |
| 7614 | N | ND2 | ASN | 1005 | 72.574 | 171.278 | 182.308 | 138.12 |
| 7615 | N | N | VAL | 1006 | 68.094 | 171.361 | 179.204 | 131.81 |
| 7616 | C | CA | VAL | 1006 | 67.394 | 171.2 | 177.931 | 131.81 |
| 7617 | C | C | VAL | 1006 | 66.238 | 172.186 | 177.832 | 131.81 |
| 7618 | O | O | VAL | 1006 | 66.02 | 172.81 | 176.785 | 131.81 |
| 7619 | C | CB | VAL | 1006 | 66.918 | 169.745 | 177.762 | 131.81 |
| 7620 | C | CG1 | VAL | 1006 | 65.945 | 169.634 | 176.6 | 131.81 |
| 7621 | C | CG2 | VAL | 1006 | 68.107 | 168.823 | 177.546 | 131.81 |
| 7622 | N | N | TRP | 1007 | 65.481 | 172.342 | 178.92 | 133.96 |
| 7623 | C | CA | TRP | 1007 | 64.398 | 173.32 | 178.935 | 133.96 |
| 7624 | C | C | TRP | 1007 | 64.921 | 174.734 | 178.71 | 133.96 |
| 7625 | O | O | TRP | 1007 | 64.338 | 175.508 | 177.938 | 133.96 |
| 7626 | C | CB | TRP | 1007 | 63.643 | 173.233 | 180.259 | 133.96 |
| 7627 | C | CG | TRP | 1007 | 62.64 | 174.317 | 180.435 | 133.96 |
| 7628 | C | CD1 | TRP | 1007 | 61.395 | 174.385 | 179.885 | 133.96 |
| 7629 | C | CD2 | TRP | 1007 | 62.804 | 175.506 | 181.211 | 133.96 |
| 7630 | N | NE1 | TRP | 1007 | 60.769 | 175.544 | 180.278 | 133.96 |
| 7631 | C | CE2 | TRP | 1007 | 61.616 | 176.25 | 181.092 | 133.96 |
| 7632 | C | CE3 | TRP | 1007 | 63.843 | 176.014 | 181.997 | 133.96 |
| 7633 | C | CZ2 | TRP | 1007 | 61.436 | 177.473 | 181.732 | 133.96 |
| 7634 | C | CZ3 | TRP | 1007 | 63.662 | 177.227 | 182.631 | 133.96 |
| 7635 | C | CH2 | TRP | 1007 | 62.469 | 177.942 | 182.496 | 133.96 |
| 7636 | N | N | ALA | 1008 | 66.018 | 175.091 | 179.382 | 132.48 |
| 7637 | C | CA | ALA | 1008 | 66.588 | 176.423 | 179.214 | 132.48 |
| 7638 | C | C | ALA | 1008 | 67.065 | 176.641 | 177.784 | 132.48 |
| 7639 | O | O | ALA | 1008 | 66.869 | 177.72 | 177.214 | 132.48 |
| 7640 | C | CB | ALA | 1008 | 67.734 | 176.633 | 180.203 | 132.48 |
| 7641 | N | N | ARG | 1009 | 67.695 | 175.626 | 177.189 | 130.16 |
| 7642 | C | CA | ARG | 1009 | 68.138 | 175.735 | 175.804 | 130.16 |
| 7643 | C | C | ARG | 1009 | 66.958 | 175.922 | 174.862 | 130.16 |
| 7644 | O | O | ARG | 1009 | 67.022 | 176.729 | 173.927 | 130.16 |
| 7645 | C | CB | ARG | 1009 | 68.928 | 174.492 | 175.404 | 130.16 |
| 7646 | C | CG | ARG | 1009 | 69.536 | 174.579 | 174.018 | 130.16 |
| 7647 | C | CD | ARG | 1009 | 70.331 | 173.333 | 173.706 | 130.16 |
| 7648 | N | NE | ARG | 1009 | 69.474 | 172.156 | 173.625 | 130.16 |
| 7649 | C | CZ | ARG | 1009 | 69.499 | 171.147 | 174.485 | 130.16 |
| 7650 | N | NH1 | ARG | 1009 | 70.309 | 171.149 | 175.531 | 130.16 |
| 7651 | N | NH2 | ARG | 1009 | 68.684 | 170.113 | 174.296 | 130.16 |
| 7652 | N | N | ASP | 1010 | 65.88 | 175.168 | 175.082 | 128.45 |
| 7653 | C | CA | ASP | 1010 | 64.7 | 175.309 | 174.238 | 128.45 |
| 7654 | C | C | ASP | 1010 | 64.112 | 176.707 | 174.355 | 128.45 |
| 7655 | O | O | ASP | 1010 | 63.74 | 177.321 | 173.348 | 128.45 |
| 7656 | C | CB | ASP | 1010 | 63.662 | 174.252 | 174.611 | 128.45 |
| 7657 | C | CG | ASP | 1010 | 64.069 | 172.858 | 174.175 | 128.45 |
| 7658 | O | OD1 | ASP | 1010 | 64.822 | 172.74 | 173.186 | 128.45 |
| 7659 | O | OD2 | ASP | 1010 | 63.637 | 171.881 | 174.822 | 128.45 |
| 7660 | N | N | ARG | 1011 | 64.042 | 177.239 | 175.576 | 125.59 |
| 7661 | C | CA | ARG | 1011 | 63.508 | 178.585 | 175.755 | 125.59 |
| 7662 | C | C | ARG | 1011 | 64.41 | 179.635 | 175.116 | 125.59 |
| 7663 | O | O | ARG | 1011 | 63.921 | 180.627 | 174.565 | 125.59 |
| 7664 | C | CB | ARG | 1011 | 63.294 | 178.873 | 177.241 | 125.59 |
| 7665 | C | CG | ARG | 1011 | 61.857 | 178.656 | 177.699 | 125.59 |
| 7666 | C | CD | ARG | 1011 | 61.398 | 177.232 | 177.43 | 125.59 |
| 7667 | N | NE | ARG | 1011 | 59.987 | 177.039 | 177.744 | 125.59 |
| 7668 | C | CZ | ARG | 1011 | 59.278 | 175.983 | 177.373 | 125.59 |
| 7669 | N | NH1 | ARG | 1011 | 59.818 | 175 | 176.671 | 125.59 |
| 7670 | N | NH2 | ARG | 1011 | 57.994 | 175.911 | 177.712 | 125.59 |
| 7671 | N | N | ILE | 1012 | 65.729 | 179.439 | 175.177 | 120.25 |
| 7672 | C | CA | ILE | 1012 | 66.642 | 180.403 | 174.57 | 120.25 |
| 7673 | C | C | ILE | 1012 | 66.534 | 180.365 | 173.048 | 120.25 |
| 7674 | O | O | ILE | 1012 | 66.558 | 181.409 | 172.386 | 120.25 |
| 7675 | C | CB | ILE | 1012 | 68.081 | 180.158 | 175.057 | 120.25 |
| 7676 | C | CG1 | ILE | 1012 | 68.202 | 180.539 | 176.534 | 120.25 |
| 7677 | C | CG2 | ILE | 1012 | 69.077 | 180.949 | 174.226 | 120.25 |
| 7678 | C | CD1 | ILE | 1012 | 69.582 | 180.353 | 177.106 | 120.25 |
| 7679 | N | N | ARG | 1013 | 66.41 | 179.168 | 172.469 | 121.71 |
| 7680 | C | CA | ARG | 1013 | 66.123 | 179.068 | 171.039 | 121.71 |
| 7681 | C | C | ARG | 1013 | 64.802 | 179.737 | 170.689 | 121.71 |
| 7682 | O | O | ARG | 1013 | 64.679 | 180.364 | 169.63 | 121.71 |
| 7683 | C | CB | ARG | 1013 | 66.128 | 177.605 | 170.598 | 121.71 |
| 7684 | C | CG | ARG | 1013 | 67.474 | 176.938 | 170.761 | 121.71 |
| 7685 | C | CD | ARG | 1013 | 68.425 | 177.453 | 169.669 | 121.71 |
| 7686 | N | NE | ARG | 1013 | 69.417 | 178.465 | 170.042 | 121.71 |
| 7687 | C | CZ | ARG | 1013 | 70.072 | 178.564 | 171.194 | 121.71 |
| 7688 | N | NH1 | ARG | 1013 | 69.957 | 177.654 | 172.148 | 121.71 |
| 7689 | N | NH2 | ARG | 1013 | 70.896 | 179.591 | 171.381 | 121.71 |
| 7690 | N | N | GLN | 1014 | 63.801 | 179.613 | 171.559 | 118.04 |
| 7691 | C | CA | GLN | 1014 | 62.566 | 180.356 | 171.347 | 118.04 |
| 7692 | C | C | GLN | 1014 | 62.798 | 181.857 | 171.446 | 118.04 |
| 7693 | O | O | GLN | 1014 | 62.059 | 182.639 | 170.839 | 118.04 |
| 7694 | C | CB | GLN | 1014 | 61.502 | 179.913 | 172.35 | 118.04 |
| 7695 | C | CG | GLN | 1014 | 61.012 | 178.495 | 172.124 | 118.04 |
| 7696 | C | CD | GLN | 1014 | 60.363 | 178.318 | 170.768 | 118.04 |
| 7697 | O | OE1 | GLN | 1014 | 60.898 | 177.632 | 169.898 | 118.04 |
| 7698 | N | NE2 | GLN | 1014 | 59.205 | 178.939 | 170.579 | 118.04 |
| 7699 | N | N | GLY | 1015 | 63.815 | 182.275 | 172.196 | 116.62 |
| 7700 | C | CA | GLY | 1015 | 64.142 | 183.676 | 172.355 | 116.62 |
| 7701 | C | C | GLY | 1015 | 63.968 | 184.217 | 173.757 | 116.62 |
| 7702 | O | O | GLY | 1015 | 64.124 | 185.426 | 173.96 | 116.62 |
| 7703 | N | N | TRP | 1016 | 63.647 | 183.365 | 174.727 | 118.72 |
| 7704 | C | CA | TRP | 1016 | 63.439 | 183.816 | 176.095 | 118.72 |
| 7705 | C | C | TRP | 1016 | 64.741 | 184.317 | 176.707 | 118.72 |
| 7706 | O | O | TRP | 1016 | 65.834 | 183.859 | 176.364 | 118.72 |
| 7707 | C | CB | TRP | 1016 | 62.873 | 182.683 | 176.949 | 118.72 |
| 7708 | C | CG | TRP | 1016 | 61.473 | 182.307 | 176.602 | 118.72 |
| 7709 | C | CD1 | TRP | 1016 | 61.069 | 181.52 | 175.565 | 118.72 |
| 7710 | C | CD2 | TRP | 1016 | 60.286 | 182.694 | 177.3 | 118.72 |
| 7711 | N | NE1 | TRP | 1016 | 59.701 | 181.397 | 175.57 | 118.72 |
| 7712 | C | CE2 | TRP | 1016 | 59.196 | 182.11 | 176.626 | 118.72 |
| 7713 | C | CE3 | TRP | 1016 | 60.038 | 183.484 | 178.427 | 118.72 |
| 7714 | C | CZ2 | TRP | 1016 | 57.88 | 182.287 | 177.042 | 118.72 |
| 7715 | C | CZ3 | TRP | 1016 | 58.731 | 183.658 | 178.839 | 118.72 |
| 7716 | C | CH2 | TRP | 1016 | 57.669 | 183.063 | 178.148 | 118.72 |
| 7717 | N | N | THR | 1017 | 64.617 | 185.274 | 177.625 | 106.56 |
| 7718 | C | CA | THR | 1017 | 65.77 | 185.76 | 178.368 | 106.56 |
| 7719 | C | C | THR | 1017 | 65.387 | 185.924 | 179.831 | 106.56 |
| 7720 | O | O | THR | 1017 | 64.214 | 186.098 | 180.167 | 106.56 |
| 7721 | C | CB | THR | 1017 | 66.305 | 187.077 | 177.792 | 106.56 |
| 7722 | O | OG1 | THR | 1017 | 67.52 | 187.437 | 178.461 | 106.56 |
| 7723 | C | CG2 | THR | 1017 | 65.287 | 188.181 | 177.951 | 106.56 |
| 7724 | N | N | TYR | 1018 | 66.39 | 185.852 | 180.701 | 107.48 |
| 7725 | C | CA | TYR | 1018 | 66.136 | 185.845 | 182.134 | 107.48 |
| 7726 | C | C | TYR | 1018 | 65.584 | 187.187 | 182.602 | 107.48 |
| 7727 | O | O | TYR | 1018 | 65.921 | 188.248 | 182.07 | 107.48 |
| 7728 | C | CB | TYR | 1018 | 67.413 | 185.513 | 182.905 | 107.48 |
| 7729 | C | CG | TYR | 1018 | 67.343 | 185.864 | 184.373 | 107.48 |
| 7730 | C | CD1 | TYR | 1018 | 66.607 | 185.087 | 185.256 | 107.48 |
| 7731 | C | CD2 | TYR | 1018 | 68.004 | 186.977 | 184.875 | 107.48 |
| 7732 | C | CE1 | TYR | 1018 | 66.535 | 185.404 | 186.597 | 107.48 |
| 7733 | C | CE2 | TYR | 1018 | 67.937 | 187.302 | 186.216 | 107.48 |
| 7734 | C | CZ | TYR | 1018 | 67.201 | 186.512 | 187.072 | 107.48 |
| 7735 | O | OH | TYR | 1018 | 67.131 | 186.83 | 188.408 | 107.48 |
| 7736 | N | N | GLY | 1019 | 64.719 | 187.125 | 183.614 | 109.48 |
| 7737 | C | CA | GLY | 1019 | 64.16 | 188.312 | 184.228 | 109.48 |
| 7738 | C | C | GLY | 1019 | 63.784 | 188.02 | 185.664 | 109.48 |
| 7739 | O | O | GLY | 1019 | 63.891 | 186.888 | 186.138 | 109.48 |
| 7740 | N | N | ILE | 1020 | 63.355 | 189.068 | 186.367 | 107.17 |
| 7741 | C | CA | ILE | 1020 | 62.934 | 188.895 | 187.754 | 107.17 |
| 7742 | C | C | ILE | 1020 | 61.583 | 188.193 | 187.827 | 107.17 |
| 7743 | O | O | ILE | 1020 | 61.332 | 187.396 | 188.738 | 107.17 |
| 7744 | C | CB | ILE | 1020 | 62.914 | 190.248 | 188.487 | 107.17 |
| 7745 | C | CG1 | ILE | 1020 | 62.046 | 191.26 | 187.734 | 107.17 |
| 7746 | C | CG2 | ILE | 1020 | 64.328 | 190.766 | 188.682 | 107.17 |
| 7747 | C | CD1 | ILE | 1020 | 61.726 | 192.502 | 188.534 | 107.17 |
| 7748 | N | N | GLN | 1021 | 60.694 | 188.474 | 186.877 | 109.49 |
| 7749 | C | CA | GLN | 1021 | 59.344 | 187.934 | 186.912 | 109.49 |
| 7750 | C | C | GLN | 1021 | 58.862 | 187.709 | 185.486 | 109.49 |
| 7751 | O | O | GLN | 1021 | 59.43 | 188.236 | 184.526 | 109.49 |
| 7752 | C | CB | GLN | 1021 | 58.402 | 188.86 | 187.695 | 109.49 |
| 7753 | C | CG | GLN | 1021 | 58.222 | 190.248 | 187.096 | 109.49 |
| 7754 | C | CD | GLN | 1021 | 57.14 | 190.295 | 186.039 | 109.49 |
| 7755 | O | OE1 | GLN | 1021 | 56.171 | 189.541 | 186.096 | 109.49 |
| 7756 | N | NE2 | GLN | 1021 | 57.303 | 191.181 | 185.063 | 109.49 |
| 7757 | N | N | GLN | 1022 | 57.805 | 186.911 | 185.36 | 114.61 |
| 7758 | C | CA | GLN | 1022 | 57.328 | 186.491 | 184.048 | 114.61 |
| 7759 | C | C | GLN | 1022 | 56.737 | 187.664 | 183.277 | 114.61 |
| 7760 | O | O | GLN | 1022 | 55.763 | 188.284 | 183.712 | 114.61 |
| 7761 | C | CB | GLN | 1022 | 56.29 | 185.379 | 184.199 | 114.61 |
| 7762 | C | CG | GLN | 1022 | 55.584 | 184.971 | 182.905 | 114.61 |
| 7763 | C | CD | GLN | 1022 | 56.505 | 184.348 | 181.861 | 114.61 |
| 7764 | O | OE1 | GLN | 1022 | 57.724 | 184.292 | 182.028 | 114.61 |
| 7765 | N | NE2 | GLN | 1022 | 55.913 | 183.876 | 180.771 | 114.61 |
| 7766 | N | N | ASP | 1023 | 57.322 | 187.953 | 182.116 | 110.03 |
| 7767 | C | CA | ASP | 1023 | 56.827 | 188.995 | 181.22 | 110.03 |
| 7768 | C | C | ASP | 1023 | 56.693 | 188.379 | 179.835 | 110.03 |
| 7769 | O | O | ASP | 1023 | 57.7 | 188.127 | 179.168 | 110.03 |
| 7770 | C | CB | ASP | 1023 | 57.761 | 190.203 | 181.195 | 110.03 |
| 7771 | C | CG | ASP | 1023 | 57.191 | 191.366 | 180.405 | 110.03 |
| 7772 | O | OD1 | ASP | 1023 | 57.076 | 191.251 | 179.166 | 110.03 |
| 7773 | O | OD2 | ASP | 1023 | 56.856 | 192.398 | 181.024 | 110.03 |
| 7774 | N | N | VAL | 1024 | 55.452 | 188.129 | 179.413 | 108.87 |
| 7775 | C | CA | VAL | 1024 | 55.221 | 187.506 | 178.115 | 108.87 |
| 7776 | C | C | VAL | 1024 | 55.564 | 188.465 | 176.981 | 108.87 |
| 7777 | O | O | VAL | 1024 | 56.048 | 188.041 | 175.924 | 108.87 |
| 7778 | C | CB | VAL | 1024 | 53.77 | 187 | 178.018 | 108.87 |
| 7779 | C | CG1 | VAL | 1024 | 53.549 | 185.852 | 178.989 | 108.87 |
| 7780 | C | CG2 | VAL | 1024 | 52.789 | 188.127 | 178.302 | 108.87 |
| 7781 | N | N | LYS | 1025 | 55.316 | 189.762 | 177.172 | 107.89 |
| 7782 | C | CA | LYS | 1025 | 55.556 | 190.73 | 176.106 | 107.89 |
| 7783 | C | C | LYS | 1025 | 57.037 | 190.817 | 175.761 | 107.89 |
| 7784 | O | O | LYS | 1025 | 57.419 | 190.76 | 174.586 | 107.89 |
| 7785 | C | CB | LYS | 1025 | 55.016 | 192.102 | 176.513 | 107.89 |
| 7786 | C | CG | LYS | 1025 | 53.56 | 192.336 | 176.144 | 107.89 |
| 7787 | C | CD | LYS | 1025 | 52.622 | 191.602 | 177.088 | 107.89 |
| 7788 | C | CE | LYS | 1025 | 52.681 | 192.184 | 178.49 | 107.89 |
| 7789 | N | NZ | LYS | 1025 | 51.724 | 191.508 | 179.409 | 107.89 |
| 7790 | N | N | ASN | 1026 | 57.888 | 190.951 | 176.774 | 108.61 |
| 7791 | C | CA | ASN | 1026 | 59.327 | 191.044 | 176.57 | 108.61 |
| 7792 | C | C | ASN | 1026 | 60.017 | 189.688 | 176.619 | 108.61 |
| 7793 | O | O | ASN | 1026 | 61.242 | 189.629 | 176.477 | 108.61 |
| 7794 | C | CB | ASN | 1026 | 59.945 | 191.979 | 177.612 | 108.61 |
| 7795 | C | CG | ASN | 1026 | 59.228 | 193.31 | 177.694 | 108.61 |
| 7796 | O | OD1 | ASN | 1026 | 58.455 | 193.667 | 176.805 | 108.61 |
| 7797 | N | ND2 | ASN | 1026 | 59.48 | 194.054 | 178.765 | 108.61 |
| 7798 | N | N | ARG | 1027 | 59.258 | 188.608 | 176.812 | 113.82 |
| 7799 | C | CA | ARG | 1027 | 59.795 | 187.249 | 176.875 | 113.82 |
| 7800 | C | C | ARG | 1027 | 60.886 | 187.14 | 177.941 | 113.82 |
| 7801 | O | O | ARG | 1027 | 62.001 | 186.667 | 177.689 | 113.82 |
| 7802 | C | CB | ARG | 1027 | 60.298 | 186.795 | 175.504 | 113.82 |
| 7803 | C | CG | ARG | 1027 | 60.013 | 185.334 | 175.209 | 113.82 |
| 7804 | C | CD | ARG | 1027 | 60.71 | 184.874 | 173.947 | 113.82 |
| 7805 | N | NE | ARG | 1027 | 60.113 | 185.457 | 172.751 | 113.82 |
| 7806 | C | CZ | ARG | 1027 | 59.362 | 184.787 | 171.889 | 113.82 |
| 7807 | N | NH1 | ARG | 1027 | 59.094 | 183.502 | 172.058 | 113.82 |
| 7808 | N | NH2 | ARG | 1027 | 58.867 | 185.421 | 170.83 | 113.82 |
| 7809 | N | N | ARG | 1028 | 60.548 | 187.602 | 179.142 | 107.72 |
| 7810 | C | CA | ARG | 1028 | 61.411 | 187.517 | 180.311 | 107.72 |
| 7811 | C | C | ARG | 1028 | 60.894 | 186.407 | 181.215 | 107.72 |
| 7812 | O | O | ARG | 1028 | 59.725 | 186.418 | 181.611 | 107.72 |
| 7813 | C | CB | ARG | 1028 | 61.447 | 188.842 | 181.074 | 107.72 |
| 7814 | C | CG | ARG | 1028 | 61.663 | 190.07 | 180.207 | 107.72 |
| 7815 | C | CD | ARG | 1028 | 63.109 | 190.197 | 179.768 | 107.72 |
| 7816 | N | NE | ARG | 1028 | 64.036 | 190.259 | 180.891 | 107.72 |
| 7817 | C | CZ | ARG | 1028 | 64.334 | 191.364 | 181.559 | 107.72 |
| 7818 | N | NH1 | ARG | 1028 | 63.789 | 192.528 | 181.247 | 107.72 |
| 7819 | N | NH2 | ARG | 1028 | 65.203 | 191.301 | 182.564 | 107.72 |
| 7820 | N | N | ASN | 1029 | 61.768 | 185.465 | 181.543 | 119.07 |
| 7821 | C | CA | ASN | 1029 | 61.424 | 184.306 | 182.345 | 119.07 |
| 7822 | C | C | ASN | 1029 | 62.165 | 184.365 | 183.67 | 119.07 |
| 7823 | O | O | ASN | 1029 | 63.387 | 184.559 | 183.678 | 119.07 |
| 7824 | C | CB | ASN | 1029 | 61.781 | 183.018 | 181.594 | 119.07 |
| 7825 | C | CG | ASN | 1029 | 61.129 | 181.789 | 182.191 | 119.07 |
| 7826 | O | OD1 | ASN | 1029 | 60.456 | 181.859 | 183.217 | 119.07 |
| 7827 | N | ND2 | ASN | 1029 | 61.311 | 180.654 | 181.532 | 119.07 |
| 7828 | N | N | PRO | 1030 | 61.467 | 184.279 | 184.805 | 121.93 |
| 7829 | C | CA | PRO | 1030 | 62.177 | 184.312 | 186.092 | 121.93 |
| 7830 | C | C | PRO | 1030 | 62.994 | 183.064 | 186.366 | 121.93 |
| 7831 | O | O | PRO | 1030 | 63.999 | 183.142 | 187.082 | 121.93 |
| 7832 | C | CB | PRO | 1030 | 61.046 | 184.482 | 187.117 | 121.93 |
| 7833 | C | CG | PRO | 1030 | 59.817 | 184.015 | 186.413 | 121.93 |
| 7834 | C | CD | PRO | 1030 | 60.009 | 184.375 | 184.976 | 121.93 |
| 7835 | N | N | ARG | 1031 | 62.599 | 181.918 | 185.817 | 136.32 |
| 7836 | C | CA | ARG | 1031 | 63.222 | 180.648 | 186.163 | 136.32 |
| 7837 | C | C | ARG | 1031 | 64.466 | 180.356 | 185.328 | 136.32 |
| 7838 | O | O | ARG | 1031 | 65.175 | 179.385 | 185.612 | 136.32 |
| 7839 | C | CB | ARG | 1031 | 62.185 | 179.522 | 186.02 | 136.32 |
| 7840 | C | CG | ARG | 1031 | 62.634 | 178.14 | 186.48 | 136.32 |
| 7841 | C | CD | ARG | 1031 | 61.514 | 177.118 | 186.363 | 136.32 |
| 7842 | N | NE | ARG | 1031 | 61.92 | 175.808 | 186.859 | 136.32 |
| 7843 | C | CZ | ARG | 1031 | 62.487 | 174.865 | 186.12 | 136.32 |
| 7844 | N | NH1 | ARG | 1031 | 62.726 | 175.047 | 184.832 | 136.32 |
| 7845 | N | NH2 | ARG | 1031 | 62.821 | 173.709 | 186.687 | 136.32 |
| 7846 | N | N | LEU | 1032 | 64.779 | 181.194 | 184.34 | 125.78 |
| 7847 | C | CA | LEU | 1032 | 65.906 | 180.934 | 183.449 | 125.78 |
| 7848 | C | C | LEU | 1032 | 67.229 | 181.186 | 184.164 | 125.78 |
| 7849 | O | O | LEU | 1032 | 67.94 | 182.153 | 183.876 | 125.78 |
| 7850 | C | CB | LEU | 1032 | 65.805 | 181.795 | 182.191 | 125.78 |
| 7851 | C | CG | LEU | 1032 | 65.036 | 181.185 | 181.019 | 125.78 |
| 7852 | C | CD1 | LEU | 1032 | 64.975 | 182.162 | 179.86 | 125.78 |
| 7853 | C | CD2 | LEU | 1032 | 65.673 | 179.875 | 180.587 | 125.78 |
| 7854 | N | N | VAL | 1033 | 67.559 | 180.304 | 185.101 | 131 |
| 7855 | C | CA | VAL | 1033 | 68.823 | 180.354 | 185.834 | 131 |
| 7856 | C | C | VAL | 1033 | 69.402 | 178.948 | 185.877 | 131 |
| 7857 | O | O | VAL | 1033 | 68.685 | 177.961 | 185.656 | 131 |
| 7858 | C | CB | VAL | 1033 | 68.636 | 180.913 | 187.264 | 131 |
| 7859 | C | CG1 | VAL | 1033 | 68.173 | 182.358 | 187.221 | 131 |
| 7860 | C | CG2 | VAL | 1033 | 67.657 | 180.055 | 188.046 | 131 |
| 7861 | N | N | PRO | 1034 | 70.706 | 178.822 | 186.133 | 131.72 |
| 7862 | C | CA | PRO | 1034 | 71.29 | 177.484 | 186.294 | 131.72 |
| 7863 | C | C | PRO | 1034 | 70.633 | 176.727 | 187.44 | 131.72 |
| 7864 | O | O | PRO | 1034 | 70.253 | 177.306 | 188.46 | 131.72 |
| 7865 | C | CB | PRO | 1034 | 72.77 | 177.769 | 186.581 | 131.72 |
| 7866 | C | CG | PRO | 1034 | 72.844 | 179.23 | 186.911 | 131.72 |
| 7867 | C | CD | PRO | 1034 | 71.735 | 179.873 | 186.152 | 131.72 |
| 7868 | N | N | TYR | 1035 | 70.502 | 175.41 | 187.253 | 143.13 |
| 7869 | C | CA | TYR | 1035 | 69.766 | 174.586 | 188.209 | 143.13 |
| 7870 | C | C | TYR | 1035 | 70.405 | 174.618 | 189.591 | 143.13 |
| 7871 | O | O | TYR | 1035 | 69.702 | 174.519 | 190.604 | 143.13 |
| 7872 | C | CB | TYR | 1035 | 69.664 | 173.151 | 187.683 | 143.13 |
| 7873 | C | CG | TYR | 1035 | 68.826 | 172.204 | 188.525 | 143.13 |
| 7874 | C | CD1 | TYR | 1035 | 67.55 | 171.834 | 188.12 | 143.13 |
| 7875 | C | CD2 | TYR | 1035 | 69.332 | 171.634 | 189.692 | 143.13 |
| 7876 | C | CE1 | TYR | 1035 | 66.782 | 170.962 | 188.871 | 143.13 |
| 7877 | C | CE2 | TYR | 1035 | 68.571 | 170.761 | 190.45 | 143.13 |
| 7878 | C | CZ | TYR | 1035 | 67.298 | 170.428 | 190.033 | 143.13 |
| 7879 | O | OH | TYR | 1035 | 66.539 | 169.557 | 190.779 | 143.13 |
| 7880 | N | N | THR | 1036 | 71.73 | 174.76 | 189.655 | 137.31 |
| 7881 | C | CA | THR | 1036 | 72.403 | 174.811 | 190.948 | 137.31 |
| 7882 | C | C | THR | 1036 | 71.888 | 175.967 | 191.795 | 137.31 |
| 7883 | O | O | THR | 1036 | 71.798 | 175.852 | 193.023 | 137.31 |
| 7884 | C | CB | THR | 1036 | 73.914 | 174.929 | 190.749 | 137.31 |
| 7885 | O | OG1 | THR | 1036 | 74.21 | 176.148 | 190.057 | 137.31 |
| 7886 | C | CG2 | THR | 1036 | 74.436 | 173.754 | 189.936 | 137.31 |
| 7887 | N | N | LEU | 1037 | 71.538 | 177.082 | 191.16 | 138.34 |
| 7888 | C | CA | LEU | 1037 | 71.005 | 178.248 | 191.847 | 138.34 |
| 7889 | C | C | LEU | 1037 | 69.486 | 178.348 | 191.754 | 138.34 |
| 7890 | O | O | LEU | 1037 | 68.924 | 179.385 | 192.119 | 138.34 |
| 7891 | C | CB | LEU | 1037 | 71.641 | 179.522 | 191.285 | 138.34 |
| 7892 | C | CG | LEU | 1037 | 73.141 | 179.691 | 191.529 | 138.34 |
| 7893 | C | CD1 | LEU | 1037 | 73.665 | 180.92 | 190.801 | 138.34 |
| 7894 | C | CD2 | LEU | 1037 | 73.444 | 179.77 | 193.018 | 138.34 |
| 7895 | N | N | LEU | 1038 | 68.815 | 177.304 | 191.276 | 141.13 |
| 7896 | C | CA | LEU | 1038 | 67.38 | 177.362 | 191.042 | 141.13 |
| 7897 | C | C | LEU | 1038 | 66.613 | 177.456 | 192.361 | 141.13 |
| 7898 | O | O | LEU | 1038 | 67.114 | 177.102 | 193.431 | 141.13 |
| 7899 | C | CB | LEU | 1038 | 66.925 | 176.135 | 190.249 | 141.13 |
| 7900 | C | CG | LEU | 1038 | 65.52 | 176.143 | 189.646 | 141.13 |
| 7901 | C | CD1 | LEU | 1038 | 65.376 | 177.292 | 188.667 | 141.13 |
| 7902 | C | CD2 | LEU | 1038 | 65.22 | 174.82 | 188.963 | 141.13 |
| 7903 | N | N | ASP | 1039 | 65.383 | 177.959 | 192.272 | 148.25 |
| 7904 | C | CA | ASP | 1039 | 64.518 | 178.049 | 193.438 | 148.25 |
| 7905 | C | C | ASP | 1039 | 64.113 | 176.658 | 193.918 | 148.25 |
| 7906 | O | O | ASP | 1039 | 64.089 | 175.691 | 193.153 | 148.25 |
| 7907 | C | CB | ASP | 1039 | 63.276 | 178.888 | 193.127 | 148.25 |
| 7908 | C | CG | ASP | 1039 | 62.489 | 178.357 | 191.943 | 148.25 |
| 7909 | O | OD1 | ASP | 1039 | 62.968 | 177.42 | 191.272 | 148.25 |
| 7910 | O | OD2 | ASP | 1039 | 61.386 | 178.881 | 191.683 | 148.25 |
| 7911 | N | N | ASP | 1040 | 63.798 | 176.569 | 195.211 | 149.98 |
| 7912 | C | CA | ASP | 1040 | 63.552 | 175.269 | 195.828 | 149.98 |
| 7913 | C | C | ASP | 1040 | 62.278 | 174.614 | 195.302 | 149.98 |
| 7914 | O | O | ASP | 1040 | 62.27 | 173.411 | 195.02 | 149.98 |
| 7915 | C | CB | ASP | 1040 | 63.495 | 175.417 | 197.349 | 149.98 |
| 7916 | C | CG | ASP | 1040 | 62.426 | 176.396 | 197.806 | 149.98 |
| 7917 | O | OD1 | ASP | 1040 | 61.798 | 177.046 | 196.943 | 149.98 |
| 7918 | O | OD2 | ASP | 1040 | 62.215 | 176.515 | 199.031 | 149.98 |
| 7919 | N | N | ARG | 1041 | 61.193 | 175.381 | 195.166 | 148.91 |
| 7920 | C | CA | ARG | 1041 | 59.891 | 174.778 | 194.891 | 148.91 |
| 7921 | C | C | ARG | 1041 | 59.863 | 174.104 | 193.523 | 148.91 |
| 7922 | O | O | ARG | 1041 | 59.492 | 172.93 | 193.404 | 148.91 |
| 7923 | C | CB | ARG | 1041 | 58.791 | 175.836 | 194.987 | 148.91 |
| 7924 | C | CG | ARG | 1041 | 57.382 | 175.264 | 195.109 | 148.91 |
| 7925 | C | CD | ARG | 1041 | 56.702 | 175.106 | 193.757 | 148.91 |
| 7926 | N | NE | ARG | 1041 | 56.524 | 176.377 | 193.068 | 148.91 |
| 7927 | C | CZ | ARG | 1041 | 56.233 | 176.492 | 191.779 | 148.91 |
| 7928 | N | NH1 | ARG | 1041 | 56.082 | 175.428 | 191.007 | 148.91 |
| 7929 | N | NH2 | ARG | 1041 | 56.092 | 177.705 | 191.251 | 148.91 |
| 7930 | N | N | THR | 1042 | 60.247 | 174.837 | 192.475 | 151.28 |
| 7931 | C | CA | THR | 1042 | 60.167 | 174.289 | 191.124 | 151.28 |
| 7932 | C | C | THR | 1042 | 61.148 | 173.141 | 190.931 | 151.28 |
| 7933 | O | O | THR | 1042 | 60.819 | 172.135 | 190.288 | 151.28 |
| 7934 | C | CB | THR | 1042 | 60.421 | 175.386 | 190.092 | 151.28 |
| 7935 | O | OG1 | THR | 1042 | 61.776 | 175.84 | 190.2 | 151.28 |
| 7936 | C | CG2 | THR | 1042 | 59.479 | 176.557 | 190.321 | 151.28 |
| 7937 | N | N | LYS | 1043 | 62.366 | 173.281 | 191.463 | 149.39 |
| 7938 | C | CA | LYS | 1043 | 63.32 | 172.185 | 191.364 | 149.39 |
| 7939 | C | C | LYS | 1043 | 62.829 | 170.969 | 192.132 | 149.39 |
| 7940 | O | O | LYS | 1043 | 63.004 | 169.84 | 191.669 | 149.39 |
| 7941 | C | CB | LYS | 1043 | 64.705 | 172.626 | 191.846 | 149.39 |
| 7942 | C | CG | LYS | 1043 | 64.911 | 172.648 | 193.352 | 149.39 |
| 7943 | C | CD | LYS | 1043 | 66.391 | 172.648 | 193.695 | 149.39 |
| 7944 | C | CE | LYS | 1043 | 67.058 | 173.935 | 193.242 | 149.39 |
| 7945 | N | NZ | LYS | 1043 | 68.474 | 174.03 | 193.693 | 149.39 |
| 7946 | N | N | LYS | 1044 | 62.173 | 171.177 | 193.277 | 155.01 |
| 7947 | C | CA | LYS | 1044 | 61.587 | 170.058 | 194.007 | 155.01 |
| 7948 | C | C | LYS | 1044 | 60.498 | 169.378 | 193.186 | 155.01 |
| 7949 | O | O | LYS | 1044 | 60.416 | 168.145 | 193.15 | 155.01 |
| 7950 | C | CB | LYS | 1044 | 61.029 | 170.54 | 195.346 | 155.01 |
| 7951 | C | CG | LYS | 1044 | 60.456 | 169.441 | 196.228 | 155.01 |
| 7952 | C | CD | LYS | 1044 | 61.551 | 168.533 | 196.763 | 155.01 |
| 7953 | C | CE | LYS | 1044 | 60.999 | 167.531 | 197.764 | 155.01 |
| 7954 | N | NZ | LYS | 1044 | 60.112 | 166.525 | 197.117 | 155.01 |
| 7955 | N | N | SER | 1045 | 59.656 | 170.167 | 192.515 | 154.74 |
| 7956 | C | CA | SER | 1045 | 58.574 | 169.597 | 191.716 | 154.74 |
| 7957 | C | C | SER | 1045 | 59.117 | 168.766 | 190.558 | 154.74 |
| 7958 | O | O | SER | 1045 | 58.723 | 167.606 | 190.367 | 154.74 |
| 7959 | C | CB | SER | 1045 | 57.663 | 170.712 | 191.202 | 154.74 |
| 7960 | O | OG | SER | 1045 | 58.177 | 171.278 | 190.009 | 154.74 |
| 7961 | N | N | ASN | 1046 | 60.03 | 169.344 | 189.774 | 152.74 |
| 7962 | C | CA | ASN | 1046 | 60.611 | 168.598 | 188.661 | 152.74 |
| 7963 | C | C | ASN | 1046 | 61.378 | 167.383 | 189.165 | 152.74 |
| 7964 | O | O | ASN | 1046 | 61.333 | 166.306 | 188.555 | 152.74 |
| 7965 | C | CB | ASN | 1046 | 61.519 | 169.505 | 187.83 | 152.74 |
| 7966 | C | CG | ASN | 1046 | 60.875 | 170.839 | 187.508 | 152.74 |
| 7967 | O | OD1 | ASN | 1046 | 61.518 | 171.886 | 187.585 | 152.74 |
| 7968 | N | ND2 | ASN | 1046 | 59.598 | 170.808 | 187.149 | 152.74 |
| 7969 | N | N | LYS | 1047 | 62.072 | 167.537 | 190.294 | 152.3 |
| 7970 | C | CA | LYS | 1047 | 62.866 | 166.46 | 190.859 | 152.3 |
| 7971 | C | C | LYS | 1047 | 61.987 | 165.293 | 191.284 | 152.3 |
| 7972 | O | O | LYS | 1047 | 62.34 | 164.131 | 191.058 | 152.3 |
| 7973 | C | CB | LYS | 1047 | 63.664 | 167.011 | 192.04 | 152.3 |
| 7974 | C | CG | LYS | 1047 | 64.678 | 166.078 | 192.646 | 152.3 |
| 7975 | C | CD | LYS | 1047 | 65.891 | 165.961 | 191.749 | 152.3 |
| 7976 | C | CE | LYS | 1047 | 67.036 | 165.308 | 192.489 | 152.3 |
| 7977 | N | NZ | LYS | 1047 | 66.551 | 164.279 | 193.452 | 152.3 |
| 7978 | N | N | ASP | 1048 | 60.833 | 165.578 | 191.896 | 158.86 |
| 7979 | C | CA | ASP | 1048 | 59.935 | 164.495 | 192.282 | 158.86 |
| 7980 | C | C | ASP | 1048 | 59.287 | 163.853 | 191.066 | 158.86 |
| 7981 | O | O | ASP | 1048 | 59.114 | 162.628 | 191.029 | 158.86 |
| 7982 | C | CB | ASP | 1048 | 58.877 | 164.981 | 193.285 | 158.86 |
| 7983 | C | CG | ASP | 1048 | 57.881 | 165.965 | 192.692 | 158.86 |
| 7984 | O | OD1 | ASP | 1048 | 57.201 | 165.636 | 191.697 | 158.86 |
| 7985 | O | OD2 | ASP | 1048 | 57.75 | 167.067 | 193.262 | 158.86 |
| 7986 | N | N | SER | 1049 | 58.94 | 164.652 | 190.054 | 158.47 |
| 7987 | C | CA | SER | 1049 | 58.393 | 164.069 | 188.834 | 158.47 |
| 7988 | C | C | SER | 1049 | 59.377 | 163.079 | 188.224 | 158.47 |
| 7989 | O | O | SER | 1049 | 59.005 | 161.962 | 187.837 | 158.47 |
| 7990 | C | CB | SER | 1049 | 58.056 | 165.172 | 187.832 | 158.47 |
| 7991 | O | OG | SER | 1049 | 59.174 | 166.006 | 187.597 | 158.47 |
| 7992 | N | N | LEU | 1050 | 60.652 | 163.457 | 188.165 | 155.57 |
| 7993 | C | CA | LEU | 1050 | 61.626 | 162.591 | 187.515 | 155.57 |
| 7994 | C | C | LEU | 1050 | 62.051 | 161.428 | 188.412 | 155.57 |
| 7995 | O | O | LEU | 1050 | 62.377 | 160.346 | 187.905 | 155.57 |
| 7996 | C | CB | LEU | 1050 | 62.811 | 163.424 | 187.032 | 155.57 |
| 7997 | C | CG | LEU | 1050 | 63.674 | 164.207 | 188.014 | 155.57 |
| 7998 | C | CD1 | LEU | 1050 | 64.737 | 163.345 | 188.649 | 155.57 |
| 7999 | C | CD2 | LEU | 1050 | 64.279 | 165.382 | 187.286 | 155.57 |
| 8000 | N | N | ARG | 1051 | 62.041 | 161.599 | 189.744 | 156.42 |
| 8001 | C | CA | ARG | 1051 | 62.264 | 160.413 | 190.567 | 156.42 |
| 8002 | C | C | ARG | 1051 | 61.116 | 159.431 | 190.406 | 156.42 |
| 8003 | O | O | ARG | 1051 | 61.336 | 158.218 | 190.428 | 156.42 |
| 8004 | C | CB | ARG | 1051 | 62.459 | 160.695 | 192.067 | 156.42 |
| 8005 | C | CG | ARG | 1051 | 61.289 | 161.273 | 192.864 | 156.42 |
| 8006 | C | CD | ARG | 1051 | 61.686 | 161.429 | 194.354 | 156.42 |
| 8007 | N | NE | ARG | 1051 | 62.755 | 162.365 | 194.683 | 156.42 |
| 8008 | C | CZ | ARG | 1051 | 62.581 | 163.655 | 194.935 | 156.42 |
| 8009 | N | NH1 | ARG | 1051 | 61.374 | 164.184 | 195.025 | 156.42 |
| 8010 | N | NH2 | ARG | 1051 | 63.644 | 164.417 | 195.173 | 156.42 |
| 8011 | N | N | GLU | 1052 | 59.888 | 159.932 | 190.246 | 160.07 |
| 8012 | C | CA | GLU | 1052 | 58.764 | 159.048 | 189.957 | 160.07 |
| 8013 | C | C | GLU | 1052 | 58.962 | 158.323 | 188.634 | 160.07 |
| 8014 | O | O | GLU | 1052 | 58.665 | 157.13 | 188.521 | 160.07 |
| 8015 | C | CB | GLU | 1052 | 57.457 | 159.84 | 189.944 | 160.07 |
| 8016 | C | CG | GLU | 1052 | 56.213 | 158.964 | 189.922 | 160.07 |
| 8017 | C | CD | GLU | 1052 | 55.971 | 158.25 | 191.237 | 160.07 |
| 8018 | O | OE1 | GLU | 1052 | 56.411 | 158.764 | 192.286 | 160.07 |
| 8019 | O | OE2 | GLU | 1052 | 55.341 | 157.171 | 191.222 | 160.07 |
| 8020 | N | N | ALA | 1053 | 59.452 | 159.032 | 187.616 | 158.31 |
| 8021 | C | CA | ALA | 1053 | 59.713 | 158.379 | 186.334 | 158.31 |
| 8022 | C | C | ALA | 1053 | 60.752 | 157.267 | 186.474 | 158.31 |
| 8023 | O | O | ALA | 1053 | 60.546 | 156.141 | 185.994 | 158.31 |
| 8024 | C | CB | ALA | 1053 | 60.167 | 159.412 | 185.303 | 158.31 |
| 8025 | N | N | VAL | 1054 | 61.868 | 157.56 | 187.146 | 154.17 |
| 8026 | C | CA | VAL | 1054 | 62.92 | 156.56 | 187.313 | 154.17 |
| 8027 | C | C | VAL | 1054 | 62.417 | 155.39 | 188.152 | 154.17 |
| 8028 | O | O | VAL | 1054 | 62.732 | 154.225 | 187.876 | 154.17 |
| 8029 | C | CB | VAL | 1054 | 64.177 | 157.204 | 187.927 | 154.17 |
| 8030 | C | CG1 | VAL | 1054 | 65.245 | 156.153 | 188.184 | 154.17 |
| 8031 | C | CG2 | VAL | 1054 | 64.713 | 158.294 | 187.011 | 154.17 |
| 8032 | N | N | ARG | 1055 | 61.62 | 155.678 | 189.182 | 155.46 |
| 8033 | C | CA | ARG | 1055 | 61.1 | 154.619 | 190.035 | 155.46 |
| 8034 | C | C | ARG | 1055 | 60.08 | 153.759 | 189.304 | 155.46 |
| 8035 | O | O | ARG | 1055 | 59.987 | 152.56 | 189.569 | 155.46 |
| 8036 | C | CB | ARG | 1055 | 60.492 | 155.218 | 191.303 | 155.46 |
| 8037 | C | CG | ARG | 1055 | 60.155 | 154.189 | 192.363 | 155.46 |
| 8038 | C | CD | ARG | 1055 | 61.421 | 153.657 | 193.014 | 155.46 |
| 8039 | N | NE | ARG | 1055 | 62.064 | 154.643 | 193.874 | 155.46 |
| 8040 | C | CZ | ARG | 1055 | 61.718 | 154.88 | 195.132 | 155.46 |
| 8041 | N | NH1 | ARG | 1055 | 60.733 | 154.216 | 195.715 | 155.46 |
| 8042 | N | NH2 | ARG | 1055 | 62.378 | 155.805 | 195.823 | 155.46 |
| 8043 | N | N | THR | 1056 | 59.315 | 154.339 | 188.378 | 160.35 |
| 8044 | C | CA | THR | 1056 | 58.412 | 153.528 | 187.568 | 160.35 |
| 8045 | C | C | THR | 1056 | 59.191 | 152.643 | 186.603 | 160.35 |
| 8046 | O | O | THR | 1056 | 58.804 | 151.494 | 186.35 | 160.35 |
| 8047 | C | CB | THR | 1056 | 57.436 | 154.425 | 186.809 | 160.35 |
| 8048 | O | OG1 | THR | 1056 | 56.6 | 155.12 | 187.743 | 160.35 |
| 8049 | C | CG2 | THR | 1056 | 56.558 | 153.595 | 185.887 | 160.35 |
| 8050 | N | N | LEU | 1057 | 60.295 | 153.159 | 186.059 | 154.78 |
| 8051 | C | CA | LEU | 1057 | 61.16 | 152.309 | 185.245 | 154.78 |
| 8052 | C | C | LEU | 1057 | 61.704 | 151.146 | 186.071 | 154.78 |
| 8053 | O | O | LEU | 1057 | 61.708 | 149.993 | 185.616 | 154.78 |
| 8054 | C | CB | LEU | 1057 | 62.29 | 153.148 | 184.643 | 154.78 |
| 8055 | C | CG | LEU | 1057 | 63.241 | 152.54 | 183.606 | 154.78 |
| 8056 | C | CD1 | LEU | 1057 | 63.798 | 153.638 | 182.713 | 154.78 |
| 8057 | C | CD2 | LEU | 1057 | 64.379 | 151.784 | 184.258 | 154.78 |
| 8058 | N | N | LEU | 1058 | 62.138 | 151.426 | 187.302 | 155.36 |
| 8059 | C | CA | LEU | 1058 | 62.588 | 150.361 | 188.195 | 155.36 |
| 8060 | C | C | LEU | 1058 | 61.456 | 149.401 | 188.544 | 155.36 |
| 8061 | O | O | LEU | 1058 | 61.692 | 148.205 | 188.746 | 155.36 |
| 8062 | C | CB | LEU | 1058 | 63.184 | 150.958 | 189.47 | 155.36 |
| 8063 | C | CG | LEU | 1058 | 64.472 | 151.767 | 189.324 | 155.36 |
| 8064 | C | CD1 | LEU | 1058 | 64.853 | 152.405 | 190.649 | 155.36 |
| 8065 | C | CD2 | LEU | 1058 | 65.589 | 150.869 | 188.826 | 155.36 |
| 8066 | N | N | GLY | 1059 | 60.228 | 149.91 | 188.647 | 159.8 |
| 8067 | C | CA | GLY | 1059 | 59.096 | 149.045 | 188.931 | 159.8 |
| 8068 | C | C | GLY | 1059 | 58.812 | 148.079 | 187.799 | 159.8 |
| 8069 | O | O | GLY | 1059 | 58.535 | 146.899 | 188.03 | 159.8 |
| 8070 | N | N | TYR | 1060 | 58.872 | 148.567 | 186.558 | 162.62 |
| 8071 | C | CA | TYR | 1060 | 58.85 | 147.65 | 185.423 | 162.62 |
| 8072 | C | C | TYR | 1060 | 60.082 | 146.757 | 185.39 | 162.62 |
| 8073 | O | O | TYR | 1060 | 60.064 | 145.719 | 184.718 | 162.62 |
| 8074 | C | CB | TYR | 1060 | 58.699 | 148.411 | 184.106 | 162.62 |
| 8075 | C | CG | TYR | 1060 | 57.255 | 148.638 | 183.717 | 162.62 |
| 8076 | C | CD1 | TYR | 1060 | 56.231 | 148.431 | 184.632 | 162.62 |
| 8077 | C | CD2 | TYR | 1060 | 56.912 | 149.022 | 182.428 | 162.62 |
| 8078 | C | CE1 | TYR | 1060 | 54.908 | 148.623 | 184.282 | 162.62 |
| 8079 | C | CE2 | TYR | 1060 | 55.591 | 149.215 | 182.068 | 162.62 |
| 8080 | C | CZ | TYR | 1060 | 54.594 | 149.015 | 182.998 | 162.62 |
| 8081 | O | OH | TYR | 1060 | 53.279 | 149.207 | 182.644 | 162.62 |
| 8082 | N | N | GLY | 1061 | 61.149 | 147.134 | 186.092 | 157.95 |
| 8083 | C | CA | GLY | 1061 | 62.197 | 146.199 | 186.449 | 157.95 |
| 8084 | C | C | GLY | 1061 | 63.443 | 146.243 | 185.597 | 157.95 |
| 8085 | O | O | GLY | 1061 | 64.35 | 145.431 | 185.82 | 157.95 |
| 8086 | N | N | TYR | 1062 | 63.529 | 147.154 | 184.634 | 158.59 |
| 8087 | C | CA | TYR | 1062 | 64.717 | 147.238 | 183.793 | 158.59 |
| 8088 | C | C | TYR | 1062 | 65.838 | 147.862 | 184.613 | 158.59 |
| 8089 | O | O | TYR | 1062 | 65.927 | 149.085 | 184.751 | 158.59 |
| 8090 | C | CB | TYR | 1062 | 64.414 | 148.03 | 182.526 | 158.59 |
| 8091 | C | CG | TYR | 1062 | 63.459 | 147.295 | 181.614 | 158.59 |
| 8092 | C | CD1 | TYR | 1062 | 63.201 | 145.944 | 181.805 | 158.59 |
| 8093 | C | CD2 | TYR | 1062 | 62.808 | 147.945 | 180.575 | 158.59 |
| 8094 | C | CE1 | TYR | 1062 | 62.33 | 145.261 | 180.993 | 158.59 |
| 8095 | C | CE2 | TYR | 1062 | 61.93 | 147.263 | 179.749 | 158.59 |
| 8096 | C | CZ | TYR | 1062 | 61.698 | 145.919 | 179.966 | 158.59 |
| 8097 | O | OH | TYR | 1062 | 60.831 | 145.216 | 179.163 | 158.59 |
| 8098 | N | N | ASN | 1063 | 66.697 | 147.01 | 185.162 | 149.24 |
| 8099 | C | CA | ASN | 1063 | 67.651 | 147.463 | 186.163 | 149.24 |
| 8100 | C | C | ASN | 1063 | 68.742 | 148.324 | 185.538 | 149.24 |
| 8101 | O | O | ASN | 1063 | 69.173 | 148.103 | 184.4 | 149.24 |
| 8102 | C | CB | ASN | 1063 | 68.257 | 146.275 | 186.917 | 149.24 |
| 8103 | C | CG | ASN | 1063 | 68.888 | 145.249 | 186 | 149.24 |
| 8104 | O | OD1 | ASN | 1063 | 68.927 | 145.419 | 184.786 | 149.24 |
| 8105 | N | ND2 | ASN | 1063 | 69.389 | 144.168 | 186.586 | 149.24 |
| 8106 | N | N | LEU | 1064 | 69.174 | 149.328 | 186.299 | 148.93 |
| 8107 | C | CA | LEU | 1064 | 70.152 | 150.31 | 185.841 | 148.93 |
| 8108 | C | C | LEU | 1064 | 71.554 | 149.788 | 186.116 | 148.93 |
| 8109 | O | O | LEU | 1064 | 72.132 | 150.039 | 187.175 | 148.93 |
| 8110 | C | CB | LEU | 1064 | 69.935 | 151.655 | 186.527 | 148.93 |
| 8111 | C | CG | LEU | 1064 | 68.883 | 152.625 | 185.984 | 148.93 |
| 8112 | C | CD1 | LEU | 1064 | 69.27 | 153.071 | 184.588 | 148.93 |
| 8113 | C | CD2 | LEU | 1064 | 67.49 | 152.027 | 185.996 | 148.93 |
| 8114 | N | N | GLU | 1065 | 72.114 | 149.062 | 185.149 | 146.78 |
| 8115 | C | CA | GLU | 1065 | 73.533 | 148.72 | 185.186 | 146.78 |
| 8116 | C | C | GLU | 1065 | 74.321 | 149.974 | 184.815 | 146.78 |
| 8117 | O | C | GLU | 1065 | 74.729 | 150.19 | 183.672 | 146.78 |
| 8118 | C | CB | GLU | 1065 | 73.842 | 147.553 | 184.26 | 146.78 |
| 8119 | C | CG | GLU | 1065 | 73.48 | 146.193 | 184.825 | 146.78 |
| 8120 | C | CD | GLU | 1065 | 72.072 | 145.77 | 184.478 | 146.78 |
| 8121 | O | OE1 | GLU | 1065 | 71.406 | 146.494 | 183.709 | 146.78 |
| 8122 | O | OE2 | GLU | 1065 | 71.637 | 144.708 | 184.968 | 146.78 |
| 8123 | N | N | ALA | 1066 | 74.505 | 150.823 | 185.811 | 144.43 |
| 8124 | C | CA | ALA | 1066 | 75.123 | 152.123 | 185.638 | 144.43 |
| 8125 | C | C | ALA | 1066 | 76.524 | 152.142 | 186.239 | 144.43 |
| 8126 | O | O | ALA | 1066 | 76.804 | 151.426 | 187.204 | 144.43 |
| 8127 | C | CB | ALA | 1066 | 74.273 | 153.203 | 186.299 | 144.43 |
| 8128 | N | N | PRO | 1067 | 77.421 | 152.953 | 185.688 | 145.31 |
| 8129 | C | CA | PRO | 1067 | 78.74 | 153.119 | 186.295 | 145.31 |
| 8130 | C | C | PRO | 1067 | 78.689 | 154.096 | 187.461 | 145.31 |
| 8131 | O | O | PRO | 1067 | 77.736 | 154.858 | 187.634 | 145.31 |
| 8132 | C | CB | PRO | 1067 | 79.58 | 153.677 | 185.144 | 145.31 |
| 8133 | C | CG | PRO | 1067 | 78.601 | 154.498 | 184.37 | 145.31 |
| 8134 | C | CD | PRO | 1067 | 77.268 | 153.781 | 184.478 | 145.31 |
| 8135 | N | N | ASP | 1068 | 79.743 | 154.057 | 188.268 | 145.93 |
| 8136 | C | CA | ASP | 1068 | 79.905 | 154.978 | 189.385 | 145.93 |
| 8137 | C | C | ASP | 1068 | 80.904 | 156.065 | 189.003 | 145.93 |
| 8138 | O | O | ASP | 1068 | 81.915 | 155.79 | 188.347 | 145.93 |
| 8139 | C | CB | ASP | 1068 | 80.317 | 154.235 | 190.661 | 145.93 |
| 8140 | C | CG | ASP | 1068 | 81.716 | 153.631 | 190.591 | 145.93 |
| 8141 | O | OD1 | ASP | 1068 | 82.297 | 153.525 | 189.492 | 145.93 |
| 8142 | O | OD2 | ASP | 1068 | 82.241 | 153.254 | 191.66 | 145.93 |
| 8143 | N | N | GLN | 1069 | 80.592 | 157.305 | 189.368 | 145.39 |
| 8144 | C | CA | GLN | 1069 | 81.451 | 158.443 | 189.052 | 145.39 |
| 8145 | C | C | GLN | 1069 | 82.436 | 158.621 | 190.199 | 145.39 |
| 8146 | O | O | GLN | 1069 | 82.098 | 159.181 | 191.245 | 145.39 |
| 8147 | C | CB | GLN | 1069 | 80.622 | 159.701 | 188.821 | 145.39 |
| 8148 | C | CG | GLN | 1069 | 81.454 | 160.945 | 188.555 | 145.39 |
| 8149 | C | CD | GLN | 1069 | 82.577 | 160.693 | 187.567 | 145.39 |
| 8150 | O | OE1 | GLN | 1069 | 82.348 | 160.212 | 186.457 | 145.39 |
| 8151 | N | NE2 | GLN | 1069 | 83.801 | 161.013 | 187.97 | 145.39 |
| 8152 | N | N | ASP | 1070 | 83.659 | 158.138 | 190.004 | 148.64 |
| 8153 | C | CA | ASP | 1070 | 84.686 | 158.279 | 191.023 | 148.64 |
| 8154 | C | C | ASP | 1070 | 85.1 | 159.739 | 191.161 | 148.64 |
| 8155 | O | O | ASP | 1070 | 84.97 | 160.537 | 190.229 | 148.64 |
| 8156 | C | CB | ASP | 1070 | 85.901 | 157.417 | 190.68 | 148.64 |
| 8157 | C | CG | ASP | 1070 | 85.666 | 155.945 | 190.951 | 148.64 |
| 8158 | O | OD1 | ASP | 1070 | 85.912 | 155.503 | 192.093 | 148.64 |
| 8159 | O | OD2 | ASP | 1070 | 85.235 | 155.23 | 190.023 | 148.64 |
| 8160 | N | N | HIS | 1071 | 85.591 | 160.087 | 192.348 | 164.22 |
| 8161 | C | CA | HIS | 1071 | 86.035 | 161.451 | 192.598 | 164.22 |
| 8162 | C | C | HIS | 1071 | 87.214 | 161.797 | 191.698 | 164.22 |
| 8163 | O | O | HIS | 1071 | 88.188 | 161.044 | 191.61 | 164.22 |
| 8164 | C | CB | HIS | 1071 | 86.42 | 161.619 | 194.068 | 164.22 |
| 8165 | C | CG | HIS | 1071 | 86.729 | 163.032 | 194.452 | 164.22 |
| 8166 | N | ND1 | HIS | 1071 | 87.984 | 163.585 | 194.313 | 164.22 |
| 8167 | C | CD2 | HIS | 1071 | 85.944 | 164.007 | 194.969 | 164.22 |
| 8168 | C | CE1 | HIS | 1071 | 87.959 | 164.838 | 194.729 | 164.22 |
| 8169 | N | NE2 | HIS | 1071 | 86.733 | 165.12 | 195.132 | 164.22 |
| 8170 | N | N | ALA | 1072 | 87.12 | 162.94 | 191.027 | 178.88 |
| 8171 | C | CA | ALA | 1072 | 88.154 | 163.405 | 190.114 | 178.88 |
| 8172 | C | C | ALA | 1072 | 88.796 | 164.668 | 190.67 | 178.88 |
| 8173 | O | O | ALA | 1072 | 88.094 | 165.609 | 191.055 | 178.88 |
| 8174 | C | CB | ALA | 1072 | 87.576 | 163.671 | 188.724 | 178.88 |
| 8175 | N | N | ALA | 1073 | 90.125 | 164.684 | 190.713 | 194.36 |
| 8176 | C | CA | ALA | 1073 | 90.842 | 165.86 | 191.184 | 194.36 |
| 8177 | C | C | ALA | 1073 | 90.707 | 167 | 190.183 | 194.36 |
| 8178 | O | O | ALA | 1073 | 90.858 | 166.806 | 188.973 | 194.36 |
| 8179 | C | CB | ALA | 1073 | 92.316 | 165.528 | 191.413 | 194.36 |
| 8180 | N | N | ARG | 1074 | 90.419 | 168.194 | 190.694 | 201.94 |
| 8181 | C | CA | ARG | 1074 | 90.271 | 169.39 | 189.877 | 201.94 |
| 8182 | C | C | ARG | 1074 | 91.183 | 170.475 | 190.43 | 201.94 |
| 8183 | O | O | ARG | 1074 | 91.153 | 170.764 | 191.631 | 201.94 |
| 8184 | C | CB | ARG | 1074 | 88.811 | 169.855 | 189.85 | 201.94 |
| 8185 | C | CG | ARG | 1074 | 88.539 | 171.055 | 188.957 | 201.94 |
| 8186 | C | CD | ARG | 1074 | 87.057 | 171.407 | 188.957 | 201.94 |
| 8187 | N | NE | ARG | 1074 | 86.578 | 171.813 | 190.273 | 201.94 |
| 8188 | C | CZ | ARG | 1074 | 86.686 | 173.039 | 190.764 | 201.94 |
| 8189 | N | NH1 | ARG | 1074 | 87.237 | 174.018 | 190.065 | 201.94 |
| 8190 | N | NH2 | ARG | 1074 | 86.224 | 173.293 | 191.986 | 201.94 |
| 8191 | N | N | ALA | 1075 | 91.995 | 171.07 | 189.551 | 202.15 |
| 8192 | C | CA | ALA | 1075 | 93.01 | 172.021 | 189.992 | 202.15 |
| 8193 | C | C | ALA | 1075 | 92.4 | 173.268 | 190.621 | 202.15 |
| 8194 | O | O | ALA | 1075 | 93.055 | 173.921 | 191.442 | 202.15 |
| 8195 | C | CB | ALA | 1075 | 93.909 | 172.411 | 188.818 | 202.15 |
| 8196 | N | N | GLU | 1076 | 91.18 | 173.633 | 190.216 | 190.56 |
| 8197 | C | CA | GLU | 1076 | 90.386 | 174.687 | 190.843 | 190.56 |
| 8198 | C | C | GLU | 1076 | 90.947 | 176.073 | 190.528 | 190.56 |
| 8199 | O | O | GLU | 1076 | 90.291 | 177.091 | 190.778 | 190.56 |
| 8200 | C | CB | GLU | 1076 | 90.29 | 174.428 | 192.359 | 190.56 |
| 8201 | C | CG | GLU | 1076 | 89.915 | 175.613 | 193.241 | 190.56 |
| 8202 | C | CD | GLU | 1076 | 88.448 | 175.98 | 193.136 | 190.56 |
| 8203 | O | OE1 | GLU | 1076 | 87.635 | 175.095 | 192.8 | 190.56 |
| 8204 | O | OE2 | GLU | 1076 | 88.11 | 177.156 | 193.386 | 190.56 |

¹atom site ID number as assigned in mmCIF file for RCSB PBD structure 7UA1;
²element symbol representing atom species;
³atom identifier assigned in mmCIF file for RCSB PBD structure 7UA1;
⁴encompassing residue type;
⁵encompassing residue number;
⁶⁻⁸Atom-site coordinates in angstroms specified according to a set of orthogonal Cartesian axes related to the cell axes; Isotropic atomic displacement parameter, or equivalent isotropic atomic displacement parameter, B_{eq}, calculated from the anisotropic displacement parameters, where: B_{eq} = (1/3) sumᵢ[sumⱼ(B^{ij} Aᵢ Aⱼ a*ᵢ a*ⱼ)]; A = the real space cell lengths; a* = the reciprocal space cell lengths; and B^{ij} = 8 pi² U^{ij}.

TABLE 4
Three-dimensional atomic coordinates of Compound 1.

| | type_ | label_ | | | | B_iso_ |
|---|---|---|---|---|---|---|
| Id¹ | symbol² | atom_id³ | Cartn_x⁶ | Cartn_y⁷ | Cartn_z⁸ | or_equiv⁹ |
| 138400 | C | C01 | 50.572 | 176.272 | 179.982 | 0.5 |
| 138401 | C | C03 | 50.159 | 173.915 | 179.952 | 0.5 |
| 138402 | C | C04 | 50.151 | 173.364 | 181.224 | 0.5 |
| 138403 | C | C05 | 50.624 | 172.075 | 181.449 | 0.5 |
| 138404 | C | C06 | 51.112 | 171.343 | 180.349 | 0.5 |
| 138405 | C | C07 | 51.122 | 171.906 | 179.077 | 0.5 |
| 138406 | C | C08 | 50.643 | 173.184 | 178.878 | 0.5 |
| 138407 | C | C10 | 53.121 | 170.019 | 181.711 | 0.5 |
| 138408 | C | C11 | 52.652 | 170.105 | 183.135 | 0.5 |
| 138409 | C | C13 | 50.617 | 171.488 | 182.839 | 0.5 |
| 138410 | C | C14 | 52.107 | 171.869 | 184.785 | 0.5 |
| 138411 | C | C15 | 52.944 | 173.123 | 184.842 | 0.5 |
| 138412 | C | C16 | 52.464 | 174.307 | 184.309 | 0.5 |
| 138413 | C | C17 | 53.225 | 175.461 | 184.339 | 0.5 |
| 138414 | C | C18 | 54.492 | 175.447 | 184.897 | 0.5 |
| 138415 | C | C19 | 54.981 | 174.265 | 185.425 | 0.5 |
| 138416 | C | C20 | 54.215 | 173.112 | 185.392 | 0.5 |
| 138417 | C | C21 | 55.317 | 176.694 | 184.929 | 0.5 |
| 138418 | N | N12 | 51.976 | 171.382 | 183.4 | 0.5 |
| 138419 | 0 | O02 | 49.681 | 175.185 | 179.755 | 0.5 |
| 138420 | 0 | O22 | 55.932 | 177.008 | 183.895 | 0.5 |
| 138421 | 0 | O23 | 55.341 | 177.346 | 185.986 | 0.5 |
| 138422 | S | S09 | 51.724 | 169.708 | 180.612 | 0.5 |

^{1-3,6-9}See description for TABLE 3 above.

TABLE 5
Three-dimensional atomic coordinates of ATP.

| | type_ | label_ | | | | B_iso_ |
|---|---|---|---|---|---|---|
| Id1 | symbol² | atom_id³ | Cartn_x⁶ | Cartn_y⁷ | Cartn_z⁸ | or_equiv⁹ |
| 138369 | P | PG | 57.947 | 176.674 | 179.663 | 139.1 |
| 138370 | O | O1G | 58.257 | 178.124 | 179.946 | 139.1 |
| 138371 | O | O2G | 58.591 | 176.144 | 178.405 | 139.1 |
| 138372 | O | O3G | 58.08 | 175.792 | 180.877 | 139.1 |
| 138373 | P | PB | 55.52 | 175.306 | 179.197 | 139.1 |
| 138374 | O | O1B | 54.28 | 175.621 | 178.396 | 139.1 |
| 138375 | O | O2B | 56.411 | 174.167 | 178.771 | 139.1 |
| 138376 | O | O3B | 56.375 | 176.666 | 179.323 | 139.1 |
| 138377 | P | PA | 53.956 | 173.944 | 181.032 | 139.1 |
| 138378 | O | O1A | 53.819 | 173.014 | 179.856 | 139.1 |
| 138379 | O | O2A | 52.75 | 174.679 | 181.516 | 139.1 |
| 138380 | O | O3A | 55.083 | 175.036 | 180.716 | 139.1 |
| 138381 | O | O5′ | 54.615 | 173.193 | 182.284 | 139.1 |
| 138382 | C | C5' | 55.879 | 173.641 | 182.753 | 139.1 |
| 138383 | C | C4′ | 56.876 | 172.499 | 182.649 | 139.1 |
| 138384 | O | O4′ | 56.489 | 171.592 | 181.613 | 139.1 |
| 138385 | C | C3′ | 58.263 | 173.017 | 182.302 | 139.1 |
| 138386 | O | O3′ | 59.167 | 172.826 | 183.394 | 139.1 |
| 138387 | C | C2′ | 58.716 | 172.217 | 181.1 | 139.1 |
| 138388 | O | O2 | 59.974 | 171.595 | 181.367 | 139.1 |
| 138389 | C | C1′ | 57.633 | 171.177 | 180.863 | 139.1 |
| 138390 | N | N9 | 57.35 | 171.147 | 179.412 | 139.1 |
| 138391 | C | C8 | 58.24 | 171.501 | 178.47 | 139.1 |
| 138392 | N | N7 | 57.716 | 171.381 | 177.229 | 139.1 |
| 138393 | C | C5 | 56.46 | 170.936 | 177.373 | 139.1 |
| 138394 | C | C6 | 55.361 | 170.597 | 176.454 | 139.1 |
| 138395 | N | N6 | 55.545 | 170.724 | 175.122 | 139.1 |
| 138396 | N | N1 | 54.2 | 170.162 | 176.986 | 139.1 |
| 138397 | C | C2 | 54.043 | 170.048 | 178.314 | 139.1 |
| 138398 | N | N3 | 55.006 | 170.333 | 179.203 | 139.1 |
| 138399 | C | C4 | 56.217 | 170.782 | 178.809 | 139.1 |

^{1-3,6-9}See description for TABLE 3 above.

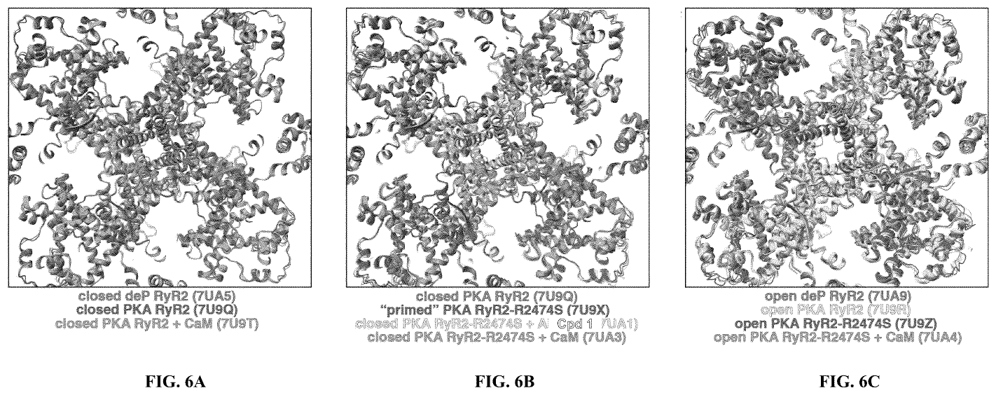

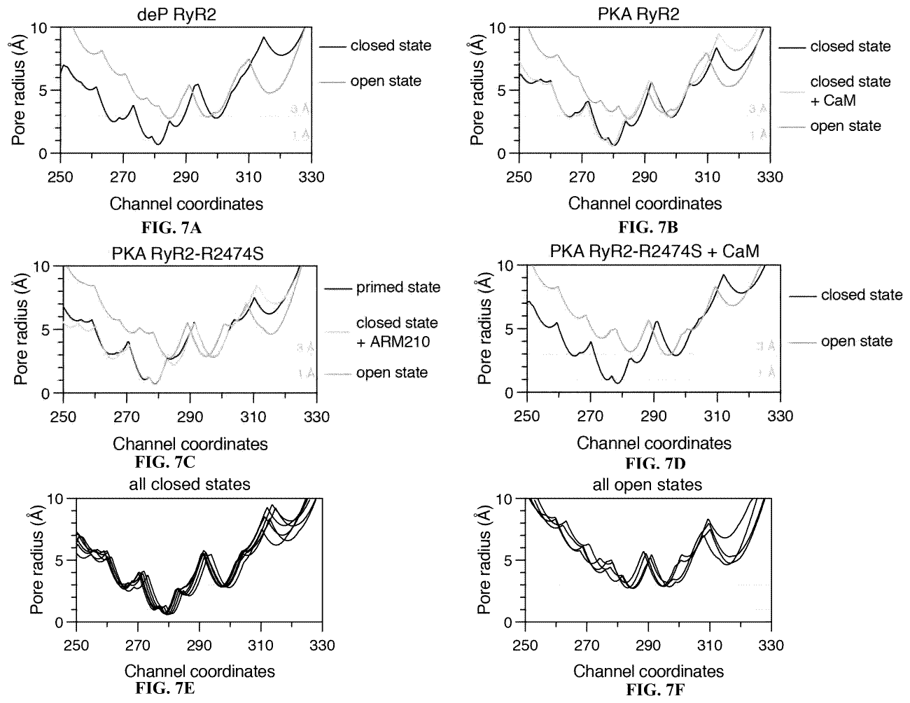

Comparison of the pore region from all the structures showed no differences in the closed states and no differences in the open states. The comparison suggests that the methodology implemented is reproducible. FIGS. 6A-6C depict aligned atomic models of all structures focusing on the pore and TM domain. FIGS. 7A-7F show pore radii estimation for each structure calculated with HOLE. Channel coordinates are arbitrary and correlation among structures are approximate. Visual and HOLE analysis suggest that no differences are found in the pore of the closed states and no differences are found in the pore of the open states.

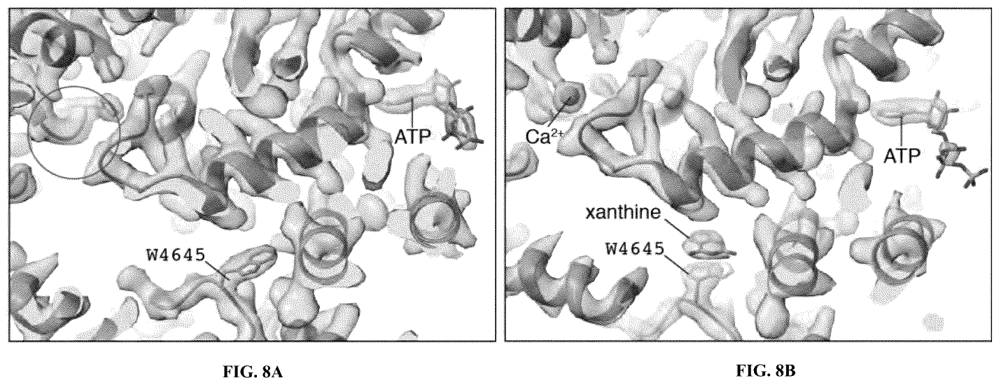

Of the three activators present in the sample, only ATP was detected in the closed state structures (FIG. 8A). All three activators Ca²⁺, ATP, and xanthine were present in the open state structures (FIG. 8B), suggesting that the binding of the additional activators, Ca²⁺ and xanthine, promotes opening of the channel. FIG. 8A and FIG. 8B depict models of RyR2 with the respective cryo-EM maps centered on the ligand binding site of the closed (FIG. 8A) and open (FIG. 8B) state of representative RyR2 structures.

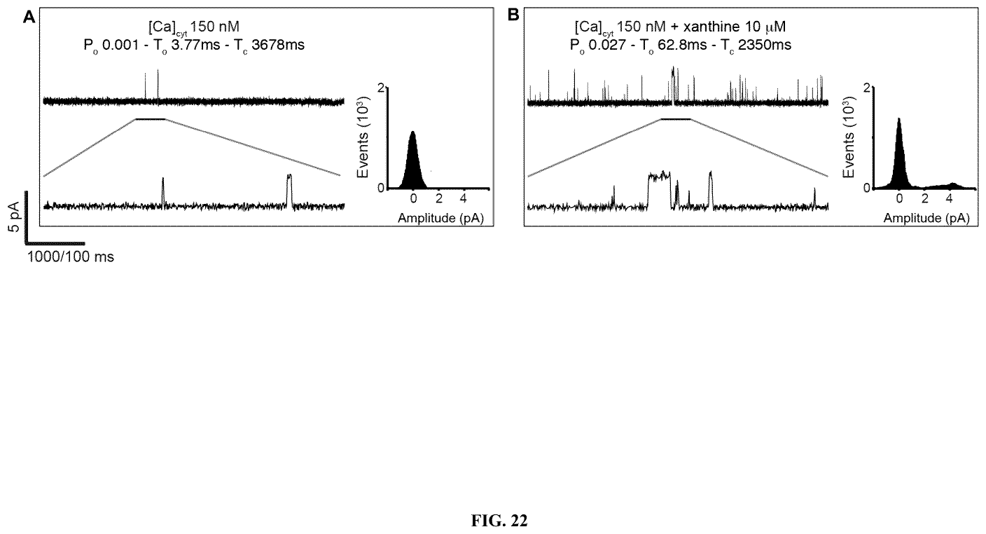

Xanthine occupied the site formed by W4645, I4926, and Y4944. To confirm the functional role of xanthine, single-channel experiments were performed showing that, at presumed physiological concentrations during exercise (10 μM), a ˜25-fold increase was observed in the open probability of RyR2 in the presence of xanthine (see FIG. 22A, FIG. 22B, EXAMPLE 7).

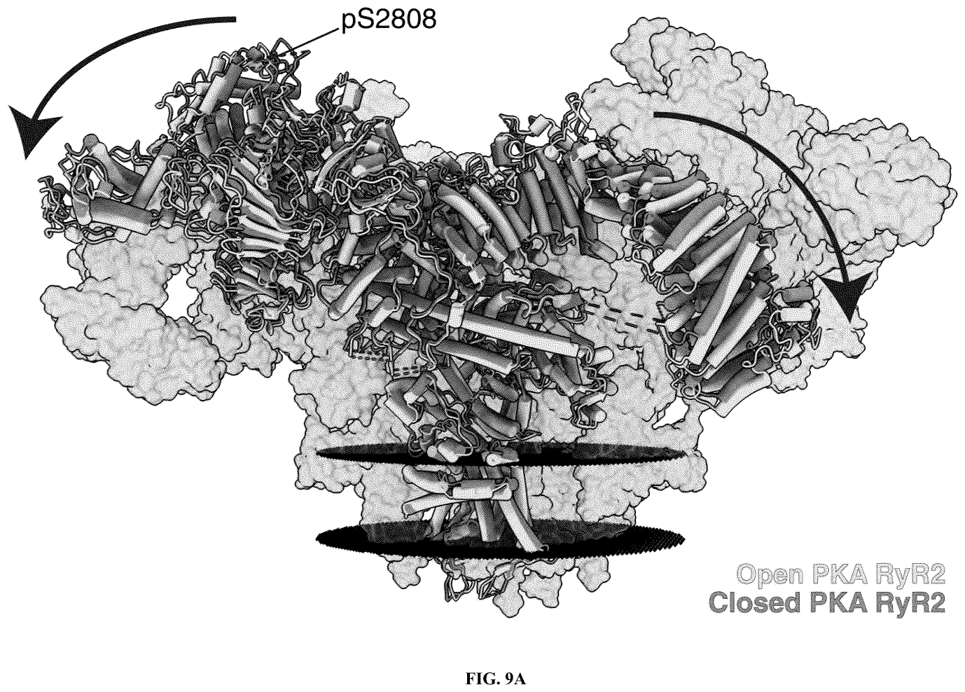

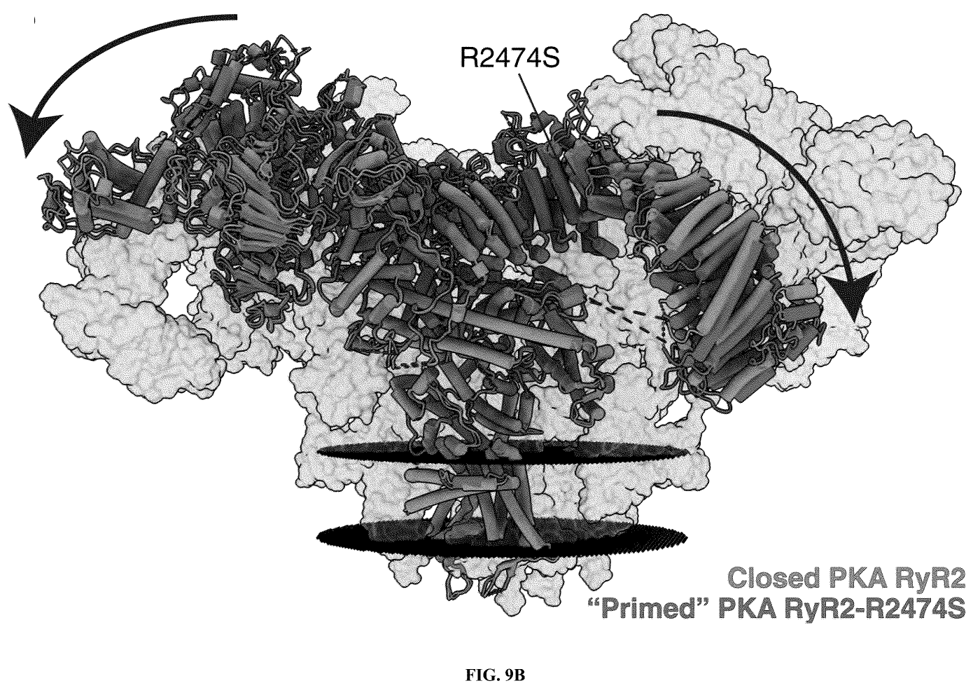

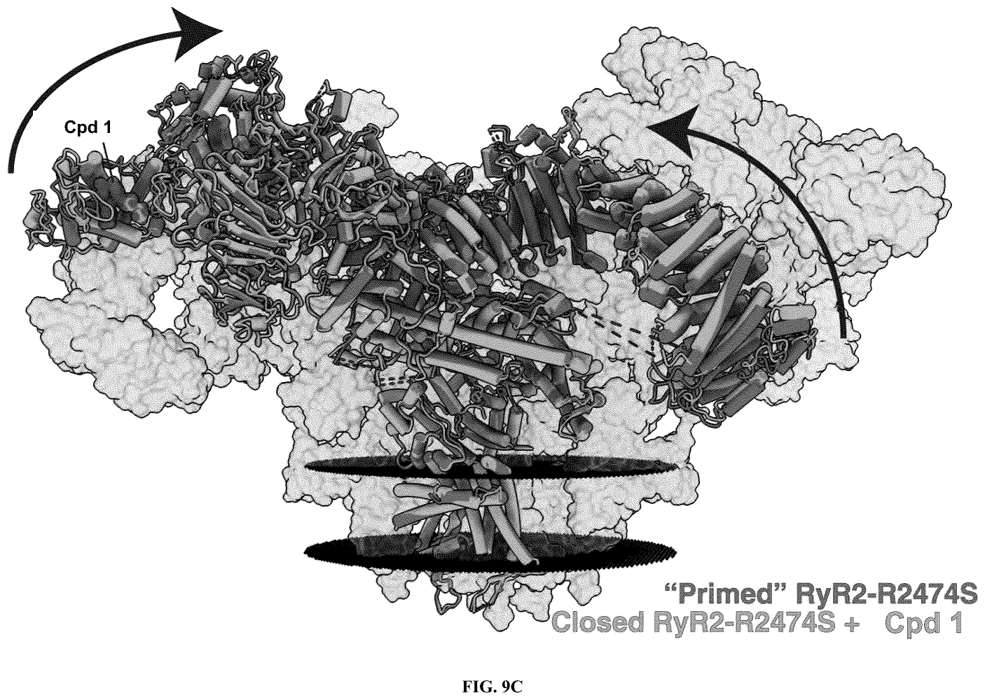

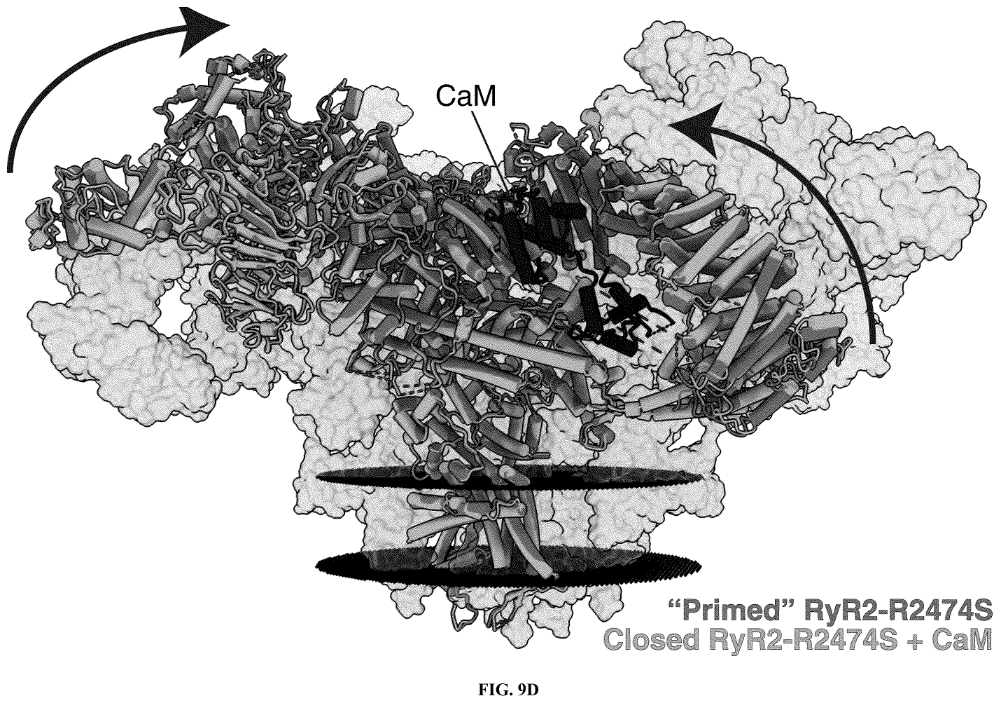

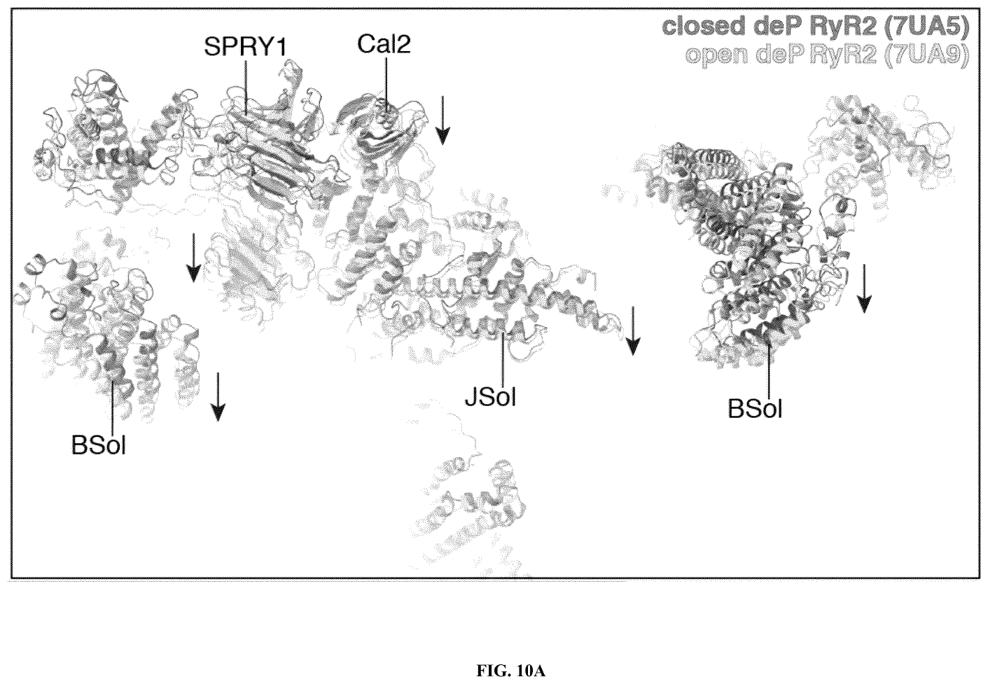

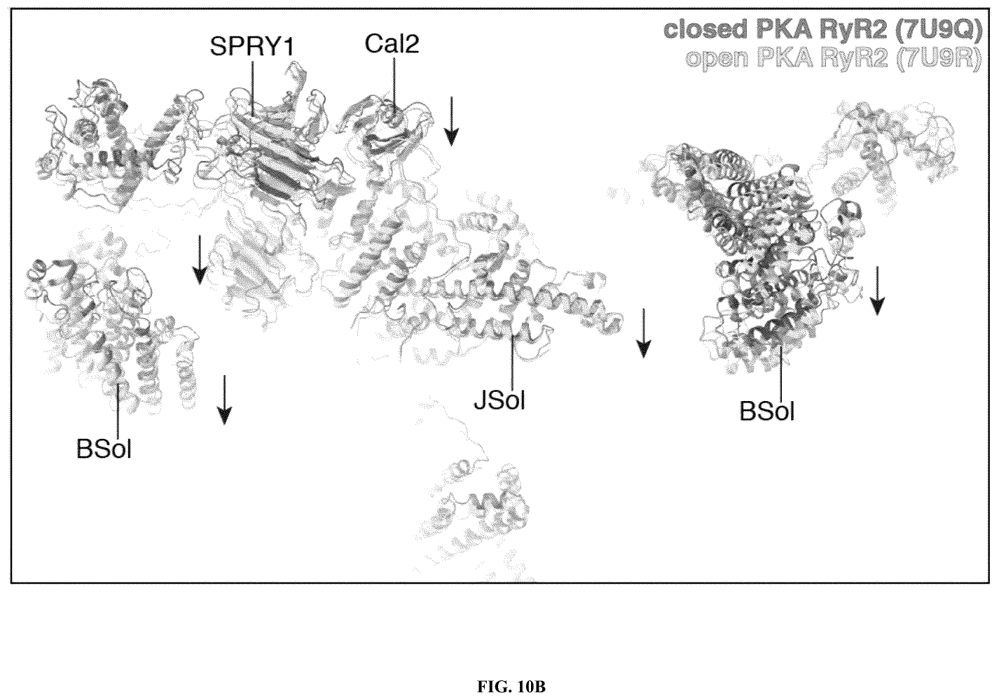

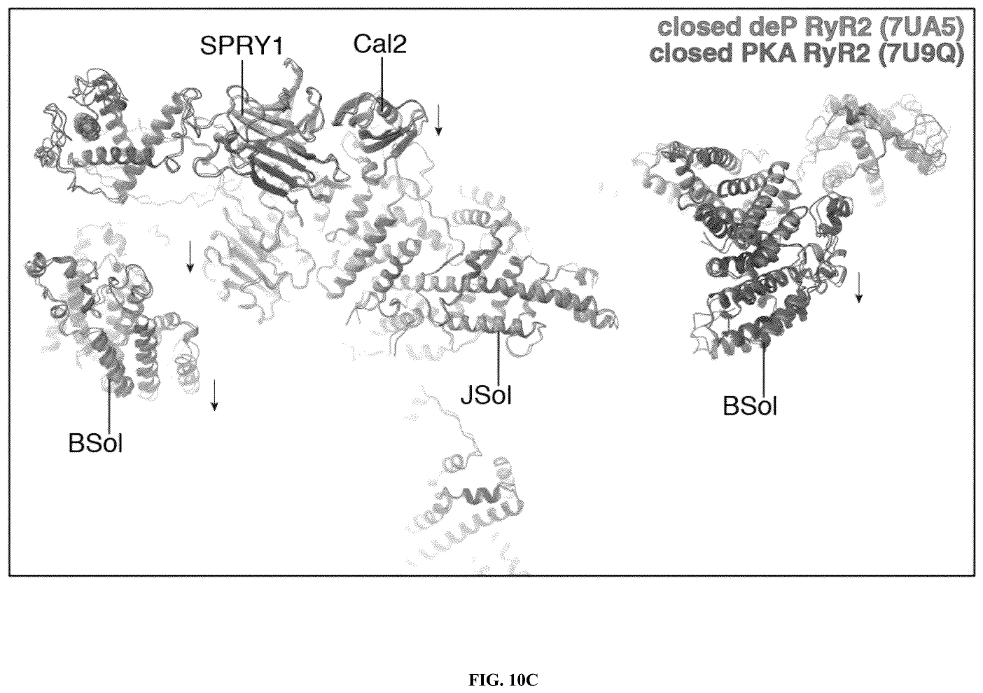

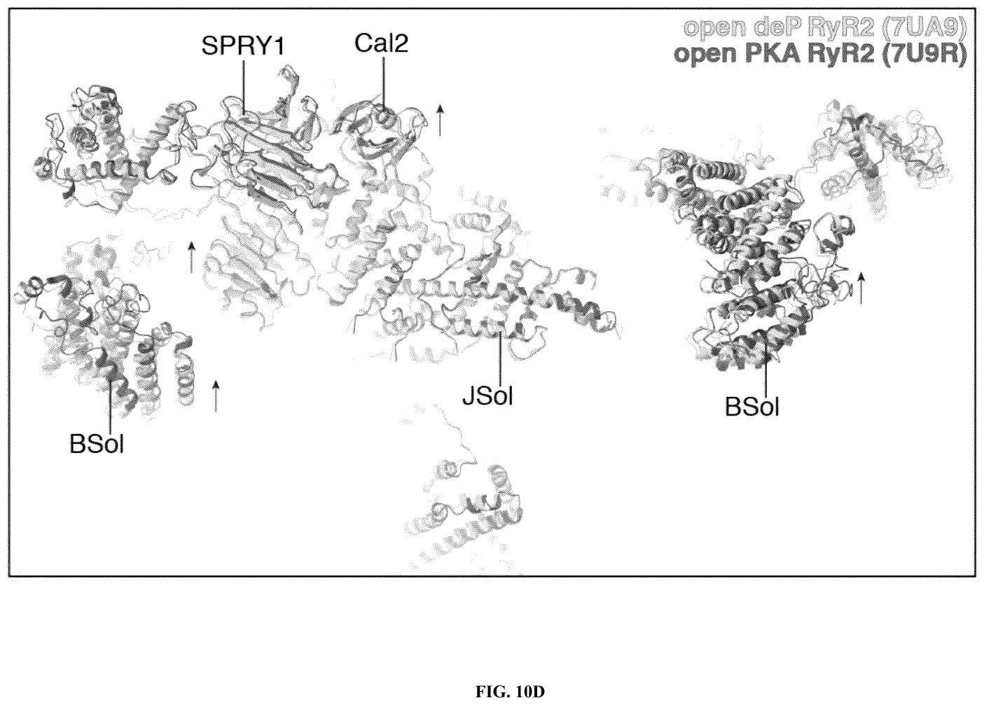

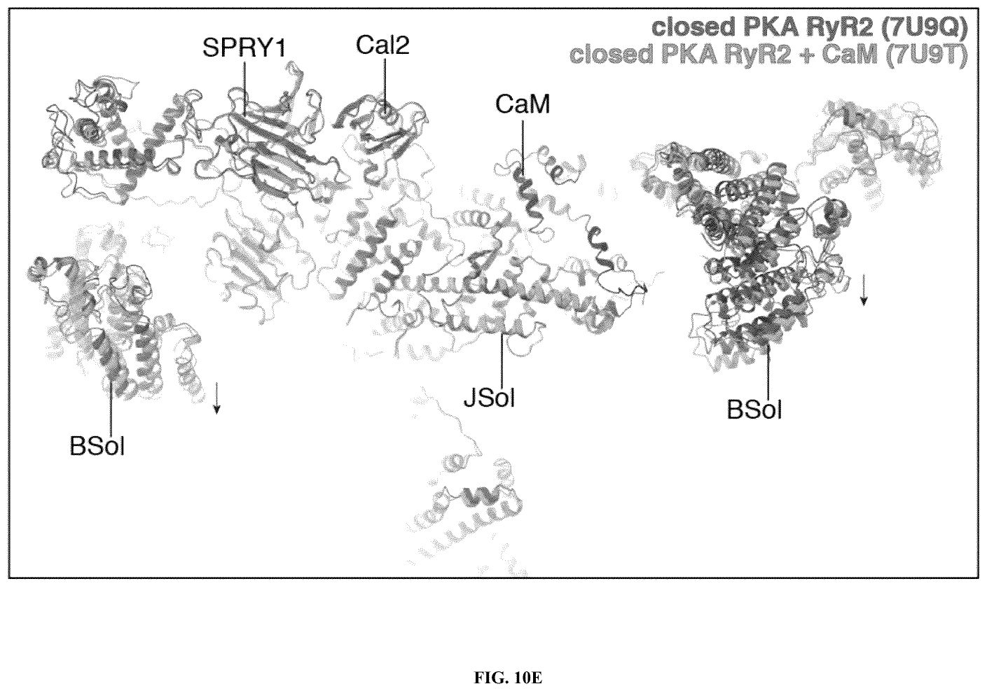

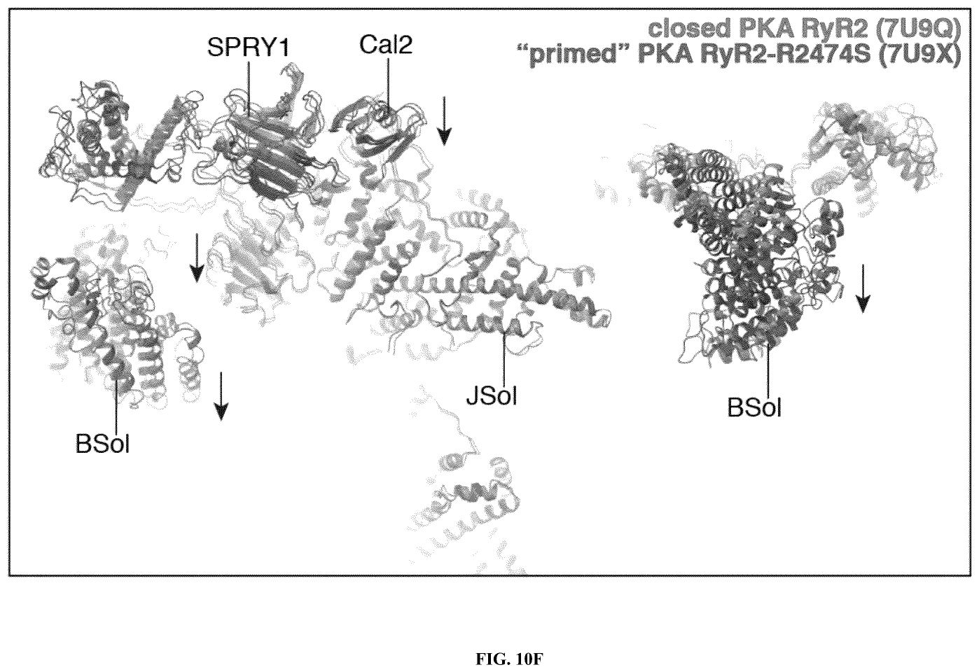

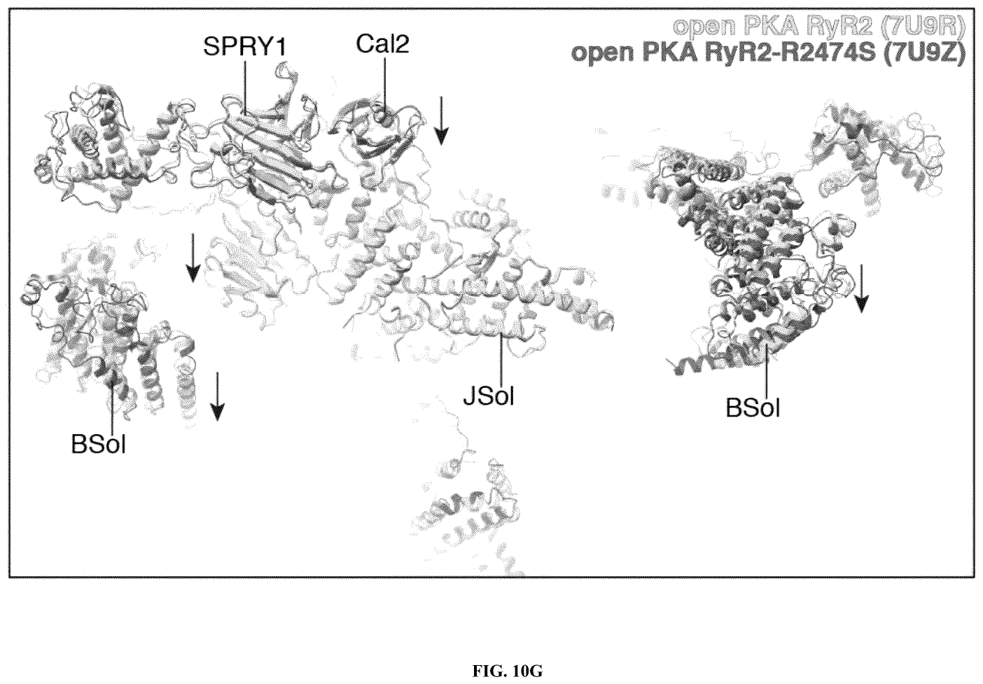

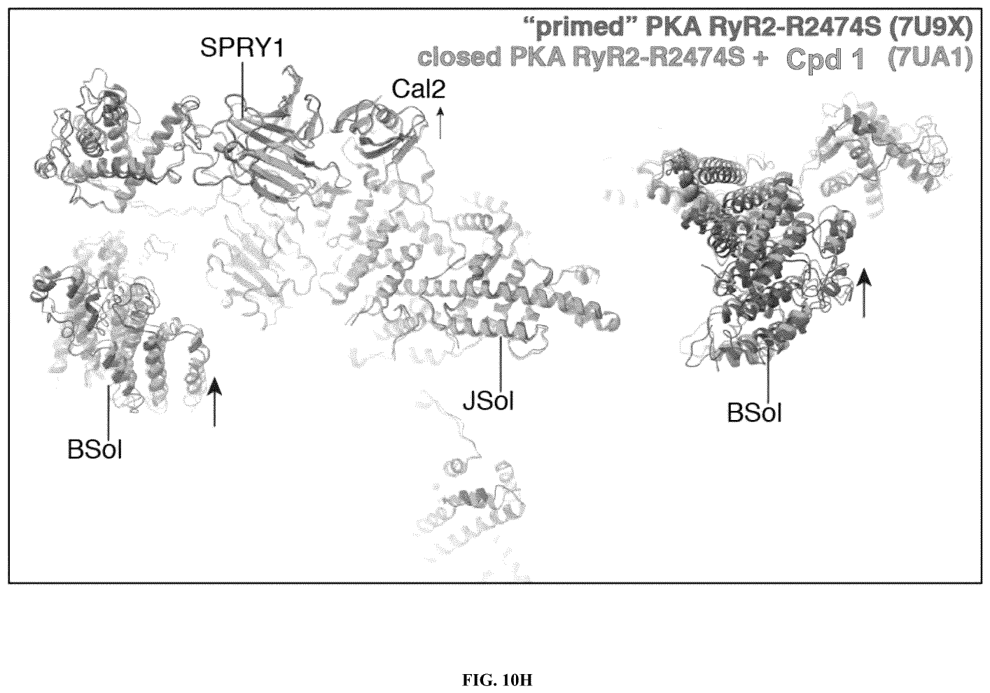

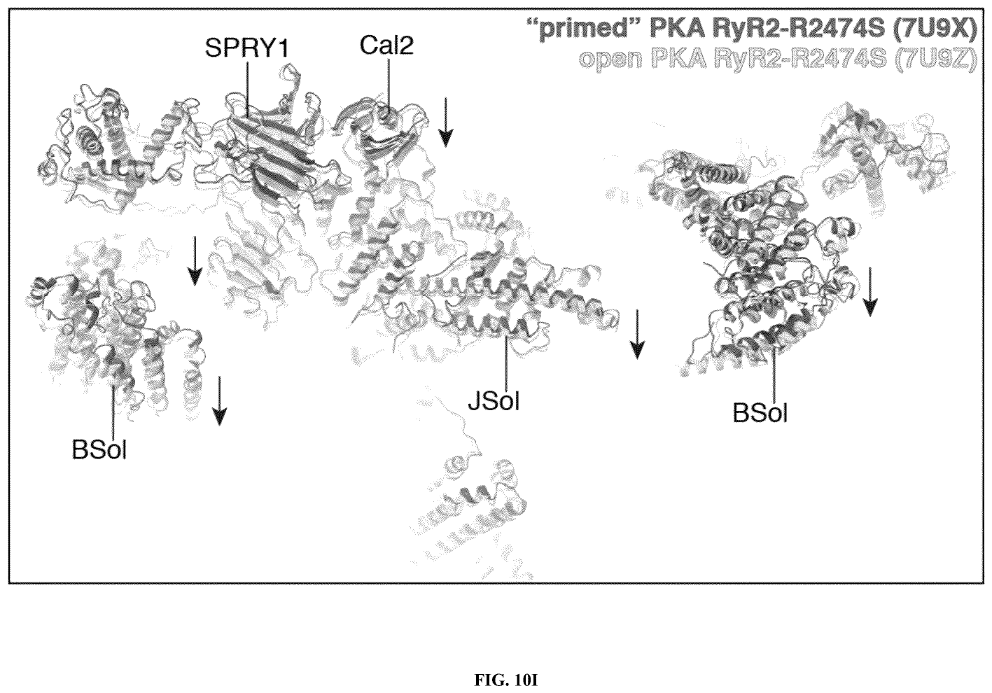

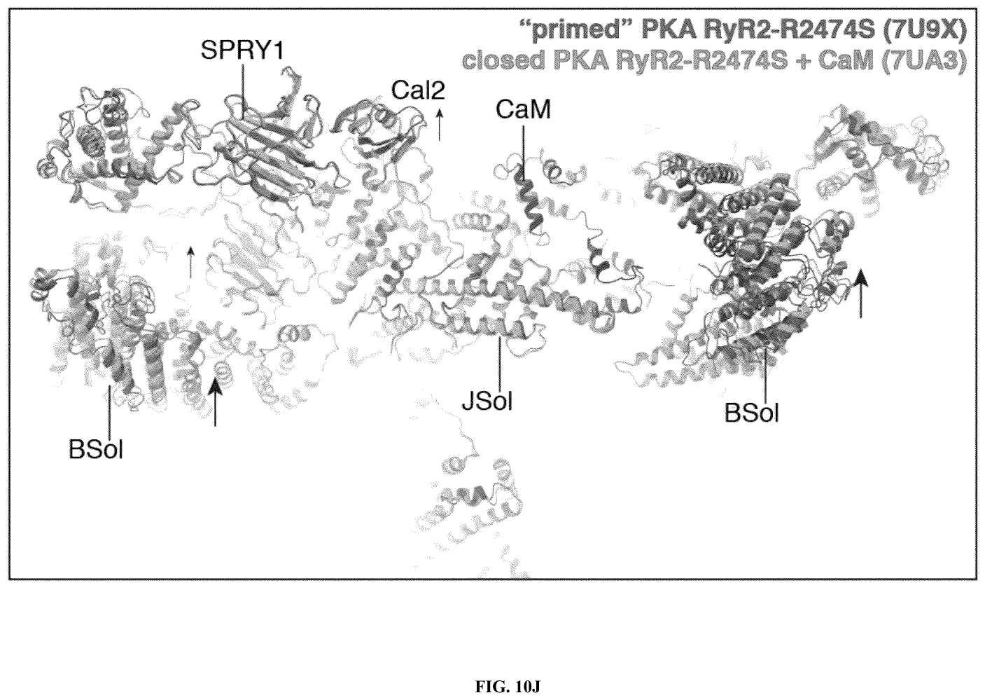

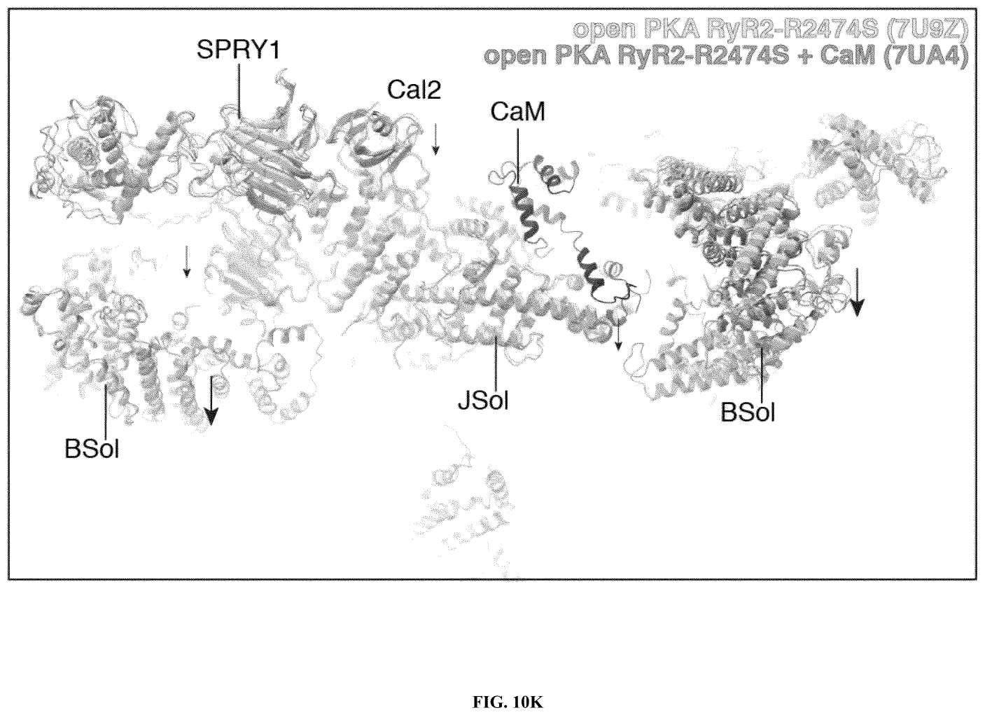

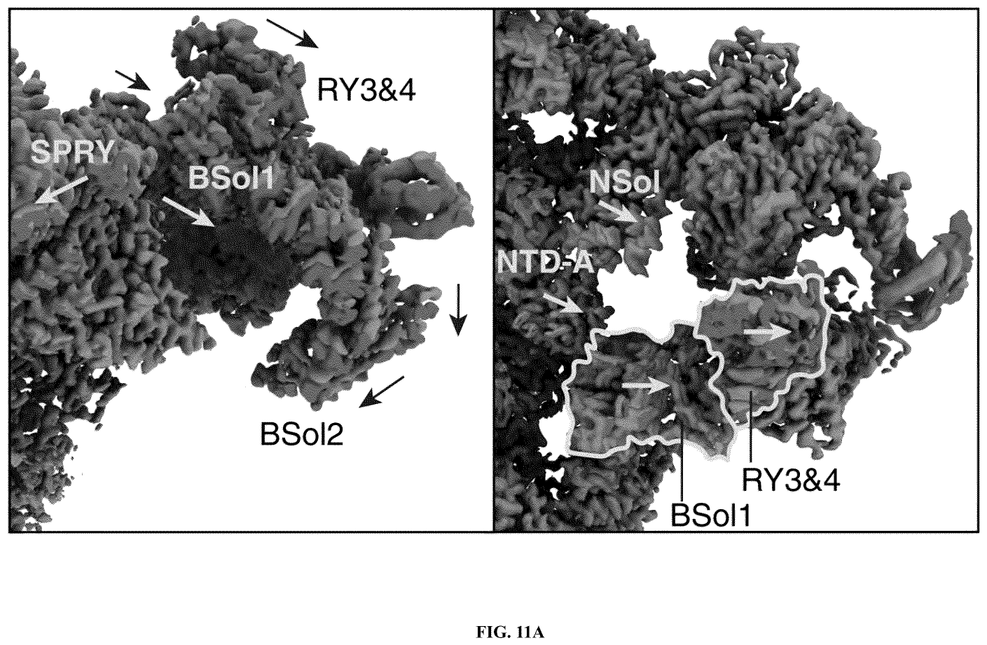

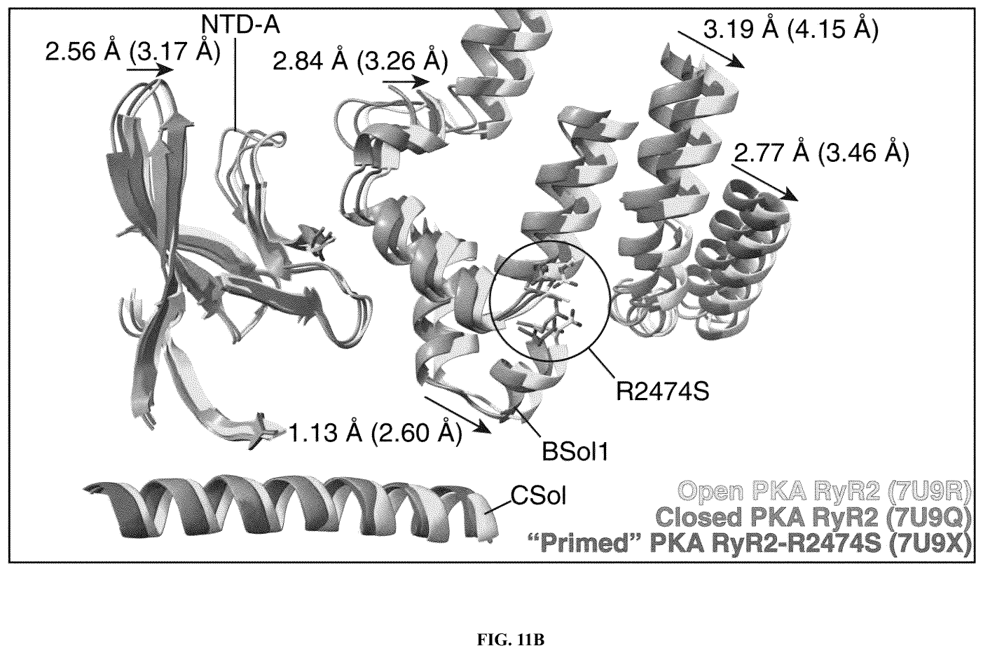

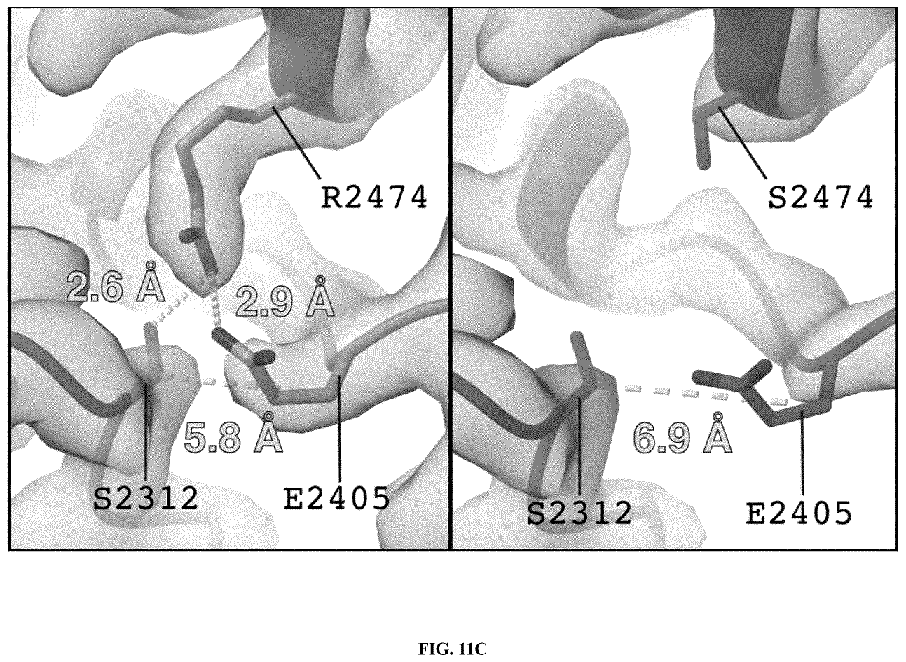

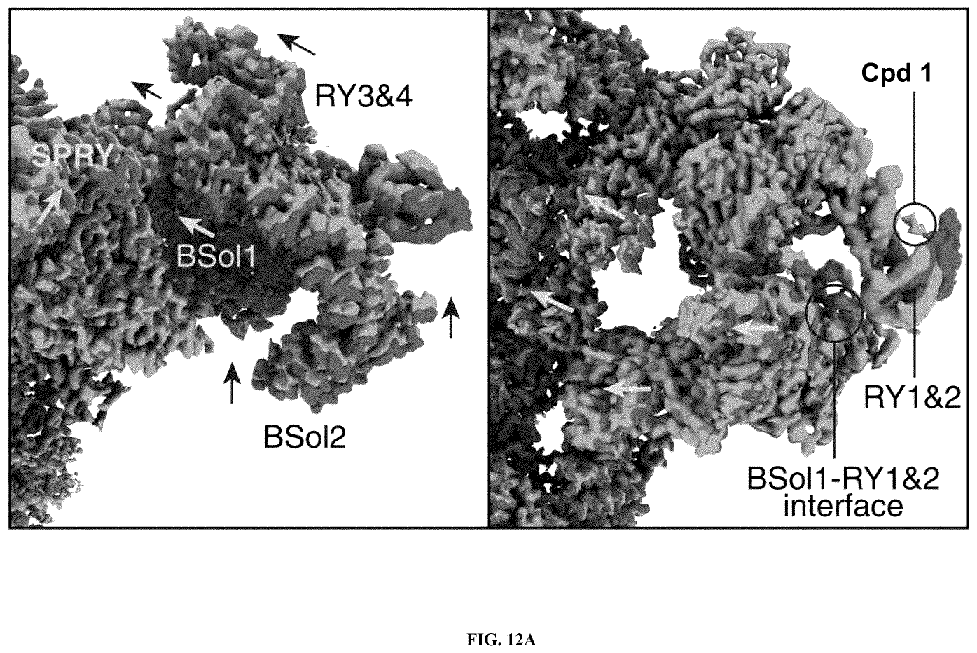

To understand the mechanism underlying CPVT better, structures of PKA-phosphorylated RyR2 (open, closed), PKA-phosphorylated RyR2-R2474S (“primed”), PKA-phosphorylated RyR2-R2474S bound to Compound 1 (closed), and PKA-phosphorylated RyR2-R2474S bound to CaM (closed) were compared. (FIG. 9, FIG. 10).

FIGS. 9A-9D depict cryo-EM reconstructions of human RyR2 showing that the CPVT mutant RyR2-R2474S predisposes the channel to assume the primed state, and treatment with the ryanodine receptor channel modulator Compound 1 and CaM restores the channel back toward the closed state.

FIG. 9A shows overlapped models of open PKA-phosphorylated RyR2 (P-RyR2-0, PDB: 7U9R, yellow) and closed PKA-phosphorylated RyR2 (P-RyR2-C, PDB: 7U9Q, gray). The arrows show that the cytosolic shell of the PKA-phosphorylated RyR2 shifted downward and outward when going from the closed to the open state. To facilitate visualization, only the front protomer is shown in colors, while the other three protomers are shown as gray transparent volumes. The positions of the sarcoplasmic reticular membranes are shown as black discs. Conditions included 10 mM ATP, 150 nM free Ca²⁺, and 500 μM xanthine.

FIG. 9B shows overlapped models of closed PKA-phosphorylated RyR2 (P-RyR2-C, PDB: 7U9Q, gray) and primed PKA-phosphorylated RyR2-R2474S (P-RyR2-R2474S-Pr, PDB: 7U9X, magenta). The arrows show that the cytosolic shell of RyR2-R2474S shifted downward and outward compared to closed PKA-phosphorylated RyR2, similar to the structural changes observed for PKA-phosphorylated RyR2 going from the closed state to the open state. This intermediate between closed and open states is defined as the primed state.

FIG. 9C shows overlapped models of primed PKA-phosphorylated RyR2-R2474S (P-RyR2-R2474S-Pr, PDB: 7U9X, magenta) and closed PKA-phosphorylated RyR2-R2474S+Compound 1 (P-RyR2-R2474S+Cpd1-C, PDB: 7UA1, cyan). The arrows show that the cytosolic shell of PKA-phosphorylated RyR2-R2474S+Compound 1 cytosolic domain shifted upward and inward compared to the RyR2-R2474S reversing the primed state back toward the closed state.

FIG. 9D shows overlapped models of primed PKA-phosphorylated RyR2-R2474S (P-RyR2-R2474S-Pr, PDB: 7U9X, magenta) and closed PKA-phosphorylated RyR2-R2474S+CaM (P-RyR2-R2474S+CaM-C, PDB: 7UA3, cyan). Similar to the effects of Compound 1, CaM reversed the primed state back toward the closed state.

FIGS. 10A-10K depict pairwise comparisons of the cytosolic domains of all structures. Domains are labelled. Conformational changes are shown with arrows. Sizes of the arrows represent the amount of changes observed.

As seen, the cytosolic shell of the PKA-phosphorylated RyR2 was shifted downward and outward when going from the closed state (P-RyR2-C, PDB: 7U9Q) to the open state (P-RyR2-0 PDB: 7U9R) (FIG. 9A and FIG. 10B).

The cytosolic shell of primed PKA-phosphorylated RyR2-R2474S (P-RyR2-R2474S-Pr, PDB: 7U9X) was shifted downward and outward compared to closed PKA-phosphorylated RyR2. This observation suggests that the CPVT mutant RyR2-R2474S is in a primed state (FIG. 9B and FIG. 10F). This primed state presents a structure that is approximately halfway between the closed and open states of PKA-phosphorylated RyR2. The same is true for the open state; the cytosolic shell of open PKA-phosphorylated RyR2-R2474S (P-RyR2-R2474S-O, PDB: 7U9Z) was also shifted downward and outward compared to open PKA-phosphorylated RyR2 (P-RyR2-0, FIG. 10G). This observation suggests that the CPVT-related cytosolic shell destabilization is independent of the state of the pore.

The PKA-phosphorylated RyR2-R2474S in the presence of Compound 1 (P-RyR2-R2474S+Cpd1-C; PDB: 7UA1)—a benzothiazepine derivative that effectively lessens a likelihood of ventricular tachycardiac and sudden cardiac death in murine models of CPVT-exhibited an upward and inward shift of the cytosolic shell compared to primed PKA-phosphorylated RyR2-R2474S (P-RyR2-R2474S-Pr), thus reversing the primed state back toward the closed state of the channel (FIG. 9C and FIG. 10H). The PKA-phosphorylated RyR2-R2474S in the presence of CaM (P-RyR2-R2474S+CaM-C; PDB: 7UA3) exhibited a similar but less pronounced shift of the cytosolic shell upward and inward compared to primed PKA-phosphorylated RyR2-R2474S (P-RyR2-R2474S-Pr), also reversing the primed state back toward the closed state of the channel similar to the effects of Compound 1 (FIG. 9D and FIG. 10J). A comparison of the cytosolic shell of primed PKA-phosphorylated RyR2-R2474S (P-RyR2-R2474S-Pr, PDB: 7U9X) and the cytosolic shell of open PKA-phosphorylated RyR2-R2474S (P-RyR2-R2474S-O, PDB: 7U9Z) are depicted in FIG. 10I.

### Compound I Stabilization of Closed State of the PKA-Phosphorylated RyR2-R2474S CPVT Variant.

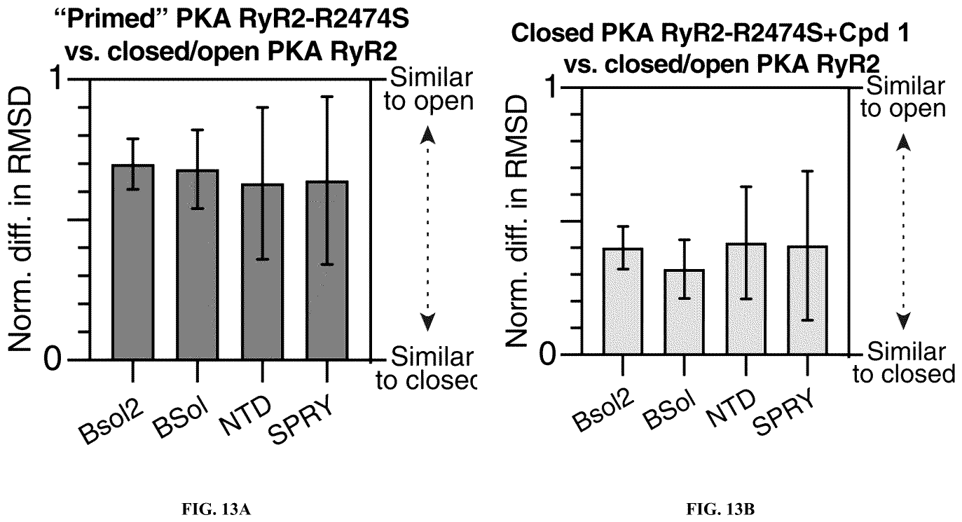

FIG. 11A depicts cryo-EM maps of closed PKA-phosphorylated RyR2 (gray) and primed PKA-phosphorylated RyR2-R2474S (magenta) from the side (left) and top (right) views. Conformation changes are shown with arrows. FIG. 13A depicts normalized differences in RMSD of the primed PKA-phosphorylated RyR2-R2474S. FIG. 11C provides a close-up view of the region around residue 2474 of closed PKA-phosphorylated RyR2 (left) and primed PKA-phosphorylated RyR2-R2474S (right). Distances are measured between Cβ atoms. FIG. 11B depicts aligned models of closed PKA-phosphorylated RyR2 (PDB: 7U9Q, gray), open PKA-phosphorylated RyR2 (PDB: 7U9R, yellow), and primed PKA-phosphorylated RyR2-R2474S (PDB: 7U9X, magenta). Conformational changes are shown with arrows. Distances between closed PKA-phosphorylated RyR2 and primed PKA-phosphorylated RyR2-R2474S, and between closed and open PKA-phosphorylated RyR2 (in parentheses) are labeled.

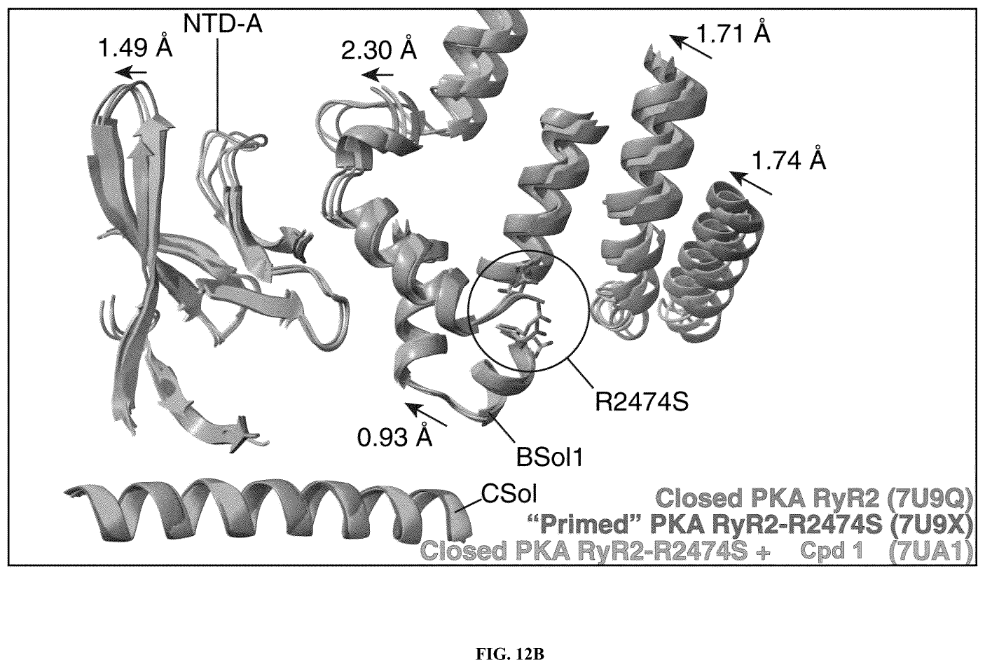

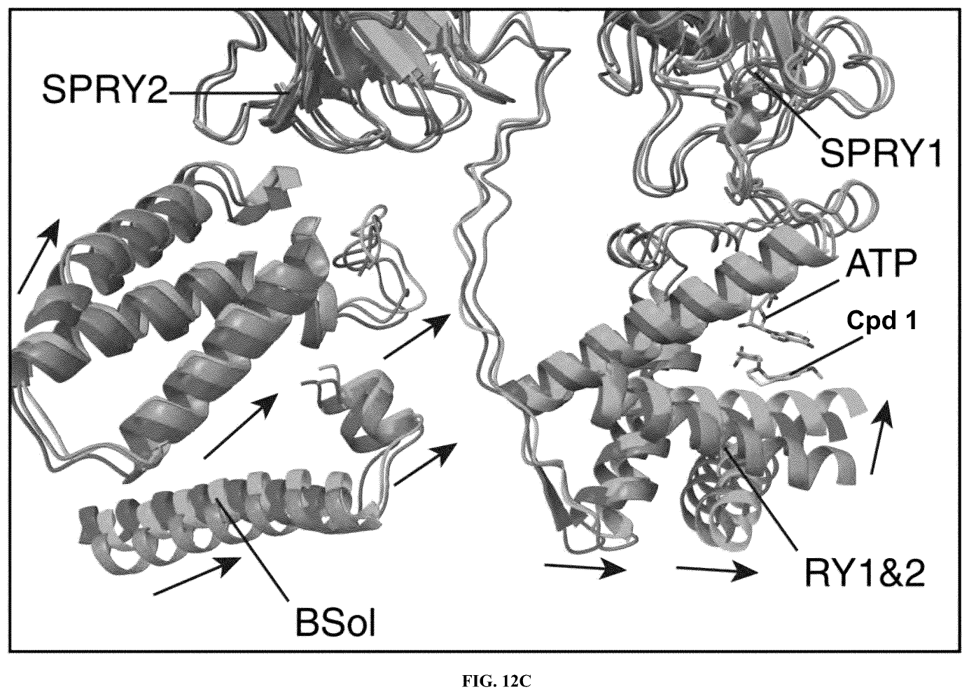

FIG. 12A and FIG. 12B are identical to FIG. 11A and FIG. 11B respectively, except that each includes closed PKA-phosphorylated RyR2-R2474S+Compound 1 (P-RyR2-R2474S+Cpd1-C, PDB: 7UA1, cyan). In FIG. 12B, Distances between primed PKA-phosphorylated RyR2-R2474S and closed PKA-phosphorylated RyR2-R2474S+Compound 1 are labeled. Changes introduced by the R2474S mutation are partially reversed by the addition of Compound 1, shifting the structure towards a closed state. In FIG. 12A, right panel, the densities of Compound 1 and BSol1-RY1&2 interface are highlighted. FIG. 13B depicts normalized differences in RMSD of the closed PKA-phosphorylated RyR2-R2474S+Cpd1.

FIG. 12C shows aligned models of closed PKA-phosphorylated RyR2 (P-RyR2-C, PDB: 7U9Q, gray), primed PKA-phosphorylated RyR2-R2474S (P-RyR2-R2474S-Pr, PDB: 7U9X, magenta), and closed PKA-phosphorylated RyR2-R2474S+Compound 1 (P-RyR2-R2474S+Cpd1-C, PDB: 7UA1, cyan). Conformational changes of the RY1&2 and BSol domains are shown with arrows.

Visual inspection of the cryo-EM maps shows conformation changes that suggest a primed state of the PKA-phosphorylated RyR2-R2474S channels (FIG. 11A). To quantify the structural changes in the primed PKA-phosphorylated RyR2-R2474S channels, the normalized differences in root mean square deviation (RMSD) between the primed PKA-phosphorylated RyR2-R2474S (P-RyR2-R2474S-Pr, PDB: 7U9X) and closed (P-RyR2-R2474S-C PDB: 7U9Q) and open (P-RyR2-0, PDB: 7U9R) PKA-phosphorylated RyR2 (FIG. 13A and TABLE 6) were measured.

TABLE 6 shows the differences in normalized RMSD analysis between primed PKA RyR2-R2474S vs. closed/open PKA RyR2.

TABLE 6

| RMSD Cα (Å) | BSol2 | BSol | NTD | SPRY | CSol |
|---|---|---|---|---|---|
| primed P-RyR2-R2474S | 5.17 ± | 3.28 ± | 1.49 ± | 1.38 ± | 0.50 |
| (PDB:7U9X) vs closed | 0.50 | 0.50 | 0.50 | 0.50 | |
| P-RyR2-C (PDB:7U9Q) | | | | | |
| primed P-RyR2-R2474S | 2.23 ± | 1.55 ± | 0.87 ± | 0.77 ± | 0.40 |
| (PDB:7U9X) vs P-RyR2-O | 0.40 | 0.40 | 0.40 | 0.40 | |
| (PDB:7U9R) | | | | | |
| Normalized difference | 0.70 ± | 0.68 ± | 0.63 ± | 0.64 ± | |
| in RMSD | 0.09 | 0.14 | 0.27 | 0.30 | |

TABLE 7 shows the differences in normalized RMSD analysis between Closed PKA RyR2-R2474S+Compound 1 vs. closed/open PKA RyR2.

TABLE 7

| RMSD Cα (Å) | BSol2 | BSol | NTD | SPRY | CSol |
|---|---|---|---|---|---|
| P-RyR2-R2474S + Cpd1* | 3.08 ± | 1.61 ± | 1.20 ± | 0.88 ± | 0.53 |
| (PDB:7UA1) vs P-RyR2-C | 0.53 | 0.53 | 0.53 | 0.53 | |
| (PDB:7U9Q) | | | | | |
| P-RyR2-R2474S + Cpd1* | 4.63 ± | 3.36 ± | 1.66 ± | 1.25 ± | 0.41 |
| (PDB:7UA1) vs P-RyR2-O | 0.41 | 0.41 | 0.41 | 0.41 | |
| (PDB:7U9R) | | | | | |
| Normalized difference | 0.40 ± | 0.32 ± | 0.42 ± | 0.41 ± | |
| in RMSD | 0.08 | 0.11 | 0.21 | 0.28 | |

*Cpd 1 shifts the primed P-RyR2-R2474S toward the closed state. Thus, P-RyR2-R2474S + Cpd1 can be referred to herein as having a closed channel pore.

TABLE 8 shows the differences in normalized RMSD analysis between Closed PKA RyR2 vs. closed/open dephosphorylated RyR2.

TABLE 8

| RMSD Cα (Å) | BSol2 | BSol | NTD | SPRY | CSol |
|---|---|---|---|---|---|
| P-RyR2-C (PDB:7U9Q) vs | 1.73 ± | 0.74 ± | 0.58 ± | 1.29 ± | 0.50 |
| DeP-RyR2-C (PDB:7UA5) | 0.50 | 0.50 | 0.50 | 0.50 | |
| P-RyR2-C (PDB:7U9Q) vs | 5.38 ± | 2.19 ± | 5.09 ± | 6.24 ± | 0.77 |
| DeP-RyR2-O (PDB:7UA9) | 0.77 | 0.77 | 0.77 | 0.77 | |
| Normalized difference | 0.24 ± | 0.25 ± | 0.10 ± | 0.17 ± | |
| in RMSD | 0.08 | 0.19 | 0.09 | 0.07 | |

TABLE 9 shows the differences in normalized RMSD analysis between closed state with stabilized RY3&4 vs. closed state with destabilized RY3&4/open state.

TABLE 9

| RMSD Cα (Å) | SPRY3 | calstabin-2 | JSol | CSol | BSol1 |
|---|---|---|---|---|---|
| closed state with | 1.80 ± | 2.40 ± | 1.94 ± | 1.66 ± | 0.36 |
| destabilized RY3&4 vs | 0.36 | 0.36 | 0.36 | 0.36 | |
| closed state with | | | | | |
| stabilized RY3&4 | | | | | |
| open state vs closed | 3.90 ± | 3.69 ± | 2.65 ± | 3.75 ± | 0.61 |
| state with stabilized | 0.61 | 0.61 | 0.61 | 0.61 | |
| RY3&4 | | | | | |
| Normalized difference | 0.32 ± | 0.39 ± | 0.42 ± | 0.31 ± | |
| in RMSD | 0.07 | 0.07 | 0.10 | 0.08 | |

In TABLES 6-9, RMSD difference values close to 0 indicated that the conformation is similar to the closed state, whereas values close to 1 indicated that the conformation is similar to the open state. Normalization allowed the direct comparison between different domains. RMSD analysis showed an average value of ˜0.7, which indicates that the cytosolic domains of the primed PKA-phosphorylated RyR2-R2474S were in nearly open positions, reducing the energetic barrier of the primed state to adopt a fully open state. Thus, the primed PKA-phosphorylated RyR2-R2474S is more readily activated and promotes an SR Ca²⁺ leak during diastole when the activating [Ca²⁺]_{cyt} is very low (˜150 nM) and the WT channels are tightly closed.

Analyzing in detail the region of the mutation, the Arg to Ser mutation was readily identified because of the shortened side-chain density for the mutant residue S2474 compared to the WT residue R2474 (FIG. 11C). R2474 stabilizes the surrounding structure via interactions with S2312 and E2405. In the mutant channel, this network is disrupted and the distance between residues S2312 and E2405 increases from 5.8 to 6.9 Å (FIG. 11C). The increased distances observed in the RyR2-R2474S mutant channel affects the helix-loop-helix motif formed by residues 2406 to 2418, which propagates to the adjacent NTD-A domain (FIG. 11B). Therefore, a single mutation destabilizes the interaction between the NTD and the BSol, which leads to destabilization of the entire cytosolic shell (NTD, SPRY, JSol, and BSol domains).

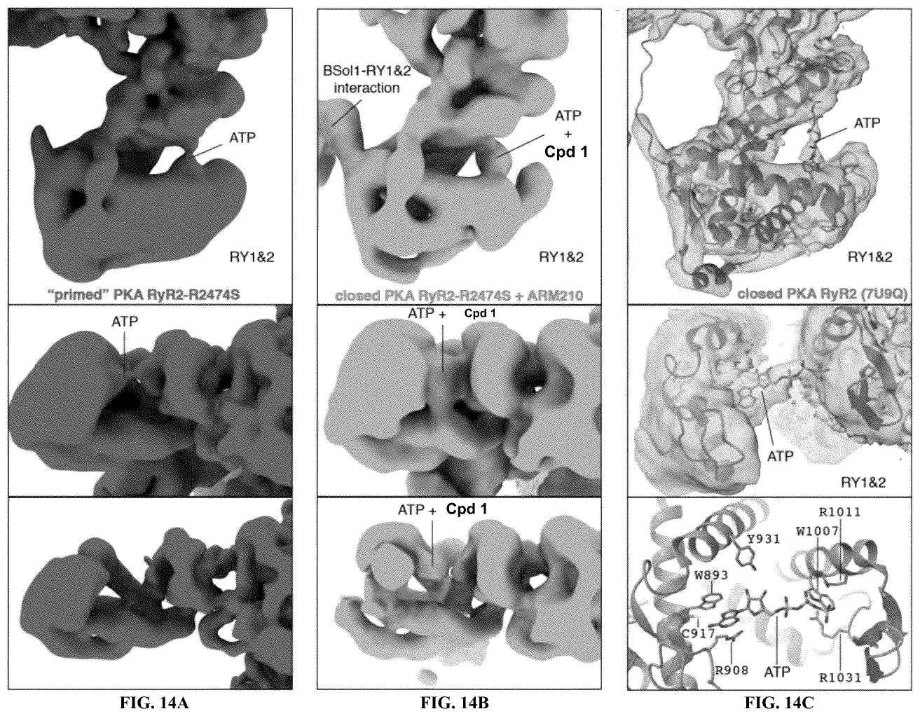

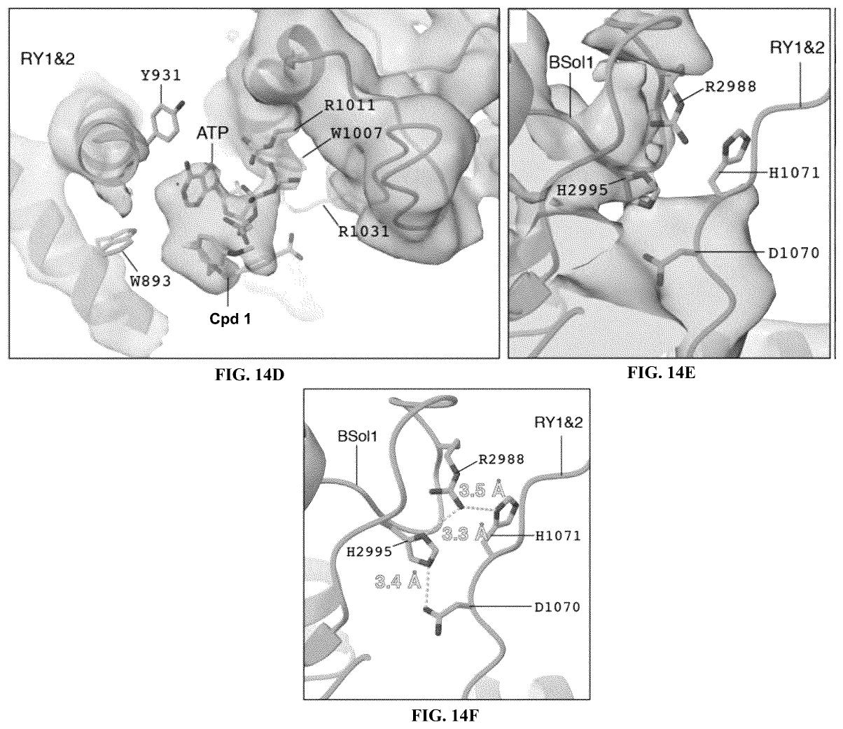

FIG. 14A and FIG. 14B depict cryo-EM maps of local refinement cryoSPRAC jobs before 3D variability of primed PKA RyR2-R2474S (magenta), and closed PKA RyR2-R2474S+Compound 1 (cyan) from different views and map levels. For better interpretation, a softening gaussian was applied to the cryo-EM maps that make evident the different size of the densities inside the RY1&2 cleft. ATP and Compound 1 potential densities are labelled.

FIG. 14C shows the closed PKA RyR2 model (PDB:7U9Q) with the aligned cryo-EM map centered on the RY1&2 domain from the top (top) and side (middle) views. Clear density is observed in the cleft of the RY1&2 domain. A molecule of ATP was fitted in the density as previously observed in RyR1. (bottom) Atomic model centered on the RY1&2 domain showing the residues that are potentially involved in ATP binding.

FIG. 14D shows a close up of closed PKA R2474S RyR2+Compound 1 (P-RyR2-R2474S-C, PDB:7UA1) centered on the ryanodine receptor channel modulator binding site. The precise placement of ATP and Compound 1 was hampered due to local low resolution.

FIG. 14E shows closed PKA RyR2-R2474S+Compound 1 (P-RyR2-R2474S-C, PDB:7UA1) centered on the BSol1-RY1&2 interface. Candidate residues involved in the BSol1-RY1&2 interaction are labeled. FIG. 14F depicts the same comparison provided in FIG. 14E but with distances between sidechains of candidate residues labeled in yellow.

Incubation with Compound 1 partially reversed the primed state of PKA-phosphorylated RyR2-R2474S, placing the channel in a state closer to that of the closed PKA-phosphorylated RyR2 (FIG. 12A and FIG. 12B). In this case, the RMSD analysis of closed PKA-phosphorylated RyR2-R2474S+Compound 1 (P-RyR2-R2474S-C, PDB: 7UA1) showed a reduction of more than 50% toward the closed state in some domains compared to primed PKA-phosphorylated RyR2-R2474S (P-RyR2-R2474S-Pr) (FIG. 13B and TABLE 7). Analyzing in detail the cryo-EM map of closed PKA-phosphorylated RyR2-R2474S in the presence of Compound 1, a clear density in the cleft of the RY1&2 domain was detected (FIG. 12A, FIG. 14A, and FIG. 14B). Therefore, Compound 1, when bound to PKA-phosphorylated RyR2-R2474S channels, shifts the “primed” state of the P-RyR2-R2474S channel pores at least partially towards a closed state.

Analysis of the rest of the cryo-EM map showed no additional densities that could correspond to Compound 1. Compound 1 density was absent in the particles in the open state. This observation suggests that Compound 1 and congeners thereof act by stabilizing the closed state of RyR2. In the absence of Compound 1, the RY1&2 domain of RyR2 showed a weak density that was attributed to one ATP molecule (FIG. 14A and FIG. 14C) (PDB: 6UHH). In the presence of Compound 1, this density was stronger and larger than one ATP molecule and suggests that at least two molecules are present (ATP and Compound 1; FIG. 14D). In addition, the RY1&2 domain adopts a conformation that closes around both ATP and Compound 1 (FIG. 12C). In comparing PKA-phosphorylated RyR2-R2474S atomic models in the absence and presence of Compound 1 (PDB: 7U9X versus PDB: 7UA1), the largest changes were observed not in the contiguous SPRY1 domain, but instead in the adjacent BSol domain (FIG. 12C). The interface between the BSol1 and the RY1&2 domains showed enhanced density. This observation suggests that the transduction of the signal from the Compound 1-loaded RY1&2 domain is through the stabilization of the BSol1 domains (FIG. 12A, FIG. 14B, and FIG. 14E). On the basis of the model, the interface between the BSol1 and RY1&2 domains would be stabilized by His²⁹⁹⁵ in the BSol1 domain and Asp¹⁰⁷⁰ in the RY1&2 domain. Arg²⁹⁸⁸ (BSol1) and His¹⁰⁷¹ (RY1&2) are also available to be involved in strengthening the BSol1-RY1&2 interaction (FIG. 14E and FIG. 14F).

### CaM Stabilization of the Closed PKA-Phosphorylated RyR2-R2474S CPVT Variant.

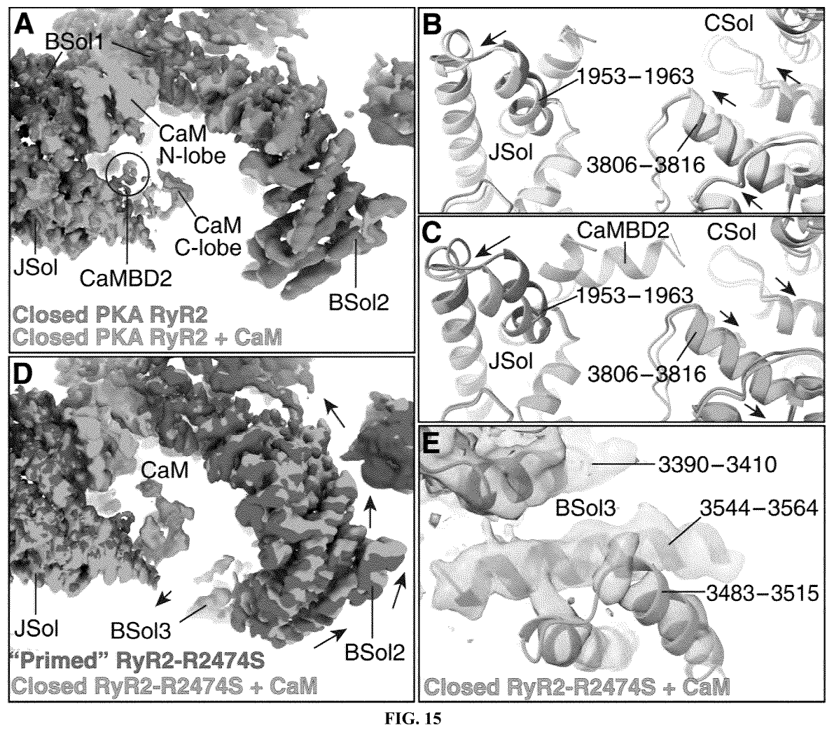

To ascertain the role of CaM in CPVT, the structure of closed PKA-phosphorylated RyR2+CaM (P-RyR2-C, PDB: 7U9T) and closed PKA-phosphorylated RyR2-R2474S+CaM (P-RyR2-R2474S+CaM-C, PDB: 7UA3) was analyzed. FIG. 15, Panel A shows aligned cryo-EM maps of closed PKA-phosphorylated RyR2 (P-RyR2-C, gray) and closed PKA-phosphorylated RyR2+CaM (cyan). FIG. 15, Panel B depicts JSol and CSol models of closed PKA-phosphorylated RyR2 (PDB: 7U9Q, gray) and open PKA-phosphorylated RyR2 (PDB: 7U9R, yellow). Conformation changes are shown with arrows. FIG. 15, Panel C depicts JSol and CSol models of closed PKA-phosphorylated RyR2 (PDB: 7U9Q, gray) and closed PKA-phosphorylated RyR2+CaM (PDB: 7U9T, cyan). Conformation changes are shown with arrows. FIG. 15, Panel D shows aligned cryo-EM maps of primed PKA-phosphorylated RyR2-R2474S (magenta) and closed PKA-phosphorylated RyR2-R2474S+CaM (cyan). Conformation changes are shown with arrows. CaM reverses the changes in the BSol2 domain introduced by the mutation by stabilizing the BSol3 domain. FIG. 15, Panel E depicts a model with cryo-EM map of closed PKA-phosphorylated RyR2-R2474S+CaM (PDB: 7UA3) centered on the BSol3 domain that is stabilized by CaM.

At a free Ca²⁺ concentration of 150 nM, CaM binds to PKA-phosphorylated RyR2 and PKA-phosphorylated RyR2-R2474S in the extended conformation that corresponds to the apo-CaM state (FIG. 15, Panel A and FIG. 10E). In the closed PKA-phosphorylated RyR2+CaM (PDB: 7U9T), the apo-CaM N-lobe binds to the BSol1 domain but does not substantially affect the BSol1 conformation. This observation suggests an exclusive binding role for the N-lobe. The C-lobe binds to CaMBD2 or helix α-1 (3594 to 3604) and pushes the JSol domain outward, although this change is not propagated to the rest of the domain. CaM-bound helix α-1 also interacts with the α9 helix of the CSol (3806 to 3816), inducing small changes to the CSol domain in the opposite direction of the open state (FIG. 15, Panel B and Panel C). Although small, the changes observed in the CSol domain show that apo-CaM may counteract signals from the cytosolic domains, such as the phosphorylation of the RY3&4 domain and the changes resulting from CPVT-causing mutations found in the NTD and BSol domains. Not surprisingly, no particles in the open state showed substantial density of bound apo-CaM, confirming that binding of apo-CaM stabilizes the closed state.

In the case of closed PKA-phosphorylated RyR2-R2474S+CaM (P-RyR2-R2474S+CaM-C, PDB: 7UA3), apo-CaM reverses the changes in the BSol2 domain of the closed state introduced by the RyR2-R2474S mutation (FIG. 15, Panel D and FIG. 10J). This observation may be a result of apo-CaM stabilizing the BSol3 domain (FIG. 15, Panel D and Panel E), which interacts with the SPRY2, JSol, and CSol domains stabilizing the whole BSol domain. This finding is surprising because the BSol3 domain was not detected in any previous RyR2 structures. Unlike PKA-phosphorylated RyR2+CaM, apo-CaM bound to the open PKA-phosphorylated RyR2-R2474S+CaM (P-RyR2-R2474S+CaM-0, PDB: 7UA4), where the cytosolic domains were further shifted downward and outward enhancing the open state (FIG. 10K). This observation suggests that, in the presence of the activator xanthine, apo-CaM could also act as an activator of PKA-phosphorylated RyR2-R2474S, in agreement with an increased number of particles found for the open state in this dataset (60% compared to 10 to 20% for the other conditions). The JSol of open PKA-phosphorylated RyR2-R2474S+CaM (P-RyR2-R2474S+CaM-0) (FIG. 10K), which is in direct contact with apo-CaM, was shifted downward, enhancing and stabilizing the open state.

### PKA Phosphorylation of RyR2 and the Structure-Function of the RY3&4 Domain.

To determine the effect that PKA treatment has on the structures of RyR2 and the phosphorylation RY3&4 domain, WT RyR2 was pretreated with phosphatase X to obtain a completely dephosphorylated control (FIG. 1A). The structures of the dephosphorylated (DeP-RyR2) and PKA-phosphorylated (P-RyR2) RyR2 channels were compared.

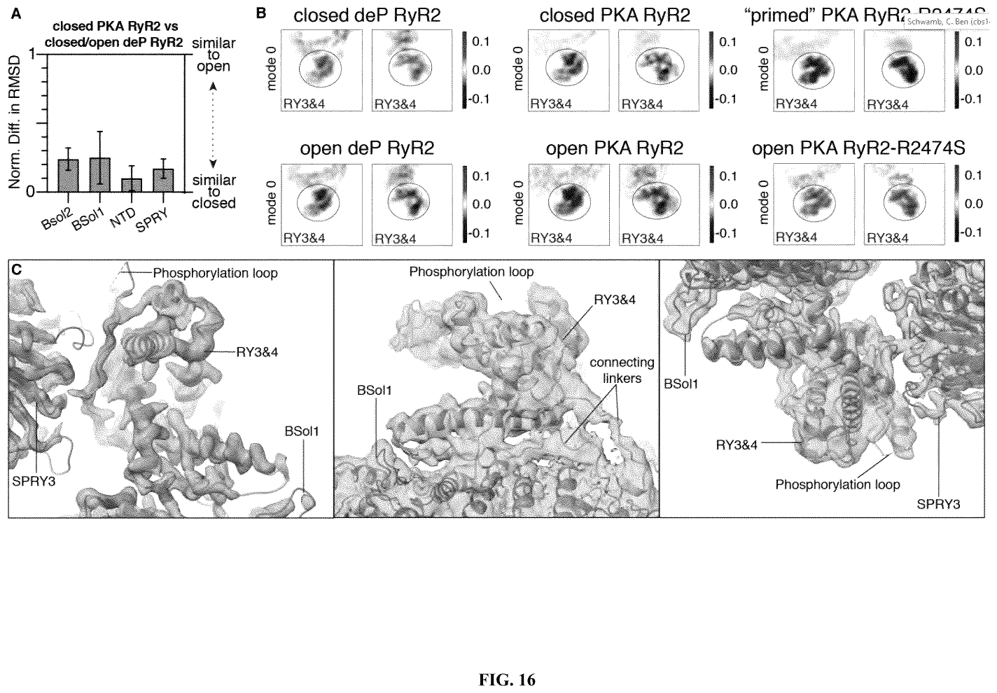

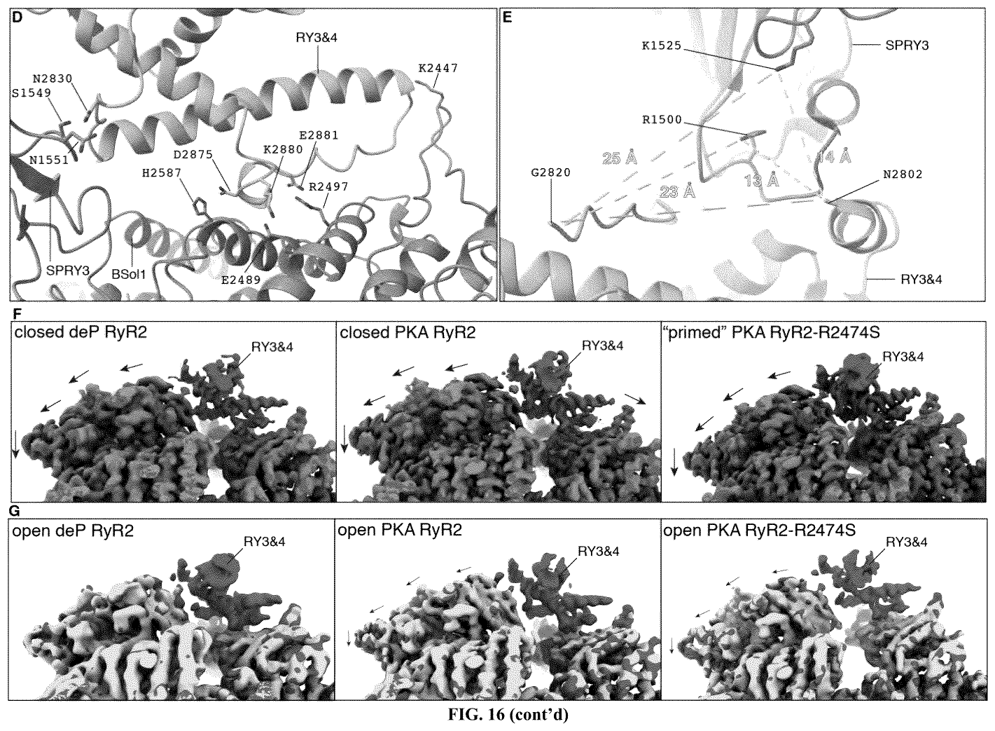

FIG. 16, Panel A illustrates a RMSD analysis of the closed PKA phosphorylated RyR2 (P-RyR2-C, PDB:7U9Q) vs. the closed (DeP-RyR2-C, PDB:7UA5) and open (DeP-RyR2-0, PDB:7UA9) states of dephosphorylated RyR2. The value of the normalized difference in RMSD is shown inside each bar. The analysis was performed on “primed” PKA RyR2-R2474S (P-RyR2-R2474S-P) because this dataset presents the most particles and best local refinement resolution. FIG. 16, Panel B depicts a 3D variability analysis of the RyR2 structures showing that the dynamic behavior of the RY3&4 domain is independent of the phosphorylation state, pore state, and mutation state. FIG. 16, Panel C shows views of the primed PKA RyR2-R2474S model (P-RyR2-R2474S-Pr, PDB:7U9X) with the aligned cryo-EM map centered on the RY3&4 domain. Connecting linkers to the BSol1 domain and missing phosphorylation loop are labeled. FIG. 16, Panel D shows the primed PKA RyR2-R2474S model (P-RyR2-R2474S-Pr, PDB:7U9X) centered on the interface between the RY3&4 and BSol1/SPRY3 domains. Residues that are close enough to generate salt bridges or hydrogen bonds are highlighted. The primary residues involved in the RY3&4-BSol1 interaction are charged residues of the RY3&4 region 2875-2881. The primary residue involved in the RY3&4-SPRY3 interaction is the polar N₂₈₃₀. FIG. 16, Panel E shows the atomic model of the RY3&4 domain of P-RyR2-R2474S-Pr centered on the phosphorylation loop. Distances between the terminal residues of the phosphorylation loop (N2802 and G2820) and the nearest positive residues (R1500 and K1525) are shown in yellow. RyR2-N2802, the closest detectable residue to the phosphorylation site, was 13 Å and 14 Å from RyR2-R1500 and RyR2-K1525, respectively. These distances allowed RyR2-pS2808 to interact with either residue, strengthening the interaction between RY3&4 and SRPY3 and stabilizing this conformation. FIG. 16, Panel F depicts cryo-EM maps of the particles in the closed state with destabilized RY3&4 domain (gray) and stabilized RY3&4 domain (magenta). A downward shift in surrounding domains can be observed. Individual domains are labelled. FIG. 16, Panel G depicts cryo-EM maps of the particles in the open state with destabilized RY3&4 domain (gray) and stabilized RY3&4 domain (magenta). A smaller downward shift in surrounding domains was observed, except for the deP RyR2 where no changes are observed. Individual domains are labelled.

Analysis of the global structure showed that the cytosolic shell of the closed PKA-phosphorylated RyR2 (P-RyR2-C, PDB: 7U9Q) showed a small shift outward and downward compared to the closed dephosphorylated RyR2 (DeP-RyR2-C, PDB: 7UA5; FIGS. 10A-10D). RMSD analysis showed values of ˜0.2 mainly in the BSol and SPRY domains, which are adjacent to the RY3&4 phosphorylation domain where the residue 52808 is located (FIG. 16, Panel A, and TABLE 8). This result suggests that PKA phosphorylation has a priming effect. These changes would reduce the global energetic barrier for reaching the open state, thus sensitizing the channel to CICR. This conformational change is smaller than the one introduced by the CPVT mutation (˜0.2 versus ˜0.7). The cytosolic shell conformation remains closer to the closed state for PKA phosphorylation than for the CPVT mutation, in agreement with the physiological role of PKA phosphorylation and the pathological role of the CPVT mutation.

The RY3&4 phosphorylation domain has not been previously resolved in the cryo-EM structures of any RyRs due to the intrinsic dynamic behavior. Under the hypothesis that this dynamic nature of the RY3&4 domain would be necessary for interacting with modulator enzymes such as PKA and protein phosphatase 1 (PP1), 3D variability analysis centered on the BSol containing the RY3&4 domain was performed.

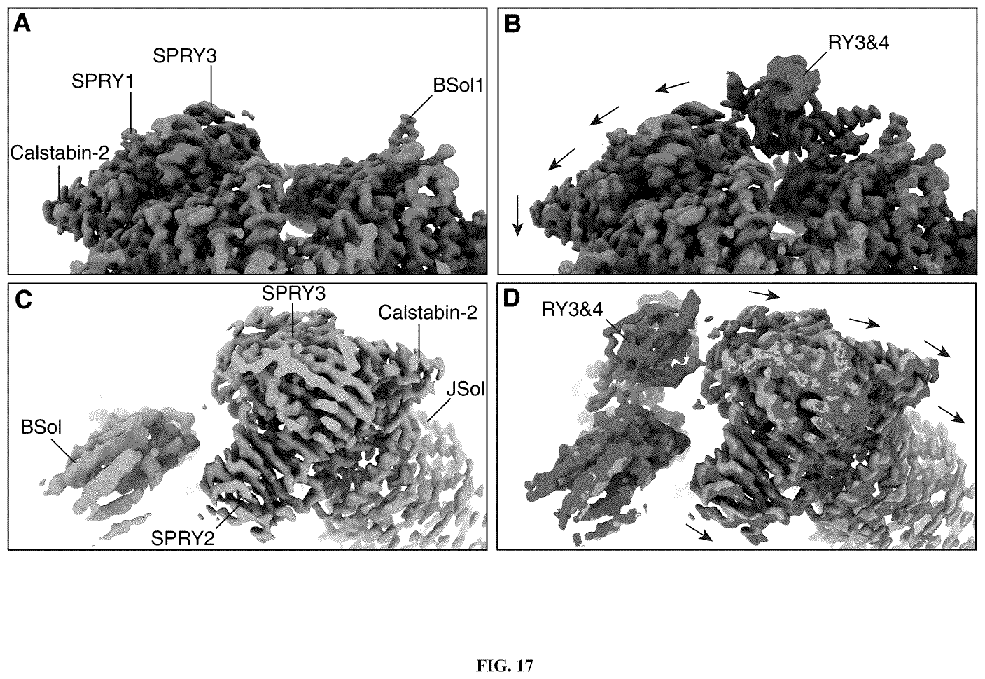

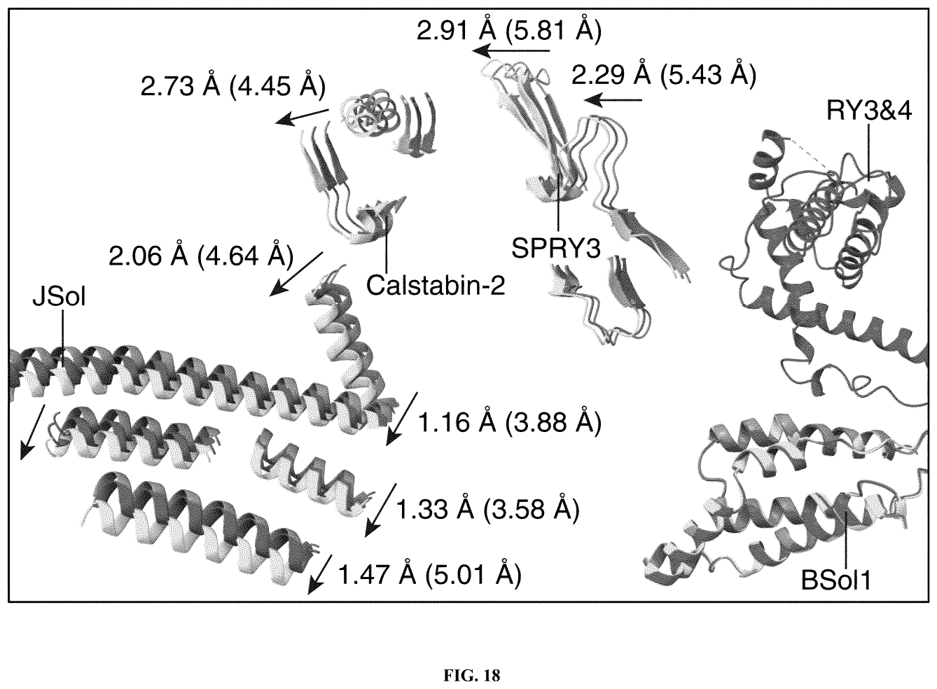

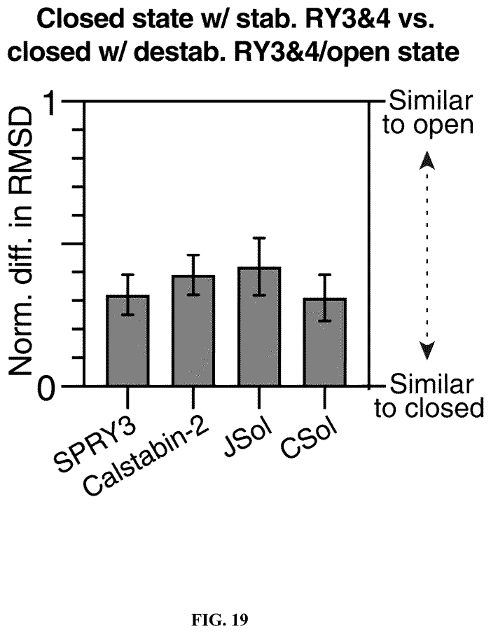

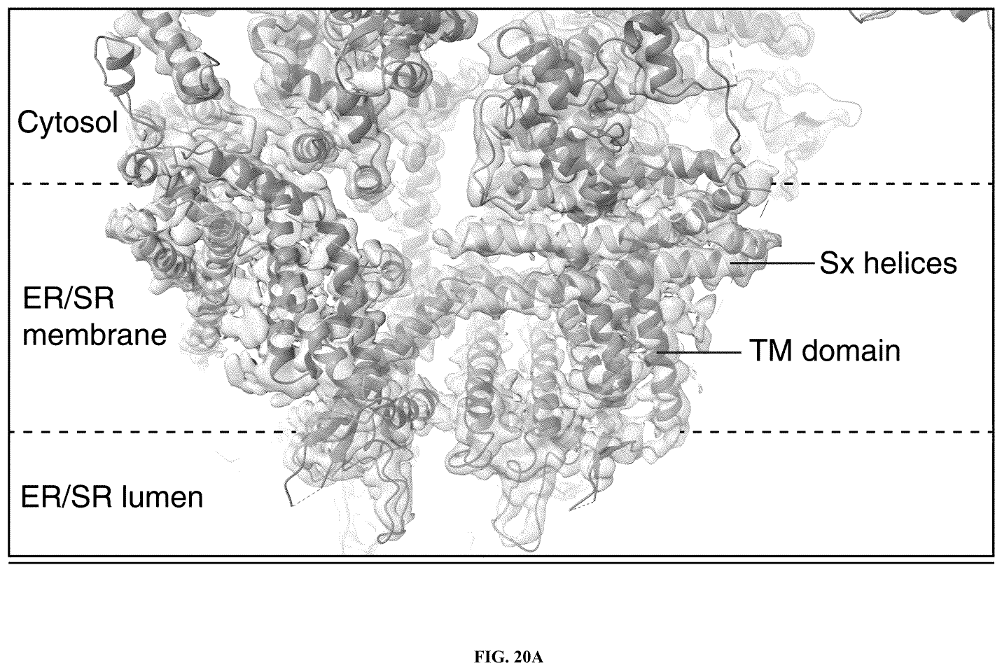

FIG. 17, Panel A and Panel B are cryo-EM maps of the closed particles with destabilized (gray) and stabilized (magenta) RY3&4 domain. A downward shift in surrounding domains was observed. Individual domains are labeled. FIG. 17, Panel C and Panel D provide different points of view of the cryo-EM maps depicted in Panel A and Panel B, respectively. FIG. 18 shows aligned models of the closed state with destabilized (gray) and stabilized (magenta) RY3&4 domain, and open state (yellow). Models were aligned at the BSol1 domains to facilitate interpretation of conformational changes. When the RY3&4 domain is stabilized, the changes are in the same direction as the open state. Shown with arrows are the distance between the closed state with destabilized RY3&4 domain and the closed state with stabilized RY3&4 domain, and between the closed state with destabilized RY3&4 domain and the open state (in parentheses). FIG. 19 is a chart illustrating an RMSD analysis of the closed state with stabilized RY3&4 domain. This analysis was performed on primed PKA-phosphorylated RyR2-R2474S because this dataset presents the most particles and best local refinement resolution.

This 3D variability analysis revealed two distinct populations: one where the RY3&4 domain was stabilized and could be resolved, and one where there was no substantial density, suggesting that RY3&4 is detached from the BSol domain (FIG. 17, Panels A-D). In other words, in the same RyR2 particle, which has four RY3&4 domains, some of the RY3&4 domains are stabilized and some are destabilized. This behavior was observed in all the structures. This observation suggests that the stabilities are independent of the phosphorylation state, closed/open state, and WT/CPVT mutation (FIG. 16, Panel B). The distribution of stabilized versus destabilized RY3&4 domains is roughly 50/50, but the limitations of this methodology prevent a precise measurement of the particle distribution.

To improve the local cryo-EM map of the RY3&4 domain, symmetry expanded particles with the stabilized RY3&4 domain were clustered and analyzed. The resulting cryo-EM map with a local resolution of 2.90 Å allowed an atomic model of this domain to be constructed with high confidence (FIG. 16, Panel C). On the basis of the model, weak interactions between the RY3&4 and BSol1 domains and almost no interaction between the RY3&4 and SPRY3 domains were observed (FIG. 16, Panel D). Phosphorylation of RyR2-S2808 may strengthen the interaction between the RY3&4 and SPRY3 domains due to the introduction of a salt bridge between the negatively charged RyR2-pS2808 and either the positively charged RyR2-R1500 or RyR2-K1525 in SPRY3 (FIG. 16, Panel E). The same conclusion can be obtained when analyzing the residue RyR2-S2814, which is preferentially phosphorylated by CaM-dependent protein kinase II (CaMKII). Hence, phosphorylation of either RyR2-S2808 or RyR2-S2814 may stabilize this conformation of the RY3&4 phosphorylation domain. However, no extra densities around these residues were found. Thus, the interaction is still very dynamic.

The interaction between the stabilization of the RY3&4 domain and the surrounding RyR2 structure was also analyzed. Comparison to the destabilized RY3&4 domain in the closed state showed that stabilization of the RY3&4 domain increased the distance between the adjacent BSol1 and SPRY3 domains, adopting a conformation that is closer to the open conformation (FIG. 17 and FIG. 16, Panel E). This movement is propagated from the SPRY3 domain to the adjacent JSol and CSol domains. To quantify those changes, normalized difference in RMSD between whole domains (FIG. 18, FIG. 19, and TABLE 9) was compared. The RMSD analysis revealed values in the range of 0.3 to 0.4, confirming that the stabilization of the RY3&4 domain had a priming effect. In the case of the open channels, stabilization of the RY3&4 domain showed no effect on open dephosphorylated RyR2 but exhibited a small effect on open PKA-phosphorylated RyR2 and open PKA-phosphorylated RyR2-R2474S. The observation confirmed that the adopted conformation is similar to the open state and that PKA phosphorylation still has a priming effect on the open states (FIG. 16, Panel G). Binding of accessory proteins to the phosphorylation loop could shift the stabilization distribution of the RY3&4 domain to either completely destabilized or completely stabilized. Phosphomimic substitution at RyR2-S2814 has been shown to induce the formation of an a helix, and this could be a substrate for protein-protein interaction that could further stabilize or destabilize the RY3&4 domain.

### Auxiliary Intramembrane Helices.

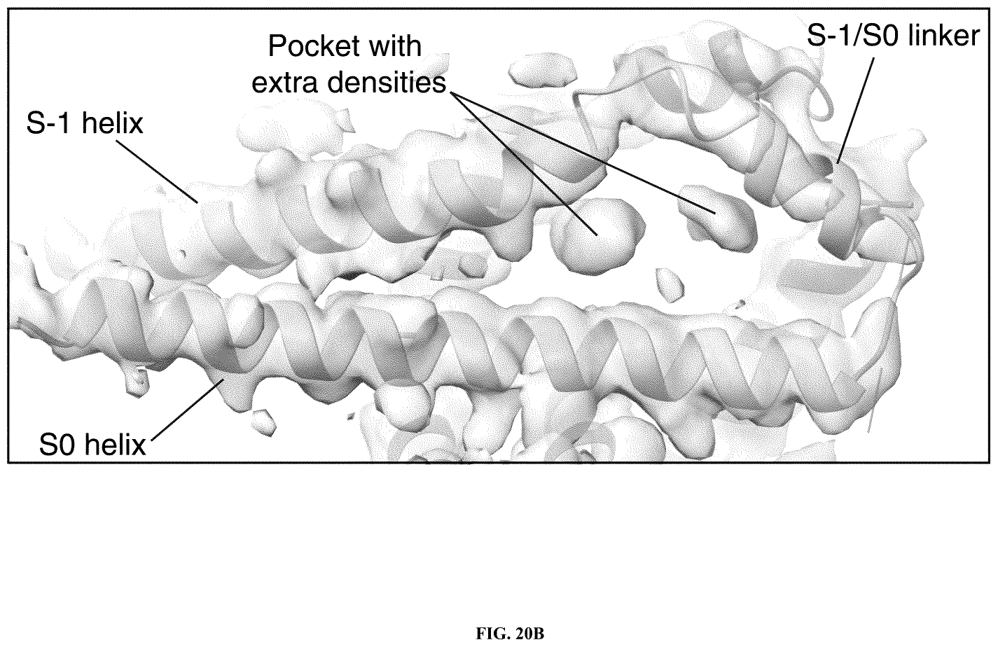

Two intramembrane helices laterally positioned and encircling the TM domain were observed. FIG. 20A and FIG. 20B show a model with overlapped cryo-EM map of PKA-phosphorylated RyR2 (PDB: 7U9Q) highlighting the auxiliary helices (Sx) helices from the side view (FIG. 20A) and bottom view (FIG. 20B). Auxiliary helices and pocket with extra densities are labeled.

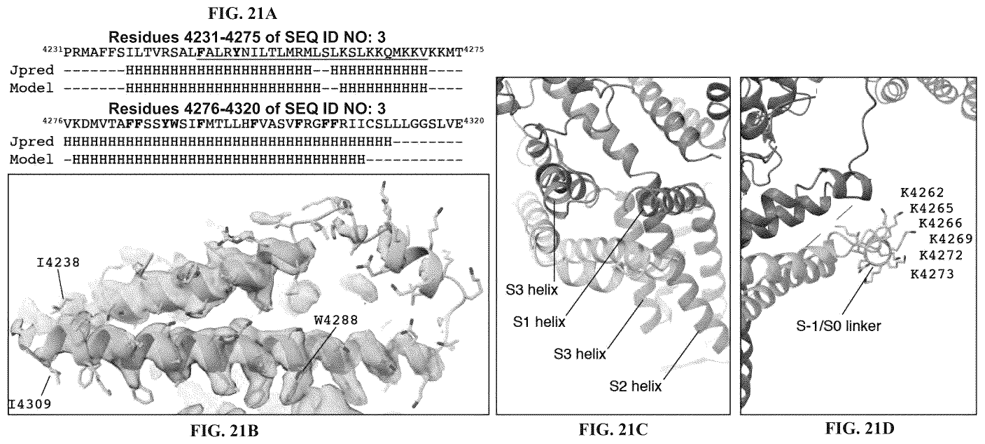

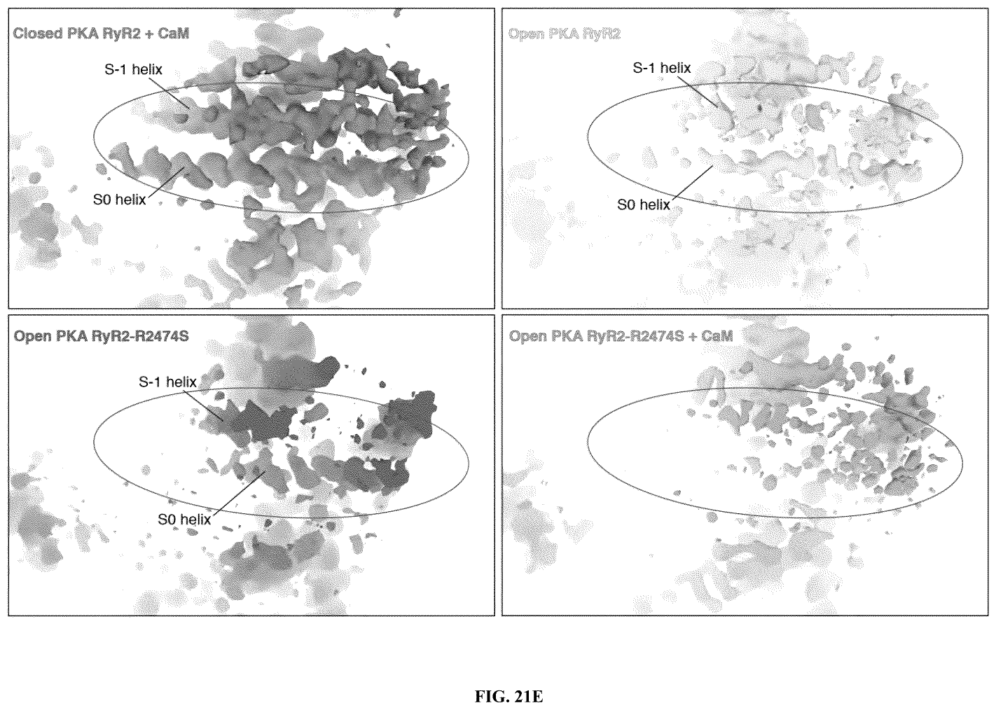

Previous cryo-EM analyses of RyR2 attributed these densities to detergent and lipids. These auxiliary helices (Sx) were predicted to exist in the RyR2 sequence upstream of the six transmembrane helices that form the pore (S1 to S6). The Jpred4 algorithm was used to predict the secondary structure of the fragment, which was previously reported to encompass these helices. FIG. 21A shows sequence alignment of residues 4231-4320 between the secondary structure predicted by Jpred and the secondary structure from the cryo-EM-resolved model. Aromatic residues used for model building are highlighted in bold. CaMBD3 sequence is underlined. FIG. 21B depicts the RyR2 model highlighting the Sx helices. Sidechains are displayed to show the good fitting of the model. Pocket with extra densities are labelled. FIG. 21C depicts RyR2 model highlighting the interaction between the Sx helices and the neighbor helical elements. FIG. 21D depicts RyR2 model highlighting the lysine rich linker. FIG. 21E depicts Cryo-EM maps of closed PKA RyR2+CaM (gray), open PKA RyR2 (yellow), open PKA RyR2-R2474S (magenta), and open PKA RyR2-R2474S+CaM (cyan) centered on the Sx helices.

Predicted Sx helices were modeled because of the presence of well-resolved densities for large side chains, including W4288 as well as several tyrosine and phenylalanine residues (FIG. 21B). Since the helices are upstream of the S1 to S6 helices, they were named S-1 (4238 to 4259), S-1/S0 linker (4262 to 4271), and S0 (4277 to 4309). In addition, a pocket formed by the interactions with helices S1, S2, and S3 was detected, where two densities were found that could correspond to detergent molecules or protein fragments (FIG. 20B). The S-1/S0 linker contained several positively charged lysine residues and is positioned at the cytosolic surface of the SR membrane, where these lysine residues would be able to interact with the negatively charged phospholipid head groups or other transmembrane proteins (FIG. 21D). The Sx density is strong in the closed states but weaker in the open states (FIG. 21E). This observation suggests that the Sx density plays a role in stabilizing the RyR2 closed state. The CaM binding motif CaMBD3 is also contained in this sequence. In the presence of CaM, the Sx density was absent only in the open state. This finding suggests that, in the closed state of RyR2, the CaMBD3 motif would be inaccessible to CaM, but in the open state of RyR2, it would interact with CaM and completely destabilize the Sx helices.

### RyR2 Leak in CPVT

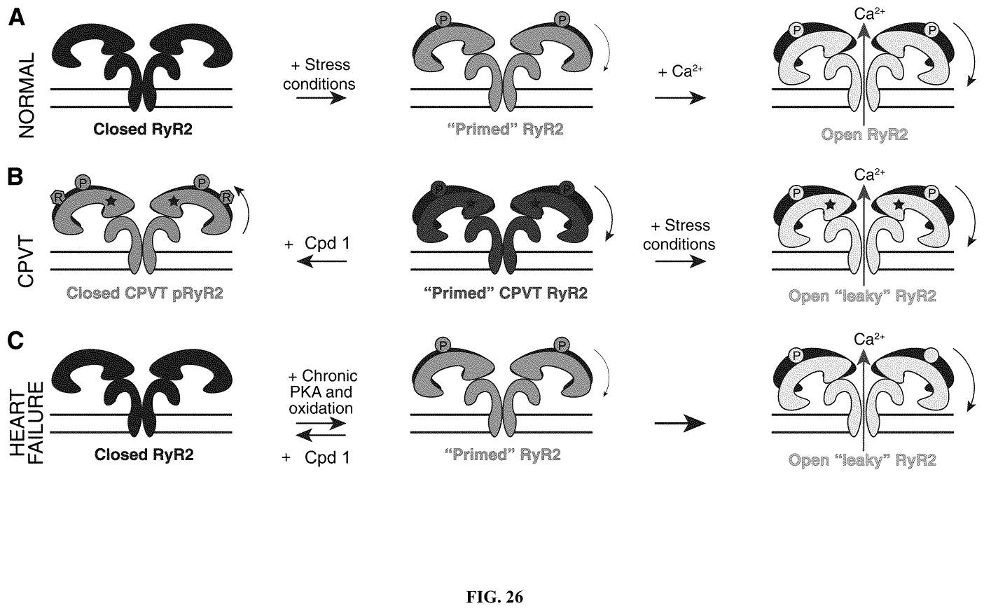

In CPVT patients, SR Ca²⁺ leak occurs via mutant RyR2 channels during diastole when the heart is supposed to be electrically silent, resulting in afterdepolarizations, arrhythmias, and eventually sudden cardiac death. Intense exercise and adrenergic stimulation can cause two independent but synergistic events that affect RyR2. The β-adrenergic response to exercise (i) results in PKA phosphorylation of RyR2 mainly at S2808 and (ii) activates SR Ca²⁺ uptake via SERCA2a, thus increasing the SR Ca²⁺ load, and the driving force for Ca²⁺ leak out of the membrane via RyR2 channels. By virtue of being in a primed state, the CPVT variant RyR2-R2474S is likely more sensitive to channel-activating posttranslational modifications (e.g., phosphorylation, nitrosylation or oxidation), resulting in a diastolic SR Ca²⁺ leak that can trigger fatal cardiac arrhythmias during intense exercise (FIG. 26 A and B).

FIG. 26 illustrates the proposed mechanism of CPVT-related RyR2 variants, other gain-of-function mutants, and heart failure. FIG. 26, Panel A is a schematic representation of the normal function of RyR2. FIG. 26, Panel B is a schematic representation of the CPVT-related Ca²⁺ leak during diastole under intense exercise or stress conditions. In the case of CPVT variants, the resting state is already in a primed state. This state correlates with the higher open probability during exercise or stress conditions. Such conditions can result in opening during diastole, afterdepolarizations, arrhythmias, and sudden cardiac death. This pathological state can be reversed by treatment with Compound 1. This basal primed state scenario could be a shared mechanism among other RyR1 and RyR2 gain-of-function mutants. FIG. 26, Panel C is a schematic representation of the heart failure-related primed state and Ca²⁺ leak.

As demonstrated herein, the CPVT mutation R2474S can put the channel into a primed state that is independent of the binding of activators including Ca²⁺. Thus, the mutant CPVT channel RyR2-R2474S is able to be inappropriately activated during diastole when the [Ca²⁺]_{cyt} is too low to activate the WT RyR2 channel. This inappropriate activation of the mutant channel results in diastolic SR Ca²⁺ leak that can trigger fatal cardiac arrhythmias. Stabilizing effects of Compound 1 and CaM through different mechanisms are demonstrated herein. One involves the stabilization of the BSol domain through the interaction with the Compound 1-bound RY1&2 domain, and the other occurs via the stabilization of the CSol and BSol3 domains through the binding of CaM to CAMBD2 and the BSol domain.

As demonstrated herein, the CPVT mutation can disrupt local interactions that destabilize the BSol domain and induces a primed state. This primed state leads to inappropriate opening of RyR2 channels that can be reversed by treatment with the RyR2 stabilizer Compound 1. Compound 1 binds to a cleft in the RY1&2 domain where can stabilize interactions between residues that are required to reduce flexibility between domains, particularly with the BSol1 domain, of the cytosolic shell. The net effect of Compound 1 binding is to stabilize the overall channel structure closer to the closed state. This renders the channel less likely to be inappropriately activated during diastole when the conditions favor the closed state of the channel (e.g., low non-activating [Ca²⁺]cyt).

### Example 7: Single Channel Recordings

To confirm the functional role of xanthine in RyR2, single-channel experiments were performed. ER vesicles from HEK293 cells expressing RyR2 were prepared by homogenizing cell pellets on ice using a Teflon glass homogenizer with two volumes of solution containing 20 mM tris-maleate (pH 7.4), 1 mM EDTA, 1 mM DTT, and protease inhibitors (Roche). Homogenate was then spun by centrifuge at 4000 g for 15 min at 4° C., and the resulting supernatant was spun by centrifuge at 40,000 g for 30 min at 4° C. The final pellet, containing the ER fractions, was resuspended and aliquoted in 250 mM sucrose, 10 mM Mops (pH 7.4), 1 mM EDTA, 1 mM DTT, and protease inhibitors. Samples were frozen in liquid nitrogen and stored at −80° C.

ER vesicles were fused to planar lipid bilayers formed by painting a lipid mixture of phosphatidylethanolamine and PC (Avanti Polar Lipids) in a 5:3 ratio in decane across a 200-am hole in polysulfonate cups (Warner Instruments) separating two chambers. The trans chamber (1.0 ml), representing the intra-SR (luminal) compartment, was connected to the head stage input of a bilayer voltage clamp amplifier. The cis chamber (1.0 ml), representing the cytoplasmic compartment, was held at virtual ground. The following asymmetrical solutions were used: for the cis solution, 1 mM EGTA, 250/125 mM Hepes/tris, 50 mM KCl (pH 7.35); for the trans solution, 53 mM Ca(OH)₂, 50 mM KCl, 250 mM Hepes (pH 7.35). The concentration of free Ca²⁺ in the cis chamber was calculated as previously described. ER vesicles were added to the cis side, and fusion with the lipid bilayer was induced by making the cis side hyperosmotic by the addition of 400 to 500 mM KCl. After the appearance of potassium and chloride channels, the cis side was perfused with the cis solution. At the end of each experiment, 10 μM ryanodine was added to block the RyR2 channel. Single-channel currents were recorded at 0 mV using Bilayer Clamp BC-525D (Warner Instruments), filtered at 1 kHz using Low-Pass Bessel Filter 8 Pole (Warner Instruments), and digitized at 4 kHz. All experiments were performed at room temperature (23° C.). Data acquisition was performed by using Digidata 1322A and Axoscope 10.1 software (Axon Instruments). The recordings were analyzed using Clampfit 10.1 (Molecular Devices) and GraphPad Prism software.

This experiment determined that at presumed physiological concentrations during exercise (10 μM), there was a ˜25-fold increase in the open probability of RyR2 in the presence of xanthine FIG. 22 shows single-channel current recordings traces from recombinant RyR2 at Ca²⁺ 150 nM, before (Panel A) and after (Panel B) the addition of xanthine 10 μM. Opening events were recorded as an upward deflection. Pₒ: opening probability, To: meantime open, Tc: meantime closed.

### Example 8: Telemetric Electrocardiogram Recordings in Ryr2^{R2474S/WT} Mice

Ryr2^{R2474S/WT} mice undergo sustained ventricular tachycardia and sudden cardiac death after exercise and epinephrine. This experiment evaluated the effect of Compound 1 treatment on frequency of ventricular tachycardia and sudden cardiac death in Ryr2^{R2474S/WT} mice.

Ryr2^{R2474S/WT} mice were implanted with radio telemetry transmitters (Data Sciences International). Briefly, the transmitter (PhysioTel, ETA-F10 transmitter) was inserted in mice subcutaneously along the back under general anesthesia (5% inhaled isoflurane). Two electrocardiogram (ECG) electrodes were placed hypodermically in the region of the right shoulder (negative pole) and toward the lower left chest (positive pole) to approximate lead II of the Einthoven surface ECG. During the procedure, respiratory and cardiac rhythm, adequacy of anesthetic depth, muscle relaxation, body temperature, and analgesia were monitored to avoid anesthesia-related complications. Postoperative pain was considered during a 1-week post-implantation period, and carprofen (5 mg/kg, subcutaneously) was given. A minimum period of 2 weeks was allowed for recovery from the surgery. Animals were housed in individual stainless-steel cages for telemetry recordings. Environmental parameters were recorded continuously and maintained within a fixed range: room temperature at 15° C. to 21° C. and 45 to 65% relative humidity. The artificial day/night cycle was 12-hour light/12-hour dark with light on at 0700 hours. Drinking water was provided ad libitum. Solid diet (300 g) was given daily in the morning. ECG waveforms were continuously recorded at a sampling rate of 2000 Hz using a signal transmitter-receiver (RPC-1) connected to a data acquisition system (Ponemah system, Data Sciences International). Compound 1 (50 mg/kg per day) was given in drinking water for 2 weeks before ECG recordings. Mice were recorded for 1 hour at baseline and then given epinephrine injection (1 mg/kg, intraperitoneally) and recorded for another 2 hours. The animals used in the study were maintained and studied according to protocols approved by the Institutional Animal Care and Use Committee of Columbia University (reference no. AC-AABP1551).

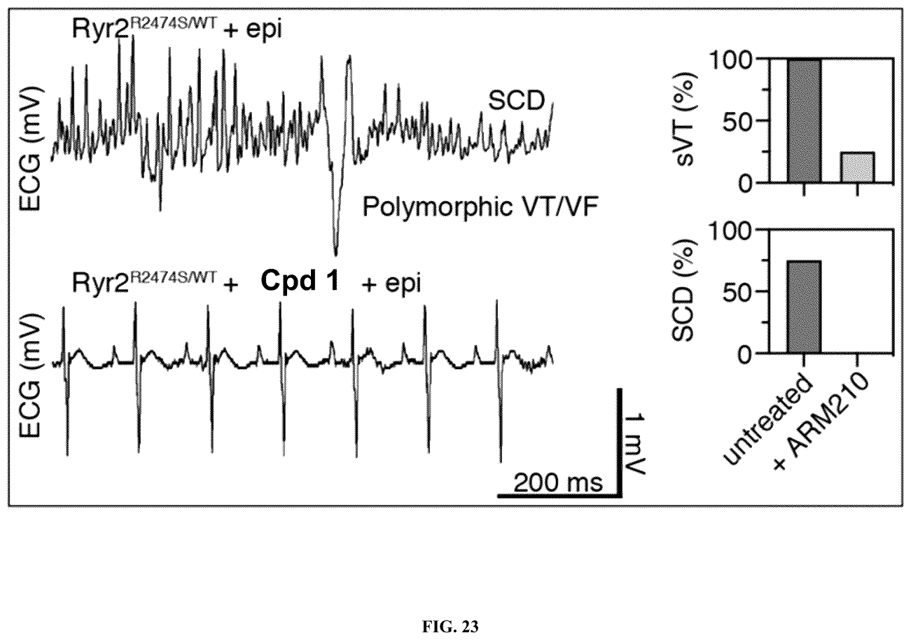

This experiment determined that treatment with Compound 1 reduced the frequency of ventricular tachycardia and prevented sudden cardiac death in Ryr^{2R2474S/WT} mice. FIG. 23 depicts representative telemetric electrocardiogram (ECG) recordings of Ryr^{2R2474S/WT} mice (n=4) during arrhythmia provocation stress testing by epinephrine injection (1 mg/kg epinephrine). Catecholamine injection and exercise resulted in rapid sustained ventricular tachycardia (sVT) and sudden cardiac death (SCD). Compound 1 (50 mg/kg/day in drinking water) treatment prevented sVT and SCD.

### Example 9: SR Ca²⁺ Leak Assay in Ryr2^{R2474S/WT} Mice Heart Microsomes

Ca²⁺ leak was measured in microsomes from heart lysates isolated from control Ryr^{2R2474S/WT} mice, Ryr^{2R2474S/WT} mice treated with epinephrine, and Ryr^{2R2474S/WT} mice treated with epinephrine and Compound 1 in EXAMPLE 8. 10704.1 Cardiac muscle SR microsomes were prepared by homogenizing heart samples on ice using a Teflon glass homogenizer (50 times) with 2 volumes of 20 mM tris-maleate (pH 7.4), 1 mM EDTA, 1 mM DTT, and protease inhibitors (Roche). Homogenate was then spun by centrifuge at 4000 g for 15 min at 4° C., and the resultant supernatant was spun by centrifuge at 50,000 g for 45 min at 4° C. Pellets were resuspended in lysis buffer containing 300 mM sucrose. Microsomes (5 g/ml) were diluted into a buffer (pH 7.2) containing 8 mM K-phosphocreatine, and creatine kinase (2 U/ml), mixed with 3 μM Fluo-4 and added to multiple wells of a 96-well plate. Ca²⁺ loading of the microsomes was initiated by adding 1 mM ATP. After Ca²⁺ uptake (50 s), 3 μM thapsigargin was added to inhibit the Ca²⁺ reuptake by SERCA. SR Ca²⁺ leak was measured by the increase in intensity of the Fluo-4 signal (measured in a Tecan fluorescence plate reader). Ca²⁺ leak was quantified as the difference between the average Fluo-4 signal before and after addition of thapsigargin. Graphs were plotted with GraphPad Prism software.

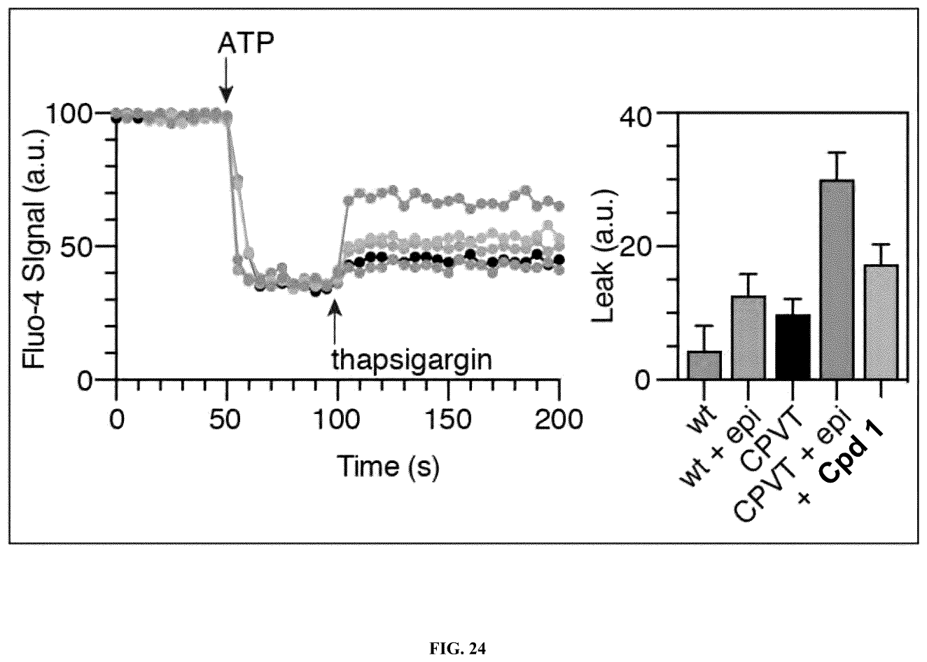

This experiment determined that Compound 1 treatment prevented SR Ca²⁺ leak via RyR2-R2474S channels. FIG. 24 shows SR Ca²⁺ leak measured in microsomes from Ryr^{2R2474S/WT} mouse heart lysates. The Ca²⁺ leak was compared for hearts isolated from control Ryr^{2R2474S/WT} mice (gray), Ryr^{2R2474S/WT} mice treated with epinephrine (magenta), and Ryr^{2R2474S/WT} mice treated with epinephrine and Compound 1 (cyan). Bar graphs represent the quantification of the increase in Fluo-4 signal/s over the first 5 seconds after addition of thapsigargin. N=2 in each group.

### Example 10: Mass Spectrometry Analyses

Mass spectroscopic analysis of the hyperphosphorylated channels prepared in EXAMPLE 5 was conducted to determine phosphorylation sites.

ER vesicles from HEK293 cells expressing RyR2 were separated on 4 to 12% gradient SDS-PAGE and sent for mass spectrometry analysis to in-house Columbia Proteomics Shared Resource (HICCC). Protein gel slices were excised, and in-gel digestion was performed. Digested peptides were collected and further extracted from gel slices in extraction buffer (1:2 ratio by volume of 5% formic acid:acetonitrile) at high speed, shaking in an air thermostat. Peptides were separated within 80 min at a flow rate of 400 nl/min on a reversed-phase C18 column with an integrated CaptiveSpray Emitter (25 cm×75 m, 1.6 m, IonOpticks). Mobile phases A and B were with 0.1% formic acid in water and 0.1% formic acid in acetonitrile. The fraction of B was linearly increased from 2 to 23% within 70 min, followed by an increase to 35% within 10 min and a further increase to 80% before reequilibration. The timsTOF Pro was operated in parallel accumulation-serial fragmentation (PASEF) mode with the following settings: mass range, 100 to 1700 mass/charge ratio (m/z); 1/K0 start, 0.6 V s/cm²; end, 1.6 V s/cm²; ramp time, 100 ms; lock duty cycle to 100%; capillary voltage, 1600 V; dry gas, 3 l/min; and dry temperature, 200° C. PASEF settings: 10 Tandem Mass Spectrometry frames (1.16 s duty cycle); charge range, 0 to 5; active exclusion for 0.4 min; target intensity, 20,000; intensity threshold, 2500; and collision-induced dissociation collision energy, 59 eV. A polygon filter was applied to the m/z and ion mobility plane to select features most likely representing peptide precursors rather than singly charged background ions. Acquired PASEF raw files were analyzed using the MaxQuant environment v.2.0.1.0 and Andromeda for database searches. MaxQuant was configured to search with the reference human proteome database downloaded from UniProt. The following modifications were used for protein identification and quantification: Carbamidomethylation of cysteine residues (+57.021 Da) was set as static modifications, while the oxidation of methionine residues (+15.995 Da), deamidation (+0.984) on asparagine and glutamine, and phosphorylation (+79.966) on serine, threonine, and tyrosine were set as a variable modification. Results obtained from MaxQuant, Phospho (STY) sites table was used for RyR2 phospho-site quantification. Sequence coverage was obtained with the software Scaffold 5.

TABLE 10 and TABLE 11 are mass-spectroscopy tables indicating the phosphopeptides detected before and after PKA phosphorylation of recombinant human RyR2 samples.

TABLE 10

| | | | | | | Score |
|---|---|---|---|---|---|---|
| Position | | | | | | for |
| within | Localization | Score | | | Delta | local- |
| RyR2 | probability | difference | log₁₀PEP | Score | score | ization |
| 16 | 1.00 | 64.13 | −6.34 | 67.66 | 51.88 | 67.66 |
| 1495 | 0.86 | 7.94 | −3.18 | 62.49 | 53.34 | 62.49 |
| 1856 | 0.97 | 18.05 | −14.55 | 64.40 | 52.48 | 64.40 |
| 1863 | 0.54 | 0.89 | −4.61 | 60.59 | 56.99 | 40.37 |
| 1869 | 1.00 | 35.49 | −3.87 | 82.73 | 77.89 | 69.82 |
| 2363 | 0.97 | 17.27 | −4.17 | 93.50 | 53.12 | 93.50 |
| 2368 | 0.60 | 3.79 | −4.66 | 75.69 | 59.00 | 75.69 |
| 2804 | 0.73 | 4.26 | −34.70 | 167.56 | 158.97 | 167.56 |
| 2806 | 1.00 | 28.36 | −80.37 | 285.07 | 257.49 | 157.38 |
| 2810 | 0.67 | 3.11 | −8.92 | 102.55 | 97.74 | 102.55 |
| 2811 | 0.74 | 4.22 | −2.73 | 81.90 | 80.43 | 81.90 |
| 2814 | 0.98 | 18.09 | −2.48 | 100.27 | 94.94 | 100.27 |
| 2822 | 1.00 | 40.37 | −5.53 | 121.74 | 119.20 | 121.74 |

TABLE 11

| Position | Intensity | Intensity | Intensity | Intensity |
|---|---|---|---|---|
| within RyR2 | control 1 | control 2 | control 3 | PKA |
| 16 | 56,978 | — | 63,044 | 70,955 |
| 1495 | 12,130 | — | — | — |
| 1856 | — | — | — | 155,050 |
| 1863 | — | — | 81,407 | — |
| 1869 | 218,050 | 215,750 | 349,580 | 270,440 |
| 2363 | 68,221 | — | 38,508 | 33,431 |
| 2368 | 45,015 | — | — | — |
| 2804 | 530,280 | — | — | — |
| 2808 | 3,944,000 | 3,623,900 | 567,320 | 3,385,300 |
| 2810 | 1,332,700 | — | — | — |
| 2811 | — | — | 80,673 | — |
| 2814 | 29,077 | — | — | 55,669 |
| 2822 | 29,678 | — | 32,822 | 28,207 |

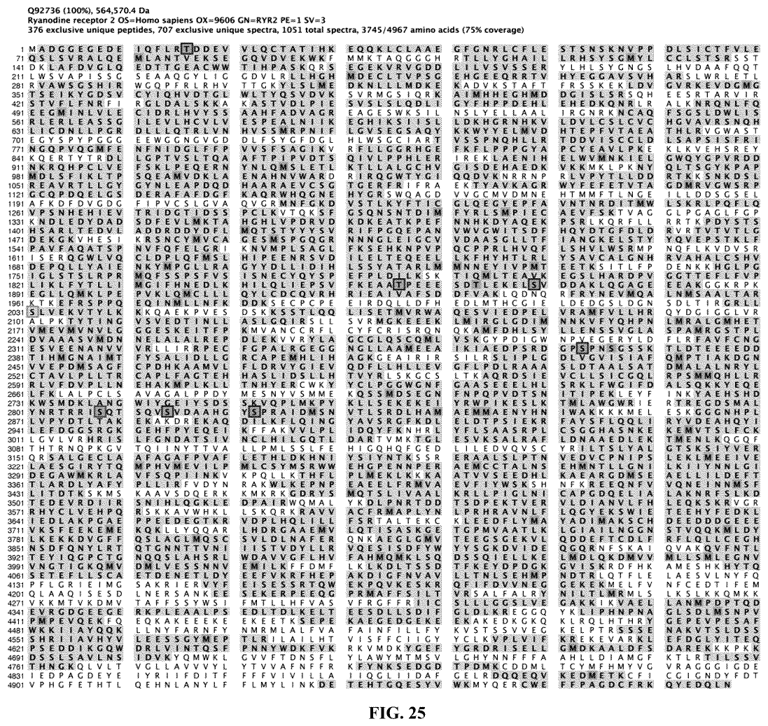

The analysis revealed that RyR2-S2808 is the main detected phosphorylated peptide before and after PKA phosphorylation. Other detected residues, with good localization probability and score difference, were considered secondary phosphorylation sites (TABLE 10 and TABLE 11, underlined values). Mass-spectrometry is a semi-quantitative technique, indicating that intensities can be only compared within the same sample (within the same column). FIG. 25 illustrates sequence coverage of RyR2 provided in this experiment. Sequence coverage of 75% was consistently obtained for all samples. Detected peptides are highlighted in orange. Detected residues with oxidative or phosphorylation modifications are highlighted in green. High-confidence phosphorylated residues are framed in black, RyR2-S2808 is framed in red, and RyR2-S2031 is framed in cyan.

The results showed that RyR2-S2808 was the major and only significantly PKA-phosphorylated site in RyR2 treated with PKA (coverage of 75%; FIG. 25). A secondary site with one order of magnitude lower intensity at RyR2-S1869 and other phosphorylation sites with even lower intensity at RyR2-T16, RyR2-S1856, RyR2-S2363, RyR2-S2814, and RyR2-S2822 (TABLE 10 and TABLE 11) were detected. No phosphorylation of RyR2-S2031 (FIG. 25) was detected.

1.-248. (canceled)

249. A composition comprising a complex suspended in a solid medium comprising vitreous ice, wherein the complex comprises a protein and a synthetic compound, wherein the protein is a ryanodine receptor 2 protein (RyR2) or a mutant thereof.

250. The composition of claim 249, wherein the composition is prepared by a process, the process comprising vitrifying an aqueous solution that is applied to an electron microscopy grid, wherein the aqueous solution comprises the complex.

251. The composition of claim 250, wherein the aqueous solution further comprises one or more or each of a buffering agent, a phospholipid, a zwitterionic surfactant, a disulfide-reducing agent, a protease inhibitor, or a xanthine alkaloid.

252. The composition of claim 250, wherein the aqueous solution further comprises one or more or each of a Ca²⁺ ion, sodium adenosine triphosphate (NaATP), cyclic adenosine monophosphate (cAMP), or calmodulin.

253. The composition of claim 249, wherein the complex further comprises one or more or each of calmodulin, calstabin, a xanthine alkaloid or a Ca²⁺ ion.

254. The composition of claim 249, wherein the protein is wild type RyR2, a mutant RyR2 or a post-translationally modified RyR2 protein, wherein the post-translationally modified RyR2 protein is a phosphorylated, oxidized or nitrosylated RyR2 or is associated with heart failure.

255. The composition of claim 249, wherein the protein is a mutant RyR2 containing at least one mutation that is associated with Catecholaminergic Polymorphic Ventricular Tachycardia (CPVT).

256. The composition of claim 255, wherein the mutation is RyR2-R2474S, RyR2-R420Q, or RyR2-R420W.

257. The composition of claim 249, wherein the protein is a mutant RyR2 or a post-translationally modified RyR2 protein, wherein the mutation or the post-translational modification destabilizes an interaction between NTD and BSol domains of the RyR2 protein; or wherein the mutation or the post-translational modification destabilizes a cytosolic shell of the RyR2 protein, wherein the cytosolic shell comprises NTD, SPRY, JSol and BSol domains of the RyR2 proteins.

258. The composition of claim 249, wherein the protein is a tetramer of RyR2 monomers, wherein each RyR2 monomer is a peptide according to SEQ ID NO: 3 or SEQ ID NO: 4.

259. The composition of claim 249, wherein the complex further comprises a nucleoside-containing molecule.

260. The composition of claim 259, wherein the nucleoside-containing molecule and the synthetic compound bind a RYR domain of the protein, wherein the RYR domain is a RY1&2 domain.

261. The composition of claim 260, wherein the RY1&2 domain has a three-dimensional structure according to TABLE 3.

262. The composition of claim 259, wherein the nucleoside-containing molecule is a purine nucleoside-containing molecule, a nucleotide or nucleoside polyphosphate, or an adenosine triphosphate (ATP) molecule.

263. The composition of claim 262, wherein the nucleoside-containing molecule is an adenosine triphosphate (ATP) molecule, wherein the ATP molecule forms a pi-stacking interaction with the synthetic compound or molecule has a three-dimensional conformation according to TABLE 5.

264. The composition of claim 263, wherein the ATP molecule cooperatively binds the protein with the synthetic compound, or wherein the ATP molecule forms a pi-stacking interaction with the synthetic compound.

265. The composition of claim 249, wherein the complex further comprises a second nucleoside-containing molecule bound to a C-terminal domain of the RyR1 protein, wherein the second nucleoside-containing molecule is a second ATP molecule.

266. The composition of claim 249, wherein the synthetic compound comprises a benzazepane, benzothiazepane, or benzodiazepane moiety.

267. The composition of claim 249, wherein the synthetic compound is a compound of Formula (I): 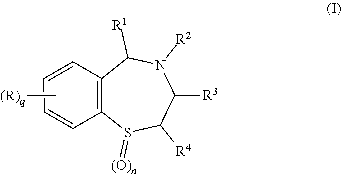
wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, S(═O)alkyl, or OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NHNR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, —NH₂, —NHNH₂, or —NHOH;
each X is halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H;
or R¹⁵ and R¹⁶ together with the N to which R¹⁵ and R¹⁶ are bonded form a heterocycle that is substituted or unsubstituted;
n is 0, 1, or 2;
q is 0, 1, 2, 3, or 4;
t is 1, 2, 3, 4, 5, or 6; and
m is 1, 2, 3, or 4,
or a pharmaceutically-acceptable salt thereof.

268. The composition of claim 249, wherein the synthetic compound is a compound of Formula (I-k): 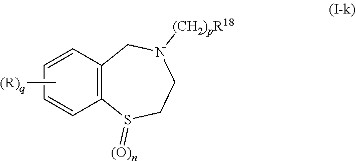
wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, S(═O)alkyl, or OS(═O)₂CF₃;
R¹⁸ is alkyl, aryl, cycloalkyl, or heterocyclyl, each of which is independently substituted or unsubstituted; or —NR¹⁵R¹⁶, —C(═O)NR¹⁵R¹⁶, —(C═O)OR¹⁵, or —OR¹⁵;
q is 0, 1, 2, 3, or 4;
p is 1, 2, 3, 4, 5, 6, 7, 8 9, or 10; and
n is 0, 1, or 2,
or a pharmaceutically-acceptable salt thereof.

269. The composition of claim 249, wherein the synthetic compound is: 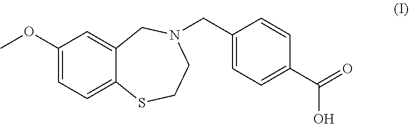 [SMILES: COc1ccc2c(c1)CN(Cc1ccc(C(=O)O)cc1)CCS2]
or an ionized form thereof.

270. The composition of claim 269, wherein the synthetic compound has a three-dimensional conformation according to TABLE 4.

271. A method of determining a binding site of a synthetic compound in a protein, the method comprising subjecting a composition of claim 249 to single-particle cryogenic electron microscopy analysis, wherein the structure of the of protein obtained by single-particle cryogenic electron microscopy analysis has a resolution from about 2 Å to about 3.5.

272. A method for predicting a docked position of a target ligand in a binding site of a biomolecule, the method comprising:
receiving a template ligand-biomolecule structure, the template ligand-biomolecule structure comprising a template ligand docked in the binding site of the biomolecule;
comparing a pharmacophore model of the template ligand to a pharmacophore model of the target ligand;
overlapping the pharmacophore model of the target ligand with the pharmacophore model of the template ligand while the template ligand is in the binding site of the biomolecule; and
predicting the docked position of the target ligand in the binding site of the biomolecule based on a position of the pharmacophore model of the target ligand when overlapped with the pharmacophore model of the template ligand,
wherein the template ligand-biomolecule structure is obtained by a process comprising subjecting a complex of the biomolecule and the template ligand to single-particle cryogenic electron microscopy analysis,
wherein the biomolecule is a ryanodine receptor 2 protein (RyR2) or a mutant thereof and the template ligand is a synthetic compound, and
wherein the complex of the biomolecule and the template ligand is obtained by the process to prepare the composition of claim 250.

273. The method of claim 272, wherein the biomolecule is a RY1&2 domain of RyR2, wherein the RY1&2 domain comprises a structure according to TABLE 3.

274. The method of claim 272, wherein the template ligand has a three-dimensional conformation according to TABLE 4.

275. The method of claim 273, wherein the RY1&2 domain further comprises an ATP molecule having a three-dimensional conformation according to TABLE 5.

276. The method of claim 272, wherein the template ligand is 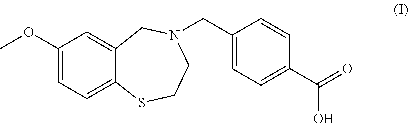 [SMILES: COc1ccc2c(c1)CN(Cc1ccc(C(=O)O)cc1)CCS2]
or an ionized form thereof.

277. A method of identifying a plurality of potential lead compounds, the method comprising the steps of:
(a) analyzing, using a computer system, an initial lead compound known to bind to a biomolecular target, the analyzing comprising partitioning, by providing a database of known reactions, the initial lead compound into atoms defining partitioned lead compound comprising a lead compound core and atoms defining a lead compound non-core, wherein the initial lead compound is partitioned using a computational retrosynthetic analysis of the initial lead compound;
(b) identifying, using the computer system, a plurality of alternative cores to replace the lead compound core in the initial lead compound, thereby generating a plurality of potential lead compounds each having a respective one of the plurality of alternative cores;
(c) calculating, using the computer system, a difference in binding free energy between the partitioned lead compound and each potential lead compound;
(d) predicting, using the computer system, whether each potential lead compound will bind to the biomolecular target and identifying a predicted active set of potential lead compounds based on the prediction;
(e) obtaining a synthesized set of at least some of the potential leads of the predicted active set to establish a first of potential lead compounds; and
(f) determining, empirically, an activity of each of the first set of synthesized potential lead compounds,
wherein the biomolecular target is a ryanodine receptor 2 protein (RyR2) or a mutant thereof and the initial lead compound is a synthetic compound, and
wherein the structure of the biomolecular target used in the predicting of (d) is obtained by a process comprising subjecting a complex of the biomolecular target and the initial lead compound to single particle cryogenic electron microscopy analysis, and
wherein the complex of the biomolecular structure and the initial lead compound is obtained by the process to prepare the composition of claim 250.

278. A method for pharmaceutical drug discovery, comprising:
identifying an initial lead compound for binding to a biomolecular target;
using the method of claim 278 to identify a predicted active set of potential lead compounds for binding to the biomolecular target based on the initial lead compound;
selecting one or more of the predicted active set of potential lead compounds for synthesis; and
assaying the one or more synthesized selected compounds to assess each synthesized selected compounds suitability for in vivo use as a pharmaceutical compound,
wherein the biomolecular target is a RY1&2 domain of RyR2, and the structure of the biomolecular target used in the predicting of (d) is obtained by a process comprising subjecting a complex of the biomolecular target and the initial lead compound to single-particle cryogenic electron microscopy analysis.

279. A computer-implemented method of quantifying binding affinity between a ligand and a receptor molecule, the method comprising:
receiving by one or more computers, data representing a ligand molecule,
receiving by one or more computers, data representing a receptor molecule domain,
using the data representing the ligand molecule and the data representing the receptor molecule domain in computer analysis to identify ring structure within the ligand, the ring structure being an entire ring or a fused ring;
using the data representative of the identified ligand ring structure to designate a first ring face and a second ring face opposite to the first ring face, and classifying the ring structure by:
a) determining proximity of receptor atoms to atoms on the first face of the ligand ring; and
b) determining proximity of receptor atoms to atoms on the second face of the ligand ring; and
c) determining solvation of the first face of the ligand ring and solvation of the second face of the ligand ring;
classifying the identified ligand ring structure as buried, solvent exposed or having a single face exposed to solvent based on receptor atom proximity to and solvation of the first ring face and receptor atom proximity to and solvation of the second ring face;
quantifying the binding affinity between the ligand and the receptor molecule domain based at least in part on the classification of the ring structure; and
displaying, via computer, information related to the classification of the ring structure,
wherein the receptor molecule domain is a RY1&2 domain of RyR2 protein or a mutant thereof, wherein the data representing a ligand molecule and the data representing a receptor molecule domain are obtained by a process comprising subjecting a complex comprising the ligand molecule and the receptor molecule domain to single-particle cryogenic electron microscopy analysis, and
wherein the ligand molecule is a synthetic compound, and wherein the complex is obtained by the process to prepare the composition of claim 250.

280. A method of identifying a compound having RyR2 modulatory activity, the method comprising:
(a) determining an open probability (Pₒ) of a RyR2 protein;
(b) contacting the RyR2 protein with a test compound;
(c) determining an open probability (Pₒ) of the RyR2 protein in the presence of the test compound; and
(d) determining a difference between the Pₒ of the RyR2 protein in the presence and absence of the test compound;
wherein a reduction in the Pₒ of the RyR2 protein in the presence of the test compound relative to the Pₒ of the RyR2 protein in the absence of the test compound is indicative of the compound having RyR2 modulatory activity.

281. The method of claim 280, wherein the RyR2 protein is a mutated or a post-translationally modified RyR2 protein, wherein the test compound preferentially binds to the mutated or post-translationally modified RyR2 relative to wild-type RyR2.

282. A method for identifying a compound having RyR2 modulatory activity, comprising:
(a) contacting a RyR2 protein with a ligand having known RyR2 modulatory activity to create a mixture, wherein the RyR2 protein is a leaky RyR2, the leaky RyR2 comprising mutant RyR2 protein, post-translationally modified RyR2, or a combination thereof,
(b) contacting the mixture of step (a) with a test compound; and
(c) determining the ability of the test compound to displace the ligand from the RyR2 protein.

283. The method of claim 282, wherein the ligand is radiolabeled and generates a signal, wherein determining the ability of the test compound to displace the ligand from the RyR2 protein comprises determining a change in the signal.

284. The method of claim 282, wherein the RyR2 protein is a mutant RyR2, wherein the mutant RyR2 contains at least one mutation that is associated with Catecholaminergic Polymorphic Ventricular Tachycardia (CPVT).


SEQUENCE LISTING (4 sequences)

SEQ ID NO: 1 - protein, 149 aa, Homo sapiens [sequence omitted]

SEQ ID NO: 2 - protein, 108 aa, Homo sapiens
MGVEIETISPGDGRTFPKKGQTCVVHYTGMLQNGKKFDSSRDRNKPFKFRIGKQEVIKGFEEGAAQMSLGQRAKLTCTPDVAYGATGHPGVIPPNATLIFDVELLNLE

SEQ ID NO: 3 - protein, 4967 aa, Homo sapiens
MADGGEGEDEIQFLRTDDEVVLQCTATIHKEQQKLCLAAEGFGNRLCFLESTSNSKNVPPDLSICTFVLEQSLSVRALQEMLANTVEKSEGQVDVEKWKFMMKTAQGGGHRTLLYGHAILLRHSYSGMYLCCLSTSRSSTDKLAFDVGLQEDTTGEACWWTIHPASKQRSEGEKVRVGDDLILVSVSSERYLHLSYGNGSLHVDAAFQQTLWSVAPISSGSEAAQGYLIGGDVLRLLHGHMDECLTVPSGEHGEEQRRTVHYEGGAVSVHARSLWRLETLRVAWSGSHIRWGQPFRLRHVTTGKYLSLMEDKNLLLMDKEKADVKSTAFTFRSSKEKLDVGVRKEVDGMGTSEIKYGDSVCYIQHVDTGLWLTYQSVDVKSVRMGSIQRKAIMHHEGHMDDGISLSRSQHEESRTARVIRSTVFLFNRFIRGLDALSKKAKASTVDLPIESVSLSLQDLIGYFHPPDEHLEHEDKQNRLRALKNRQNLFQEEGMINLVLECIDRLHVYSSAAHFADVAGREAGESWKSILNSLYELLAALIRGNRKNCAQFSGSLDWLISRLERLEASSGILEVLHCVLVESPEALNIIKEGHIKSIISLLDKHGRNHKVLDVLCSLCVCHGVAVRSNQHLICDNLLPGRDLLLQTRLVNHVSSMRPNIFLGVSEGSAQYKKWYYELMVDHTEPFVTAEATHLRVGWASTEGYSPYPGGGEEWGGNGVGDDLFSYGFDGLHLWSGCIARTVSSPNQHLLRTDDVISCCLDLSAPSISFRINGQPVQGMFENFNIDGLFFPVVSFSAGIKVRFLLGGRHGEFKFLPPPGYAPCYEAVLPKEKLKVEHSREYKQERTYTRDLLGPTVSLTQAAFTPIPVDTSQIVLPPHLERIREKLAENIHELWVMNKIELGWQYGPVRDDNKRQHPCLVEFSKLPEQERNYNLQMSLETLKTLLALGCHVGISDEHAEDKVKKMKLPKNYQLTSGYKPAPMDLSFIKLTPSQEAMVDKLAENAHNVWARDRIRQGWTYGIQQDVKNRRNPRLVPYTLLDDRTKKSNKDSLREAVRTLLGYGYNLEAPDQDHAARAEVCSGTGERFRIFRAEKTYAVKAGRWYFEFETVTAGDMRVGWSRPGCQPDQELGSDERAFAFDGFKAQRWHQGNEHYGRSWQAGDVVGCMVDMNEHTMMFTLNGEILLDDSGSELAFKDFDVGDGFIPVCSLGVAQVGRMNFGKDVSTLKYFTICGLQEGYEPFAVNTNRDITMWLSKRLPQFLQVPSNHEHIEVTRIDGTIDSSPCLKVTQKSFGSQNSNTDIMFYRLSMPIECAEVFSKTVAGGLPGAGLFGPKNDLEDYDADSDFEVLMKTAHGHLVPDRVDKDKEATKPEFNNHKDYAQEKPSRLKQRFLLRRTKPDYSTSHSARLTEDVLADDRDDYDFLMQTSTYYYSVRIFPGQEPANVWVGWITSDFHQYDTGFDLDRVRTVTVTLGDEKGKVHESIKRSNCYMVCAGESMSPGQGRNNNGLEIGCVVDAASGLLTFIANGKELSTYYQVEPSTKLFPAVFAQATSPNVFQFELGRIKNVMPLSAGLFKSEHKNPVPQCPPRLHVQFLSHVLWSRMPNQFLKVDVSRISERQGWLVQCLDPLQFMSLHIPEENRSVDILELTEQEELLKFHYHTLRLYSAVCALGNHRVAHALCSHVDEPQLLYAIENKYMPGLLRAGYYDLLIDIHLSSYATARLMMNNEYIVPMTEETKSITLFPDENKKHGLPGIGLSTSLRPRMQFSSPSFVSISNECYQYSPEFPLDILKSKTIQMLTEAVKEGSLHARDPVGGTTEFLFVPLIKLFYTLLIMGIFHNEDLKHILQLIEPSVFKEAATPEEESDTLEKELSVDDAKLQGAGEEEAKGGKRPKEGLLQMKLPEPVKLQMCLLLQYLCDCQVRHRIEAIVAFSDDFVAKLQDNQRFRYNEVMQALNMSAALTARKTKEFRSPPQEQINMLLNFKDDKSECPCPEEIRDQLLDFHEDLMTHCGIELDEDGSLDGNSDLTIRGRLLSLVEKVTYLKKKQAEKPVESDSKKSSTLQQLISETMVRWAQESVIEDPELVRAMFVLLHRQYDGIGGLVRALPKTYTINGVSVEDTINLLASLGQIRSLLSVRMGKEEEKLMIRGLGDIMNNKVFYQHPNLMRALGMHETVMEVMVNVLGGGESKEITFPKMVANCCRFLCYFCRISRQNQKAMFDHLSYLLENSSVGLASPAMRGSTPLDVAAASVMDNNELALALREPDLEKVVRYLAGCGLQSCQMLVSKGYPDIGWNPVEGERYLDFLRFAVFCNGESVEENANVVVRLLIRRPECFGPALRGEGGNGLLAAMEEAIKIAEDPSRDGPSPNSGSSKTLDTEEEEDDTIHMGNAIMTFYSALIDLLGRCAPEMHLIHAGKGEAIRIRSILRSLIPLGDLVGVISIAFQMPTIAKDGNVVEPDMSAGFCPDHKAAMVLFLDRVYGIEVQDFLLHLLEVGFLPDLRAAASLDTAALSATDMALALNRYLCTAVLPLLTRCAPLFAGTEHHASLIDSLLHTVYRLSKGCSLTKAQRDSIEVCLLSICGQLRPSMMQHLLRRLVFDVPLLNEHAKMPLKLLTNHYERCWKYYCLPGGWGNFGAASEEELHLSRKLFWGIFDALSQKKYEQELFKLALPCLSAVAGALPPDYMESNYVSMMEKQSSMDSEGNFNPQPVDTSNITIPEKLEYFINKYAEHSHDKWSMDKLANGWIYGEIYSDSSKVQPLMKPYKLLSEKEKEIYRWPIKESLKTMLAWGWRIERTREGDSMALYNRTRRISQTSQVSVDAAHGYSPRAIDMSNVTLSRDLHAMAEMMAENYHNIWAKKKKMELESKGGGNHPLLVPYDTLTAKEKAKDREKAQDILKFLQINGYAVSRGFKDLELDTPSIEKRFAYSFLQQLIRYVDEAHQYILEFDGGSRGKGEHFPYEQEIKFFAKVVLPLIDQYFKNHRLYFLSAASRPLCSGGHASNKEKEMVTSLFCKLGVLVRHRISLFGNDATSIVNCLHILGQTLDARTVMKTGLESVKSALRAFLDNAAEDLEKTMENLKQGQFTHTRNQPKGVTQIINYTTVALLPMLSSLFEHIGQHQFGEDLILEDVQVSCYRILTSLYALGTSKSIYVERQRSALGECLAAFAGAFPVAFLETHLDKHNIYSIYNTKSSRERAALSLPTNVEDVCPNIPSLEKLMEEIVELAESGIRYTQMPHVMEVILPMLCSYMSRWWEHGPENNPERAEMCCTALNSEHMNTLLGNILKIIYNNLGIDEGAWMKRLAVFSQPIINKVKPQLLKTHFLPLMEKLKKKAATVVSEEDHLKAEARGDMSEAELLILDEFTTLARDLYAFYPLLIRFVDYNRAKWLKEPNPEAEELFRMVAEVFIYWSKSHNFKREEQNFVVQNEINNMSFLITDTKSKMSKAAVSDQERKKMKRKGDRYSMQTSLIVAALKRLLPIGLNICAPGDQELIALAKNRFSLKDTEDEVRDIIRSNIHLQGKLEDPAIRWQMALYKDLPNRTDDTSDPEKTVERVLDIANVLFHLEQKSKRVGRRHYCLVEHPQRSKKAVWHKLLSKQRKRAVVACFRMAPLYNLPRHRAVNLFLQGYEKSWIETEEHYFEDKLIEDLAKPGAEPPEEDEGTKRVDPLHQLILLFSRTALTEKCKLEEDFLYMAYADIMAKSCHDEEDDDGEEEVKSFEEKEMEKQKLLYQQARLHDRGAAEMVLQTISASKGETGPMVAATLKLGIAILNGGNSTVQQKMLDYLKEKKDVGFFQSLAGLMQSCSVLDLNAFERQNKAEGLGMVTEEGSGEKVLQDDEFTCDLFRFLQLLCEGHNSDFQNYLRTQTGNNTTVNIIISTVDYLLRVQESISDFYWYYSGKDVIDEQGQRNFSKAIQVAKQVFNTLTEYIQGPCTGNQQSLAHSRLWDAVVGFLHVFAHMQMKLSQDSSQIELLKELMDLQKDMVVMLLSMLEGNVVNGTIGKQMVDMLVESSNNVEMILKFFDMFLKLKDLTSSDTFKEYDPDGKGVISKRDFHKAMESHKHYTQSETEFLLSCAETDENETLDYEEFVKRFHEPAKDIGFNVAVLLTNLSEHMPNDTRLQTFLELAESVLNYFQPFLGRIEIMGSAKRIERVYFEISESSRTQWEKPQVKESKRQFIFDVVNEGGEKEKMELFVNFCEDTIFEMQLAAQISESDLNERSANKEESEKERPEEQGPRMAFFSILTVRSALFALRYNILTLMRMLSLKSLKKQMKKVKKMTVKDMVTAFFSSYWSIFMTLLHFVASVFRGFFRIICSLLLGGSLVEGAKKIKVAELLANMPDPTQDEVRGDGEEGERKPLEAALPSEDLTDLKELTEESDLLSDIFGLDLKREGGQYKLIPHNPNAGLSDLMSNPVPMPEVQEKFQEQKAKEEEKEEKEETKSEPEKAEGEDGEKEEKAKEDKGKQKLRQLHTHRYGEPEVPESAFWKKIIAYQQKLLNYFARNFYNMRMLALFVAFAINFILLFYKVSTSSVVEGKELPTRSSSENAKVTSLDSSSHRIIAVHYVLEESSGYMEPTLRILAILHTVISFFCIIGYYCLKVPLVIFKREKEVARKLEFDGLYITEQPSEDDIKGQWDRLVINTQSFPNNYWDKFVKRKVMDKYGEFYGRDRISELLGMDKAALDFSDAREKKKPKKDSSLSAVLNSIDVKYQMWKLGVVFTDNSFLYLAWYMTMSVLGHYNNFFFAAHLLDIAMGFKTLRTILSSVTHNGKQLVLTVGLLAVVVYLYTVVAFNFFRKFYNKSEDGDTPDMKCDDMLTCYMFHMYVGVRAGGGIGDEIEDPAGDEYEIYRIIFDITFFFFVIVILLAIIQGLIIDAFGELRDQQEQVKEDMETKCFICGIGNDYFDTVPHGFETHTLQEHNLANYLFFLMYLINKDETEHTGQESYVWKMYQERCWEFFPAGDCFRKQYEDQLN

SEQ ID NO: 4 - protein, 4967 aa, Homo sapiens
MADGGEGEDEIQFLRTDDEVVLQCTATIHKEQQKLCLAAEGFGNRLCFLESTSNSKNVPPDLSICTFVLEQSLSVRALQEMLANTVEKSEGQVDVEKWKFMMKTAQGGGHRTLLYGHAILLRHSYSGMYLCCLSTSRSSTDKLAFDVGLQEDTTGEACWWTIHPASKQRSEGEKVRVGDDLILVSVSSERYLHLSYGNGSLHVDAAFQQTLWSVAPISSGSEAAQGYLIGGDVLRLLHGHMDECLTVPSGEHGEEQRRTVHYEGGAVSVHARSLWRLETLRVAWSGSHIRWGQPFRLRHVTTGKYLSLMEDKNLLLMDKEKADVKSTAFTFRSSKEKLDVGVRKEVDGMGTSEIKYGDSVCYIQHVDTGLWLTYQSVDVKSVRMGSIQRKAIMHHEGHMDDGISLSRSQHEESRTARVIRSTVFLFNRFIRGLDALSKKAKASTVDLPIESVSLSLQDLIGYFHPPDEHLEHEDKQNRLRALKNRQNLFQEEGMINLVLECIDRLHVYSSAAHFADVAGREAGESWKSILNSLYELLAALIRGNRKNCAQFSGSLDWLISRLERLEASSGILEVLHCVLVESPEALNIIKEGHIKSIISLLDKHGRNHKVLDVLCSLCVCHGVAVRSNQHLICDNLLPGRDLLLQTRLVNHVSSMRPNIFLGVSEGSAQYKKWYYELMVDHTEPFVTAEATHLRVGWASTEGYSPYPGGGEEWGGNGVGDDLFSYGFDGLHLWSGCIARTVSSPNQHLLRTDDVISCCLDLSAPSISFRINGQPVQGMFENFNIDGLFFPVVSFSAGIKVRFLLGGRHGEFKFLPPPGYAPCYEAVLPKEKLKVEHSREYKQERTYTRDLLGPTVSLTQAAFTPIPVDTSQIVLPPHLERIREKLAENIHELWVMNKIELGWQYGPVRDDNKRQHPCLVEFSKLPEQERNYNLQMSLETLKTLLALGCHVGISDEHAEDKVKKMKLPKNYQLTSGYKPAPMDLSFIKLTPSQEAMVDKLAENAHNVWARDRIRQGWTYGIQQDVKNRRNPRLVPYTLLDDRTKKSNKDSLREAVRTLLGYGYNLEAPDQDHAARAEVCSGTGERFRIFRAEKTYAVKAGRWYFEFETVTAGDMRVGWSRPGCQPDQELGSDERAFAFDGFKAQRWHQGNEHYGRSWQAGDVVGCMVDMNEHTMMFTLNGEILLDDSGSELAFKDFDVGDGFIPVCSLGVAQVGRMNFGKDVSTLKYFTICGLQEGYEPFAVNTNRDITMWLSKRLPQFLQVPSNHEHIEVTRIDGTIDSSPCLKVTQKSFGSQNSNTDIMFYRLSMPIECAEVFSKTVAGGLPGAGLFGPKNDLEDYDADSDFEVLMKTAHGHLVPDRVDKDKEATKPEFNNHKDYAQEKPSRLKQRFLLRRTKPDYSTSHSARLTEDVLADDRDDYDFLMQTSTYYYSVRIFPGQEPANVWVGWITSDFHQYDTGFDLDRVRTVTVTLGDEKGKVHESIKRSNCYMVCAGESMSPGQGRNNNGLEIGCVVDAASGLLTFIANGKELSTYYQVEPSTKLFPAVFAQATSPNVFQFELGRIKNVMPLSAGLFKSEHKNPVPQCPPRLHVQFLSHVLWSRMPNQFLKVDVSRISERQGWLVQCLDPLQFMSLHIPEENRSVDILELTEQEELLKFHYHTLRLYSAVCALGNHRVAHALCSHVDEPQLLYAIENKYMPGLLRAGYYDLLIDIHLSSYATARLMMNNEYIVPMTEETKSITLFPDENKKHGLPGIGLSTSLRPRMQFSSPSFVSISNECYQYSPEFPLDILKSKTIQMLTEAVKEGSLHARDPVGGTTEFLFVPLIKLFYTLLIMGIFHNEDLKHILQLIEPSVFKEAATPEEESDTLEKELSVDDAKLQGAGEEEAKGGKRPKEGLLQMKLPEPVKLQMCLLLQYLCDCQVRHRIEAIVAFSDDFVAKLQDNQRFRYNEVMQALNMSAALTARKTKEFRSPPQEQINMLLNFKDDKSECPCPEEIRDQLLDFHEDLMTHCGIELDEDGSLDGNSDLTIRGRLLSLVEKVTYLKKKQAEKPVESDSKKSSTLQQLISETMVRWAQESVIEDPELVRAMFVLLHRQYDGIGGLVRALPKTYTINGVSVEDTINLLASLGQIRSLLSVRMGKEEEKLMIRGLGDIMNNKVFYQHPNLMRALGMHETVMEVMVNVLGGGESKEITFPKMVANCCRFLCYFCRISRQNQKAMFDHLSYLLENSSVGLASPAMRGSTPLDVAAASVMDNNELALALREPDLEKVVRYLAGCGLQSCQMLVSKGYPDIGWNPVEGERYLDFLRFAVFCNGESVEENANVVVRLLIRRPECFGPALRGEGGNGLLAAMEEAIKIAEDPSRDGPSPNSGSSKTLDTEEEEDDTIHMGNAIMTFYSALIDLLGRCAPEMHLIHAGKGEAIRIRSILRSLIPLGDLVGVISIAFQMPTIAKDGNVVEPDMSAGFCPDHKAAMVLFLDSVYGIEVQDFLLHLLEVGFLPDLRAAASLDTAALSATDMALALNRYLCTAVLPLLTRCAPLFAGTEHHASLIDSLLHTVYRLSKGCSLTKAQRDSIEVCLLSICGQLRPSMMQHLLRRLVFDVPLLNEHAKMPLKLLTNHYERCWKYYCLPGGWGNFGAASEEELHLSRKLFWGIFDALSQKKYEQELFKLALPCLSAVAGALPPDYMESNYVSMMEKQSSMDSEGNFNPQPVDTSNITIPEKLEYFINKYAEHSHDKWSMDKLANGWIYGEIYSDSSKVQPLMKPYKLLSEKEKEIYRWPIKESLKTMLAWGWRIERTREGDSMALYNRTRRISQTSQVSVDAAHGYSPRAIDMSNVTLSRDLHAMAEMMAENYHNIWAKKKKMELESKGGGNHPLLVPYDTLTAKEKAKDREKAQDILKFLQINGYAVSRGFKDLELDTPSIEKRFAYSFLQQLIRYVDEAHQYILEFDGGSRGKGEHFPYEQEIKFFAKVVLPLIDQYFKNHRLYFLSAASRPLCSGGHASNKEKEMVTSLFCKLGVLVRHRISLFGNDATSIVNCLHILGQTLDARTVMKTGLESVKSALRAFLDNAAEDLEKTMENLKQGQFTHTRNQPKGVTQIINYTTVALLPMLSSLFEHIGQHQFGEDLILEDVQVSCYRILTSLYALGTSKSIYVERQRSALGECLAAFAGAFPVAFLETHLDKHNIYSIYNTKSSRERAALSLPTNVEDVCPNIPSLEKLMEEIVELAESGIRYTQMPHVMEVILPMLCSYMSRWWEHGPENNPERAEMCCTALNSEHMNTLLGNILKIIYNNLGIDEGAWMKRLAVFSQPIINKVKPQLLKTHFLPLMEKLKKKAATVVSEEDHLKAEARGDMSEAELLILDEFTTLARDLYAFYPLLIRFVDYNRAKWLKEPNPEAEELFRMVAEVFIYWSKSHNFKREEQNFVVQNEINNMSFLITDTKSKMSKAAVSDQERKKMKRKGDRYSMQTSLIVAALKRLLPIGLNICAPGDQELIALAKNRFSLKDTEDEVRDIIRSNIHLQGKLEDPAIRWQMALYKDLPNRTDDTSDPEKTVERVLDIANVLFHLEQKSKRVGRRHYCLVEHPQRSKKAVWHKLLSKQRKRAVVACFRMAPLYNLPRHRAVNLFLQGYEKSWIETEEHYFEDKLIEDLAKPGAEPPEEDEGTKRVDPLHQLILLFSRTALTEKCKLEEDFLYMAYADIMAKSCHDEEDDDGEEEVKSFEEKEMEKQKLLYQQARLHDRGAAEMVLQTISASKGETGPMVAATLKLGIAILNGGNSTVQQKMLDYLKEKKDVGFFQSLAGLMQSCSVLDLNAFERQNKAEGLGMVTEEGSGEKVLQDDEFTCDLFRFLQLLCEGHNSDFQNYLRTQTGNNTTVNIIISTVDYLLRVQESISDFYWYYSGKDVIDEQGQRNFSKAIQVAKQVFNTLTEYIQGPCTGNQQSLAHSRLWDAVVGFLHVFAHMQMKLSQDSSQIELLKELMDLQKDMVVMLLSMLEGNVVNGTIGKQMVDMLVESSNNVEMILKFFDMFLKLKDLTSSDTFKEYDPDGKGVISKRDFHKAMESHKHYTQSETEFLLSCAETDENETLDYEEFVKRFHEPAKDIGFNVAVLLTNLSEHMPNDTRLQTFLELAESVLNYFQPFLGRIEIMGSAKRIERVYFEISESSRTQWEKPQVKESKRQFIFDVVNEGGEKEKMELFVNFCEDTIFEMQLAAQISESDLNERSANKEESEKERPEEQGPRMAFFSILTVRSALFALRYNILTLMRMLSLKSLKKQMKKVKKMTVKDMVTAFFSSYWSIFMTLLHFVASVFRGFFRIICSLLLGGSLVEGAKKIKVAELLANMPDPTQDEVRGDGEEGERKPLEAALPSEDLTDLKELTEESDLLSDIFGLDLKREGGQYKLIPHNPNAGLSDLMSNPVPMPEVQEKFQEQKAKEEEKEEKEETKSEPEKAEGEDGEKEEKAKEDKGKQKLRQLHTHRYGEPEVPESAFWKKIIAYQQKLLNYFARNFYNMRMLALFVAFAINFILLFYKVSTSSVVEGKELPTRSSSENAKVTSLDSSSHRIIAVHYVLEESSGYMEPTLRILAILHTVISFFCIIGYYCLKVPLVIFKREKEVARKLEFDGLYITEQPSEDDIKGQWDRLVINTQSFPNNYWDKFVKRKVMDKYGEFYGRDRISELLGMDKAALDFSDAREKKKPKKDSSLSAVLNSIDVKYQMWKLGVVFTDNSFLYLAWYMTMSVLGHYNNFFFAAHLLDIAMGFKTLRTILSSVTHNGKQLVLTVGLLAVVVYLYTVVAFNFFRKFYNKSEDGDTPDMKCDDMLTCYMFHMYVGVRAGGGIGDEIEDPAGDEYEIYRIIFDITFFFFVIVILLAIIQGLIIDAFGELRDQQEQVKEDMETKCFICGIGNDYFDTVPHGFETHTLQEHNLANYLFFLMYLINKDETEHTGQESYVWKMYQERCWEFFPAGDCFRKQYEDQLN